SEQUENCE LISTING (143 sequences)

SEQ ID NO: 1 - protein, 1368 aa, Streptococcus pyogenes [sequence omitted]

SEQ ID NO: 2 - protein, 7 aa, unidentified
PKKKRKV

SEQ ID NO: 3 - protein, 7 aa, unidentified
PKKKRRV

SEQ ID NO: 4 - protein, 16 aa, synthetic construct
  REGION 1..16: note = synthetic construct
KRPAATKKAGQAKKKK

SEQ ID NO: 5 - protein, 20 aa, unidentified
  REGION 1..20: note = misc_feature - tat protein
GRKKRRQRRRPPQPKKKRKV

SEQ ID NO: 6 - protein, 19 aa, Hepatitis B virus
  REGION 1..19: note = misc_feature - middle S protein, partial
PLSSIFSRIGDPPKKKRKV

SEQ ID NO: 7 - protein, 24 aa, synthetic construct
  REGION 1..24: note = synthetic construct
GALFLGWLGAAGSTMGAPKKKRKV

SEQ ID NO: 8 - protein, 27 aa, synthetic construct
  REGION 1..27: note = synthetic construct
GALFLGFLGAAGSTMGAWSQPKKKRKV

SEQ ID NO: 9 - protein, 21 aa, synthetic construct
  REGION 1..21: note = synthetic construct
KETWWETWWTEWSQPKKKRKV

SEQ ID NO: 10 - DNA, 390 bp, synthetic construct
  misc_feature 1..390: note = synthetic construct
  source 1..390: mol_type = other DNA
agaatttaactgtggtcacatttgctttatcgactggcttcatctcacagctcatcttacgcaagttcgatgagtatgccagtcactttcaatttggttgaatgttcccgtgacatgcgagttctgtcgaccatgtgccgcggattgaattcctcaagggtggtgatagatgctacggtggtgatgcgcatgcgctcagtcctcatctccctcaagcaggccccgctggtgggtcggagtccctagtgaagccaccaatatagtggtcgtgtcaagcaactgtccacgctccaccctcgaggtcgtaacataaacgtactaaggcacgagtaaacaagatcgatagcaagaacatggtatagactgacggagagctcgccattagtctga

SEQ ID NO: 11 - DNA, 390 bp, synthetic construct
  misc_feature 1..390: note = synthetic construct
  source 1..390: mol_type = other DNA
agaatttaactgtggtcacatttgctttatcgactggcttcatctcacagctcatcttacgcaagttcgatgagtatgccagtcactttcaatttggttgaatgttcccgtgacatgcgagttctgtcgaccatgtgccgcggattgaattcctcaagggtggtgatagatgctacggtggtgatgcgtatgcactcagtcctcaactccctcaagcaggcgacccctgggggtcggagtccctagtgaagccaccaatatagtggtcgtgtcaagcaactgtccacgctccaccctcgaggtcgtaacataaacgtactaaggcacgagtaaacaagatcgatagcaagaacatggtatagactgacggagagctcgccattagtctga

SEQ ID NO: 12 - DNA, 390 bp, synthetic construct
  misc_feature 1..390: note = synthetic construct
  source 1..390: mol_type = other DNA
agaatttaactgtggtcacatttgctttatcgactggcttcatctcacagctcatcttacgcaagttcgatgagtatgccagtcactttcaatttggttgaatgttcccgtgacatgcgagttctgtcgaccatgtgccgcggattgaattcctcaagggtggtgatagatgctacggtggtgatgcaataaatttcagccctcatttccctcaagcaggggttactttagggtcggagtccctagtgaagccaccaatatagtggtcgtgtcaagcaactgtccacgctccaccctcgaggtcgtaacataaacgtactaaggcacgagtaaacaagatcgatagcaagaacatggtatagactgacggagagctcgccattagtctga

SEQ ID NO: 13 - DNA, 390 bp, synthetic construct
  misc_feature 1..390: note = synthetic construct
  source 1..390: mol_type = other DNA
agaatttaactgtggtcacatttgctttatcgactggcttcatctcacagctcatcttacgcaagttcgatgagtatgccagtcactttcaatttggttgaatgttcccgtgacatgcgagttctgtcgaccatgtgccgcggattgaattcctcaagggtggtgatagatgctacggtggtgatgctctccagcccactcctcatccccctcaagccggtcccaggctggggtcggagtccctagtgaagccaccaatatagtggtcgtgtcaagcaactgtccacgctccaccctcgaggtcgtaacataaacgtactaaggcacgagtaaacaagatcgatagcaagaacatggtatagactgacggagagctcgccattagtctga

SEQ ID NO: 14 - DNA, 2838 bp, synthetic construct
  misc_feature 1..2838: note = synthetic construct
  source 1..2838: mol_type = other DNA
gcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgatcgatgcggcctcgcaggccaaagatgtctcccgcatgcgctcagtcctcatctccctcaagcaggccctgctggtgcactgaagagccaccctgtgcgcgtgatatgcagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagc

SEQ ID NO: 15 - DNA, 2838 bp, synthetic construct
  misc_feature 1..2838: note = synthetic construct
  source 1..2838: mol_type = other DNA
gcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgatcgatgcggcctcgcagggcaaagaggtctcctgtatgcactcagtcctcaactccctcaagcaggcgacccttggtgcactgacaaaccgctcctgcgcgtgatatgcagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagc

SEQ ID NO: 16 - DNA, 2838 bp, synthetic construct
  misc_feature 1..2838: note = synthetic construct
  source 1..2838: mol_type = other DNA
gcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgatcgatgcggcctcaggagagggagccatgctcatctccagcccactcctcatccccctcaagccggtcccaggctgagaggctaaagcttgtctttgcgcgtgatatgcagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagc

SEQ ID NO: 17 - DNA, 2995 bp, synthetic construct
  misc_feature 1..2995: note = synthetic construct
  source 1..2995: mol_type = other DNA
gccgggcaagagcaactcggtcgccgcatacactattctcagaatgacttggttgagtactcaccagtcacagaaaagcatcttacggatggcatgacagtaagagaattatgcagtgctgccataaccatgagtgataacactgcggccaacttacttctgacaacgatcggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaacgttgcgcaaactattaactggcgaactacttactctagcttcccggcaacaattaatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaactgtcagaccaagtttactcatatatactttagattgatttaaaacttcatttttaatttaaaaggatctaggtgaagatcctttttgataatctcatgaccaaaatcccttaacgtgagttttcgttccactgagcgtcagaccccgtagaaaagatcaaaggatcttcttgagatcctttttttctgcgcgtaatctgctgcttgcaaacaaaaaaaccaccgctaccagcggtggtttgtttgccggatcaagagctaccaactctttttccgaaggtaactggcttcagcagagcgcagataccaaatactgttcttctagtgtagccgtagttaggccaccacttcaagaactctgtagcaccgccacatacctcgctctgctaatcctgttaccagtggctgcttgccagtggcgataagtcgtgtcttaccgggttggactcaagacgatagttaccggataaggcgcagcggtcgggctgaacggggggttcgtgcacacagcccagcttggagcgaacgacctacaccgaactgagatacctacagcgtgagctatgagaaagcgccacgcttcccgaagggagaaaggcggacaggtatccggtaagcggcagggtcggaacaggagagcgcacgagggagcttccagggggaaacgcctggtatctttatagtcctgtcgggtttcgccacctctgacttgagcgtcgatttttgtgatgctcgtcaggggggcggagcctatggaaaaacgccagcaacgcggcctttttacggttcctggccttttgctggccttttgctcacatgttctttcctgcgttatcccctgattctgtggataaccgtattaccgcctttgagtgagctgataccgctcgccgcagccgaacgaccgagcgcagcgagtcagtgagcgaggaagcggaagagcgcccaatacgcaaaccgcctctccccgcgcgttggccgattcattaatgcagctggcacgacaggtttcccgactggaaagcgggcagtgagcgcaacgcaattaatgtgagttagctcactcattaggcaccccaggctttacactttatgcttccggctcgtatgttgtgtggaattgtgagcggataacaatttcacacaggaaacagctatgaccatgattacgccaagcgcgcaattaaccctcactaaagggaacaaaagctgggtaccgggccccccctcgacaccagttgcattcgattcctgtttgtaattgtccaattcctgcagcccgggggatcggcagatgtagtgtttccacaggggatccactagttctagagcggccgccaccgcggtggagctccaattcgccctatagtgagtcgtattacgcgcgctcactggccgtcgttttacaacgtcgtgactgggaaaaccctggcgttacccaacttaatcgccttgcagcacatccccctttcgccagctggcgtaatagcgaagaggcccgcaccgatcgcccttcccaacagttgcgcagcctgaatggcgaatggaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcaggtggcacttttcggggaaatgtgcgcggaacccctatttgtttatttttctaaatacattcaaatatgtatccgctcatgagacaataaccctgataaatgcttcaataatattgaaaaaggaagagtatgagtattcaacatttccgtgtcgcccttattcccttttttgcggcattttgccttcctgtttttgctcacccagaaacgctggtgaaagtaaaagatgctgaagatcagttgggtgcacgagtgggttacatcgaactggatctcaacagcggtaagatccttgagagttttcgccccgaagaacgttttccaatgatgagcacttttaaagttctgctatgtggcgcggtattatcccgtattgac

SEQ ID NO: 18 - DNA, 2838 bp, synthetic construct
  misc_feature 1..2838: note = synthetic construct
  source 1..2838: mol_type = other DNA
gcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgatcgatgcggcctcaagagcttcactgagtaggattaagatattgcagatgtagtgtttccacagggtggctcttcagtgcaccagcggaacctgctgcgcgtgatatgcagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagc

SEQ ID NO: 19 - DNA, 2838 bp, synthetic construct
  misc_feature 1..2838: note = synthetic construct
  source 1..2838: mol_type = other DNA
gcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgatcgatgcggcctctctgaatagagttgggaagagatgcatacaacatatgtagtatttccacagggaatacaatggacaaatgacctcaagagcaggcgcgtgatatgcagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagc

SEQ ID NO: 20 - DNA, 2838 bp, synthetic construct
  misc_feature 1..2838: note = synthetic construct
  source 1..2838: mol_type = other DNA
gcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgatcgatgcggcctcgggcagctgcaggaataagagggatgtgaatggtaatgatggcttcaacatggcgcttgctcttcattccctgggtgtagtctgcgcgtgatatgcagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagc

SEQ ID NO: 21 - DNA, 2838 bp, synthetic construct
  misc_feature 1..2838: note = synthetic construct
  source 1..2838: mol_type = other DNA
gcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgatcgatgcggcctcctccttactgcagccgaagtccggcctcaggatgttgtcgatgaaaaagttggtggtgcggtgcagctgggccgctggctgcggcgcgtgatatgcagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagc

SEQ ID NO: 22 - DNA, 2838 bp, synthetic construct
  misc_feature 1..2838: note = synthetic construct
  source 1..2838: mol_type = other DNA
gcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgatcgatgcggcctcgtcctttcgccggccgaactcgggccgcaggatgttgtcgatgaagaagttggtgatgcggtgcgggtgctggtggttgccgggcgcgtgatatgcagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagc

SEQ ID NO: 23 - DNA, 2838 bp, synthetic construct
  misc_feature 1..2838: note = synthetic construct
  source 1..2838: mol_type = other DNA
gcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgatcgatgcggcctcggaacattggtaattaaacttaacgcctcagatttagacgaaggattgaatggggacattgtttattcattctcgaatgatacgcgcgtgatatgcagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagc

SEQ ID NO: 24 - DNA, 2838 bp, synthetic construct
  misc_feature 1..2838: note = synthetic construct
  source 1..2838: mol_type = other DNA
gcgtttctgggtgagcaaaaacaggaaggcaaaatgccgcaaaaaagggaataagggcgacacggaaatgttgaatactcatactcttcctttttcaatattattgaagcatttatcagggttattgtctcatgagcggatacatatttgaatgtatttagaaaaataaacaaataggggttccgcgcacatttccccgaaaagtgccacctaaattgtaagcgttaatattttgttaaaattcgcgttaaatttttgttaaatcagctcattttttaaccaataggccgaaatcggcaaaatcccttataaatcaaaagaatagaccgagatagggttgagtgttgttccagtttggaacaagagtccactattaaagaacgtggactccaacgtcaaagggcgaaaaaccgtctatcagggcgatggcccactacgtgaaccatcaccctaatcaagttttttggggtcgaggtgccgtaaagcactaaatcggaaccctaaagggagcccccgatttagagcttgacggggaaagccggcgaacgtggcgagaaaggaagggaagaaagcgaaaggagcgggcgctagggcgctggcaagtgtagcggtcacgctgcgcgtaaccaccacacccgccgcgcttaatgcgccgctacagggcgcgtcccattcgccattcaggctgcgcaactgttgggaagggcgatcggtgcgggcctcttcgctattacgccagctggcgaaagggggatgtgctgcaaggcgattaagttgggtaacgccagggttttcccagtcacgacgttgtaaaacgacggccagtgagcgcgcgtaatacgactcactatagggcgaattgggtacgatcgatgcggcctcggaacgctggtgattcatcccaatgcctcagatttagacgaaggcttgaatggggatattatttactccttctccagtgatgtgcgcgtgatatgcagctccagcttttgttccctttagtgagggttaattgcgcgcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaaggacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcctgactccccgtcgtgtagataactacgatacgggagggcttaccatctggccccagtgctgcaatgataccgcgagacccacgctcaccggctccagatttatcagcaataaaccagccagccggaagggccgagcgcagaagtggtcctgcaactttatccgcctccatccagtctattaattgttgccgggaagctagagtaagtagttcgccagttaatagtttgcgcaacgttgttgccattgctacaggcatcgtggtgtcacgctcgtcgtttggtatggcttcattcagctccggttcccaacgatcaaggcgagttacatgatcccccatgttgtgcaaaaaagcggttagctccttcggtcctccgatcgttgtcagaagtaagttggccgcagtgttatcactcatggttatggcagcactgcataattctcttactgtcatgccatccgtaagatgcttttctgtgactggtgagtactcaaccaagtcattctgagaatagtgtatgcggcgaccgagttgctcttgcccggcgtcaatacgggataataccgcgccacatagc

SEQ ID NO: 25 - RNA, 56 nt, synthetic construct
  misc_feature 1..56: note = synthetic construct
  source 1..56: mol_type = other RNA
agtcctcatctccctcaagcgtttaagagctatgctgttttgaatggtcccaaaac

SEQ ID NO: 26 - RNA, 86 nt, synthetic construct
  misc_feature 1..86: note = synthetic construct
  source 1..86: mol_type = other RNA
ggaaccattcaaaacagcatagcaagtttaaataaggctagtccgttatcaacttgtaaaagtggcaccgagtcggtgcttttttt

SEQ ID NO: 27 - RNA, 56 nt, synthetic construct
  misc_feature 1..56: note = synthetic construct
  source 1..56: mol_type = other RNA
tggtaatgatggcttcaacagttttagagctatgctgttttgaatggtcccaaaac

SEQ ID NO: 28 - RNA, 86 nt, synthetic construct; identical to SEQ ID NO: 26
  misc_feature 1..86: note = synthetic construct
  source 1..86: mol_type = other RNA

SEQ ID NO: 29 - RNA, 56 nt, synthetic construct; identical to SEQ ID NO: 25
  misc_feature 1..56: note = synthetic construct
  source 1..56: mol_type = other RNA

SEQ ID NO: 30 - RNA, 86 nt, synthetic construct; identical to SEQ ID NO: 26
  misc_feature 1..86: note = synthetic construct
  misc_feature 57: note = u is 5-(3-Aminoallyl)-uridine-5'-triphosphate, labeled with Cyanine5
  source 1..86: mol_type = other RNA

SEQ ID NO: 31 - RNA, 56 nt, synthetic construct; identical to SEQ ID NO: 27
  misc_feature 1..56: note = synthetic construct
  misc_feature 1..3: note = synthetic construct
  misc_feature 53..55: note = synthetic construct
  source 1..56: mol_type = other RNA

SEQ ID NO: 32 - RNA, 86 nt, synthetic construct; identical to SEQ ID NO: 26
  misc_feature 1..86: note = synthetic construct
  misc_feature 1..3: note = synthetic construct
  misc_feature 83..85: note = synthetic construct
  source 1..86: mol_type = other RNA

SEQ ID NO: 33 - RNA, 56 nt, synthetic construct; identical to SEQ ID NO: 27
  misc_feature 1..56: note = synthetic construct
  misc_feature 1..3: note = synthetic construct
  misc_feature 1..4: note = synthetic construct
  misc_feature 53..55: note = synthetic construct
  misc_feature 53..56: note = synthetic construct
  source 1..56: mol_type = other RNA

SEQ ID NO: 34 - RNA, 86 nt, synthetic construct; identical to SEQ ID NO: 26
  misc_feature 1..86: note = synthetic construct
  misc_feature 1..3: note = synthetic construct
  misc_feature 1..4: note = synthetic construct
  misc_feature 83..85: note = synthetic construct
  misc_feature 83..86: note = synthetic construct
  source 1..86: mol_type = other RNA

SEQ ID NO: 35 - RNA, 56 nt, synthetic construct; identical to SEQ ID NO: 27
  misc_feature 1..56: note = synthetic construct
  misc_feature 1..3: note = synthetic construct
  misc_feature 1..4: note = synthetic construct
  misc_feature 53..55: note = synthetic construct
  misc_feature 53..56: note = synthetic construct
  source 1..56: mol_type = other RNA

SEQ ID NO: 36 - RNA, 86 nt, synthetic construct; identical to SEQ ID NO: 26
  misc_feature 1..86: note = synthetic construct
  misc_feature 1..3: note = synthetic construct
  misc_feature 1..4: note = synthetic construct
  misc_feature 83..85: note = synthetic construct
  misc_feature 83..86: note = synthetic construct
  source 1..86: mol_type = other RNA

SEQ ID NO: 37 - RNA, 56 nt, synthetic construct; identical to SEQ ID NO: 27
  misc_feature 1..56: note = synthetic construct
  misc_feature 1: note = synthetic construct
  misc_feature 1..2: note = synthetic construct
  misc_feature 55: note = synthetic construct
  misc_feature 55..56: note = synthetic construct
  source 1..56: mol_type = other RNA

SEQ ID NO: 38 - RNA, 86 nt, synthetic construct; identical to SEQ ID NO: 26
  misc_feature 1..86: note = synthetic construct
  misc_feature 1: note = synthetic construct
  misc_feature 1..2: note = synthetic construct
  misc_feature 85: note = synthetic construct
  misc_feature 85..86: note = synthetic construct
  source 1..86: mol_type = other RNA

SEQ ID NO: 39 - RNA, 56 nt, synthetic construct; identical to SEQ ID NO: 25
  misc_feature 1..56: note = synthetic construct
  misc_feature 3: note = synthetic construct
  source 1..56: mol_type = other RNA

SEQ ID NO: 40 - RNA, 56 nt, synthetic construct; identical to SEQ ID NO: 25
  misc_feature 1..56: note = synthetic construct
  misc_feature 9: note = synthetic construct
  source 1..56: mol_type = other RNA

SEQ ID NO: 41 - RNA, 56 nt, synthetic construct; identical to SEQ ID NO: 25
  misc_feature 1..56: note = synthetic construct
  misc_feature 11: note = synthetic construct
  source 1..56: mol_type = other RNA

SEQ ID NO: 42 - RNA, 113 nt, synthetic construct
  misc_feature 1..113: note = synthetic construct
  source 1..113: mol_type = other RNA
agtcctcatctccctcaagcgtttaagagctatgctggtaacagcatagcaagtttaaataaggctagtccgttatcaacttgaaaaagtggcaccgagtcggtgcttttttt

SEQ ID NO: 43 - RNA, 100 nt, synthetic construct
  misc_feature 1..100: note = synthetic construct
  source 1..100: mol_type = other RNA
agtcctcatctccctcaagcgttttagagctagtaatagcaagttaaaataaggctagtccgttatcaacttgaaaaagtggcaccgagtcggtgctttt

SEQ ID NO: 44 - RNA, 113 nt, synthetic construct
  misc_feature 1..113: note = synthetic construct
  source 1..113: mol_type = other RNA
gcagatgtagtgtttccacagtttaagagctatgctggaaacagcatagcaagtttaaataaggctagtccgttatcaacttgaaaaagtggcaccgagtcggtgcttttttt

SEQ ID NO: 45 - RNA, 111 nt, synthetic construct
  misc_feature 1..111: note = synthetic construct
  source 1..111: mol_type = other RNA
tcctcatctccctcaagcgtttaagagctatgctggtaacagcatagcaagtttaaataaggctagtccgttatcaacttgaaaaagtggcaccgagtcggtgcttttttt

SEQ ID NO: 46 - RNA, 110 nt, synthetic construct
  misc_feature 1..110: note = synthetic construct
  source 1..110: mol_type = other RNA
cctcatctccctcaagcgtttaagagctatgctggtaacagcatagcaagtttaaataaggctagtccgttatcaacttgaaaaagtggcaccgagtcggtgcttttttt

SEQ ID NO: 47 - RNA, 114 nt, synthetic construct
  misc_feature 1..114: note = synthetic construct
  source 1..114: mol_type = other RNA
gagtcctcatctccctcaagcgtttaagagctatgctggtaacagcatagcaagtttaaataaggctagtccgttatcaacttgaaaaagtggcaccgagtcggtgcttttttt

SEQ ID NO: 48 - RNA, 115 nt, synthetic construct
  misc_feature 1..115: note = synthetic construct
  source 1..115: mol_type = other RNA
ggagtcctcatctccctcaagcgtttaagagctatgctggtaacagcatagcaagtttaaataaggctagtccgttatcaacttgaaaaagtggcaccgagtcggtgcttttttt

SEQ ID NO: 49 - RNA, 113 nt, synthetic construct
  misc_feature 1..113: note = synthetic construct
  source 1..113: mol_type = other RNA
agtcctcatctccctcaagcgtttaagagctatgctggtaacagcatagcaagtttaaataattctagtccgttatcaacttgaaaaagtggcaccgagtcggtgcttttttt

SEQ ID NO: 50 - RNA, 113 nt, synthetic construct
  misc_feature 1..113: note = synthetic construct
  source 1..113: mol_type = other RNA
agtcctcatctccctcaagcgtttaagagctatgctggtaacagcatagcaagtttaaataaaactagtccgttatcaacttgaaaaagtggcaccgagtcggtgcttttttt

SEQ ID NO: 51 - RNA, 113 nt, synthetic construct
  misc_feature 1..113: note = synthetic construct
  source 1..113: mol_type = other RNA
agtcctcatctccctcaagcgtttaagagctatgctggtaacagcatagcaagtttaaataaaactagtttgttatcaacttgaaaaagtggcaccgagtcggtgcttttttt

SEQ ID NO: 52 - RNA, 111 nt, synthetic construct
  misc_feature 1..111: note = synthetic construct
  source 1..111: mol_type = other RNA
ggactttttttagtcctcatctccctcaagcgttttagagctagaaatagcaagttaaaataaggctagtccgttatcaacttgaaaaagtggcaccgagtcggtgctttt

SEQ ID NO: 53 - RNA, 116 nt, synthetic construct
  misc_feature 1..116: note = synthetic construct
  source 1..116: mol_type = other RNA
gatgaggactttttttagtcctcatctccctcaagcgttttagagctagaaatagcaagttaaaataaggctagtccgttatcaacttgaaaaagtggcaccgagtcggtgctttt

SEQ ID NO: 54 - RNA, 111 nt, synthetic construct
  misc_feature 1..111: note = synthetic construct
  source 1..111: mol_type = other RNA
gcttgttttttagtcctcatctccctcaagcgttttagagctagaaatagcaagttaaaataaggctagtccgttatcaacttgaaaaagtggcaccgagtcggtgctttt

SEQ ID NO: 55 - RNA, 113 nt, synthetic construct; identical to SEQ ID NO: 42
  misc_feature 1..113: note = synthetic construct
  misc_feature 1: note = synthetic construct
  source 1..113: mol_type = other RNA

SEQ ID NO: 56 - RNA, 113 nt, synthetic construct; identical to SEQ ID NO: 42
  misc_feature 1..113: note = synthetic construct
  source 1..113: mol_type = other RNA

SEQ ID NO: 57 - RNA, 113 nt, synthetic construct
  misc_feature 1..113: note = synthetic construct
  misc_feature 39: note = synthetic construct
  misc_feature 39: note = n is a, c, g, or u, unknown or other
  source 1..113: mol_type = other RNA
agtcctcatctccctcaagcgtttaagagctatgctggnaacagcatagcaagtttaaataaggctagtccgttatcaacttgaaaaagtggcaccgagtcggtgcttttttt

SEQ ID NO: 58 - RNA, 113 nt, synthetic construct; identical to SEQ ID NO: 57
  misc_feature 1..113: note = synthetic construct
  misc_feature 1..3: note = synthetic construct
  misc_feature 39: note = synthetic construct
  misc_feature 39: note = n is a, c, g, or u, unknown or other
  misc_feature 110..112: note = synthetic construct
  source 1..113: mol_type = other RNA

SEQ ID NO: 59 - RNA, 113 nt, synthetic construct; identical to SEQ ID NO: 57
  misc_feature 1..113: note = synthetic construct
  misc_feature 1..3: note = synthetic construct
  misc_feature 1..4: note = synthetic construct
  misc_feature 39: note = synthetic construct
  misc_feature 39: note = n is a, c, g, or u, unknown or other
  misc_feature 110..112: note = synthetic construct
  misc_feature 110..113: note = synthetic construct
  source 1..113: mol_type = other RNA

SEQ ID NO: 60 - RNA, 113 nt, synthetic construct; identical to SEQ ID NO: 57
  misc_feature 1..113: note = synthetic construct
  misc_feature 1..3: note = synthetic construct
  misc_feature 1..4: note = synthetic construct
  misc_feature 39: note = synthetic construct
  misc_feature 39: note = n is a, c, g, or u, unknown or other
  misc_feature 109..111: note = synthetic construct
  misc_feature 109..112: note = synthetic construct
  misc_feature 110..112: note = synthetic construct
  source 1..113: mol_type = other RNA

SEQ ID NO: 61 - RNA, 102 nt, synthetic construct
  misc_feature 1..102: note = synthetic construct
  misc_feature 1: note = synthetic construct
  misc_feature 1..2: note = synthetic construct
  misc_feature 2: note = synthetic construct
  misc_feature 2: note = n is a, c, g, or u, unknown or other
  misc_feature 68: note = synthetic construct
  misc_feature 68: note = n is a, c, g, or u
  misc_feature 100: note = synthetic construct
  misc_feature 100: note = n is a, c, g, or u
  misc_feature 101: note = synthetic construct
  misc_feature 101..102: note = synthetic construct
  source 1..102: mol_type = other RNA
tngcagatgtagtgtttccacagtttaagagctagtaatagcaagtttaaataaggctagtccgttancaacttgaaaaagtggcaccgagtcggtgctntt

SEQ ID NO: 62 - RNA, 100 nt, synthetic construct
  misc_feature 1..100: note = synthetic construct
  misc_feature 1: note = synthetic construct
  misc_feature 1..2: note = synthetic construct
  misc_feature 34: note = synthetic construct
  misc_feature 34: note = n is a, c, g, or u, unknown or other
  misc_feature 74: note = synthetic construct
  misc_feature 74: note = n is a, c, g, or u, unknown or other
  misc_feature 90: note = synthetic construct
  misc_feature 90: note = n is a, c, g, or u, unknown or other
  misc_feature 99: note = synthetic construct
  misc_feature 99..100: note = synthetic construct
  source 1..100: mol_type = other RNA
gcagatgtagtgtttccacagtttaagagctagnaatagcaagtttaaataaggctagtccgttatcaacttgnaaaagtggcaccgagncggtgctttt

SEQ ID NO: 63 - RNA, 113 nt, synthetic construct
  misc_feature 1..113: note = synthetic construct
  misc_feature 1..3: note = synthetic construct
  source 1..113: mol_type = other RNA
agtcctcatctccctcaagcgtttaagagctatgctggaaacagcatagcaagtttaaataaggctagtccgttatcaacttgaaaaagtggcaccgagtcggtgcttttttt

SEQ ID NO: 64 - RNA, 113 nt, synthetic construct; identical to SEQ ID NO: 63
  misc_feature 1..113: note = synthetic construct
  misc_feature 1..4: note = synthetic construct
  source 1..113: mol_type = other RNA

SEQ ID NO: 65 - RNA, 113 nt, synthetic construct; identical to SEQ ID NO: 63
  misc_feature 1..113: note = synthetic construct
  misc_feature 1..5: note = synthetic construct
  source 1..113: mol_type = other RNA

SEQ ID NO: 66 - RNA, 113 nt, synthetic construct; identical to SEQ ID NO: 63
  misc_feature 1..113: note = synthetic construct
  misc_feature 109..113: note = synthetic construct
  source 1..113: mol_type = other RNA

SEQ ID NO: 67 - RNA, 113 nt, synthetic construct; identical to SEQ ID NO: 42
  misc_feature 1..113: note = synthetic construct
  misc_feature 20: note = synthetic construct
  source 1..113: mol_type = other RNA

SEQ ID NO: 68 - RNA, 113 nt, synthetic construct; identical to SEQ ID NO: 42
  misc_feature 1..113: note = synthetic construct
  misc_feature 19: note = synthetic construct
  source 1..113: mol_type = other RNA

SEQ ID NO: 69 - RNA, 113 nt, synthetic construct; identical to SEQ ID NO: 42
  misc_feature 1..113: note = synthetic construct
  misc_feature 18: note = synthetic construct
  source 1..113: mol_type = other RNA

SEQ ID NO: 70 - RNA, 113 nt, synthetic construct; identical to SEQ ID NO: 42
  misc_feature 1..113: note = synthetic construct
  misc_feature 17: note = synthetic construct
  source 1..113: mol_type = other RNA

SEQ ID NO: 71 - RNA, 113 nt, synthetic construct; identical to SEQ ID NO: 42
  misc_feature 1..113: note = synthetic construct
  misc_feature 17..18: note = synthetic construct
  source 1..113: mol_type = other RNA

SEQ ID NO: 72 - RNA, 113 nt, synthetic construct; identical to SEQ ID NO: 42
  misc_feature 1..113: note = synthetic construct
  misc_feature 1..3: note = synthetic construct
  misc_feature 110..112: note = synthetic construct
  source 1..113: mol_type = other RNA

SEQ ID NO: 73 - RNA, 113 nt, synthetic construct; identical to SEQ ID NO: 44
  misc_feature 1..113: note = synthetic construct
  misc_feature 1..3: note = synthetic construct
  misc_feature 110..112: note = synthetic construct
  source 1..113: mol_type = other RNA

SEQ ID NO: 74 - RNA, 113 nt, synthetic construct; identical to SEQ ID NO: 42
  misc_feature 1..113: note = synthetic construct
  misc_feature 1..20: note = synthetic construct
  source 1..113: mol_type = other RNA

SEQ ID NO: 75 - RNA, 113 nt, synthetic construct; identical to SEQ ID NO: 42
  misc_feature 1..113: note = synthetic construct
  misc_feature 1..26: note = synthetic construct
  source 1..113: mol_type = other RNA

SEQ ID NO: 76 - RNA, 113 nt, synthetic construct; identical to SEQ ID NO: 42
  misc_feature 1..113: note = synthetic construct
  misc_feature 1..37: note = synthetic construct
  source 1..113: mol_type = other RNA

SEQ ID NO: 77 - RNA, 113 nt, synthetic construct; identical to SEQ ID NO: 42
  misc_feature 1..113: note = synthetic construct
  misc_feature 1..7: note = synthetic construct
  misc_feature 9..11: note = synthetic construct
  misc_feature 13..14: note = synthetic construct
  misc_feature 20: note = synthetic construct
  misc_feature 30..31: note = synthetic construct
  misc_feature 33: note = synthetic construct
  misc_feature 35..36: note = synthetic construct
  misc_feature 42: note = synthetic construct
  misc_feature 45: note = synthetic construct
  misc_feature 76..77: note = synthetic construct
  misc_feature 80..82: note = synthetic construct
  misc_feature 90: note = synthetic construct
  misc_feature 95..96: note = synthetic construct
  misc_feature 100..101: note = synthetic construct
  misc_feature 104: note = synthetic construct
  misc_feature 106..112: note = synthetic construct
  source 1..113: mol_type = other RNA

SEQ ID NO: 78 - RNA, 113 nt, synthetic construct; identical to SEQ ID NO: 42
  misc_feature 1..113: note = synthetic construct
  misc_feature 1..7: note = synthetic construct
  misc_feature 9..11: note = synthetic construct
  misc_feature 17: note = synthetic construct
  misc_feature 27..28: note = synthetic construct
  misc_feature 30: note = synthetic construct
  misc_feature 32..33: note = synthetic construct
  misc_feature 36: note = synthetic construct
  misc_feature 39: note = synthetic construct
  misc_feature 42: note = synthetic construct
  misc_feature 44: note = synthetic construct
  misc_feature 47: note = synthetic construct
  misc_feature 57: note = synthetic construct
  misc_feature 62..63: note = synthetic construct
  misc_feature 67..68: note = synthetic construct
  misc_feature 73..74: note = synthetic construct
  misc_feature 77..78: note = synthetic construct
  misc_feature 87: note = synthetic construct
  misc_feature 90: note = synthetic construct
  misc_feature 92..93: note = synthetic construct
  misc_feature 97..98: note = synthetic construct
  misc_feature 101: note = synthetic construct
  misc_feature 103..109: note = synthetic construct
  source 1..113: mol_type = other RNA

SEQ ID NO: 79 - RNA, 113 nt, synthetic construct; identical to SEQ ID NO: 42
  misc_feature 1..113: note = synthetic construct
  misc_feature 1..7: note = synthetic construct
  misc_feature 9..11: note = synthetic construct
  misc_feature 17: note = synthetic construct
  misc_feature 27..28: note = synthetic construct
  misc_feature 30: note = synthetic construct
  misc_feature 32..33: note = synthetic construct
  misc_feature 36: note = synthetic construct
  misc_feature 39: note = synthetic construct
  misc_feature 42: note = synthetic construct
  misc_feature 44: note = synthetic construct
  misc_feature 47: note = synthetic construct
  misc_feature 57: note = synthetic construct
  misc_feature 62..63: note = synthetic construct
  misc_feature 67..68: note = synthetic construct
  misc_feature 73..74: note = synthetic construct
  misc_feature 77..78: note = synthetic construct
  misc_feature 87: note = synthetic construct
  misc_feature 90: note = synthetic construct
  misc_feature 92..93: note = synthetic construct
  misc_feature 97..98: note = synthetic construct
  misc_feature 101: note = synthetic construct
  misc_feature 103..109: note = synthetic construct
  source 1..113: mol_type = other RNA

SEQ ID NO: 80 - RNA, 113 nt, synthetic construct; identical to SEQ ID NO: 42
  misc_feature 1..113: note = synthetic construct
  misc_feature 1..3: note = synthetic construct
  misc_feature 8: note = synthetic construct
  misc_feature 17..18: note = synthetic construct
  misc_feature 21: note = synthetic construct
  misc_feature 26: note = synthetic construct
  misc_feature 28..29: note = synthetic construct
  misc_feature 32: note = synthetic construct
  misc_feature 34: note = synthetic construct
  misc_feature 37..38: note = synthetic construct
  misc_feature 40..41: note = synthetic construct
  misc_feature 43..44: note = synthetic construct
  misc_feature 46: note = synthetic construct
  misc_feature 48..49: note = synthetic construct
  misc_feature 51..52: note = synthetic construct
  misc_feature 58: note = synthetic construct
  misc_feature 61..64: note = synthetic construct
  misc_feature 67: note = synthetic construct
  misc_feature 78..79: note = synthetic construct
  misc_feature 83..89: note = synthetic construct
  misc_feature 91..92: note = synthetic construct
  misc_feature 94: note = synthetic construct
  misc_feature 97..99: note = synthetic construct
  misc_feature 102..103: note = synthetic construct
  misc_feature 105: note = synthetic construct
  source 1..113: mol_type = other RNA

SEQ ID NO: 81 - RNA, 100 nt, synthetic construct; identical to SEQ ID NO: 43
  misc_feature 1..100: note = synthetic construct
  misc_feature 1..3: note = synthetic construct
  misc_feature 8: note = synthetic construct
  misc_feature 17..18: note = synthetic construct
  misc_feature 21: note = synthetic construct
  misc_feature 26: note = synthetic construct
  misc_feature 28..29: note = synthetic construct
  misc_feature 32..33: note = synthetic construct
  misc_feature 35..36: note = synthetic construct
  misc_feature 38..39: note = synthetic construct
  misc_feature 41..42: note = synthetic construct
  misc_feature 46: note = synthetic construct
  misc_feature 48: note = synthetic construct
  misc_feature 51..54: note = synthetic construct
  misc_feature 57: note = synthetic construct
  misc_feature 68..69: note = synthetic construct
  misc_feature 73..79: note = synthetic construct
  misc_feature 81..82: note = synthetic construct
  misc_feature 84: note = synthetic construct
  misc_feature 87..89: note = synthetic construct
  misc_feature 92..93: note = synthetic construct
  misc_feature 95: note = synthetic construct
  source 1..100: mol_type = other RNA

SEQ ID NO: 82 - RNA, 113 nt, synthetic construct; identical to SEQ ID NO: 44
  misc_feature 1..113: note = synthetic construct
  misc_feature 1..3: note = synthetic construct
  misc_feature 110..112: note = synthetic construct
  source 1..113: mol_type = other RNA

SEQ ID NO: 83 - RNA, 113 nt, synthetic construct
  misc_feature 1..113: note = synthetic construct
  misc_feature 1..3: note = synthetic construct
  misc_feature 6: note = synthetic construct
  misc_feature 8: note = synthetic construct
  misc_feature 11: note = synthetic construct
  misc_feature 13..17: note = synthetic construct
  misc_feature 30..31: note = synthetic construct
  misc_feature 33: note = synthetic construct
  misc_feature 35..36: note = synthetic construct
  misc_feature 39: note = synthetic construct
  misc_feature 42: note = synthetic construct
  misc_feature 45: note = synthetic construct
  misc_feature 47: note = synthetic construct
  misc_feature 50: note = synthetic construct
  misc_feature 56: note = synthetic construct
  misc_feature 60: note = synthetic construct
  misc_feature 65..66: note = synthetic construct
  misc_feature 69..71: note = synthetic construct
  misc_feature 76..77: note = synthetic construct
  misc_feature 80..82: note = synthetic construct
  misc_feature 90: note = synthetic construct
  misc_feature 93: note = synthetic construct
  misc_feature 95..96: note = synthetic construct
  misc_feature 100..101: note = synthetic construct
  misc_feature 104: note = synthetic construct
  misc_feature 106..112: note = synthetic construct
  source 1..113: mol_type = other RNA
gcagatgtagtgtttccacagtttaagagctatgctggtaacagcatagcaagtttaaataaggctagtccgttatcaacttgaaaaagtggcaccgagtcggtgcttttttt

SEQ ID NO: 84 - RNA, 100 nt, synthetic construct
  misc_feature 1..100: note = synthetic construct
  misc_feature 1..5: note = synthetic construct
  misc_feature 26..29: note = synthetic construct
  misc_feature 32..33: note = synthetic construct
  misc_feature 35..36: note = synthetic construct
  misc_feature 38..39: note = synthetic construct
  misc_feature 41..42: note = synthetic construct
  misc_feature 46: note = synthetic construct
  misc_feature 48..49: note = synthetic construct
  misc_feature 52..54: note = synthetic construct
  misc_feature 57: note = synthetic construct
  misc_feature 68..69: note = synthetic construct
  misc_feature 73..79: note = synthetic construct
  misc_feature 81..82: note = synthetic construct
  misc_feature 84: note = synthetic construct
  misc_feature 87..89: note = synthetic construct
  misc_feature 92..93: note = synthetic construct
  misc_feature 95: note = synthetic construct
  source 1..100: mol_type = other RNA
gcagatgtagtgtttccacagttttagagctagtaatagcaagttaaaataaggctagtccgttatcaacttgaaaaagtggcaccgagtcggtgctttt

SEQ ID NO: 85 - RNA, 100 nt, synthetic construct; identical to SEQ ID NO: 84
  misc_feature 1..100: note = synthetic construct
  misc_feature 1..3: note = synthetic construct
  misc_feature 6: note = synthetic construct
  misc_feature 8: note = synthetic construct
  misc_feature 11: note = synthetic construct
  misc_feature 13..17: note = synthetic construct
  misc_feature 25: note = synthetic construct
  misc_feature 30..31: note = synthetic construct
  misc_feature 34: note = synthetic construct
  misc_feature 37: note = synthetic construct
  misc_feature 40: note = synthetic construct
  misc_feature 50: note = synthetic construct
  misc_feature 55..56: note = synthetic construct
  misc_feature 59..61: note = synthetic construct
  misc_feature 66..67: note = synthetic construct
  misc_feature 70..72: note = synthetic construct
  misc_feature 80: note = synthetic construct
  misc_feature 83: note = synthetic construct
  misc_feature 85..86: note = synthetic construct
  misc_feature 90..91: note = synthetic construct
  misc_feature 94: note = synthetic construct
  misc_feature 96..99: note = synthetic construct
  source 1..100: mol_type = other RNA

SEQ ID NO: 86 - DNA, 113 bp, synthetic construct
  misc_feature 1..113: note = synthetic construct
  misc_feature 1..20: note = synthetic construct
  source 1..113: mol_type = other DNA
  variation 22..24: note = uracil
  variation 31: note = uracil
  variation 33: note = uracil
  variation 36: note = uracil
  variation 39: note = uracil
  variation 47: note = uracil
  variation 54..56: note = uracil
  variation 60: note = uracil
  variation 66: note = uracil
  variation 69: note = uracil
  variation 73..74: note = uracil
  variation 76: note = uracil
  variation 81..82: note = uracil
  variation 90: note = uracil
  variation 100: note = uracil
  variation 104: note = uracil
  variation 107..113: note = uracil
agtcctcatctccctcaagcgtttaagagctatgctggtaacagcatagcaagtttaaataaggctagtccgttatcaacttgaaaaagtggcaccgagtcggtgcttttttt

SEQ ID NO: 87 - DNA, 113 bp, synthetic construct; identical to SEQ ID NO: 86
  misc_feature 1..113: note = synthetic construct
  misc_feature 1..26: note = synthetic construct
  source 1..113: mol_type = other DNA
  variation 31: note = uracil
  variation 33: note = uracil
  variation 36: note = uracil
  variation 39: note = uracil
  variation 47: note = uracil
  variation 54..56: note = uracil
  variation 60: note = uracil
  variation 66: note = uracil
  variation 69: note = uracil
  variation 73..74: note = uracil
  variation 76: note = uracil
  variation 81..82: note = uracil
  variation 90: note = uracil
  variation 100: note = uracil
  variation 104: note = uracil
  variation 107..113: note = uracil

SEQ ID NO: 88 - DNA, 113 bp, synthetic construct; identical to SEQ ID NO: 86
  misc_feature 1..113: note = synthetic construct
  misc_feature 1..37: note = synthetic construct
  source 1..113: mol_type = other DNA
  variation 39: note = uracil
  variation 47: note = uracil
  variation 54..56: note = uracil
  variation 60: note = uracil
  variation 66: note = uracil
  variation 69: note = uracil
  variation 73..74: note = uracil
  variation 76: note = uracil
  variation 81..82: note = uracil
  variation 90: note = uracil
  variation 100: note = uracil
  variation 104: note = uracil
  variation 107..113: note = uracil

SEQ ID NO: 89 - RNA, 113 nt, synthetic construct; identical to SEQ ID NO: 83
  misc_feature 1..113: note = synthetic construct
  misc_feature 15: note = synthetic construct
  misc_feature 15..16: note = synthetic construct
  source 1..113: mol_type = other RNA

SEQ ID NO: 90 - RNA, 113 nt, synthetic construct; identical to SEQ ID NO: 42
  misc_feature 1..113: note = synthetic construct
  misc_feature 1: note = synthetic construct
  misc_feature 1..2: note = synthetic construct
  source 1..113: mol_type = other RNA

SEQ ID NO: 91 - RNA, 113 nt, synthetic construct; identical to SEQ ID NO: 42
  misc_feature 1..113: note = synthetic construct
  misc_feature 1..2: note = synthetic construct
  misc_feature 1..3: note = synthetic construct
  source 1..113: mol_type = other RNA

SEQ ID NO: 92 - RNA, 113 nt, synthetic construct; identical to SEQ ID NO: 42
  misc_feature 1..113: note = synthetic construct
  misc_feature 1..3: note = synthetic construct
  misc_feature 1..4: note = synthetic construct
  source 1..113: mol_type = other RNA

SEQ ID NO: 93 - RNA, 113 nt, synthetic construct; identical to SEQ ID NO: 42
  misc_feature 1..113: note = synthetic construct
  misc_feature 1..4: note = synthetic construct
  misc_feature 1..5: note = synthetic construct
  source 1..113: mol_type = other RNA

SEQ ID NO: 94 - RNA, 115 nt, synthetic construct; identical to SEQ ID NO: 48
  misc_feature 1..115: note = synthetic construct
  misc_feature 1..5: note = synthetic construct
  misc_feature 1..6: note = synthetic construct
  source 1..115: mol_type = other RNA

SEQ ID NO: 95 - RNA, 113 nt, synthetic construct; identical to SEQ ID NO: 42
  misc_feature 1..113: note = synthetic construct
  misc_feature 109..112: note = synthetic construct
  misc_feature 109..113: note = synthetic construct
  source 1..113: mol_type = other RNA

SEQ ID NO: 96 - RNA, 113 nt, synthetic construct; identical to SEQ ID NO: 42
  misc_feature 1..113: note = synthetic construct
  misc_feature 108..112: note = synthetic construct
  misc_feature 108..113: note = synthetic construct
  source 1..113: mol_type = other RNA

SEQ ID NO: 97 - RNA, 114 nt, synthetic construct
  misc_feature 1..114: note = synthetic construct
  misc_feature 1: note = synthetic construct
  misc_feature 1..3: note = synthetic construct
  misc_feature 1..4: note = synthetic construct
  source 1..114: mol_type = other RNA
cagtcctcatctccctcaagcgtttaagagctatgctggtaacagcatagcaagtttaaataaggctagtccgttatcaacttgaaaaagtggcaccgagtcggtgcttttttt

SEQ ID NO: 98 - RNA, 114 nt, synthetic construct; identical to SEQ ID NO: 47
  misc_feature 1..114: note = synthetic construct
  misc_feature 1: note = synthetic construct
  misc_feature 1..3: note = synthetic construct
  misc_feature 1..4: note = synthetic construct
  source 1..114: mol_type = other RNA

SEQ ID NO: 99 - RNA, 115 nt, synthetic construct
  misc_feature 1..115: note = synthetic construct
  misc_feature 1..2: note = synthetic construct
  misc_feature 1..5: note = synthetic construct
  misc_feature 1..6: note = synthetic construct
  source 1..115: mol_type = other RNA
tcagtcctcatctccctcaagcgtttaagagctatgctggtaacagcatagcaagtttaaataaggctagtccgttatcaacttgaaaaagtggcaccgagtcggtgcttttttt

SEQ ID NO: 100 - RNA, 115 nt, synthetic construct
  misc_feature 1..115: note = synthetic construct
  misc_feature 1..2: note = synthetic construct
  misc_feature 1..5: note = synthetic construct
  misc_feature 1..6: note = synthetic construct
  source 1..115: mol_type = other RNA
agagtcctcatctccctcaagcgtttaagagctatgctggtaacagcatagcaagtttaaataaggctagtccgttatcaacttgaaaaagtggcaccgagtcggtgcttttttt

SEQ ID NO: 101 - RNA, 116 nt, synthetic construct
  misc_feature 1..116: note = synthetic construct
  misc_feature 1..3: note = synthetic construct
  misc_feature 1..7: note = synthetic construct
  misc_feature 1..8: note = synthetic construct
  misc_feature 112..115: note = synthetic construct
  misc_feature 112..116: note = synthetic construct
  source 1..116: mol_type = other RNA
ctcagtcctcatctccctcaagcgtttaagagctatgctggtaacagcatagcaagtttaaataaggctagtccgttatcaacttgaaaaagtggcaccgagtcggtgcttttttt

SEQ ID NO: 102 - RNA, 113 nt, synthetic construct; identical to SEQ ID NO: 42
  misc_feature 1..113: note = synthetic construct
  misc_feature 20: note = synthetic construct
  misc_feature 20..21: note = synthetic construct
  source 1..113: mol_type = other RNA

SEQ ID NO: 103 - RNA, 113 nt, synthetic construct; identical to SEQ ID NO: 42
  misc_feature 1..113: note = synthetic construct
  misc_feature 19..20: note = synthetic construct
  misc_feature 19..20: note = synthetic construct
  source 1..113: mol_type = other RNA

SEQ ID NO: 104 - RNA, 113 nt, synthetic construct; identical to SEQ ID NO: 42
  misc_feature 1..113: note = synthetic construct
  misc_feature 18: note = synthetic construct
  misc_feature 18..19: note = synthetic construct
  source 1..113: mol_type = other RNA

SEQ ID NO: 105 - RNA, 113 nt, synthetic construct; identical to SEQ ID NO: 42
  misc_feature 1..113: note = synthetic construct
  misc_feature 17: note = synthetic construct
  misc_feature 17..18: note = synthetic construct
  source 1..113: mol_type = other RNA

SEQ ID NO: 106 - RNA, 113 nt, synthetic construct; identical to SEQ ID NO: 42
  misc_feature 1..113: note = synthetic construct
  misc_feature 17..18: note = synthetic construct
  misc_feature 17..19: note = synthetic construct
  source 1..113: mol_type = other RNA

SEQ ID NO: 107 - RNA, 113 nt, synthetic construct; identical to SEQ ID NO: 42
  misc_feature 1..113: note = synthetic construct
  misc_feature 1..3: note = synthetic construct
  misc_feature 1..4: note = synthetic construct
  misc_feature 110..112: note = synthetic construct
  misc_feature 110..113: note = synthetic construct
  source 1..113: mol_type = other RNA

SEQ ID NO: 108 - RNA, 113 nt, synthetic construct; identical to SEQ ID NO: 44
  misc_feature 1..113: note = synthetic construct
  misc_feature 1..3: note = synthetic construct
  misc_feature 1..4: note = synthetic construct
  misc_feature 108..112: note = synthetic construct
  misc_feature 108..113: note = synthetic construct
  source 1..113: mol_type = other RNA

SEQ ID NO: 109 - RNA, 113 nt, synthetic construct; identical to SEQ ID NO: 44
  misc_feature 1..113: note = synthetic construct
  misc_feature 1..5: note = synthetic construct
  misc_feature 1..6: note = synthetic construct
  misc_feature 108..112: note = synthetic construct
  misc_feature 108..113: note = synthetic construct
  source 1..113: mol_type = other RNA

SEQ ID NO: 110 - RNA, 113 nt, synthetic construct; identical to SEQ ID NO: 42
  misc_feature 1..113: note = synthetic construct
  misc_feature 1: note = synthetic construct
  misc_feature 1..2: note = synthetic construct
  misc_feature 110..112: note = synthetic construct
  misc_feature 110..113: note = synthetic construct
  source 1..113: mol_type = other RNA

SEQ ID NO: 111 - RNA, 113 nt, synthetic construct; identical to SEQ ID NO: 42
  misc_feature 1..113: note = synthetic construct
  misc_feature 1: note = synthetic construct
  misc_feature 1..2: note = synthetic construct
  misc_feature 112: note = synthetic construct
  misc_feature 112..113: note = synthetic construct
  source 1..113: mol_type = other RNA

SEQ ID NO: 112 - RNA, 75 nt, synthetic construct
  misc_feature 1..75: note = synthetic construct
  misc_feature 1..3: note = synthetic construct
  misc_feature 1..4: note = synthetic construct
  misc_feature 72..74: note = synthetic construct
  misc_feature 72..75: note = synthetic construct
  source 1..75: mol_type = other RNA
agtcctcatctccctcaagcgtttaagagctatgctggtaacagcatagcaagtttaaataaggctagtccgttt

SEQ ID NO: 113 - RNA, 77 nt, synthetic construct
  misc_feature 1..77: note = synthetic construct
  misc_feature 1: note = synthetic construct
  misc_feature 1..2: note = synthetic construct
  misc_feature 76: note = synthetic construct
  misc_feature 76..77: note = synthetic construct
  source 1..77: mol_type = other RNA
agtcctcatctccctcaagcgtttaagagctatgctggtaacagcatagcaagtttaaataaggctagtccgttatc

SEQ ID NO: 114 - RNA, 78 nt, synthetic construct
  misc_feature 1..78: note = synthetic construct
  misc_feature 1: note = synthetic construct
  misc_feature 1..2: note = synthetic construct
  misc_feature 77: note = synthetic construct
  misc_feature 77..78: note = synthetic construct
  source 1..78: mol_type = other RNA
gagtcctcatctccctcaagcgtttaagagctatgctggtaacagcatagcaagtttaaataaggctagtccgttatc

SEQ ID NO: 115 - RNA, 113 nt, synthetic construct; identical to SEQ ID NO: 44
  misc_feature 1..113: note = synthetic construct
  misc_feature 1..3: note = synthetic construct
  misc_feature 1..4: note = synthetic construct
  misc_feature 110..112: note = synthetic construct
  misc_feature 110..113: note = synthetic construct
  source 1..113: mol_type = other RNA

SEQ ID NO: 116 - RNA, 113 nt, synthetic construct; identical to SEQ ID NO: 44
  misc_feature 1..113: note = synthetic construct
  misc_feature 1: note = synthetic construct
  misc_feature 1..2: note = synthetic construct
  misc_feature 112: note = synthetic construct
  misc_feature 112..113: note = synthetic construct
  source 1..113: mol_type = other RNA

SEQ ID NO: 117 - RNA, 113 nt, synthetic construct
  misc_feature 1..113: note = synthetic construct
  source 1..113: mol_type = other RNA
gatgttgtcgatgaaaaagtgtttaagagctatgctggtaacagcatagcaagtttaaataaggctagtccgttatcaacttgaaaaagtggcaccgagtcggtgcttttttt

SEQ ID NO: 118 - RNA, 113 nt, synthetic construct; identical to SEQ ID NO: 117
  misc_feature 1..113: note = synthetic construct
  misc_feature 15: note = synthetic construct
  source 1..113: mol_type = other RNA

SEQ ID NO: 119 - RNA, 113 nt, synthetic construct
  misc_feature 1..113: note = synthetic construct
  source 1..113: mol_type = other RNA
gatttagacgaaggattgaagtttaagagctatgctggtaacagcatagcaagtttaaataaggctagtccgttatcaacttgaaaaagtggcaccgagtcggtgcttttttt

SEQ ID NO: 120 - RNA, 113 nt, synthetic construct; identical to SEQ ID NO: 119
  misc_feature 1..113: note = synthetic construct
  misc_feature 15: note = synthetic construct
  source 1..113: mol_type = other RNA

SEQ ID NO: 121 - RNA, 113 nt, synthetic construct; identical to SEQ ID NO: 83
  misc_feature 1..113: note = synthetic construct
  misc_feature 6: note = synthetic construct
  misc_feature 8: note = synthetic construct
  misc_feature 11: note = synthetic construct
  misc_feature 13..15: note = synthetic construct
  misc_feature 31: note = synthetic construct
  misc_feature 33: note = synthetic construct
  misc_feature 36: note = synthetic construct
  misc_feature 39: note = synthetic construct
  misc_feature 47: note = synthetic construct
  misc_feature 81..82: note = synthetic construct
  misc_feature 90: note = synthetic construct
  misc_feature 104: note = synthetic construct
  misc_feature 107..112: note = synthetic construct
  source 1..113: mol_type = other RNA

SEQ ID NO: 122 - RNA, 113 nt, synthetic construct
  misc_feature 1..113: note = synthetic construct
  misc_feature 70..71: note = Z base - Z base
  misc_feature 70..71: note = n is a, c, g, or u, or unknown or other
  source 1..113: mol_type = other RNA
agtcctcatctccctcaagcgtttaagagctatgctggtaacagcatagcaagtttaaataaggctagtnngttatcaacttgaaaaagtggcaccgagtcggtgcttttttt

SEQ ID NO: 123 - RNA, 100 nt, synthetic construct
  misc_feature 1..100: note = synthetic construct
  misc_feature 1: note = synthetic construct
  misc_feature 1..2: note = synthetic construct
  misc_feature 54: note = synthetic construct
  misc_feature 57: note = synthetic construct
  misc_feature 99: note = synthetic construct
  misc_feature 99..100: note = synthetic construct
  source 1..100: mol_type = other RNA
agtcctcatctccctcaagcgtttaagagctagtaatagcaagtttaaataaggttaatccgttatcaacaagaaattgtggcaccgagtcggtgctttt

SEQ ID NO: 124 - RNA, 113 nt, synthetic construct
  misc_feature 1..113: note = synthetic construct
  misc_feature 1: note = synthetic construct
  misc_feature 1..2: note = synthetic construct
  misc_feature 64: note = synthetic construct
  misc_feature 67: note = synthetic construct
  misc_feature 112: note = synthetic construct
  misc_feature 112..113: note = synthetic construct
  source 1..113: mol_type = other RNA
agtcctcatctccctcaagcgtttaagagctatgctggtaacagcatagcaagtttaaataaggttaatccgttatcaacaagaaattgtggcaccgagtcggtgcttttttt

SEQ ID NO: 125 - RNA, 113 nt, synthetic construct
  misc_feature 1..113: note = synthetic construct
  misc_feature 95..96: note = Z base - Z base
  misc_feature 95..96: note = n is a, c, g, or u, or unknown or other
  source 1..113: mol_type = other RNA
agtcctcatctccctcaagcgtttaagagctatgctggtaacagcatagcaagtttaaataaggctagtccgttatcaacttgaaaaagtggcanngagtcggtgcttttttt

SEQ ID NO: 126 - RNA, 74 nt, synthetic construct
  misc_feature 1..74: note = synthetic construct
  misc_feature 1: note = synthetic construct
  misc_feature 1..2: note = synthetic construct
  misc_feature 73: note = synthetic construct
  misc_feature 73..74: note = synthetic construct
  source 1..74: mol_type = other RNA
agtcctcatctccctcaagcgtttaagagctatgctggtaacagcatagcaagtttaaataaggctagtccgtt

SEQ ID NO: 127 - RNA, 75 nt, synthetic construct
  misc_feature 1..75: note = synthetic construct
  misc_feature 1: note = synthetic construct
  misc_feature 1..2: note = synthetic construct
  misc_feature 74: note = synthetic construct
  misc_feature 74..75: note = synthetic construct
  source 1..75: mol_type = other RNA
agtcctcatctccctcaagcgtttaagagctatgctggtaacagcatagcaagtttaaataaggctagtccgtta

SEQ ID NO: 128 - DNA, 50 bp, synthetic construct
  misc_feature 1..50: note = synthetic construct
  source 1..50: mol_type = other DNA
ttatatgaacataactcaatttgtaaaaaagggtattggggaattcatta

SEQ ID NO: 129 - DNA, 50 bp, synthetic construct
  misc_feature 1..50: note = synthetic construct
  source 1..50: mol_type = other DNA
aatatacttgtattgagttaaacattttttcccataaccccttaagtaat

SEQ ID NO: 130 - RNA, 98 nt, synthetic construct
  misc_feature 1..98: note = synthetic construct
  source 1..98: mol_type = other RNA
ataactcaatttgtaaaaaagttttagagctatagcaagttaaaataaggtagtccgttatcaacttgaaaaagtggcaccgagtcggtgcttttttt

SEQ ID NO: 131 - RNA, 113 nt, synthetic construct; identical to SEQ ID NO: 42
  misc_feature 1..113: note = synthetic construct
  misc_feature 1..20: note = synthetic construct
  source 1..113: mol_type = other RNA

SEQ ID NO: 132 - RNA, 113 nt, synthetic construct; identical to SEQ ID NO: 125
  misc_feature 1..113: note = synthetic construct
  misc_feature 95..96: note = Z base
  misc_feature 95..96: note = n is a, c, g, or u, or unknown or other
  source 1..113: mol_type = other RNA

SEQ ID NO: 133 - RNA, 113 nt, synthetic construct; identical to SEQ ID NO: 42
  misc_feature 1..113: note = synthetic construct
  misc_feature 1..37: note = synthetic construct
  source 1..113: mol_type = other RNA

SEQ ID NO: 134 - DNA, 23 bp, synthetic construct
  misc_feature 1..23: note = synthetic construct
  source 1..23: mol_type = other DNA
ccagccaagcgcacctaatttcc

SEQ ID NO: 135 - RNA, 88 nt, synthetic construct
  misc_feature 1..88: note = synthetic construct
  misc_feature 34: note = site for fluorescent dye or label attachment
  source 1..88: mol_type = other RNA
ggaaattaggtgcgcttggcgttttagagctagaaatagcaagttaaaataaggctagtccgccatcaacttgaaaaagcggcaccga

SEQ ID NO: 136 - DNA, 23 bp, synthetic construct
  misc_feature 1..23: note = synthetic construct
  source 1..23: mol_type = other DNA
agtcctcatctccctcaagcagg

SEQ ID NO: 137 - DNA, 23 bp, synthetic construct
  misc_feature 1..23: note = synthetic construct
  source 1..23: mol_type = other DNA
cctgcttgagggagatgaggact

SEQ ID NO: 138 - DNA, 23 bp, synthetic construct
  misc_feature 1..23: note = synthetic construct
  source 1..23: mol_type = other DNA
agtcctcaactccctcaagcagg

SEQ ID NO: 139 - DNA, 23 bp, synthetic construct
  misc_feature 1..23: note = synthetic construct
  source 1..23: mol_type = other DNA
cctgcttgagggagttgaggact

SEQ ID NO: 140 - DNA, 23 bp, synthetic construct
  misc_feature 1..23: note = synthetic construct
  source 1..23: mol_type = other DNA
agccctcatttccctcaagcagg

SEQ ID NO: 141 - DNA, 23 bp, synthetic construct
  misc_feature 1..23: note = synthetic construct
  source 1..23: mol_type = other DNA
cctgcttgagggaaatgagggct

SEQ ID NO: 142 - DNA, 23 bp, synthetic construct
  misc_feature 1..23: note = synthetic construct
  source 1..23: mol_type = other DNA
actcctcatccccctcaagccgg

SEQ ID NO: 143 - DNA, 23 bp, synthetic construct
  misc_feature 1..23: note = synthetic construct
  source 1..23: mol_type = other DNA
cctgcttgagggggatgaggagt

(19) United States
(12) Patent Application Publication
(10) Pub. No.: US 2025/0092389 A1
(43) Pub. Date: Mar. 20, 2025

(54) GUIDE RNA WITH CHEMICAL MODIFICATIONS

(71) Applicant: Agilent Technologies, Inc., Santa Clara, CA (US)

(72) Inventors: Daniel E. RYAN, San Francisco, CA (US); Douglas J. DELLINGER, Boulder, CO (US); Jeffrey R. SAMPSON, San Jose, CA (US); Robert KAISER, Santa Clara, CA (US); Joel MYERSON, Berkeley, CA (US)

(21) Appl. No.: 18/883,302

(22) Filed: Sep. 12, 2024

Related U.S. Application Data
(63) Continuation of application No. 18/332,464, filed on Jun. 9, 2023.
Application No. 18/332,464 is a continuation of application No. 17/103,678, filed on Nov. 24, 2020.
Application No. 17/103,678 is a continuation of application No. 14/757,204, filed on Dec. 3, 2015, now Pat. No. 10,900,034.
(60) Provisional application No. 62/256,095, filed on Nov. 16, 2015.
(60) Provisional application No. 62/146,189, filed on Apr. 10, 2015.
(60) Provisional application No. 62/087,211, filed on Dec. 3, 2014.

(51) Int. Cl. C12N 15/11; C07H 21/02; C12N 9/22; C12N 15/113; C12N 15/90; C12Q 1/6876

(52) U.S. Cl. CPC C12N 15/111; C07H 21/02; C12N 9/22; C12N 15/113; C12N 15/907; C12Q 1/6876; C12N 2310/10; C12N 2310/20; C12N 2310/312; C12N 2310/315; C12N 2310/321; C12N 2310/322; C12N 2310/323; C12N 2310/3231; C12N 2310/333; C12N 2310/335; C12N 2310/346; C12N 2310/3517; C12N 2310/531; C12N 2320/51; C12N 2320/53; C12N 2330/31

(57) ABSTRACT

The present invention relates to modified guide RNAs and their use in clustered, regularly interspaced, short palindromic repeats (CRISPR)/CRISPR-associated (Cas) systems.

### CROSS REFERENCE TO RELATED APPLICATIONS

This application is a continuation of U.S. application Ser. No. 18/332,464, filed Jun. 9, 2023, which is a continuation of U.S. application Ser. No. 17/103,678, filed Nov. 24, 2020, which is a continuation of U.S. application Ser. No. 14/757,204, filed Dec. 3, 2015 (now issued as U.S. Pat. No. 10,900,034), which claims the benefit of U.S. Provisional Application No. 62/256,095, filed Nov. 16, 2015, U.S. Provisional Application No. 62/146,189, filed Apr. 10, 2015, and U.S. Provisional Application No. 62/087,211, filed Dec. 3, 2014, the contents of each of which is incorporated by reference in its entirety.

### SEQUENCE LISTING

The instant application contains a Sequence Listing which has been submitted electronically in ASCII format and is hereby incorporated by reference in its entirety. Said ASCII copy, created on Aug. 29, 2023, is named A2185-700921 FT-20150013-16_SL.xml and is 390,292 bytes in size.

### FIELD OF THE INVENTION

The present invention relates to the field of molecular biology. In particular, the present invention relates to the clusters of regularly interspaced short palindromic repeats (CRISPR) technology.

### BACKGROUND OF THE INVENTION

The native prokaryotic CRISPR-Cas system comprises an array of short repeats with intervening variable sequences of constant length (i.e., clusters of regularly interspaced short palindromic repeats, or “CRISPR”), and CRISPR-associated (“Cas”) proteins. The RNA of the transcribed CRISPR array is processed by a subset of the Cas proteins into small guide RNAs, which generally have two components as discussed below. There are at least three different systems: Type I, Type II and Type III. The enzymes involved in the processing of the RNA into mature crRNA are different in the 3 systems. In the native prokaryotic system, the guide RNA (“gRNA”) comprises two short, non-coding RNA species referred to as CRISPR RNA (“crRNA”) and trans-acting RNA (“tracrRNA”). In an exemplary system, the gRNA forms a complex with a Cas nuclease. The gRNA:Cas nuclease complex binds a target polynucleotide sequence having a protospacer adjacent motif (“PAM”) and a protospacer, which is a sequence complementary to a portion of the gRNA. The recognition and binding of the target polynucleotide by the gRNA: Cas nuclease complex induces cleavage of the target polynucleotide. The native CRISPR-Cas system functions as an immune system in prokaryotes, where gRNA: Cas nuclease complexes recognize and silence exogenous genetic elements in a manner analogous to RNAi in eukaryotic organisms, thereby conferring resistance to exogenous genetic elements such as plasmids and phages.

It has been demonstrated that a single-guide RNA (“sgRNA”) can replace the complex formed between the naturally-existing crRNA and tracrRNA.

Considerations relevant to developing a gRNA, including a sgRNA, include specificity, stability, and functionality. Specificity refers to the ability of a particular gRNA: Cas nuclease complex to bind to and/or cleave a desired target sequence, whereas little or no binding and/or cleavage of polynucleotides different in sequence and/or location from the desired target occurs. Thus, specificity refers to minimizing off-target effects of the gRNA: Cas nuclease complex. Stability refers to the ability of the gRNA to resist degradation by enzymes, such as nucleases, and other substances that exist in intra-cellular and extra-cellular environments. Thus, there is a need for providing gRNA, including sgRNA, having increased resistance to nucleolytic degradation, increased binding affinity for the target polynucleotide, and/or reduced off-target effects while, nonetheless, having gRNA functionality. Further considerations relevant to developing a gRNA include transfectability and immunostimulatory properties. Thus, there is a need for providing gRNA, including sgRNA, having efficient and titratable transfectability into cells, especially into the nuclei of eukaryotic cells, and having minimal or no immunostimulatory properties in the transfected cells. Another important consideration for gRNA is to provide an effective means for delivering it into and maintaining it in the intended cell, tissue, bodily fluid or organism for a duration sufficient to allow the desired gRNA functionality.

### BRIEF DESCRIPTION OF THE DRAWINGS

FIG. 1 is a set of diagrams showing a schematic model of an exemplary CRISPR-Cas system. The exemplary system shown here is a Type II system having a Cas nuclease. In this particular example, the Cas nuclease is the Cas9 nuclease. The Cas9 nuclease recognizes a PAM sequence (here, the PAM sequence is a 3-nt sequence of NGG, where N is A, G, C or T, but other PAM sequences are known to exist). The sgRNA includes a guide sequence, a crRNA sequence or segment, and tracrRNA sequence or segment. The guide sequence of the sgRNA hybridizes with the DNA target directly upstream of the PAM sequence. In the example shown here, Cas9 mediates a double-stranded break upstream of the PAM sequence (arrows).

FIG. 2A is a diagram showing an exemplary CRISPR-Cas9-mediated cleavage assay.

FIG. 2B is a table showing components and their concentrations for a biochemical cleavage assay used to generate the data in FIG. 4.

FIG. 2C is a diagram showing titration of Streptococcus pyogenes Cas9 nuclease for the biochemical cleavage assay.

FIG. 2D is a diagram showing titration of an exemplary sgRNA for the biochemical cleavage assay. In this example a sgRNA named kanC1 is targeted to a complementary sequence in the kanamycin resistance gene.

FIG. 3 shows exemplary conditions and procedures for the biochemical cleavage assay which uses purified components in vitro.

FIGS. 4A, 4B, and 4C include a table showing the data obtained using exemplary modified guide RNAs in the cleavage assay.

FIG. 5A shows an exemplary guide RNA disclosed in the application.

FIG. 5B shows an exemplary single guide RNA (sgRNA) disclosed in the application.

FIG. 6 is a table showing exemplary guide RNAs having at least two chemical modifications (e.g., a first modification and a second modification). Each number represents a modification as indicated and each “x” indicates the combination of modifications in a guide RNA. In certain embodiments, the first and second modifications are present on a single nucleotide. In certain embodiments, the first and second modifications are present on separate nucleotides.

FIG. 7 shows exemplary types of guide RNAs having at least three chemical modifications. The lower part of FIG. 7 lists several types of modifications. The table in the upper part of FIG. 7 indicates how a double modification (“double mod,” a combination of two types of modifications) can be combined with a single modification (“single mod,” one type of modification). An “x” indicates the presence of the corresponding double mod and single mod in a guide RNA.

FIGS. 8A and 8B show fluorophore-modified CLTA1 sgRNAs for in vitro testing. In FIG. 8A, the RNA sequence (SEQ ID NO: 135) of a sgRNA for CLTA1 is shown, including a position where a fluorescent dye or a label could be attached to the sgRNA. Target DNA is SEQ ID NO: 134. FIG. 8B shows a structure determined by Xray crystallography of a Cas9:sgRNA complex, as reported in Nishimasu et al., Cell (2014) 156, 1-15.

FIG. 9A shows CLTA1 sgRNAs modified with 2-thiouridine at certain locations (positions 3, 9 and 11) in an effort to improve specificity for the target CTLA1. Top strand and bottom strand sequences (respectively) of the CTLA1 targets are: ON1 (SEQ ID NOs: 136 and 137); OFF1 (SEQ ID NOs: 138 and 139); OFF2 (SEQ ID NOs: 140 and 141); and OFF3 (SEQ ID NOs: 142 and 143). FIG. 9B shows that gRNA modified with 2-thioU (SEQ ID NO: ###) can increase target specificity of the gRNAs when off-target sites involve U-G wobble pairing. In particular, the CTLA1_2thioU+11 had much lower cleavage of the off-target sequence CLTA1 OFF3, which has a T to C mutation at the 11 position in the 5′ strand. Top strand and bottom strand sequences (respectively) of the CTLA1 targets are: ON1 (SEQ ID NOs: 136 and 137); OFF1 (SEQ ID NOs: 138 and 139); and OFF3 (SEQ ID NOs: 142 and 143).

FIG. 10 shows the guide RNA scaffold secondary structure, displaying noncovalent binding interactions with amino acids of Cas9, as reported in Jiang et al., Science (2015) 348:6242, 1477-81.

FIGS. 11A and 11B illustrate experimental results showing that gene disruption in human cell lines, with high frequencies of indels and homologous recombination (HR), can be achieved using synthesized and chemically modified sgRNAs disclosed herein, as reported in Hendel et al., Nat. Biotechnol. (2015) 33:9, 985-9.

FIGS. 12A, 12B, 12C and 12D illustrate experimental results showing that chemically modified sgRNAs as described herein can be used to achieve high frequencies of gene disruption or targeted genome editing in stimulated primary human T cells as well as in CD34+ hematopoietic stem and progenitor cells (HSPCs), as reported in Hendel et al., Nat. Biotechnol. (2015) 33:9, 985-9.

### DETAILED DESCRIPTION OF THE INVENTION

This invention is based, at least in part, on an unexpected discovery that certain chemical modifications to gRNA are tolerated by the CRISPR-Cas system. In particular, certain chemical modifications believed to increase the stability of the gRNA, to alter the thermostability of a gRNA hybridization interaction, and/or to decrease the off-target effects of Cas:gRNA complexation do not substantially compromise the efficacy of Cas:gRNA binding to, nicking of, and/or cleavage of the target polynucleotide. Furthermore, certain chemical modifications are believed to provide gRNA, including sgRNA, having efficient and titratable transfectability into cells, especially into the nuclei of eukaryotic cells, and/or having minimal or no immunostimulatory properties in the transfected cells. Certain chemical modifications are believed to provide gRNA, including sgRNA, which can be effectively delivered into and maintained in the intended cell, tissue, bodily fluid or organism for a duration sufficient to allow the desired gRNA functionality.

### I. Definitions

As used herein, the term “guide RNA” generally refers to an RNA molecule (or a group of RNA molecules collectively) that can bind to a Cas protein and aid in targeting the Cas protein to a specific location within a target polynucleotide (e.g., a DNA). A guide RNA can comprise a crRNA segment and a tracrRNA segment. As used herein, the term “crRNA” or “crRNA segment” refers to an RNA molecule or portion thereof that includes a polynucleotide-targeting guide sequence, a stem sequence, and, optionally, a 5′-overhang sequence. As used herein, the term “tracrRNA” or “tracrRNA segment” refers to an RNA molecule or portion thereof that includes a protein-binding segment (e.g., the protein-binding segment is capable of interacting with a CRISPR-associated protein, such as a Cas9). The term “guide RNA” encompasses a single guide RNA (sgRNA), where the crRNA segment and the tracrRNA segment are located in the same RNA molecule. The term “guide RNA” also encompasses, collectively, a group of two or more RNA molecules, where the crRNA segment and the tracrRNA segment are located in separate RNA molecules.

The term “scaffold” refers to the portions of guide RNA molecules comprising sequences which are substantially identical or are highly conserved across natural biological species. Scaffolds include the tracrRNA segment and the portion of the crRNA segment other than the polynucleotide-targeting guide sequence at or near the 5′ end of the crRNA segment, excluding any unnatural portions comprising sequences not conserved in native crRNAs and tracrRNAs.

The term “nucleic acid”, “polynucleotide” or “oligonucleotide” refers to a DNA molecule, an RNA molecule, or analogs thereof. As used herein, the terms “nucleic acid”, “polynucleotide” and “oligonucleotide” include, but are not limited to DNA molecules such as cDNA, genomic DNA or synthetic DNA and RNA molecules such as a guide RNA, messenger RNA or synthetic RNA. Moreover, as used herein, the terms “nucleic acid” and “polynucleotide” include single-stranded and double-stranded forms.

The term “modification” in the context of an oligonucleotide or polynucleotide includes but is not limited to (a) end modifications, e.g., 5′ end modifications or 3′ end modifications, (b) nucleobase (or “base”) modifications, including replacement or removal of bases, (c) sugar modifications, including modifications at the 2′, 3′, and/or 4′ positions, and (d) backbone modifications, including modification or replacement of the phosphodiester linkages. The term “modified nucleotide” generally refers to a nucleotide having a modification to the chemical structure of one or more of the base, the sugar, and the phosphodiester linkage or backbone portions, including nucleotide phosphates.

The terms “Z” and “P” refer to the nucleotides, nucleobases, or nucleobase analogs developed by Steven Benner and colleagues as described for example in “Artificially expanded genetic information system: a new base pair with an alternative hydrogen bonding pattern” Yang, Z., Hutter, D., Sheng, P., Sismour, A. M. and Benner, S. A. (2006) Nucleic Acids Res., 34, 6095-101, the contents of which is hereby incorporated by reference in its entirety.

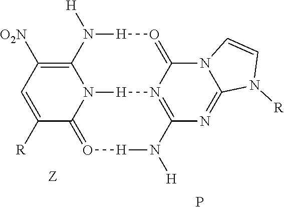

The terms “xA”, “xG”, “xC”. “xT”, or “x(A,G,C,T)” and “yA”, “yG”, “yC”, “yT”, or “y(A,G,C,T)” refer to nucleotides, nucleobases, or nucleobase analogs as described by Krueger et al in “Synthesis and Properties of Size-Expanded DNAs: Toward Designed, Functional Genetic Systems”; Andrew T. Krueger, Haige Lu, Alex H. F. Lee, and Eric T. Kool (2007) Acc. Chem. Res., 40, 141-50, the contents of which is hereby incorporated by reference in its entirety.

The term “Unstructured Nucleic Acid” or “UNA” refers to nucleotides, nucleobases, or nucleobase analogs as described in U.S. Pat. No. 7,371,580, the contents of which is hereby incorporated by reference in its entirety. An unstructured nucleic acid, or UNA, modification is also referred to as a “pseudo-complementary” nucleotide, nucleobase or nucleobase analog (see e.g., Lahoud et al. (1991) Nucl. Acids Res., 36:10, 3409-19).

The terms “PACE” and “thioPACE” refer to internucleotide phosphodiester linkage analogs containing phosphonoacetate or thiophosphonoacetate groups, respectively. These modifications belong to a broad class of compounds comprising phosphonocarboxylate moiety, phosphonocarboxylate ester moiety, thiophosphonocarboxylate moiety and thiophosphonocarboxylate ester moiety. These linkages can be described respectively by the general formulae P(CR1R2)ₙCOOR and (S)—P(CR1R2)ₙCOOR wherein n is an integer from 0 to 6 and each of R1 and R2 is independently selected from the group consisting of H, an alkyl and substituted alkyl. Some of these modifications are described by Yamada et al. in “Synthesis and Biochemical Evaluation of Phosphonoformate Oligodeoxyribonucleotides” Christina M. Yamada, Douglas J. Dellinger and Marvin H. Caruthers (2006) J. Am. Chem. Soc. 128:15, 5251-61, the contents of which is hereby incorporated by reference in its entirety.

As used herein, “modification” refers to a chemical moiety, or portion of a chemical structure, which differs from that found in unmodified ribonucleotides, namely adenosine, guanosine, cytidine, and uridine ribonucleotides. The term “modification” may refer to type of modification. For example, “same modification” means same type of modification, and “the modified nucleotides are the same” means the modified nucleotides have the same type(s) of modification while the base (A, G, C. U, etc.) may be different. Similarly, a guide RNA with “two modifications” is a guide RNA with two types of modifications, which may or may not be in the same nucleotide, and each type may appear in multiple nucleotides in the guide RNA. Similarly, a guide RNA with “three modifications” is a guide RNA with three types of modifications, which may or may not be in the same nucleotide, and each type may appear in multiple nucleotides.

As used herein, the term “target polynucleotide” or “target” refers to a polynucleotide containing a target nucleic acid sequence. A target polynucleotide may be single-stranded or double-stranded, and, in certain embodiments, is double-stranded DNA. In certain embodiments, the target polynucleotide is single-stranded RNA. A “target nucleic acid sequence” or “target sequence,” as used herein, means a specific sequence or the complement thereof that one wishes to bind to, nick, or cleave using a CRISPR system.

The term “hybridization” or “hybridizing” refers to a process where completely or partially complementary polynucleotide strands come together under suitable hybridization conditions to form a double-stranded structure or region in which the two constituent strands are joined by hydrogen bonds. As used herein, the term “partial hybridization” includes where the double-stranded structure or region contains one or more bulges or mismatches. Although hydrogen bonds typically form between adenine and thymine or adenine and uracil (A and T or A and U) or cytosine and guanine (C and G), other noncanonical base pairs may form (See e.g., Adams et al., “The Biochemistry of the Nucleic Acids,” 11th ed., 1992). It is contemplated that modified nucleotides may form hydrogen bonds that allow or promote hybridization.

The term “cleavage” or “cleaving” refers to breaking of the covalent phosphodiester linkage in the ribosylphosphodiester backbone of a polynucleotide. The terms “cleavage” or “cleaving” encompass both single-stranded breaks and double-stranded breaks. Double-stranded cleavage can occur as a result of two distinct single-stranded cleavage events. Cleavage can result in the production of either blunt ends or staggered ends.

The term “CRISPR-associated protein” or “Cas protein” refers to a wild type Cas protein, a fragment thereof, or a mutant or variant thereof. The term “Cas mutant” or “Cas variant” refers to a protein or polypeptide derivative of a wild type Cas protein, e.g., a protein having one or more point mutations, insertions, deletions, truncations, a fusion protein, or a combination thereof. In certain embodiments, the “Cas mutant” or “Cas variant” substantially retains the nuclease activity of the Cas protein. In certain embodiments, the “Cas mutant” or “Cas variant” is mutated such that one or both nuclease domains are inactive. In certain embodiments, the “Cas mutant” or “Cas variant” has nuclease activity. In certain embodiments, the “Cas mutant” or “Cas variant” lacks some or all of the nuclease activity of its wild-type counterpart.

The term “nuclease domain” of a Cas protein refers to the polypeptide sequence or domain within the protein which possesses the catalytic activity for DNA cleavage. A nuclease domain can be contained in a single polypeptide chain, or cleavage activity can result from the association of two (or more) polypeptides. A single nuclease domain may consist of more than one isolated stretch of amino acids within a given polypeptide. Examples of these domains include RuvC-like motifs (amino acids 7-22, 759-766 and 982-989 in SEQ ID NO: 1) and HNH motif (aa 837-863). See Gasiunas et al. (2012) Proc. Natl. Acad. Sci. USA, 109:39, E2579-E2586 and WO2013176772.

A synthetic guide RNA that has “gRNA functionality” is one that has one or more of the functions of naturally occurring guide RNA, such as associating with a Cas protein, or a function performed by the guide RNA in association with a Cas protein. In certain embodiments, the functionality includes binding a target polynucleotide. In certain embodiments, the functionality includes targeting a Cas protein or a gRNA: Cas protein complex to a target polynucleotide. In certain embodiments, the functionality includes nicking a target polynucleotide. In certain embodiments, the functionality includes cleaving a target polynucleotide. In certain embodiments, the functionality includes associating with or binding to a Cas protein. In certain embodiments, the functionality is any other known function of a guide RNA in a CRISPR-Cas system with a Cas protein, including an artificial CRISPR-Cas system with an engineered Cas protein. In certain embodiments, the functionality is any other function of natural guide RNA. The synthetic guide RNA may have gRNA functionality to a greater or lesser extent than a naturally occurring guide RNA. In certain embodiments, a synthetic guide RNA may have greater functionality as to one property and lesser functionality as to another property in comparison to a similar naturally occurring guide RNA.

A “Cas protein having a single-strand nicking activity” refers to a Cas protein, including a Cas mutant or Cas variant, that has reduced ability to cleave one of two strands of a dsDNA as compared to a wild type Cas protein. For example, in certain embodiments, a Cas protein having a single-strand nicking activity has a mutation (e.g., amino acid substitution) that reduces the function of the RuvC domain (or the HNH domain) and as a result reduces the ability to cleave one strand of the target DNA. Examples of such variants include the D10A, H839A/H840A, and/or N863A substitutions in S. pyogenes Cas9, and also include the same or similar substitutions at equivalent sites in Cas9 enzymes of other species.

As used herein, the term “portion” or “fragment” of a sequence refers to any portion of the sequence (e.g., a nucleotide subsequence or an amino acid subsequence) that is smaller than the complete sequence. Portions of polynucleotides can be any length, for example, at least 5, 10, 15, 20, 25, 30, 40, 50, 75, 100, 150, 200, 300 or 500 or more nucleotides in length. A portion of a guide sequence can be about 50%, 40%, 30%, 20%, 10% of the guide sequence, e.g., one-third of the guide sequence or shorter, e.g., 7, 6, 5, 4, 3, or 2 nucleotides in length.

The term “derived from” in the context of a molecule refers to a molecule isolated or made using a parent molecule or information from that parent molecule. For example, a Cas9 single mutant nickase and a Cas9 double mutant null-nuclease are derived from a wild-type Cas9 protein.

The term “substantially identical” in the context of two or more polynucleotides (or two or more polypeptides) refers to sequences or subsequences that have at least about 60%, at least about 70%, at least about 80%, at least about 90%, about 90-95%, at least about 95%, at least about 98%, at least about 99% or more nucleotide (or amino acid) sequence identity, when compared and aligned for maximum correspondence using a sequence comparison algorithm or by visual inspection. Preferably, the “substantial identity” between polynucleotides exists over a region of the polynucleotide at least about 50 nucleotides in length, at least about 100 nucleotides in length, at least about 200 nucleotides in length, at least about 300 nucleotides in length, at least about 500 nucleotides in length, or over the entire length of the polynucleotide. Preferably, the “substantial identity” between polypeptides exists over a region of the polypeptide at least about 50 amino acid residues in length, at least about 100 amino acid residues in length, or over the entire length of the polypeptide.

As disclosed herein, a number of ranges of values are provided. It is understood that each intervening value, to the tenth of the unit of the lower limit, between the upper and lower limits of that range is also specifically contemplated. Each smaller range or intervening value encompassed by a stated range is also specifically contemplated. The term “about” generally refers to plus or minus 10% of the indicated number. For example, “about 10%” may indicate a range of 9% to 11%, and “about 20” may mean from 18-22. Other meanings of “about” may be apparent from the context, such as rounding off, so, for example “about 1” may also mean from 0.5 to 1.4.

### II. CRISPR-Mediated Sequence-Specific Binding and/or Cleavage

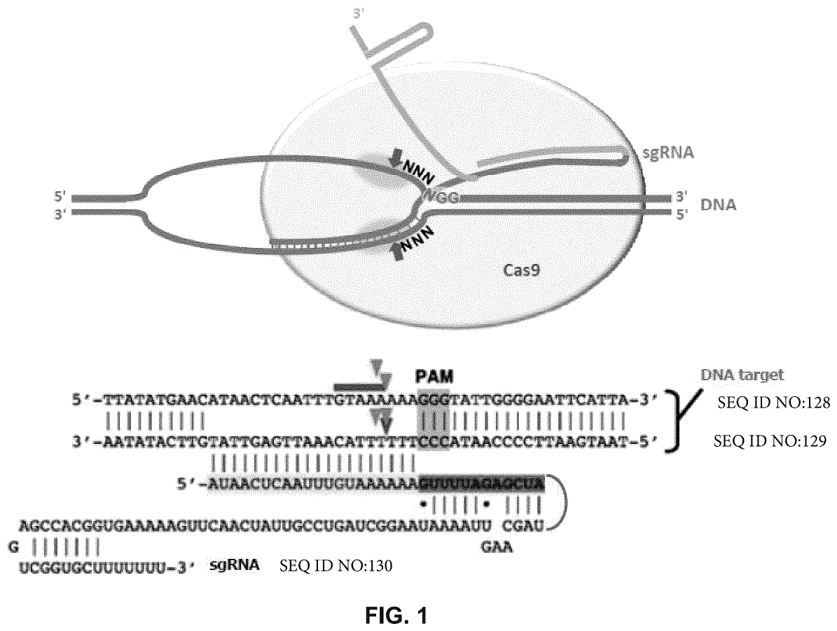

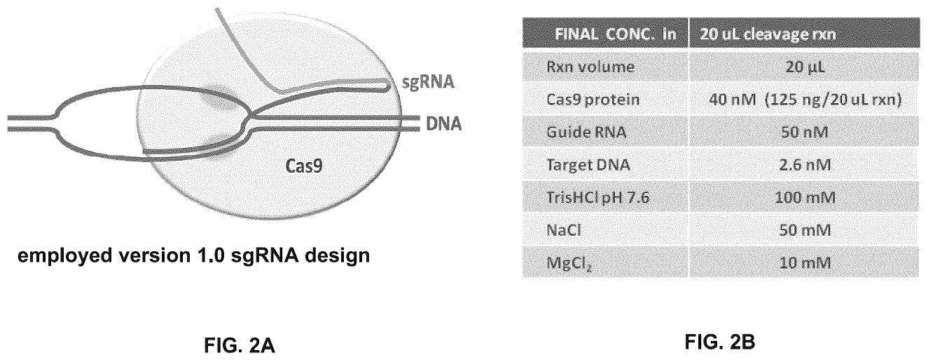

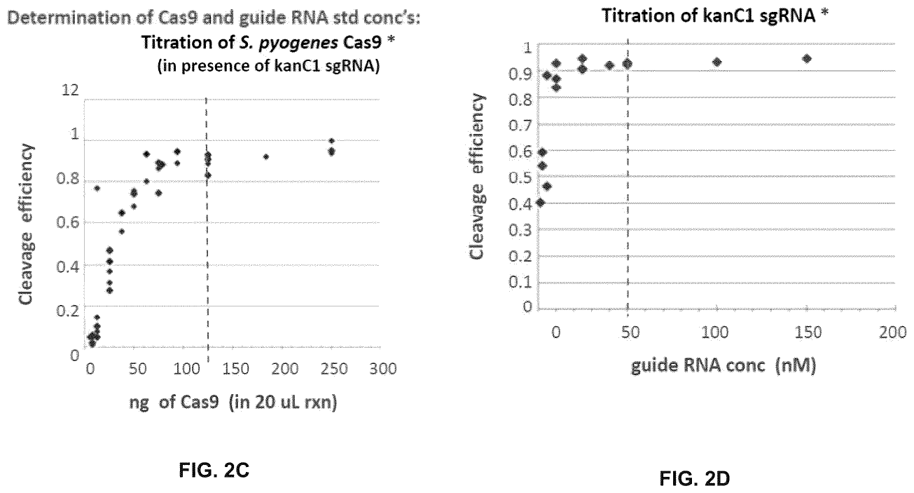

Shown in FIG. 1 is a diagram of CRISPR-Cas9-mediated sequence-specific cleavage of DNA. The guide RNA is depicted as sgRNA with an exemplary 20-nucleotide (20-nt) guide sequence (other guide sequences may be, for example, from about 15 to about 30 nts in length) within the 5′ domain, an internally positioned base-paired stem, and a 3′ domain. The guide sequence is complementary to an exemplary 20-nt target sequence in a DNA target. The stem corresponds to a repeat sequence in crRNA and is complementary to a sequence in the tracrRNA. The 3′ domain of the guide RNA corresponds to the 3′ domain of the tracrRNA that binds a Cas9 nuclease. The Cas9: guide RNA complex binds and cleaves a target DNA sequence or protospacer directly upstream of a PAM sequence recognized by Cas9. In FIG. 1, a 3-nt PAM sequence is exemplified; however other PAM sequences, including 4-nt and 5-nt PAM sequences are known. In the system exemplified in FIG. 1, both strands of the target sequence in DNA are cleaved by Cas9 at the sites indicated by arrows.

### III. Guide RNAs

In at least one aspect, the present invention comprises a chemically modified guide RNA that has guide RNA functionality. A guide RNA that comprises any nucleotide other than the four canonical ribonucleotides, namely A. C. G. and U, whether unnatural or natural (e.g., a pseudouridine, inosine or a deoxynucleotide), is a chemically modified guide RNA. Likewise a guide RNA that comprises any backbone or internucleotide linkage other than a natural phosphodiester internucleotide linkage possesses a chemical modification and therefore is a chemically modified guide RNA. In certain embodiments, the retained functionality includes binding a Cas protein. In certain embodiments, the retained functionality includes binding a target polynucleotide. In certain embodiments, the retained functionality includes targeting a Cas protein or a gRNA: Cas protein complex to a target polynucleotide. In certain embodiments, the retained functionality includes nicking a target polynucleotide by a gRNA: Cas protein complex. In certain embodiments, the retained functionality includes cleaving a target polynucleotide by a gRNA: Cas protein complex. In certain embodiments, the retained functionality is any other known function of a guide RNA in a CRISPR-Cas system with a Cas protein, including an artificial CRISPR-Cas system with an engineered Cas protein. In certain embodiments, the retained functionality is any other function of a natural guide RNA.

### A. Exemplary Modifications

In certain embodiments, a nucleotide sugar modification incorporated into the guide RNA is selected from the group consisting of 2′-O—C1-4alkyl such as 2′-O-methyl (2′-OMe), 2′-deoxy(2′—H), 2′-O—C1-3alkyl-O—C1-3alkyl such as 2′-methoxyethyl (“2′-MOE”), 2′-fluoro (“2′-F”), 2′-amino (“2′—NH2”), 2′-arabinosyl (“2′-arabino”) nucleotide, 2′-F-arabinosyl (“2′-F-arabino”) nucleotide, 2′-locked nucleic acid (“LNA”) nucleotide, 2′-unlocked nucleic acid (“ULNA”) nucleotide, a sugar in L form (“L-sugar”), and 4′-thioribosyl nucleotide. In certain embodiments, an internucleotide linkage modification incorporated into the guide RNA is selected from the group consisting of: phosphorothioate “P(S)” (P(S)), phosphonocarboxylate (P(CH2)ₙCOOR) such as phosphonoacetate “PACE” (P(CH2COO—)), thiophosphonocarboxylate ((S)P(CH2)ₙCOOR) such as thiophosphonoacetate “thioPACE” ((S)P(CH2COO—)), alkylphosphonate (P(C1-3alkyl) such as methylphosphonate —P(CH3), boranophosphate (P(BH3)), and phosphorodithioate (P(S)2).

In certain embodiments, a nucleobase (“base”) modification incorporated into the guide RNA is selected from the group consisting of: 2-thiouracil (“2-thioU”), 2-thiocytosine (“2-thioC”), 4-thiouracil (“4-thioU”), 6-thioguanine (“6-thioG”), 2-aminoadenine (“2-aminoA”), 2-aminopurine, pseudouracil, hypoxanthine, 7-deazaguanine, 7-deaza-8-azaguanine, 7-deazaadenine, 7-deaza-8-azaadenine, 5-methylcytosine (“5-methylC”), 5-methyluracil (“5-methylU”), 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil (“5-allylU”), 5-allylcytisine (“5-allylC”), 5-aminoallyluracil (“5-aminoallylU”), 5-aminoallyl-cytosine (“5-aminoallylC”), an abasic nucleotide, Z base, P base. Unstructured Nucleic Acid (“UNA”), isoguanine (“isoG”), isocytosine (“isoC”) [as described in “Enzymatic Incorporation of a New Base pair into DNA and RNA Extends the Genetic Alphabet.” Piccirilli, J. A.; Krauch, T.; Moroney, S. E.; Benner, S. A. (1990) Nature, 343, 33], 5-methyl-2-pyrimidine [as described in Rappaport, H. P. (1993) Biochemistry, 32, 3047], x(A,G,C,T) and y(A,G,C,T).

In certain embodiments, one or more isotopic modifications are introduced on the nucleotide sugar, the nucleobase, the phosphodiester linkage and/or the nucleotide phosphates. Such modifications include nucleotides comprising one or more 15N, 13C, 14C, Deuterium, 3H, 32P, 125I, 131I atoms or other atoms or elements used as tracers.

In certain embodiments, an “end” modification incorporated into the guide RNA is selected from the group consisting of: PEG (polyethyleneglycol), hydrocarbon linkers (including: heteroatom (O,S,N)-substituted hydrocarbon spacers; halo-substituted hydrocarbon spacers; keto-, carboxyl-, amido-, thionyl-, carbamoyl-, thionocarbamoyl-containing hydrocarbon spacers), spermine linkers, dyes including fluorescent dyes (for example fluoresceins, rhodamines, cyanines) attached to linkers such as for example 6-fluorescein-hexyl, quenchers (for example dabcyl, BHQ) and other labels (for example biotin, digoxigenin, acridine, streptavidin, avidin, peptides and/or proteins). In certain embodiments, an “end” modification comprises a conjugation (or ligation) of the guide RNA to another molecule comprising an oligonucleotide (comprising deoxynucleotides and/or ribonucleotides), a peptide, a protein, a sugar, an oligosaccharide, a steroid, a lipid, a folic acid, a vitamin and/or other molecule. In certain embodiments, an “end” modification incorporated into the guide RNA is located internally in the guide RNA sequence via a linker such as for example 2-(4-butylamidofluorescein) propane-1,3-diol bis(phosphodiester) linker (depicted below), which is incorporated as a phosphodiester linkage and can be incorporated anywhere between two nucleotides in the guide RNA.

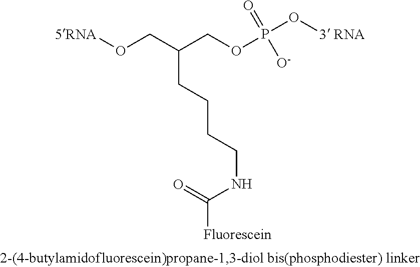

Other linkers include for example by way of illustration, but are not limited to:

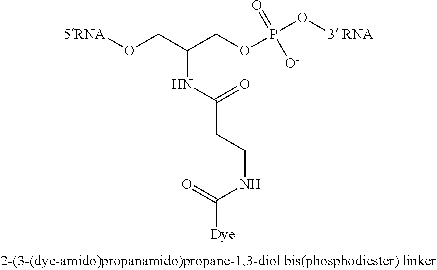

In certain embodiments, the end modification comprises a terminal functional group such as an amine, a thiol (or sulfhydryl), a hydroxyl, a carboxyl, carbonyl, thionyl, thiocarbonyl, a carbamoyl, a thiocarbamoyl, a phosphoryl, an alkene, an alkyne, an halogen or a functional group-terminated linker, either of which can be subsequently conjugated to a desired moiety, for example a fluorescent dye or a non-fluorescent label or tag or any other molecule such as for example an oligonucleotide (comprising deoxynucleotides and/or ribonucleotides, including an aptamer), an amino acid, a peptide, a protein, a sugar, an oligosaccharide, a steroid, a lipid, a folic acid, a vitamin. The conjugation employs standard chemistry well-known in the art, including but not limited to coupling via N-hydroxysuccinimide, isothiocyanate, DCC (or DCI), and/or any other standard method as described in “Bioconjugate Techniques” by Greg T. Hermanson, Publisher Elsevier Science, 3rd ed. (2013), the contents of which are incorporated herein by reference in their entireties.

In certain embodiments, the label or dye is attached or conjugated to a modified nucleotide in the gRNA. The conjugation of a fluorescent dye or other moiety such as a non-fluorescent label or tag (for example biotin, avidin, streptavidin, or moiety containing an isotopic label such as 15N,13C, 14C, Deuterium, 3H, 32P, 125I and the like) or any other molecule such as for example an oligonucleotide (comprising deoxynucleotides and/or ribonucleotides including an aptamer), an amino acid, a peptide, a protein, a sugar, an oligosaccharide, a steroid, a lipid, a folic acid, a vitamin or other molecule can be effectuated using the so-called “click” chemistry or the so-called “squarate” conjugation chemistry. The “click” chemistry refers to the [3+2] cycloaddition of an alkyne moiety with an azide moiety, leading to a triazolo linkage between the two moieties as shown in the following scheme:

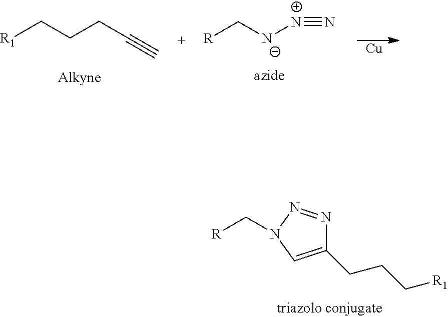

as described for example in El-Sagheer, A. H. and Brown, T. “Click chemistry with DNA”, Chem. Soc. Rev., 2010, 39, 1388-1405 and Mojibul, H. M. and XiaoHua, P., DNA-associated click chemistry, Sci. China Chem., 2014, 57:2, 215-31, the contents of which are hereby incorporated by reference in their entirety.

In certain embodiments, the conjugation can be effectuated by alternative cycloaddition such as Diels-Alder [4+2] cycloaddition of □-conjugated diene moiety with an alkene moiety.

The “squarate” conjugation chemistry links two moieties each having an amine via a squarate derivative to result in a squarate conjugate that contains a squarate moiety (see e.g., Tietze et al. (1991) Chem. Ber., 124, 1215-21, the contents of which are hereby incorporated by reference in their entirety). For example, a fluorescein containing a linker amine is conjugated to an oligoribonucleotide containing an amine through a squarate linker as described in the scheme below. An example of the squarate linker is depicted in the following scheme:

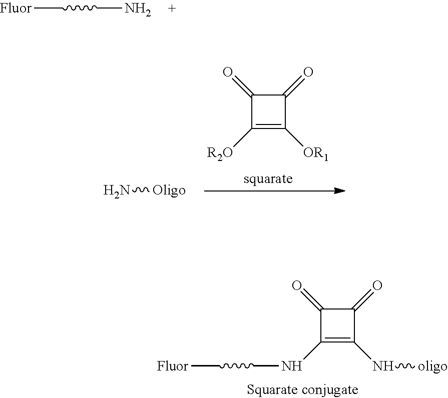

In certain embodiments, a chemical modification incorporated into the guide RNA is selected from the group consisting of 2′-O—C1-4alkyl, 2′-H, 2′-O—C1-3alkyl-O—C1-3alkyl, 2′-F, 2′—NH2, 2′-arabino, 2′-F-arabino, 4′-thioribosyl, 2-thioU, 2-thioC, 4-thioU, 6-thioG, 2-aminoA, 2-aminopurine, pseudouracil, hypoxanthine, 7-deazaguanine, 7-deaza-8-azaguanine, 7-deazaadenine, 7-deaza-8-azaadenine, 5-methylC, 5-methylU, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allylU, 5-allylC, 5-aminoallyl-uracil, 5-aminoallyl-cytosine, an abasic nucleotide (“abN”), Z, P, UNA, isoC, isoG, 5-methyl-pyrimidine, x(A,G,C,T) and y(A,G,C,T), a phosphorothioate internucleotide linkage, a phosphonoacetate internucleotide linkage, a thiophosphonoacetate internucleotide linkage, a methylphosphonate internucleotide linkage, a boranophosphate internucleotide linkage, a phosphorodithioate internucleotide linkage, 4′-thioribosyl nucleotide, a locked nucleic acid (“LNA”) nucleotide, an unlocked nucleic acid (“ULNA”) nucleotide, an alkyl spacer, a heteroalkyl (N, O, S) spacer, a 5′- and/or 3′-alkyl terminated nucleotide, a Unicap, a 5′-terminal cap known from nature, an xRNA base (analogous to “×DNA” base), an yRNA base (analogous to “yDNA” base), a PEG substituent, or a conjugated linker to a dye or non-fluorescent label (or tag) or other moiety as described above. Exemplary modified nucleotides are also depicted in Table 2.

TABLE 2
Exemplary modified nucleotides contained in a synthetic guide sequence.

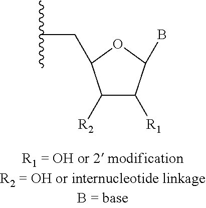

| # | R₁ | R₂ | B |
|---|---|---|---|
| A1 | OH | OH | uridine |
| A2 | OMe | OH | uridine |
| A3 | F | OH | uridine |
| A4 | Cl | OH | uridine |
| A5 | Br | OH | uridine |
| A6 | I | OH | uridine |
| A7 | NH₂ | OH | uridine |
| A8 | H | OH | uridine |
| A9 | OH | phosphodiester | uridine |
| A10 | OMe | phosphodiester | uridine |
| A11 | F | phosphodiester | uridine |
| A12 | Cl | phosphodiester | uridine |
| A13 | Br | phosphodiester | uridine |
| A14 | I | phosphodiester | uridine |
| A15 | NH₂ | phosphodiester | uridine |
| A16 | H | phosphodiester | uridine |
| A17 | OH | phosphonoacetate | uridine |
| A18 | OMe | phosphonoacetate | uridine |
| A19 | F | phosphonoacetate | uridine |
| A20 | Cl | phosphonoacetate | uridine |
| A21 | Br | phosphonoacetate | uridine |
| A22 | I | phosphonoacetate | uridine |
| A23 | NH₂ | phosphonoacetate | uridine |
| A24 | H | phosphonoacetate | uridine |
| A25 | OH | thiophosphonoacetate | uridine |
| A26 | OMe | thiophosphonoacetate | uridine |
| A27 | F | thiophosphonoacetate | uridine |
| A28 | Cl | thiophosphonoacetate | uridine |
| A29 | Br | thiophosphonoacetate | uridine |
| A30 | I | thiophosphonoacetate | uridine |
| A31 | NH₂ | thiophosphonoacetate | uridine |
| A32 | H | thiophosphonoacetate | uridine |
| A33 | OH | phosphorothioate | uridine |
| A34 | OMe | phosphorothioate | uridine |
| A35 | F | phosphorothioate | uridine |
| A36 | Cl | phosphorothioate | uridine |
| A37 | Br | phosphorothioate | uridine |
| A38 | I | phosphorothioate | uridine |
| A39 | NH₂ | phosphorothioate | uridine |
| A40 | H | phosphorothioate | uridine |
| A41 | OH | phosphorodithioate | uridine |
| A42 | OMe | phosphorodithioate | uridine |
| A43 | F | phosphorodithioate | uridine |
| A44 | Cl | phosphorodithioate | uridine |
| A45 | Br | phosphorodithioate | uridine |
| A46 | I | phosphorodithioate | uridine |
| A47 | NH₂ | phosphorodithioate | uridine |
| A48 | H | phosphorodithioate | uridine |
| A49 | OH | methylphosphonate | uridine |
| A50 | OMe | methylphosphonate | uridine |
| A51 | F | methylphosphonate | uridine |
| A52 | Cl | methylphosphonate | uridine |
| A53 | Br | methylphosphonate | uridine |
| A54 | I | methylphosphonate | uridine |
| A55 | NH₂ | methylphosphonate | uridine |
| A56 | H | methylphosphonate | uridine |
| A57 | OH | boranophosphonate | uridine |
| A58 | OMe | boranophosphonate | uridine |
| A59 | F | boranophosphonate | uridine |
| A60 | Cl | boranophosphonate | uridine |
| A61 | Br | boranophosphonate | uridine |
| A62 | I | boranophosphonate | uridine |
| A63 | NH₂ | boranophosphonate | uridine |
| A64 | H | boranophosphonate | uridine |
| B1 | OH | OH | adenosine |
| B2 | OMe | OH | adenosine |
| B3 | F | OH | adenosine |
| B4 | Cl | OH | adenosine |
| B5 | Br | OH | adenosine |
| B6 | I | OH | adenosine |
| B7 | NH₂ | OH | adenosine |
| B8 | H | OH | adenosine |
| B9 | OH | phosphodiester | adenosine |
| B10 | OMe | phosphodiester | adenosine |
| B11 | F | phosphodiester | adenosine |
| B12 | Cl | phosphodiester | adenosine |
| B13 | Br | phosphodiester | adenosine |
| B14 | I | phosphodiester | adenosine |
| B15 | NH₂ | phosphodiester | adenosine |
| B16 | H | phosphodiester | adenosine |
| B17 | OH | phosphonoacetate | adenosine |
| B18 | OMe | phosphonoacetate | adenosine |
| B19 | F | phosphonoacetate | adenosine |
| B20 | Cl | phosphonoacetate | adenosine |
| B21 | Br | phosphonoacetate | adenosine |
| B22 | I | phosphonoacetate | adenosine |
| B23 | NH₂ | phosphonoacetate | adenosine |
| B24 | H | phosphonoacetate | adenosine |
| B25 | OH | thiophosphonoacetate | adenosine |
| B26 | OMe | thiophosphonoacetate | adenosine |
| B27 | F | thiophosphonoacetate | adenosine |
| B28 | Cl | thiophosphonoacetate | adenosine |
| B29 | Br | thiophosphonoacetate | adenosine |
| B30 | I | thiophosphonoacetate | adenosine |
| B31 | NH₂ | thiophosphonoacetate | adenosine |
| B32 | H | thiophosphonoacetate | adenosine |
| B33 | OH | phosphorothioate | adenosine |
| B34 | OMe | phosphorothioate | adenosine |
| B35 | F | phosphorothioate | adenosine |
| B36 | Cl | phosphorothioate | adenosine |
| B37 | Br | phosphorothioate | adenosine |
| B38 | I | phosphorothioate | adenosine |
| B39 | NH₂ | phosphorothioate | adenosine |
| B40 | H | phosphorothioate | adenosine |
| B41 | OH | phosphorodithioate | adenosine |
| B42 | OMe | phosphorodithioate | adenosine |
| B43 | F | phosphorodithioate | adenosine |
| B44 | Cl | phosphorodithioate | adenosine |
| B45 | Br | phosphorodithioate | adenosine |
| B46 | I | phosphorodithioate | adenosine |
| B47 | NH₂ | phosphorodithioate | adenosine |
| B48 | H | phosphorodithioate | adenosine |
| B49 | OH | methylphosphonate | adenosine |
| B50 | OMe | methylphosphonate | adenosine |
| B51 | F | methylphosphonate | adenosine |
| B52 | Cl | methylphosphonate | adenosine |
| B53 | Br | methylphosphonate | adenosine |
| B54 | I | methylphosphonate | adenosine |
| B55 | NH₂ | methylphosphonate | adenosine |
| B56 | H | methylphosphonate | adenosine |
| B57 | OH | boranophosphonate | adenosine |
| B58 | OMe | boranophosphonate | adenosine |
| B59 | F | boranophosphonate | adenosine |
| B60 | Cl | boranophosphonate | adenosine |
| B61 | Br | boranophosphonate | adenosine |
| B62 | I | boranophosphonate | adenosine |
| B63 | NH₂ | boranophosphonate | adenosine |
| B64 | H | boranophosphonate | adenosine |
| C1 | OH | OH | cytidine |
| C2 | OMe | OH | cytidine |
| C3 | F | OH | cytidine |
| C4 | Cl | OH | cytidine |
| C5 | Br | OH | cytidine |
| C6 | I | OH | cytidine |
| C7 | NH₂ | OH | cytidine |
| C8 | H | OH | cytidine |
| C9 | OH | phosphodiester | cytidine |
| C10 | OMe | phosphodiester | cytidine |
| C11 | F | phosphodiester | cytidine |
| C12 | Cl | phosphodiester | cytidine |
| C13 | Br | phosphodiester | cytidine |
| C14 | I | phosphodiester | cytidine |
| C15 | NH₂ | phosphodiester | cytidine |
| C16 | H | phosphodiester | cytidine |
| C17 | OH | phosphonoacetate | cytidine |
| C18 | OMe | phosphonoacetate | cytidine |
| C19 | F | phosphonoacetate | cytidine |
| C20 | Cl | phosphonoacetate | cytidine |
| C21 | Br | phosphonoacetate | cytidine |
| C22 | I | phosphonoacetate | cytidine |
| C23 | NH₂ | phosphonoacetate | cytidine |
| C24 | H | phosphonoacetate | cytidine |
| C25 | OH | thiophosphonoacetate | cytidine |
| C26 | OMe | thiophosphonoacetate | cytidine |
| C27 | F | thiophosphonoacetate | cytidine |
| C28 | Cl | thiophosphonoacetate | cytidine |
| C29 | Br | thiophosphonoacetate | cytidine |
| C30 | I | thiophosphonoacetate | cytidine |
| C31 | NH₂ | thiophosphonoacetate | cytidine |
| C32 | H | thiophosphonoacetate | cytidine |
| C33 | OH | phosphorothioate | cytidine |
| C34 | OMe | phosphorothioate | cytidine |
| C35 | F | phosphorothioate | cytidine |
| C36 | Cl | phosphorothioate | cytidine |
| C37 | Br | phosphorothioate | cytidine |
| C38 | I | phosphorothioate | cytidine |
| C39 | NH₂ | phosphorothioate | cytidine |
| C40 | H | phosphorothioate | cytidine |
| C41 | OH | phosphorodithioate | cytidine |
| C42 | OMe | phosphorodithioate | cytidine |
| C43 | F | phosphorodithioate | cytidine |
| C44 | Cl | phosphorodithioate | cytidine |
| C45 | Br | phosphorodithioate | cytidine |
| C46 | I | phosphorodithioate | cytidine |
| C47 | NH₂ | phosphorodithioate | cytidine |
| C48 | H | phosphorodithioate | cytidine |
| C49 | OH | methylphosphonate | cytidine |
| C50 | OMe | methylphosphonate | cytidine |
| C5 | F | methylphosphonate | cytidine |
| C52 | Cl | methylphosphonate | cytidine |
| C53 | Br | methylphosphonate | cytidine |
| C54 | I | methylphosphonate | cytidine |
| C55 | NH₂ | methylphosphonate | cytidine |
| C56 | H | methylphosphonate | cytidine |
| C57 | OH | boranophosphonate | cytidine |
| C58 | OMe | boranophosphonate | cytidine |
| C59 | F | boranophosphonate | cytidine |
| C60 | Cl | boranophosphonate | cytidine |
| C61 | Br | boranophosphonate | cytidine |
| C62 | I | boranophosphonate | cytidine |
| C63 | NH₂ | boranophosphonate | cytidine |
| C64 | H | boranophosphonate | cytidine |
| D1 | OH | OH | guanosine |
| D2 | OMe | OH | guanosine |
| D3 | F | OH | guanosine |
| D4 | Cl | OH | guanosine |
| D5 | Br | OH | guanosine |
| D6 | I | OH | guanosine |
| D7 | NH₂ | OH | guanosine |
| D8 | H | OH | guanosine |
| D9 | OH | phosphodiester | guanosine |
| D10 | OMe | phosphodiester | guanosine |
| D11 | F | phosphodiester | guanosine |
| D12 | Cl | phosphodiester | guanosine |
| D13 | Br | phosphodiester | guanosine |
| D14 | I | phosphodiester | guanosine |
| D15 | NH₂ | phosphodiester | guanosine |
| D16 | H | phosphodiester | guanosine |
| D17 | OH | phosphonoacetate | guanosine |
| D18 | OMe | phosphonoacetate | guanosine |
| D19 | F | phosphonoacetate | guanosine |
| D20 | Cl | phosphonoacetate | guanosine |
| D21 | Br | phosphonoacetate | guanosine |
| D22 | I | phosphonoacetate | guanosine |
| D23 | NH₂ | phosphonoacetate | guanosine |
| D24 | H | phosphonoacetate | guanosine |
| D25 | OH | thiophosphonoacetate | guanosine |
| D26 | OMe | thiophosphonoacetate | guanosine |
| D27 | F | thiophosphonoacetate | guanosine |
| D28 | Cl | thiophosphonoacetate | guanosine |
| D29 | Br | thiophosphonoacetate | guanosine |
| D30 | I | thiophosphonoacetate | guanosine |
| D31 | NH₂ | thiophosphonoacetate | guanosine |
| D32 | H | thiophosphonoacetate | guanosine |
| D33 | OH | phosphorothioate | guanosine |
| D34 | OMe | phosphorothioate | guanosine |
| D35 | F | phosphorothioate | guanosine |
| D36 | Cl | phosphorothioate | guanosine |
| D37 | Br | phosphorothioate | guanosine |
| D38 | I | phosphorothioate | guanosine |
| D39 | NH₂ | phosphorothioate | guanosine |
| D40 | H | phosphorothioate | guanosine |
| D41 | OH | phosphorodithioate | guanosine |
| D42 | OMe | phosphorodithioate | guanosine |
| D43 | F | phosphorodithioate | guanosine |
| D44 | Cl | phosphorodithioate | guanosine |
| D45 | Br | phosphorodithioate | guanosine |
| D46 | I | phosphorodithioate | guanosine |
| D47 | NH₂ | phosphorodithioate | guanosine |
| D48 | H | phosphorodithioate | guanosine |
| D49 | OH | methylphosphonate | guanosine |
| D50 | OMe | methylphosphonate | guanosine |
| D51 | F | methylphosphonate | guanosine |
| D52 | Cl | methylphosphonate | guanosine |
| D53 | Br | methylphosphonate | guanosine |
| D54 | I | methylphosphonate | guanosine |
| D55 | NH₂ | methylphosphonate | guanosine |
| D56 | H | methylphosphonate | guanosine |
| D57 | OH | boranophosphonate | guanosine |
| D58 | OMe | boranophosphonate | guanosine |
| D59 | F | boranophosphonate | guanosine |
| D60 | Cl | boranophosphonate | guanosine |
| D61 | Br | boranophosphonate | guanosine |
| D62 | I | boranophosphonate | guanosine |
| D63 | NH₂ | boranophosphonate | guanosine |
| D64 | H | boranophosphonate | guanosine |
| E1 | OH | OH | 2-thiouridine |
| E2 | OMe | OH | 2-thiouridine |
| E3 | F | OH | 2-thiouridine |
| E4 | Cl | OH | 2-thiouridine |
| E5 | Br | OH | 2-thiouridine |
| E6 | I | OH | 2-thiouridine |
| E7 | NH₂ | OH | 2-thiouridine |
| E8 | H | OH | 2-thiouridine |
| E9 | OH | phosphodiester | 2-thiouridine |
| E10 | OMe | phosphodiester | 2-thiouridine |
| E11 | F | phosphodiester | 2-thiouridine |
| E12 | Cl | phosphodiester | 2-thiouridine |
| E13 | Br | phosphodiester | 2-thiouridine |
| E14 | I | phosphodiester | 2-thiouridine |
| E15 | NH₂ | phosphodiester | 2-thiouridine |
| E16 | H | phosphodiester | 2-thiouridine |
| E17 | OH | phosphonoacetate | 2-thiouridine |
| E18 | OMe | phosphonoacetate | 2-thiouridine |
| E19 | F | phosphonoacetate | 2-thiouridine |
| E20 | Cl | phosphonoacetate | 2-thiouridine |
| E21 | Br | phosphonoacetate | 2-thiouridine |
| E22 | I | phosphonoacetate | 2-thiouridine |
| E23 | NH₂ | phosphonoacetate | 2-thiouridine |
| E24 | H | phosphonoacetate | 2-thiouridine |
| E25 | OH | thiophosphonoacetate | 2-thiouridine |
| E26 | OMe | thiophosphonoacetate | 2-thiouridine |
| E27 | F | thiophosphonoacetate | 2-thiouridine |
| E28 | Cl | thiophosphonoacetate | 2-thiouridine |
| E29 | Br | thiophosphonoacetate | 2-thiouridine |
| E30 | I | thiophosphonoacetate | 2-thiouridine |
| E31 | NH₂ | thiophosphonoacetate | 2-thiouridine |
| E32 | H | thiophosphonoacetate | 2-thiouridine |
| E33 | OH | phosphorothioate | 2-thiouridine |
| E34 | OMe | phosphorothioate | 2-thiouridine |
| E35 | F | phosphorothioate | 2-thiouridine |
| E36 | Cl | phosphorothioate | 2-thiouridine |
| E37 | Br | phosphorothioate | 2-thiouridine |
| E38 | I | phosphorothioate | 2-thiouridine |
| E39 | NH₂ | phosphorothioate | 2-thiouridine |
| E40 | H | phosphorothioate | 2-thiouridine |
| E41 | OH | phosphorodithioate | 2-thiouridine |
| E42 | OMe | phosphorodithioate | 2-thiouridine |
| E43 | F | phosphorodithioate | 2-thiouridine |
| E44 | Cl | phosphorodithioate | 2-thiouridine |
| E45 | Br | phosphorodithioate | 2-thiouridine |
| E46 | I | phosphorodithioate | 2-thiouridine |
| E47 | NH₂ | phosphorodithioate | 2-thiouridine |
| E48 | H | phosphorodithioate | 2-thiouridine |
| E49 | OH | methylphosphonate | 2-thiouridine |
| E50 | OMe | methylphosphonate | 2-thiouridine |
| E51 | F | methylphosphonate | 2-thiouridine |
| E52 | Cl | methylphosphonate | 2-thiouridine |
| E53 | Br | methylphosphonate | 2-thiouridine |
| E54 | I | methylphosphonate | 2-thiouridine |
| E55 | NH₂ | methylphosphonate | 2-thiouridine |
| E56 | H | methylphosphonate | 2-thiouridine |
| E57 | OH | boranophosphonate | 2-thiouridine |
| E58 | OMe | boranophosphonate | 2-thiouridine |
| E59 | F | boranophosphonate | 2-thiouridine |
| E60 | Cl | boranophosphonate | 2-thiouridine |
| E61 | Br | boranophosphonate | 2-thiouridine |
| E62 | I | boranophosphonate | 2-thiouridine |
| E63 | NH₂ | boranophosphonate | 2-thiouridine |
| E64 | H | boranophosphonate | 2-thiouridine |
| F1 | OH | OH | 4-thiouridine |
| F2 | OMe | OH | 4-thiouridine |
| F3 | F | OH | 4-thiouridine |
| F4 | Cl | OH | 4-thiouridine |
| F5 | Br | OH | 4-thiouridine |
| F6 | I | OH | 4-thiouridine |
| F7 | NH₂ | OH | 4-thiouridine |
| F8 | H | OH | 4-thiouridine |
| F9 | OH | phosphodiester | 4-thiouridine |
| F10 | OMe | phosphodiester | 4-thiouridine |
| F11 | F | phosphodiester | 4-thiouridine |
| F12 | Cl | phosphodiester | 4-thiouridine |
| F13 | Br | phosphodiester | 4-thiouridine |
| F14 | I | phosphodiester | 4-thiouridine |
| F15 | NH₂ | phosphodiester | 4-thiouridine |
| F16 | H | phosphodiester | 4-thiouridine |
| F17 | OH | phosphonoacetate | 4-thiouridine |
| F18 | OMe | phosphonoacetate | 4-thiouridine |
| F19 | F | phosphonoacetate | 4-thiouridine |
| F20 | Cl | phosphonoacetate | 4-thiouridine |
| F21 | Br | phosphonoacetate | 4-thiouridine |
| F22 | I | phosphonoacetate | 4-thiouridine |
| F23 | NH₂ | phosphonoacetate | 4-thiouridine |
| F24 | H | phosphonoacetate | 4-thiouridine |
| F25 | OH | thiophosphonoacetate | 4-thiouridine |
| F26 | OMe | thiophosphonoacetate | 4-thiouridine |
| F27 | F | thiophosphonoacetate | 4-thiouridine |
| F28 | Cl | thiophosphonoacetate | 4-thiouridine |
| F29 | Br | thiophosphonoacetate | 4-thiouridine |
| F30 | I | thiophosphonoacetate | 4-thiouridine |
| F31 | NH₂ | thiophosphonoacetate | 4-thiouridine |
| F32 | H | thiophosphonoacetate | 4-thiouridine |
| F33 | OH | phosphorothioate | 4-thiouridine |
| F34 | OMe | phosphorothioate | 4-thiouridine |
| F35 | F | phosphorothioate | 4-thiouridine |
| F36 | Cl | phosphorothioate | 4-thiouridine |
| F37 | Br | phosphorothioate | 4-thiouridine |
| F38 | I | phosphorothioate | 4-thiouridine |
| F39 | NH₂ | phosphorothioate | 4-thiouridine |
| F40 | H | phosphorothioate | 4-thiouridine |
| F41 | OH | phosphorodithioate | 4-thiouridine |
| F42 | OMe | phosphorodithioate | 4-thiouridine |
| F43 | F | phosphorodithioate | 4-thiouridine |
| F44 | Cl | phosphorodithioate | 4-thiouridine |
| F45 | Br | phosphorodithioate | 4-thiouridine |
| F46 | I | phosphorodithioate | 4-thiouridine |
| F47 | NH₂ | phosphorodithioate | 4-thiouridine |
| F48 | H | phosphorodithioate | 4-thiouridine |
| F49 | OH | methylphosphonate | 4-thiouridine |
| F50 | OMe | methylphosphonate | 4-thiouridine |
| F51 | F | methylphosphonate | 4-thiouridine |
| F52 | Cl | methylphosphonate | 4-thiouridine |
| F53 | Br | methylphosphonate | 4-thiouridine |
| F54 | I | methylphosphonate | 4-thiouridine |
| F55 | NH₂ | methylphosphonate | 4-thiouridine |
| F56 | H | methylphosphonate | 4-thiouridine |
| F57 | OH | boranophosphonate | 4-thiouridine |
| F58 | OMe | boranophosphonate | 4-thiouridine |
| F59 | F | boranophosphonate | 4-thiouridine |
| F60 | Cl | boranophosphonate | 4-thiouridine |
| F61 | Br | boranophosphonate | 4-thiouridine |
| F62 | I | boranophosphonate | 4-thiouridine |
| F63 | NH₂ | boranophosphonate | 4-thiouridine |
| F64 | H | boranophosphonate | 4-thiouridine |
| G1 | OH | OH | 2-aminoadenosine |
| G2 | OMe | OH | 2-aminoadenosine |
| G3 | F | OH | 2-aminoadenosine |
| G4 | Cl | OH | 2-aminoadenosine |
| G5 | Br | OH | 2-aminoadenosine |
| G6 | I | OH | 2-aminoadenosine |
| G7 | NH₂ | OH | 2-aminoadenosine |
| G8 | H | OH | 2-aminoadenosine |
| G9 | OH | phosphodiester | 2-aminoadenosine |
| G10 | OMe | phosphodiester | 2-aminoadenosine |
| G11 | F | phosphodiester | 2-aminoadenosine |
| G12 | Cl | phosphodiester | 2-aminoadenosine |
| G13 | Br | phosphodiester | 2-aminoadenosine |
| G14 | I | phosphodiester | 2-aminoadenosine |
| G15 | NH₂ | phosphodiester | 2-aminoadenosine |
| G16 | H | phosphodiester | 2-aminoadenosine |
| G17 | OH | phosphonoacetate | 2-aminoadenosine |
| G18 | OMe | phosphonoacetate | 2-aminoadenosine |
| G19 | F | phosphonoacetate | 2-aminoadenosine |
| G20 | Cl | phosphonoacetate | 2-aminoadenosine |
| G21 | Br | phosphonoacetate | 2-aminoadenosine |
| G22 | I | phosphonoacetate | 2-aminoadenosine |
| G23 | NH₂ | phosphonoacetate | 2-aminoadenosine |
| G24 | H | phosphonoacetate | 2-aminoadenosine |
| G25 | OH | thiophosphonoacetate | 2-aminoadenosine |
| G26 | OMe | thiophosphonoacetate | 2-aminoadenosine |
| G27 | F | thiophosphonoacetate | 2-aminoadenosine |
| G28 | Cl | thiophosphonoacetate | 2-aminoadenosine |
| G29 | Br | thiophosphonoacetate | 2-aminoadenosine |
| G30 | I | thiophosphonoacetate | 2-aminoadenosine |
| G31 | NH₂ | thiophosphonoacetate | 2-aminoadenosine |
| G32 | H | thiophosphonoacetate | 2-aminoadenosine |
| G33 | OH | phosphorothioate | 2-aminoadenosine |
| G34 | OMe | phosphorothioate | 2-aminoadenosine |
| G35 | F | phosphorothioate | 2-aminoadenosine |
| G36 | Cl | phosphorothioate | 2-aminoadenosine |
| G37 | Br | phosphorothioate | 2-aminoadenosine |
| G38 | I | phosphorothioate | 2-aminoadenosine |
| G39 | NH₂ | phosphorothioate | 2-aminoadenosine |
| G40 | H | phosphorothioate | 2-aminoadenosine |
| G41 | OH | phosphorodithioate | 2-aminoadenosine |
| G42 | OMe | phosphorodithioate | 2-aminoadenosine |
| G43 | F | phosphorodithioate | 2-aminoadenosine |
| G44 | Cl | phosphorodithioate | 2-aminoadenosine |
| G45 | Br | phosphorodithioate | 2-aminoadenosine |
| G46 | I | phosphorodithioate | 2-aminoadenosine |
| G47 | NH₂ | phosphorodithioate | 2-aminoadenosine |
| G48 | H | phosphorodithioate | 2-aminoadenosine |
| G49 | OH | methylphosphonate | 2-aminoadenosine |
| G50 | OMe | methylphosphonate | 2-aminoadenosine |
| G51 | F | methylphosphonate | 2-aminoadenosine |
| G52 | Cl | methylphosphonate | 2-aminoadenosine |
| G53 | Br | methylphosphonate | 2-aminoadenosine |
| G54 | I | methylphosphonate | 2-aminoadenosine |
| G55 | NH₂ | methylphosphonate | 2-aminoadenosine |
| G56 | H | methylphosphonate | 2-aminoadenosine |
| G57 | OH | boranophosphonate | 2-aminoadenosine |
| G58 | OMe | boranophosphonate | 2-aminoadenosine |
| G59 | F | boranophosphonate | 2-aminoadenosine |
| G60 | Cl | boranophosphonate | 2-aminoadenosine |
| G61 | Br | boranophosphonate | 2-aminoadenosine |
| G62 | I | boranophosphonate | 2-aminoadenosine |
| G63 | NH₂ | boranophosphonate | 2-aminoadenosine |
| G64 | H | boranophosphonate | 2-aminoadenosine |
| H1 | OH | OH | 7-deazaguanosine |
| H2 | OMe | OH | 7-deazaguanosine |
| H3 | F | OH | 7-deazaguanosine |
| H4 | Cl | OH | 7-deazaguanosine |
| H5 | Br | OH | 7-deazaguanosine |
| H6 | I | OH | 7-deazaguanosine |
| H7 | NH₂ | OH | 7-deazaguanosine |
| H8 | H | OH | 7-deazaguanosine |
| H9 | OH | phosphodiester | 7-deazaguanosine |
| H10 | OMe | phosphodiester | 7-deazaguanosine |
| H11 | F | phosphodiester | 7-deazaguanosine |
| H12 | Cl | phosphodiester | 7-deazaguanosine |
| H13 | Br | phosphodiester | 7-deazaguanosine |
| H14 | I | phosphodiester | 7-deazaguanosine |
| H15 | NH₂ | phosphodiester | 7-deazaguanosine |
| H16 | H | phosphodiester | 7-deazaguanosine |
| H17 | OH | phosphonoacetate | 7-deazaguanosine |
| H18 | OMe | phosphonoacetate | 7-deazaguanosine |
| H19 | F | phosphonoacetate | 7-deazaguanosine |
| H20 | Cl | phosphonoacetate | 7-deazaguanosine |
| H21 | Br | phosphonoacetate | 7-deazaguanosine |
| H22 | I | phosphonoacetate | 7-deazaguanosine |
| H23 | NH₂ | phosphonoacetate | 7-deazaguanosine |
| H24 | H | phosphonoacetate | 7-deazaguanosine |
| H25 | OH | thiophosphonoacetate | 7-deazaguanosine |
| H26 | OMe | thiophosphonoacetate | 7-deazaguanosine |
| H27 | F | thiophosphonoacetate | 7-deazaguanosine |
| H28 | Cl | thiophosphonoacetate | 7-deazaguanosine |
| H29 | Br | thiophosphonoacetate | 7-deazaguanosine |
| H30 | I | thiophosphonoacetate | 7-deazaguanosine |
| H31 | NH₂ | thiophosphonoacetate | 7-deazaguanosine |
| H32 | H | thiophosphonoacetate | 7-deazaguanosine |
| H33 | OH | phosphorothioate | 7-deazaguanosine |
| H34 | OMe | phosphorothioate | 7-deazaguanosine |
| H35 | F | phosphorothioate | 7-deazaguanosine |
| H36 | Cl | phosphorothioate | 7-deazaguanosine |
| H37 | Br | phosphorothioate | 7-deazaguanosine |
| H38 | I | phosphorothioate | 7-deazaguanosine |
| H39 | NH₂ | phosphorothioate | 7-deazaguanosine |
| H40 | H | phosphorothioate | 7-deazaguanosine |
| H41 | OH | phosphorodithioate | 7-deazaguanosine |
| H42 | OMe | phosphorodithioate | 7-deazaguanosine |
| H43 | F | phosphorodithioate | 7-deazaguanosine |
| H44 | Cl | phosphorodithioate | 7-deazaguanosine |
| H45 | Br | phosphorodithioate | 7-deazaguanosine |
| H46 | I | phosphorodithioate | 7-deazaguanosine |
| H47 | NH₂ | phosphorodithioate | 7-deazaguanosine |
| H48 | H | phosphorodithioate | 7-deazaguanosine |
| H49 | OH | methylphosphonate | 7-deazaguanosine |
| H50 | OMe | methylphosphonate | 7-deazaguanosine |
| H51 | F | methylphosphonate | 7-deazaguanosine |
| H52 | Cl | methylphosphonate | 7-deazaguanosine |
| H53 | Br | methylphosphonate | 7-deazaguanosine |
| H54 | I | methylphosphonate | 7-deazaguanosine |
| H55 | NH₂ | methylphosphonate | 7-deazaguanosine |
| H56 | H | methylphosphonate | 7-deazaguanosine |
| H57 | OH | boranophosphonate | 7-deazaguanosine |
| H58 | OMe | boranophosphonate | 7-deazaguanosine |
| H59 | F | boranophosphonate | 7-deazaguanosine |
| H60 | Cl | boranophosphonate | 7-deazaguanosine |
| H61 | Br | boranophosphonate | 7-deazaguanosine |
| H62 | I | boranophosphonate | 7-deazaguanosine |
| H63 | NH₂ | boranophosphonate | 7-deazaguanosine |
| H64 | H | boranophosphonate | 7-deazaguanosine |
| I1 | OH | OH | inosine |
| I2 | OMe | OH | inosine |
| I3 | F | OH | inosine |
| I4 | Cl | OH | inosine |
| I5 | Br | OH | inosine |
| I6 | I | OH | inosine |
| I7 | NH₂ | OH | inosine |
| I8 | H | OH | inosine |
| I9 | OH | phosphodiester | inosine |
| I10 | OMe | phosphodiester | inosine |
| I11 | F | phosphodiester | inosine |
| I12 | Cl | phosphodiester | inosine |
| I13 | Br | phosphodiester | inosine |
| I14 | I | phosphodiester | inosine |
| I15 | NH₂ | phosphodiester | inosine |
| I16 | H | phosphodiester | inosine |
| I17 | OH | phosphonoacetate | inosine |
| I18 | OMe | phosphonoacetate | inosine |
| I19 | F | phosphonoacetate | inosine |
| I20 | Cl | phosphonoacetate | inosine |
| I21 | Br | phosphonoacetate | inosine |
| I22 | I | phosphonoacetate | inosine |
| I23 | NH₂ | phosphonoacetate | inosine |
| I24 | H | phosphonoacetate | inosine |
| 125 | OH | thiophosphonoacetate | inosine |
| I26 | OMe | thiophosphonoacetate | inosine |
| I27 | F | thiophosphonoacetate | inosine |
| I28 | Cl | thiophosphonoacetate | inosine |
| I29 | Br | thiophosphonoacetate | inosine |
| I30 | I | thiophosphonoacetate | inosine |
| I31 | NH₂ | thiophosphonoacetate | inosine |
| I32 | H | thiophosphonoacetate | inosine |
| I33 | OH | phosphorothioate | inosine |
| I34 | OMe | phosphorothioate | inosine |
| I35 | F | phosphorothioate | inosine |
| I36 | Cl | phosphorothioate | inosine |
| I37 | Br | phosphorothioate | inosine |
| I38 | I | phosphorothioate | inosine |
| I39 | NH₂ | phosphorothioate | inosine |
| 140 | H | phosphorothioate | inosine |
| I41 | OH | phosphorodithioate | inosine |
| I42 | OMe | phosphorodithioate | inosine |
| I43 | F | phosphorodithioate | inosine |
| I44 | Cl | phosphorodithioate | inosine |
| I45 | Br | phosphorodithioate | inosine |
| I46 | I | phosphorodithioate | inosine |
| I47 | NH₂ | phosphorodithioate | inosine |
| I48 | H | phosphorodithioate | inosine |
| I49 | OH | methylphosphonate | inosine |
| I50 | OMe | methylphosphonate | inosine |
| I51 | F | methylphosphonate | inosine |
| I52 | Cl | methylphosphonate | inosine |
| I53 | Br | methylphosphonate | inosine |
| I54 | I | methylphosphonate | inosine |
| I55 | NH₂ | methylphosphonate | inosine |
| I56 | H | methylphosphonate | inosine |
| I57 | OH | boranophosphonate | inosine |
| I58 | OMe | boranophosphonate | inosine |
| I59 | F | boranophosphonate | inosine |
| I60 | Cl | boranophosphonate | inosine |
| I61 | Br | boranophosphonate | inosine |
| I62 | I | boranophosphonate | inosine |
| I63 | NH₂ | boranophosphonate | inosine |
| I64 | H | boranophosphonate | inosine |
| J1 | OH | OH | 5-methylcytidine |
| J2 | OMe | OH | 5-methylcytidine |
| J3 | F | OH | 5-methylcytidine |
| J4 | Cl | OH | 5-methylcytidine |
| J5 | Br | OH | 5-methylcytidine |
| J6 | I | OH | 5-methylcytidine |
| J7 | NH₂ | OH | 5-methylcytidine |
| J8 | H | OH | 5-methylcytidine |
| J9 | OH | phosphodiester | 5-methylcytidine |
| J10 | OMe | phosphodiester | 5-methylcytidine |
| J11 | F | phosphodiester | 5-methylcytidine |
| J12 | Cl | phosphodiester | 5-methylcytidine |
| J13 | Br | phosphodiester | 5-methylcytidine |
| J14 | I | phosphodiester | 5-methylcytidine |
| J15 | NH₂ | phosphodiester | 5-methylcytidine |
| J16 | H | phosphodiester | 5-methylcytidine |
| J17 | OH | phosphonoacetate | 5-methylcytidine |
| J18 | OMe | phosphonoacetate | 5-methylcytidine |
| J19 | F | phosphonoacetate | 5-methylcytidine |
| J20 | Cl | phosphonoacetate | 5-methylcytidine |
| J21 | Br | phosphonoacetate | 5-methylcytidine |
| J22 | I | phosphonoacetate | 5-methylcytidine |
| J23 | NH₂ | phosphonoacetate | 5-methylcytidine |
| J24 | H | phosphonoacetate | 5-methylcytidine |
| J25 | OH | thiophosphonoacetate | 5-methylcytidine |
| J26 | OMe | thiophosphonoacetate | 5-methylcytidine |
| J27 | F | thiophosphonoacetate | 5-methylcytidine |
| J28 | Cl | thiophosphonoacetate | 5-methylcytidine |
| J29 | Br | thiophosphonoacetate | 5-methylcytidine |
| J30 | I | thiophosphonoacetate | 5-methylcytidine |
| J31 | NH₂ | thiophosphonoacetate | 5-methylcytidine |
| J32 | H | thiophosphonoacetate | 5-methylcytidine |
| J33 | OH | phosphorothioate | 5-methylcytidine |
| J34 | OMe | phosphorothioate | 5-methylcytidine |
| J35 | F | phosphorothioate | 5-methylcytidine |
| J36 | Cl | phosphorothioate | 5-methylcytidine |
| J37 | Br | phosphorothioate | 5-methylcytidine |
| J38 | I | phosphorothioate | 5-methylcytidine |
| J39 | NH₂ | phosphorothioate | 5-methylcytidine |
| J40 | H | phosphorothioate | 5-methylcytidine |
| J41 | OH | phosphorodithioate | 5-methylcytidine |
| J42 | OMe | phosphorodithioate | 5-methylcytidine |
| J43 | F | phosphorodithioate | 5-methylcytidine |
| J44 | Cl | phosphorodithioate | 5-methylcytidine |
| J45 | Br | phosphorodithioate | 5-methylcytidine |
| J46 | I | phosphorodithioate | 5-methylcytidine |
| J47 | NH₂ | phosphorodithioate | 5-methylcytidine |
| J48 | H | phosphorodithioate | 5-methylcytidine |
| J49 | OH | methylphosphonate | 5-methylcytidine |
| J50 | OMe | methylphosphonate | 5-methylcytidine |
| J51 | F | methylphosphonate | 5-methylcytidine |
| J52 | Cl | methylphosphonate | 5-methylcytidine |
| J53 | Br | methylphosphonate | 5-methylcytidine |
| J54 | I | methylphosphonate | 5-methylcytidine |
| J55 | NH₂ | methylphosphonate | 5-methylcytidine |
| J56 | H | methylphosphonate | 5-methylcytidine |
| J57 | OH | boranophosphonate | 5-methylcytidine |
| J58 | OMe | boranophosphonate | 5-methylcytidine |
| J59 | F | boranophosphonate | 5-methylcytidine |
| J60 | Cl | boranophosphonate | 5-methylcytidine |
| J61 | Br | boranophosphonate | 5-methylcytidine |
| J62 | I | boranophosphonate | 5-methylcytidine |
| J63 | NH₂ | boranophosphonate | 5-methylcytidine |
| J64 | H | boranophosphonate | 5-methylcytidine |
| K1 | OH | OH | 5-aminoallyluridine |
| K2 | OMe | OH | 5-aminoallyluridine |
| K3 | F | OH | 5-aminoallyluridine |
| K4 | Cl | OH | 5-aminoallyluridine |
| K5 | Br | OH | 5-aminoallyluridine |
| K6 | I | OH | 5-aminoallyluridine |
| K7 | NH₂ | OH | 5-aminoallyluridine |
| K8 | H | OH | 5-aminoallyluridine |
| K9 | OH | phosphodiester | 5-aminoallyluridine |
| K10 | OMe | phosphodiester | 5-aminoallyluridine |
| K11 | F | phosphodiester | 5-aminoallyluridine |
| K12 | Cl | phosphodiester | 5-aminoallyluridine |
| K13 | Br | phosphodiester | 5-aminoallyluridine |
| K14 | I | phosphodiester | 5-aminoallyluridine |
| K15 | NH₂ | phosphodiester | 5-aminoallyluridine |
| K16 | H | phosphodiester | 5-aminoallyluridine |
| K17 | OH | phosphonoacetate | 5-aminoallyluridine |
| K18 | OMe | phosphonoacetate | 5-aminoallyluridine |
| K19 | F | phosphonoacetate | 5-aminoallyluridine |
| K20 | Cl | phosphonoacetate | 5-aminoallyluridine |
| K21 | Br | phosphonoacetate | 5-aminoallyluridine |
| K22 | I | phosphonoacetate | 5-aminoallyluridine |
| K23 | NH₂ | phosphonoacetate | 5-aminoallyluridine |
| K24 | H | phosphonoacetate | 5-aminoallyluridine |
| K25 | OH | thiophosphonoacetate | 5-aminoallyluridine |
| K26 | OMe | thiophosphonoacetate | 5-aminoallyluridine |
| K27 | F | thiophosphonoacetate | 5-aminoallyluridine |
| K28 | Cl | thiophosphonoacetate | 5-aminoallyluridine |
| K29 | Br | thiophosphonoacetate | 5-aminoallyluridine |
| K30 | I | thiophosphonoacetate | 5-aminoallyluridine |
| K31 | NH₂ | thiophosphonoacetate | 5-aminoallyluridine |
| K32 | H | thiophosphonoacetate | 5-aminoallyluridine |
| K33 | OH | phosphorothioate | 5-aminoallyluridine |
| K34 | OMe | phosphorothioate | 5-aminoallyluridine |
| K35 | F | phosphorothioate | 5-aminoallyluridine |
| K36 | Cl | phosphorothioate | 5-aminoallyluridine |
| K37 | Br | phosphorothioate | 5-aminoallyluridine |
| K38 | I | phosphorothioate | 5-aminoallyluridine |
| K39 | NH₂ | phosphorothioate | 5-aminoallyluridine |
| K40 | H | phosphorothioate | 5-aminoallyluridine |
| K41 | OH | phosphorodithioate | 5-aminoallyluridine |
| K42 | OMe | phosphorodithioate | 5-aminoallyluridine |
| K43 | F | phosphorodithioate | 5-aminoallyluridine |
| K44 | Cl | phosphorodithioate | 5-aminoallyluridine |
| K45 | Br | phosphorodithioate | 5-aminoallyluridine |
| K46 | I | phosphorodithioate | 5-aminoallyluridine |
| K47 | NH₂ | phosphorodithioate | 5-aminoallyluridine |
| K48 | H | phosphorodithioate | 5-aminoallyluridine |
| K49 | OH | methylphosphonate | 5-aminoallyluridine |
| K50 | OMe | methylphosphonate | 5-aminoallyluridine |
| K51 | F | methylphosphonate | 5-aminoallyluridine |
| K52 | Cl | methylphosphonate | 5-aminoallyluridine |
| K53 | Br | methylphosphonate | 5-aminoallyluridine |
| K54 | I | methylphosphonate | 5-aminoallyluridine |
| K55 | NH₂ | methylphosphonate | 5-aminoallyluridine |
| K56 | H | methylphosphonate | 5-aminoallyluridine |
| K57 | OH | boranophosphonate | 5-aminoallyluridine |
| K58 | OMe | boranophosphonate | 5-aminoallyluridine |
| K59 | F | boranophosphonate | 5-aminoallyluridine |
| K60 | Cl | boranophosphonate | 5-aminoallyluridine |
| K61 | Br | boranophosphonate | 5-aminoallyluridine |
| K62 | I | boranophosphonate | 5-aminoallyluridine |
| K63 | NH₂ | boranophosphonate | 5-aminoallyluridine |
| K64 | H | boranophosphonate | 5-aminoallyluridine |
| L1 | OH | OH | 5-methyluridine |
| L2 | OMe | OH | 5-methyluridine |
| L3 | F | OH | 5-methyluridine |
| L4 | Cl | OH | 5-methyluridine |
| L5 | Br | OH | 5-methyluridine |
| L6 | I | OH | 5-methyluridine |
| L7 | NH₂ | OH | 5-methyluridine |
| L8 | H | OH | 5-methyluridine |
| L9 | OH | phosphodiester | 5-methyluridine |
| L10 | OMe | phosphodiester | 5-methyluridine |
| L11 | F | phosphodiester | 5-methyluridine |
| L12 | Cl | phosphodiester | 5-methyluridine |
| L13 | Br | phosphodiester | 5-methyluridine |
| L14 | I | phosphodiester | 5-methyluridine |
| L15 | NH₂ | phosphodiester | 5-methyluridine |
| L16 | H | phosphodiester | 5-methyluridine |
| L17 | OH | phosphonoacetate | 5-methyluridine |
| L18 | OMe | phosphonoacetate | 5-methyluridine |
| L19 | F | phosphonoacetate | 5-methyluridine |
| L20 | Cl | phosphonoacetate | 5-methyluridine |
| L21 | Br | phosphonoacetate | 5-methyluridine |
| L22 | I | phosphonoacetate | 5-methyluridine |
| L23 | NH₂ | phosphonoacetate | 5-methyluridine |
| L24 | H | phosphonoacetate | 5-methyluridine |
| L25 | OH | thiophosphonoacetate | 5-methyluridine |
| L26 | OMe | thiophosphonoacetate | 5-methyluridine |
| L27 | F | thiophosphonoacetate | 5-methyluridine |
| L28 | Cl | thiophosphonoacetate | 5-methyluridine |
| L29 | Br | thiophosphonoacetate | 5-methyluridine |
| L30 | I | thiophosphonoacetate | 5-methyluridine |
| L31 | NH₂ | thiophosphonoacetate | 5-methyluridine |
| L32 | H | thiophosphonoacetate | 5-methyluridine |
| L33 | OH | phosphorothioate | 5-methyluridine |
| L34 | OMe | phosphorothioate | 5-methyluridine |
| L35 | F | phosphorothioate | 5-methyluridine |
| L36 | Cl | phosphorothioate | 5-methyluridine |
| L37 | Br | phosphorothioate | 5-methyluridine |
| L38 | I | phosphorothioate | 5-methyluridine |
| L39 | NH₂ | phosphorothioate | 5-methyluridine |
| L40 | H | phosphorothioate | 5-methyluridine |
| L41 | OH | phosphorodithioate | 5-methyluridine |
| L42 | OMe | phosphorodithioate | 5-methyluridine |
| L43 | F | phosphorodithioate | 5-methyluridine |
| L44 | Cl | phosphorodithioate | 5-methyluridine |
| L45 | Br | phosphorodithioate | 5-methyluridine |
| L46 | I | phosphorodithioate | 5-methyluridine |
| L47 | NH₂ | phosphorodithioate | 5-methyluridine |
| L48 | H | phosphorodithioate | 5-methyluridine |
| L49 | OH | methylphosphonate | 5-methyluridine |
| L50 | OMe | methylphosphonate | 5-methyluridine |
| L51 | F | methylphosphonate | 5-methyluridine |
| L52 | Cl | methylphosphonate | 5-methyluridine |
| L53 | Br | methylphosphonate | 5-methyluridine |
| L54 | I | methylphosphonate | 5-methyluridine |
| L55 | NH₂ | methylphosphonate | 5-methyluridine |
| L56 | H | methylphosphonate | 5-methyluridine |
| L57 | OH | boranophosphonate | 5-methyluridine |
| L58 | OMe | boranophosphonate | 5-methyluridine |
| L59 | F | boranophosphonate | 5-methyluridine |
| L60 | Cl | boranophosphonate | 5-methyluridine |
| L61 | Br | boranophosphonate | 5-methyluridine |
| L62 | I | boranophosphonate | 5-methyluridine |
| L63 | NH₂ | boranophosphonate | 5-methyluridine |
| L64 | H | boranophosphonate | 5-methyluridine |

As described herein, certain unnatural base pairs (e.g., isoG and isoC, Z base and P base; see Zhang et al. (2015) J. Am. Chem. Soc.) may be advantageous for affecting the thermostability of the guide RNA secondary structure. These modifications can be used to prevent misfolding of the guide RNA scaffold with other domains of a guide RNA sequence.

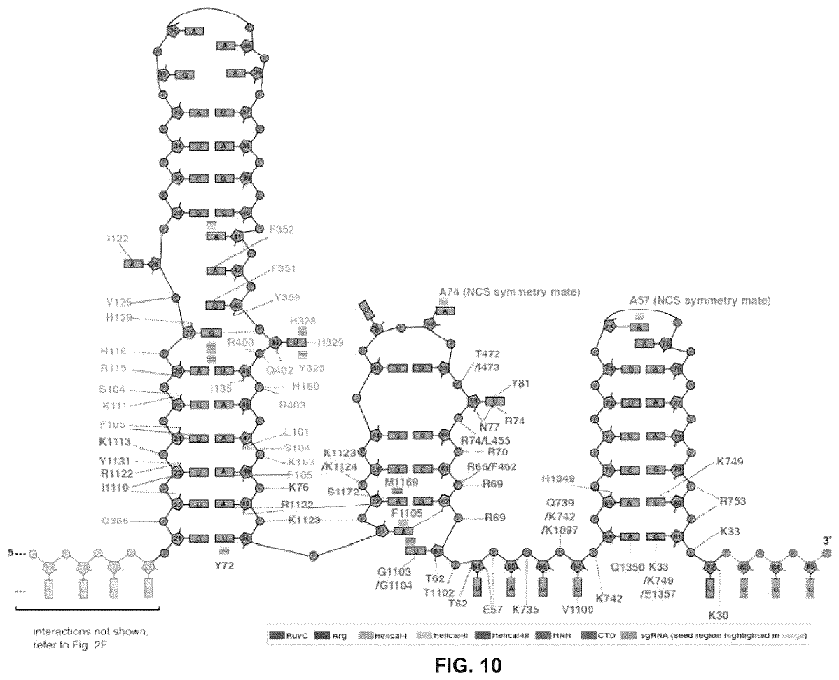

Recent guide RNA:Cas9 protein structural information (FIG. 10, as reported in Jiang et al. 2015, Science, 348, 1477-81) and in vivo/in vitro functional mutation studies (see, e.g., Briner et al. 2014, Mol. Cell, 56, 333-9) indicate that the guide RNA scaffold is predominantly structurally conserved. This reinforces the importance of correct folding of the conserved domain of guide RNAs for functionality with Cas9. FIG. 10 shows the guide RNA scaffold secondary structure, displaying interactions with amino acids of Cas9. Most of the guide RNA nitrogenous bases are not involved in binding interactions with Cas9 protein.

The flanking sequences of the sgRNA scaffold increase the likelihood of misfolding and hence misfunction. The 20nt guide targeting sequence, 5′ of the scaffold region, is user-specified for each target, thus the likelihood of misfolding is variable or target-specific. Also, many emerging CRISPR-Cas applications append functional sequences 3′ of the scaffold, such as CRISPRdisplay (Schechner et al., Nat. Methods 2015) and CRISPR-i/-a (Chen et al., Cell 2013), which are riboswitches or aptamers that also need to correctly and independently fold to function properly. To ensure that each of the functional domains (i.e. targeting guide, scaffold, aptamer) of a given sgRNA folds in a modular, independent manner, the structurally conserved scaffold base pairs can be substituted with unnatural, orthogonal base pairs (e.g., isoG and isoC; Z base and P base), and in some embodiments, substituted exclusively with unnatural, orthogonal base pairs. This ensures that the sgRNA scaffold sequences will not stably interact in a secondary structure with elements of the target-pairing guide sequence or other non-native domains incorporated in the guide RNA such as any aptamer sequences or any non-native 5′ or 3′ overhangs on the guide RNA. Alternatively, the unnatural, orthogonal base pairs mentioned above could be incorporated in any non-native overhangs or aptamers that may be present, thus to prevent secondary structures involving misfolding of the scaffold sequence(s).

### B. Guide RNA with at Least One Modification

In one aspect, the present technology provides a guide RNA having at least one modification, constituting a modified gRNA.

In certain embodiments, the modified gRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 modified nucleotides. In other embodiments, the modified gRNA comprises at least 55, 60, 65, 70, 75, 80, 85, 90, 95, 100, 110, 120, 130 or 140 modified nucleotides. In certain embodiments, all nucleotides are modified. In certain embodiments, all the modifications are the same. In certain embodiments, all the modified nucleotides have the same type of modification. In certain embodiments, the modified gRNA comprises a combination of differently modified nucleotides. In certain embodiments, the modified gRNA comprises two or more modified nucleotides. In certain embodiments, the modified gRNA comprises three or more modified nucleotides. In certain embodiments, the modified nucleotides are arranged contiguously. In certain embodiments, the modified gRNA comprises at least one contiguous stretch of modified nucleotides. In certain embodiments, the modified gRNA comprises a contiguous stretch of at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 modified nucleotides. Each modified nucleotide may independently comprise one or more types of modifications. In certain embodiments, no modified nucleotides are contiguous, or some but not all are contiguous, in the sequence of the modified gRNA.

In certain embodiments, the modification is within the 5′ portion of the guide RNA. In certain embodiments, the modification is within the first five (5) nucleotides of the 5′ portion of the guide RNA. In certain embodiments, the modification is within the first three (3) nucleotides of the 5′ portion of the guide RNA. In certain embodiments, the modification is within the 3′ portion of the guide RNA. In certain embodiments, the modification is within the last five (5) nucleotides of the 3′ portion of the guide RNA. In certain embodiments, the modification is within the last three (3) nucleotides of the 3′ portion of the guide RNA. In certain embodiments, the modification is within the internal region (i.e., between the 5′ end and the 3′ end) of the guide RNA.

In certain embodiments, the modification is incorporated in the 5′ portion or the 3′ portion of the guide RNA, particularly within the first 5 or 10 nucleotides of the 5′ portion or within the last 5 or 10 nucleotides of the 3′ portion to, for example, protect the RNA from degradation by nucleases or for other purposes. In some other embodiments, the modification is in both the 5′ portion and the 3′ portion of the guide RNA, particularly within the first 5 or 10 nucleotides of the 5′ portion and within the last 5 or 10 nucleotides of the 3′ portion to, for example, protect the RNA from degradation by nucleases or for other purposes. In certain embodiments, more than one type of modification is present in both the 5′ portion and the 3′ portion of the guide RNA. In certain embodiments, the modifications are located at the 5′ end, at the 3′ end, and within the internal sequence of the guide RNA. In certain embodiments, a guide RNA comprises 40 or fewer, alternatively 20 or fewer, alternatively 15 or fewer, alternatively 10 or fewer, alternatively 5 or fewer, alternatively 3 or fewer deoxyribonucleotide residues in the 5′ or 3′ portion of the guide RNA.

In certain embodiments, the modification is within the crRNA segment of the guide RNA. In certain embodiments, the modification is within the guide sequence of the crRNA. In certain embodiments, the modification is within the first five (5) nucleotides of the crRNA segment. In certain embodiments, the modification is within the first three (3) nucleotides of the crRNA segment. In certain embodiments, the modification is within a 5′-overhang on the crRNA segment. In certain embodiments, the modification is within the tracrRNA segment of the guide RNA. In certain embodiments, the modification is within the last five (5) nucleotides of the tracrRNA segment of the guide RNA. In certain embodiments, the modification is within the last three (3) nucleotides of the tracrRNA segment of the guide RNA. In certain embodiments, when the guide RNA is a single guide RNA, the modification is located within the loop of the guide RNA. In certain embodiments, one or more modifications is within the loop L region. In certain embodiments, the modification comprises a dye, a non-fluorescent label, or a tag conjugated to a linker incorporated between two nucleotides as described above, for example by conjugation to a 2-(3-(dye/label/tag-amido) propanamido) propane-1,3-diol bis(phosphodiester) linker or to a modified base of a nucleotide in the loop or L region.

In certain embodiments, the modification comprises an end modification, such as a 5′ end modification or a 3′ end modification. Examples of end modifications include, but are not limited to phosphorylation (as natural phosphate or polyphosphate or as modified phosphonate groups such as for example, alkylphosphonate, phosphonocarboxylate, phosphonoacetate, boranophosphate, phosphorothioate, phosphorodithioate and the like), biotinylation, conjugating or conjugated molecules, linkers, dyes, labels, tags, functional groups (such as for example but not limited to 5′-amino, 5′-thio, 5′-amido, 5′ carboxy and the like), inverted linkages, or hydrocarbon moieties which may comprise ether, polyethylene glycol (PEG), ester, hydroxyl, aryl, halo, phosphodiester, bicyclic, heterocyclic or other organic functional group. In certain embodiments, the end modification comprises dimethoxytrityl.

In certain embodiments, the modification comprises a modified base. As used herein, “unmodified” bases include the purine bases adenine (A) and guanine (G), and the pyrimidine bases thymine (T), cytosine (C) and uracil (U). Examples of modified bases include, but are not limited to, synthetic and natural bases such as 2-thioU, 2-thioC, 4-thioU, 6-thioG, 2-aminoA, 2-aminoP, pseudouracil, hypoxanthine, 7-deazaguanine, 7-deaza-8-azaguanine, 7-deazaadenine, 7-deaza-8-azaadenine, 5-methylC. 5-methylU, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allylU, 5-allylC, 5-aminoallyl-uracil, and 5-aminoallyl-cytosine. In certain embodiments, the modification comprises an abasic nucleotide. In certain embodiments, the modification comprises a nonstandard purine or pyrimidine structure, such as Z or P, isoC or isoG, UNA, 5-methylpyrimidine, x(A,G,C,T) or y(A,G,C,T). In certain embodiments, the modified gRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 modified bases. In other embodiments, the modified gRNA comprises at least 55, 60, 65, 70, 75, 80, 85, 90, 95, 100, 110, 120, 130 or 140 modified bases. In certain embodiments, all bases in a gRNA are modified.

In certain embodiments, the modification comprises a modified sugar. Examples of modified sugars include, but are not limited to, sugars having modifications at the 2′ position or modifications at the 4′ position. For example, in certain embodiments, the sugar comprises 2′-O—C1-4alkyl, such as 2′-O-methyl (2′-OMe). In certain embodiments, the sugar comprises 2′-O—C1-3alkyl-O—C1-3alkyl, such as 2′-methoxyethoxy(2′—O—CH2CH2OCH3) also known as 2′-O-(2-methoxyethyl) or 2′-MOE. In certain embodiments, the sugar comprises 2′-halo, such as 2′-F. 2′-Br, 2′-C1, or 2′-I. In certain embodiments, the sugar comprises 2′—NH2. In certain embodiments, the sugar comprises 2′-H (e.g., a deoxynucleotide). In certain embodiments, the sugar comprises 2′-arabino or 2′-F-arabino. In certain embodiments, the sugar comprises 2′-LNA or 2′-ULNA. In certain embodiments, the sugar comprises a 4′-thioribosyl. In certain embodiments, the modified gRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 modified sugars. In other embodiments, the modified gRNA comprises at least 55, 60, 65, 70, 75, 80, 85, 90, 95, 100, 110, 120, 130 or 140 modified sugars. In certain embodiments, all sugars in a gRNA are modified.

In certain embodiments, the modification comprises a modified backbone (i.e., an internucleotide linkage other than a natural phosphodiester). Examples of modified internucleotide linkages include, but are not limited to, a phosphorothioate internucleotide linkage, a chiral phosphorothioate internucleotide linkage, a phosphorodithioate internucleotide linkage, a boranophosphate internucleotide linkage, a C1-4alkyl phosphonate internucleotide linkage such as a methylphosphonate internucleotide linkage, a boranophosphate internucleotide linkage, a phosphonocarboxylate internucleotide linkage such as a phosphonoacetate internucleotide linkage, a phosphonocarboxylate ester internucleotide linkage such as a phosphonoacetate ester internucleotide linkage, a thiophosphonocarboxylate internucleotide linkage such as for example a thiophosphonoacetate internucleotide linkage, a thiophosphonocarboxylate ester internucleotide linkage such as a thiophosphonoacetate ester internucleotide linkage. Various salts, mixed salts and free acid forms are also included. In certain embodiments, the modified gRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 modified internucleotide linkages. In other embodiments, the modified gRNA comprises at least 55, 60, 65, 70, 75, 80, 85, 90, 95, 100, 110, 120, 130 or 140 modified internucleotide linkages. In certain embodiments, all internucleotide linkages in a gRNA are modified.

In certain embodiments, the modification is a 2′-O—C1-4alkyl, 2′-H, 2′-O—C1-3alkyl-O—C1-3alkyl, 2′-F, 2′—NH2, 2′-arabino, 2′-F-arabino, 2′-LNA, 2′-ULNA, 4′-thioribosyl, 2-thioU, 2-thioC, 4-thioU, 6-thioG, 2-aminoA, 2-aminoP, pseudouracil, hypoxanthine, 7-deazaguanine, 7-deaza-8-azaguanine, 7-deazaadenine, 7-deaza-8-azaadenine, 5-MeC, 5-MeU, 5-hydroxymethylcytosine, 5-hydroxymethyluracil, 5,6-dehydrouracil. 5-propynylcytosine, 5-propynyluracil, 5-ethynylcytosine, 5-ethynyluracil, 5-allylU, 5-allylC, 5-aminoallyl-uracil, 5-aminoallyl-cytosine, an abasic nucleotide, Z, P, UNA, isoC, isoG, 5-methyl-pyrimidine, x(A,G,C,T), y(A,G,C,T), a 3′-phosphorothioate group, a 3′-phosphonoacetate group, a 3′-phosphonoacetate ester group, a 3′-thiophosphonoacetate group, a 3′-thiophosphonoacetate ester group, a 3′-methylphosphonate group, a 3′-boranophosphate group, a 3′-phosphorodithioate group, or combinations thereof.

In certain embodiments, the modified nucleotide comprises a 2′-O-methyl-3′-phosphorothioate. In certain embodiments, the modified nucleotide comprises a 2′-O-methyl-3′-phosphonoacetate. In certain embodiments, the modified nucleotide comprises a 2′-O-methyl-3′-thiophosphonoacetate. In certain embodiments, the modified nucleotide comprises a Z base. In certain embodiments, the modified nucleotide comprises a 2′-halo-3′-phosphorothioate. In certain embodiments, the modified nucleotide comprises a 2′-halo-3′-phosphonoacetate. In certain embodiments, the modified nucleotide comprises a 2′-halo-3′-thiophosphonoacetate. In certain embodiments, the modified nucleotide comprises a 2′-fluoro-3′-phosphorothioate. In certain embodiments, the modified nucleotide comprises a 2′-fluoro-3′-phosphonoacetate. In certain embodiments, the modified nucleotide comprises a 2′-fluoro-3′-thiophosphonoacetate.

In certain embodiments, the guide RNA comprises an oligonucleotide represented by Formula (I):

W—Y or Y—W (I)
wherein W represents a nucleotide or a stretch of nucleotides of the oligonucleotide comprising at least one modification and Y represents an unmodified portion of the oligonucleotide.

In certain embodiments. W is within the 5′ portion of the guide RNA. In certain embodiments. W is at least partially within the first five (5) nucleotides of the 5′ portion of the guide RNA. In certain embodiments, W is at least partially within the first three (3) nucleotides of the 5′ portion of the guide RNA. In certain embodiments, W is within the 3′ portion of the guide RNA. In certain embodiments, W is at least partially within the last five (5) nucleotides of the 3′ portion of the guide RNA. In certain embodiments, W is at least partially within the last three (3) nucleotides of the 3′ portion of the guide RNA. In certain embodiments, W is within the internal region (i.e., between the 5′ end and the 3′ end) of the guide RNA.

In certain embodiments, W comprises an end modification, such as a 5′ end modification or a 3′ end modification as described above. In certain embodiments, the end modification comprises dimethoxytrityl.

In certain embodiments, W comprises a modified base as described above. In certain embodiments, W comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 modified bases. In other embodiments, W comprises at least 55, 60, 65, 70, 75, 80, 85, 90, 95, 100, 110, 120, 130 or 140 modified bases. In certain embodiments, all bases in a gRNA are modified.

In certain embodiments, W comprises a modified sugar as described above. In certain embodiments, W comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 modified sugars. In other embodiments, W comprises at least 55, 60, 65, 70, 75, 80, 85, 90, 95, 100, 110, 120, 130 or 140 modified sugars. In certain embodiments, all sugars in a gRNA are modified.

In certain embodiments, W comprises a modified backbone (i.e., an internucleotide linkage other than a phosphodiester) as described above. In certain embodiments, W comprises more than one, such as 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50 modified internucleotide linkages. In other embodiments, W comprises at least 55, 60, 65, 70, 75, 80, 85, 90, 95, 100, 110, 120, 130 or 140 modified internucleotide linkages. In certain embodiments, all internucleotide linkages in a gRNA are modified.

In certain embodiments, W comprises a 2′-O—C1-4alkyl, 2′-H, 2′-O—C1-3alkyl-O—C1-3alkyl, 2′-F, 2′—NH2, 2′-arabino, 2′-F-arabino, 2′-LNA, 2′-ULNA, 4′-thioribosyl, 2-thioU, 2-thioC, 4-thioU, 6-thioG, 2-aminoA, 2-aminoP, pseudouracil, hypoxanthine, 7-deazaguanine, 7-deaza-8-azaguanine, 7-deazaadenine, 7-deaza-8-azaadenine, 5-MeC, 5-MeU, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allylU, 5-allylC, 5-aminoallyl-uracil, 5-aminoallyl-cytosine, abasic nucleotides, Z, P, UNA, isoC, isoG, 5-methyl-pyrimidine, x(A,G,C,T), y(A,G,C,T), a phosphorothioate internucleotide linkage, a phosphonoacetate internucleotide linkage, a phosphonoacetate ester internucleotide linkage, a thiophosphonoacetate internucleotide linkage, a thiophosphonoacetate ester internucleotide linkage a methylphosphonate internucleotide linkage, a boranophosphate internucleotide linkage, a phosphorodithioate internucleotide linkage, or combinations thereof.

In certain embodiments, W comprises a 2′-O-methyl and a 3′-phosphorothioate group on the same nucleotide. In certain embodiments, W comprises a 2′-O-methyl and a 3′-phosphonoacetate group on the same nucleotide. In certain embodiments, W comprises a 2′-O-methyl and 3′-thiophosphonoacetate group on the same nucleotide. In certain embodiments, W comprises a 2′-F and a 3′-phosphorothioate group on the same nucleotide. In certain embodiments, W comprises a 2′-F and a 3′-phosphonoacetate group on the same nucleotide. In certain embodiments, W comprises a 2′-F and 3′-thiophosphonoacetate group on the same nucleotide.

In certain embodiments, W comprises 1, 2, 3, 4, 5, 6, 7, 8, 9, 10, 11, 12, 13, 14, 15, 16, 17, 18, 19 or 20 modified nucleotides. In certain embodiments, each of the modified nucleotides comprises the same modification. In certain embodiments, W comprises a combination of variously modified nucleotides. In certain embodiments, W comprises two or more modified nucleotides. In certain embodiments, W comprises three or more modified nucleotides. In certain embodiments, the modified nucleotides are not arranged contiguously in the sequence, or at least not entirely, as one or more unmodified nucleotides may intercede. In certain embodiments, the modified nucleotides are arranged contiguously. In certain embodiments, W comprises at least one contiguous stretch of modified nucleotides. In certain embodiments, W comprises a contiguous stretch of at least three (3) modified nucleotides. In certain embodiments, W comprises a contiguous stretch of at least four (4) modified nucleotides. In certain embodiments, W comprises a contiguous stretch of at least five (5) modified nucleotides.

In certain embodiments, the guide RNA comprises an oligonucleotide represented by Formula (II):

MₘNₙ (II)
wherein each N independently represents an unmodified ribonucleotide;
wherein each M represents a modified nucleotide and is independently selected from the group consisting of a 2′-O-methyl ribonucleotide, a 3′-P(S) ribonucleotide, a 3′-PACE ribonucleotide, a 3′-thioPACE ribonucleotide, a 2′-O-methyl-3′-P(S)-ribonucleotide, a 2′-O-methyl-3′-PACE ribonucleotide, a 2′-O-methyl-3′-thioPACE ribonucleotide, a Z nucleotide, and a 2′-deoxynucleotide;
wherein each M is at any position of the sequence of the guide RNA;
wherein any given M is the same or different from any other M, and any given N is the same or different from any other N; and
wherein each of m and n are independently selected from an integer between 0 and 219, provided that 50<m+n≤220, and m is not 0.

In some embodiments, m+n<150.

In certain embodiments, each M is modified with one or more moieties independently selected from the group consisting of 2′-F, 2-thiouracil, 4-thiouracil, 2-aminoadenine, hypoxanthine, 5-methylcytosine, 5-methyluracil, 5-allylaminouracil, squarate linkage, a triazolo linkage, and a 2-(4-butylamidofluorescein) propane-1,3-diol bis(phosphodiester) linkage. In some embodiments, M comprises a dye attached through a linker.

In certain embodiments, each M is independently selected from the group consisting of a 2′-O-methyl ribonucleotide, a 2′-O-methyl-3′-P(S) ribonucleotide, a 2′-O-methyl-3′-PACE ribonucleotide, and a 2′-O-methyl-3′-thioPACE ribonucleotide. In certain embodiments, each M is independently selected from the group consisting of a 2′-O-methyl-3′-PACE ribonucleotide and a 2′-O-methyl-3′-thioPACE ribonucleotide.

In certain embodiments, where m>1, any given M is the same or different from any other M. In certain embodiments, where m>1, each M has the same modification.

In certain embodiments, each M is a 2′-O-methyl-3′-PACE ribonucleotide, m is selected from an integer between 1 and 10, each N is independently selected from the group consisting of A, U, C, and G, and n is selected from an integer between 1 and 149, provided 50<m+n≤150. In certain embodiments, each M is a 2′-O-methyl-3′-PACE ribonucleotide, m is selected from an integer between 1 and 5, each N is independently selected from the group consisting of A, U, C, and G, and n is selected from an integer between 1 and 149, provided 50<m+n≤150. In certain embodiments, each M is a 2′-O-methyl-3′-PACE ribonucleotide, m is selected from an integer between 2 and 5, each N is independently selected from the group consisting of A, U, C, and G, and n is selected from an integer between 1 and 148, provided 50<m+n≤150. In certain embodiments, m is 1. In certain embodiments, m is 2. In certain embodiments, m is 3. In certain embodiments, m is 4. In certain embodiments, m is 5.

In certain embodiments, each M is a 2′-O-methyl-3′-thioPACE ribonucleotide, m is selected from an integer between 1 and 10, each N is independently selected from the group consisting of A, U, C, and G, and n is selected from an integer between 1 and 149, provided 50<m+n≤150. In certain embodiments, each M is a 2′-O-methyl-3′-thioPACE ribonucleotide, m is selected from an integer between 1 and 5, each N is independently selected from the group consisting of A, U, C, and G, and n is selected from an integer between 1 and 149, provided 50<m+n≤150. In certain embodiments, each M is a 2′-O-methyl-3′-thioPACE ribonucleotide, m is selected from an integer between 2 and 5, each N is independently selected from the group consisting of A, U, C, and G, and n is selected from an integer between 1 and 148, provided 50<m+n≤150. In certain embodiments, m is 1. In certain embodiments, m is 2. In certain embodiments, m is 3. In certain embodiments, m is 4. In certain embodiments, m is 5.

In certain embodiments, each M is a 2′-O-methyl ribonucleotide, m is selected from an integer between 1 and 40, each N is independently selected from the group consisting of A. U. C, and G, and n is selected from an integer between 1 and 149, provided 50<m+n≤150. In certain embodiments, each M is a 2′-O-methyl ribonucleotide, m is selected from an integer between 1 and 25, each N is independently selected from the group consisting of A. U. C, and G, and n is selected from an integer between 1 and 149, provided 50<m+n≤150. In certain embodiments, each M is a 2′-O-methyl ribonucleotide, m is selected from an integer between 1 and 20, each N is independently selected from the group consisting of A, U, C, and G, and n is selected from an integer between 1 and 149, provided 50<m+n≤150. In certain embodiments, m is 1. In certain embodiments, m is 2. In certain embodiments, m is 3. In certain embodiments, m is 4. In certain embodiments, m is 5. In certain embodiments, m is 10. In certain embodiments, m is 15. In certain embodiments, m is 20. In certain embodiments, m is 30. In certain embodiments, m is 40.

In certain embodiments, each M is a 2′-deoxynucleotide, m is selected from an integer between 1 and 30, each N is independently selected from the group consisting of A, U, C, and G, and n is selected from an integer between 1 and 149, provided 50<m+n≤150. In certain embodiments, each M is 2′-deoxynucleotide, m is selected from an integer between 1 and 20, each N is independently selected from the group consisting of A, U, C, and G, and n is selected from an integer between 1 and 149, provided 50<m+n≤150. In certain embodiments, m is 5. In certain embodiments, m is 10. In certain embodiments, m is 15. In certain embodiments, m is 20. In certain embodiments, m is 30.

In certain embodiments, each M is a 2′-O-methyl-3′-P(S) ribonucleotide, m is selected from an integer between 1 and 10, each N is independently selected from the group consisting of A. U. C, and G, and n is selected from an integer between 1 and 149, provided 50<m+n≤150. In certain embodiments, each M is a 2′-O-methyl-3′-P(S) ribonucleotide, m is selected from an integer between 1 and 5, each N is independently selected from the group consisting of A, U, C, and G, and n is selected from an integer between 1 and 149, provided 50<m+n≤150. In certain embodiments, m is 1. In certain embodiments, m is 2. In certain embodiments, m is 3. In certain embodiments, m is 4. In certain embodiments, m is 5.

In certain embodiments, each M is a Z nucleotide, m is selected from an integer between 1 and 10, each N is independently selected from the group consisting of A. U. C, and G, and n is selected from an integer between 1 and 149, provided 50<m+n≤150. In certain embodiments, each M is a Z nucleotide, m is selected from an integer between 1 and 5, each N is independently selected from the group consisting of A. U. C, and G, and n is selected from an integer between 1 and 149, provided 50<m+n≤150. In certain embodiments, m is 1. In certain embodiments, m is 2. In certain embodiments, m is 3. In certain embodiments, m is 4. In certain embodiments, m is 5.

In certain embodiments, the modification is a stability-altering modification. In certain embodiments, the modification increases nuclease resistance of the guide RNA relative to a guide RNA without the modification, thus it enhances the guide RNA stability. In certain embodiments, the stability-altering modification is a stability-enhancing modification. For example, in certain embodiments, the stability-enhancing modification comprises a 2′-O-methyl or a 2′-O—C1-4alkyl nucleotide. In certain embodiments, the stability-enhancing modification comprises a 2′-halo nucleotide, such as 2′-F, 2′-Br, 2′-C1, or 2′-I. In certain embodiments, the stability-enhancing modification comprises a 2′MOE or a 2′-O—C1-3alkyl-O—C1-3alkyl. In certain embodiments, the stability-enhancing modification comprises a 2′—NH2 nucleotide. In certain embodiments, the stability-enhancing modification comprises a 2′-H (or 2′-deoxy) nucleotide. In certain embodiments, the stability-enhancing modification comprises a 2′-arabino or a 2′-F-arabino. In certain embodiments, the stability-enhancing modification comprises a 4′-thioribosyl sugar moiety. In certain embodiments, the stability-enhancing modification comprises a 3′-phosphorothioate group. In certain embodiments, the stability-enhancing modification comprises a 3′-phosphonoacetate group. In certain embodiments, the stability-enhancing modification comprises a nucleotide containing a 3′-thiophosphonoacetate group. In certain embodiments, the stability-enhancing modification comprises a nucleotide containing a 3′-methylphosphonate group. In certain embodiments, the stability-enhancing modification comprises a nucleotide containing a 3′-boranophosphate group. In certain embodiments, the stability-enhancing modification comprises a nucleotide containing a 3′-phosphorodithioate group. In certain embodiments, the stability-enhancing modification comprises a locked nucleic acid (“LNA”) nucleotide. In certain embodiments, the stability-enhancing modification comprises an unlocked nucleic acid (“ULNA”) nucleotide.

In certain embodiments, the stability-enhancing modification comprises a 2′-O-methyl and a 3′-phosphorothioate group on the same nucleotide. In certain embodiments, the stability-enhancing modification comprises a 2′-O-methyl and a 3′-phosphonoacetate group on the same nucleotide. In certain embodiments, the stability-enhancing modification comprises a 2′-O-methyl and a 3′-thiophosphonoacetate group on the same nucleotide. In certain embodiments, the stability-enhancing modification comprises a 2′-fluoro and a 3′-phosphorothioate group on the same nucleotide. In certain embodiments, the stability-enhancing modification comprises a 2′-fluoro and a 3′-phosphonoacetate group on the same nucleotide. In certain embodiments, the stability-enhancing modification comprises a 2′-fluoro and a 3′-thiophosphonoacetate group on the same nucleotide.

In certain embodiments, the modification is a specificity-altering modification. In some embodiments, specificity enhancement may be achieved by enhancing on-target binding and/or cleavage, or reducing off-target binding and/or cleavage, or a combination of both. In some other embodiments, specificity reduction may be achieved, for example, by reducing on-target binding and/or cleavage, or increasing off-target binding and/or cleavage, or a combination of both.

In certain embodiments, the specificity-altering modification comprises a 2′-O-methyl. In certain embodiments, the specificity-altering modification comprises a 2′-halo, such as 2′-fluoro.

In certain embodiments, the specificity-altering modification comprises a 2-thiouracil base (2-thioU). In certain embodiments, the specificity-altering modification comprises 2-thioC. In certain embodiments, the specificity-altering modification comprises 4-thioU. In certain embodiments, the specificity-altering modification comprises 6-thioG. In certain embodiments, the specificity-altering modification comprises 2-aminoA. In certain embodiments, the specificity-altering modification comprises a 2-aminopurine. In certain embodiments, the specificity-altering modification comprises pseudouracil. In certain embodiments, the specificity-altering modification comprises hypoxanthine. In certain embodiments, the specificity-altering modification comprises 7-deazaguanine. In certain embodiments, the specificity-altering modification comprises 7-deaza-8-azaguanine. In certain embodiments, the specificity-altering modification comprises 7-deazaadenine. In certain embodiments, the specificity-altering modification comprises 7-deaza-8-azaadenine. In certain embodiments, the specificity-altering modification comprises 5-methylC. In certain embodiments, the specificity-altering modification comprises 5-methylU. In certain embodiments, the specificity-altering modification comprises 5-hydroxymethylcytosine. In certain embodiments, the specificity-altering modification comprises 5-hydroxymethyluracil. In certain embodiments, the specificity-altering modification comprises 5,6-dehydrouracil. In certain embodiments, the specificity-altering modification comprises 5-propynylcytosine. In certain embodiments, the specificity-altering modification comprises 5-propynyluracil. In certain embodiments, the specificity-altering modification comprises 5-ethynylcytosine. In certain embodiments, the specificity-altering modification comprises 5-ethynyluracil. In certain embodiments, the specificity-altering modification comprises 5-allylU. In certain embodiments, the specificity-altering modification comprises 5-allylC. In certain embodiments, the specificity-altering modification comprises 5-aminoallylU. In certain embodiments, the specificity-altering modification comprises 5-aminoallylC. In certain embodiments, the specificity-altering modification comprises an abasic nucleotide. In certain embodiments, the specificity-altering modification comprises a Z base. In certain embodiments, the specificity-altering modification comprises P base. In certain embodiments, the specificity-altering modification comprises a UNA base. In certain embodiments, the specificity-altering modification comprises isoC. In certain embodiments, the specificity-altering modification comprises isoG. In certain embodiments, the specificity-altering modification comprises 5-methyl-pyrimidine. In certain embodiments, the specificity-altering modification comprises x(A,G,C,T). In certain embodiments, the specificity-altering modification comprises y(A,G,C,T).

In certain embodiments, the specificity-altering modification comprises a phosphorothioate internucleotide linkage. In certain embodiments, the specificity-altering modification comprises a phosphonoacetate internucleotide linkage. In certain embodiments, the specificity-altering modification comprises a thiophosphonoacetate internucleotide linkage. In certain embodiments, the specificity-altering modification comprises a methylphosphonate internucleotide linkage. In certain embodiments, the specificity-altering modification comprises a boranophosphate internucleotide linkage. In certain embodiments, the specificity-altering modification comprises a phosphorodithioate internucleotide linkage. In certain embodiments, the specificity-altering modification comprises a ULNA. In certain embodiments, the specificity-altering modification comprises an LNA.

In certain embodiments, the modification alters RNA base pairing by, for example, altering the melting temperature (Tm) of the guide RNA relative to a guide RNA without the modification. In certain embodiments, the modification lowers the Tm of the guide RNA relative to a guide RNA without the modification. In certain embodiments, the modification raises the Tm of the guide RNA relative to a guide RNA without the modification.

In certain embodiments, the specificity-altering modification lowers the Tm of a base pairing interaction. In certain embodiments, the modification that lowers the Tm of the base pairing interaction is a 2′-deoxy, as it is well-known in the art that DNA/DNA base pairs have lower Tm than their respective counterpart in RNA/DNA duplexes. In certain embodiments, the modification that lowers the Tm of the base pairing interaction is 2-thiouracil, which slightly lowers Tm of G-U wobble pair. In certain embodiments, the modification that lowers the Tm of the base pairing interaction is a phosphorothioate internucleotide linkage or a phosphorodithioate internucleotide linkage, which lower the Tm by ˜0.5° C. per modification. In certain embodiments, the modification that lowers the Tm of the base pairing interaction is a boranophosphate internucleotide linkage, which lowers the Tm by ˜0.5-0.8° C. per modification. In certain embodiments, the modification that lowers the Tm of the base pairing interaction is a phosphonoacetate internucleotide linkage, which lowers the Tm by ˜1.3° C. per modification. In certain embodiments, the modification that lowers the Tm of the base pairing interaction is unlocked nucleic acid (“ULNA”), which lowers the Tm by ˜5-8° C. per modification. In certain embodiments, the modification that lowers the Tm of the base pairing interaction is 2′-O-methyl-3′-methylphosphonate.

In certain embodiments, the specificity-altering modification raises the Tm of a base pairing interaction. In certain embodiments, the modification that raises the Tm of the base pairing interaction is a 2′-O-methyl, which raises Tm by ˜0.5-0.7° C. per modification. In certain embodiments, the modification that raises the Tm of the base pairing interaction is a 2′-F, which raises Tm by ˜1° C. per modification. In certain embodiments, the modification that raises the Tm of the base pairing interaction is a 2-thiouracil, which raises Tm of A-U pair (and, as noted above, slightly lowers Tm of G-U wobble pair). In certain embodiments, the modification that raises the Tm of the base pairing interaction is a 4-thiouracil, which raises Tm of G-U wobble pair and slightly raises Tm of A-U pair. In certain embodiments, the modification that raises the Tm of the base pairing interaction is a 2-amino-adenine, which raises Tm of its base pairing with U by ˜1° C. per modification. In certain embodiments, the modification that raises the Tm of the base pairing interaction is a 5-methyl-uracil (5-methylU) (see, e.g., Wang & Kool (1995) Biochemistry, 34, 4125-32). In certain embodiments, the modification that raises the Tm of the base pairing interaction is a 5-methyl-cytosine (5-methylC). In certain embodiments, the modification that raises the Tm of the base pairing interaction is a locked nucleic acid (“LNA”), which raises Tm by 2-10° C. per modification.

In certain embodiments, the modification alters transfection efficiency of the guide RNA relative to a guide RNA without the modification. In certain embodiments, the modification increases transfection efficiency of the guide RNA relative to a guide RNA without the modification. In certain embodiments, the modification decreases transfection efficiency of the guide RNA relative to a guide RNA without the modification. In certain embodiments, the modification neutralizes the anionic charge on phosphate to allow passive diffusion into cells. In certain embodiments, the charge-neutralizing modification comprises a phosphonoacetate alkyl ester internucleotide linkage, such as a phosphonoacetate methyl ester internucleotide linkage.

In certain embodiments, the modification alters the immunostimulatory effect of the guide RNA relative to a guide RNA without the modification. It was initially discovered that unmethylated bacterial DNA and synthetic analogs thereof are ligands for TLR9 (see Hemmi et al. (2000) Nature, 408, 740-5). The stimulation of TLR9 can be mitigated in the dinucleotide motif for example by modifying the C and G residues. The use of 5-methylcytosine, 2-aminocytosine, 2-thiocytosine, 5-methylisocytosine, P nucleobase (6-(β-D-2′-Deoxyribofuranosyl)-3,4-dihydro-8H-pyrimido[4,5-c][1,2]oxazin-7-one), and 2′-O-methylcytosine all result in loss or decrease in TLR9 stimulation. In certain embodiments, use of 6-thioguanine, 2,6-diaminopurine, 2-aminopurine, xanthosine, inosine, 7-deazaxanthosine, isoguanine, 8-oxoguanine, nebularine, 8-bromoguanine, K-nucleobase (2-amino-N-methoxyadenosine), and/or 2′-O-methylguanine can result in loss or decrease in TLR9 stimulation. In some embodiments, use of phosphodiester modifications can lower or eliminate the TLR9 response. Typically, synthetically incorporated phosphorothioates can decrease the TLR9 response to a limited extent, as is thought to result from the presence of two stereoisomers of each phosphorothioate in synthetic RNA. However, it has been shown that phosphorothioate-modified DNA lacking CpG motifs stimulate TLR9 to a rather small extent. The negative charge on the phosphorus is an important element for recognition by TLR9 and therefore removing the negative charge using alkylphosphonates can result in loss or decrease in TLR9 stimulation. The use of phosphonoacetate (PACE) internucleotide linkages between deoxynucleosides in 5′ and 3′ terminal sequences can significantly increase the TLR9 response; however, the use of thiophosphonoacetate (thioPACE) internucleotide linkages between deoxynucleosides in 5′ and 3′ terminal sequences can result in loss or decrease in TLR9 stimulation. In certain embodiments, use of sugar modifications that the favor C3′-endo conformation such as 2′-O-methyl modifications can be incorporated at 5′ and 3′ termini to decrease the TLR9 response. TLR7 and TLR8 can be stimulated by molecules containing 7-deazaguanine and by single-stranded RNA (see, e.g., Heil et al. (2004) Science, 303, 1526-9). TLR3 has been implicated in cellular immunoresponses to virus-derived double-stranded RNA. In certain embodiments, these TLR responses can be mitigated for example by using 2′-O-methyl modifications, modified phosphodiester linkages containing sulfur, or modifications that decrease internucleotide negative charge such as methylphosphonate and/or phosphonoacetate internucleotide linkages.

In certain embodiments, the modification enhances stability and specificity of the guide RNA relative to a guide RNA without the modification. In certain embodiments, the modification enhances stability and transfection efficiency of the guide RNA relative to a guide RNA without the modification. In certain embodiments, the modification enhances specificity and transfection efficiency of the guide RNA relative to a guide RNA without the modification. In certain embodiments, the modification enhances the overall efficacy of the guide RNA relative to a guide RNA without the modification.

### C. Guide RNA with a Combination of Modifications

In one aspect, the present technology provides a guide RNA having a combination of two or more modifications.

In certain embodiments, the two modifications are on the same nucleotide (for example, one nucleotide comprises a 2′-O-methyl and a 3′-thiophosphonoacetate moiety). In other embodiments, the two modifications are on two different nucleotides (for example, one nucleotide has a 2-thioU base and another nucleotide has a 2′-O-methyl group).

In certain embodiments, each modification in the guide RNA is the same. In certain embodiments, at least one modification in the guide RNA is different from at least one other modification in the guide RNA. In certain embodiments, a single nucleotide within the guide RNA possesses two or more modifications.

In certain embodiments, the guide RNA comprises a combination of different types of modifications, and at least one type in the combination exists in multiple places in the guide RNA In certain embodiments, at least one type in the combination appears 1, 2, 3, 4, 5, 6, 7, 8, 9, 10, 11, 12, 13, 14, 15, 16, 17, 18, 19 or 20 times in the guide RNA.

In certain embodiments, at least one type of the modifications in the combination appears in two or more modified nucleotides. In certain embodiments, at least one type of the modifications in the combination appears in three or more modified nucleotides. In certain embodiments, the modified nucleotides are not arranged contiguously in the sequence, or at least not entirely, as one or more unmodified nucleotides may intercede. In certain embodiments, the modified nucleotides are arranged contiguously. In certain embodiments, the guide RNA comprises a stretch of contiguous modified nucleotides of the same type. In certain embodiments, the stretch has at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 modified nucleotides.

In certain embodiments, at least one type of the modifications in the combination is within the 5′ portion of the guide RNA. In certain embodiments, at least one type of the modifications in the combination is within the first five (5) nucleotides of the 5′ portion of the guide RNA. In certain embodiments, at least one type of the modifications in the combination is within the first three (3) nucleotides of the 5′ portion of the guide RNA. In certain embodiments, at least one type of the modifications in the combination is within the 3′ portion of the guide RNA. In certain embodiments, at least one type of the modifications in the combination is within the last five (5) nucleotides of the 3′ portion of the guide RNA. In certain embodiments, at least one type of the modifications in the combination is within the last three (3) nucleotides of the 3′ portion of the guide RNA. In certain embodiments, at least one type of the modifications in the combination is within the internal region (i.e., between the 5′ end and the 3′ end) of the guide RNA.

In certain embodiments, at least one type of the modifications in the combination is incorporated in the 5′ portion or 3′ portion of the guide RNA, particularly within the first 5 or 10 nucleotides of the 5′ portion or within the last 5 or 10 nucleotides of the 3′ portion to, for example, protect the RNA from degradation by nucleases or for other purposes. In certain embodiments, at least one type of the modifications in the combination is in the 5′ portion and at least one type of the modifications in the combination is in the 3′ portion of the guide RNA, particularly within the first 5 or 10 nucleotides of the 5′ portion and within the last 5 or 10 nucleotides of the 3′ portion to, for example, protect the RNA from degradation by nucleases or for other purposes. In certain embodiments, a guide RNA comprises 20 or fewer, alternatively 15 or fewer, alternatively 15 or fewer, alternatively 10 or fewer, alternatively 5 or fewer, alternatively 3 or fewer deoxyribonucleotide residues in the 5′ portion of the guide RNA.

In certain embodiments, at least one type of the modifications in the combination is within the crRNA segment of the guide RNA. In certain embodiments, at least one type of the modifications in the combination is within the guide sequence of the crRNA. In certain embodiments, at least one type of the modifications in the combination is within the first five (5) nucleotides of the crRNA segment. In certain embodiments, at least one type of the modifications in the combination is within the first three (3) nucleotides of the crRNA segment. In certain embodiments, at least one type of the modifications in the combination is within the tracrRNA segment of the guide RNA. In certain embodiments, at least one type of the modifications in the combination is within the last five (5) nucleotides of the tracrRNA segment of the guide RNA. In certain embodiments, at least one type of the modifications in the combination is within the last three (3) nucleotides of the tracrRNA segment of the guide RNA.

In certain embodiments, a first type of modification in the combination is within the 5′ portion of the guide RNA and a second type of modification in the combination is within the internal region (i.e., between the 5′ end and the 3′ end) of the guide RNA. In certain embodiments, the first type of modification is within the first five (5) nucleotides of the 5′ portion of the guide RNA. In certain embodiments, the first type of modification is within the first three (3) nucleotides of the 5′ portion of the guide RNA.

In certain embodiments, a first type of modification in the combination is within the internal region (i.e., between the 5′ end and the 3′ end) of the guide RNA and a second type of modification in the combination is within the 3′ portion of the guide RNA. In certain embodiments, the second type of modification is within the last five (5) nucleotides of the 3′ portion of the guide RNA. In certain embodiments, the second type of modification is within the last three (3) nucleotides of the 3′ portion of the guide RNA.

In certain embodiments, a first type of modification in the combination is within the 5′ portion of the guide RNA and a second type of modification in the combination is within the 3′ portion of the guide RNA. In certain embodiments, the first type of modification is within the first five (5) nucleotides of the 5′ portion of the guide RNA. In certain embodiments, the first type of modification is within the first three (3) nucleotides of the 5′ portion of the guide RNA. In certain embodiments, the second type of modification is within the last five (5) nucleotides of the 3′ portion of the guide RNA. In certain embodiments, the second type of modification is within the last three (3) nucleotides of the 3′ portion of the guide RNA.

In certain embodiments, a first type of modification in the combination is within the 5′ portion of the guide RNA, a second type of modification in the combination is within the internal region (i.e., between the 5′ end and the 3′ end) of the guide RNA, and a third type of modification in the combination is within the 3′ portion of the guide RNA. In certain embodiments, the first type of modification is within the first five (5) nucleotides of the 5′ portion of the guide RNA. In certain embodiments, the first type of modification is within the first three (3) nucleotides of the 5′ portion of the guide RNA. In certain embodiments, the third type of modification is within the last five (5) nucleotides of the 3′ portion of the guide RNA. In certain embodiments, the third type of modification is within the last three (3) nucleotides of the 3′ portion of the guide RNA.

In certain embodiments, a first type of modification in the combination is within the crRNA segment of the guide RNA and a second type of modification in the combination is within the tracr segment. In certain embodiments, the first type of modification is within the guide sequence of the crRNA. In certain embodiments, the first type of modification is within the first five (5) nucleotides of the crRNA segment. In certain embodiments, the first type of modification is within the first three (3) nucleotides of the crRNA segment. In certain embodiments, the second type of modification is within the last five (5) nucleotides of the tracrRNA segment of the guide RNA. In certain embodiments, the second type of modification is within the last three (3) nucleotides of the tracrRNA segment of the guide RNA.

In certain embodiments, a first type and a second type of modification in the combination are within the crRNA segment of the guide RNA. In certain embodiments, the first type of modification is within the guide sequence of the crRNA. In certain embodiments, the first type of modification is within the first five (5) nucleotides of the crRNA segment. In certain embodiments, the first type of modification is within the first three (3) nucleotides of the crRNA segment.

In certain embodiments, a first type and a second type of modification in the combination are within the crRNA segment of the guide RNA and a third type of modification in the combination is within the tracr segment. In certain embodiments, the first type of modification is within the guide sequence of the crRNA. In certain embodiments, the first type of modification is within the first five (5) nucleotides of the crRNA segment. In certain embodiments, the first type of modification is within the first three (3) nucleotides of the crRNA segment. In certain embodiments, the third type of modification is within the last five (5) nucleotides of the tracrRNA segment of the guide RNA. In certain embodiments, the third type of modification is within the last three (3) nucleotides of the tracrRNA segment of the guide RNA.

In certain embodiments, at least one of the modifications in the combination comprises an end modification, such as a 5′ end modification or a 3′ end modification as described above. In certain embodiments, the end modification comprises dimethoxytrityl.

In certain embodiments, at least one of the modifications in the combination comprises a modified base. In certain embodiments, the modified gRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 modified bases. In other embodiments, the modified gRNA comprises at least 55, 60, 65, 70, 75, 80, 85, 90, 95, 100, 110, 120, 130 or 140 modified bases. In certain embodiments, all bases in a gRNA are modified.

In certain embodiments, at least one of the modifications in the combination comprises a modified sugar. In certain embodiments, the modified gRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 modified sugars. In other embodiments, the modified gRNA comprises at least 55, 60, 65, 70, 75, 80, 85, 90, 95, 100, 110, 120, 130 or 140 modified sugars. In certain embodiments, all sugars in a gRNA are modified.

In certain embodiments, at least one of the modifications in the combination comprises a modified backbone (i.e., an internucleotide linkage other than a natural phosphodiester). In certain embodiments, the modified gRNA comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 modified internucleotide linkages. In other embodiments, the modified gRNA comprises at least 55, 60, 65, 70, 75, 80, 85, 90, 95, 100, 110, 120, 130 or 140 modified internucleotide linkages. In certain embodiments, all internucleotide linkages in a gRNA are modified.

In certain embodiments, at least one of the modifications in the combination comprises a 2′-O-methyl, a 2′-fluoro, a 2′-amino, a 2′-deoxy, a 2′-arabino, a 2′-F-arabino, a 2-thiouracil, a 2-aminoadenine, a 5-methylcytosine, a 5-aminoallyluracil, a Z base, a 3′-phosphorothioate, a 3′-phosphonoacetate, a 3′-phosphonoacetate ester, a 3′-thiophosphonoacetate, 3′-thiophosphonoacetate ester, a 3′-methylphosphonate, a 3′-boranophosphate, a 3′-phosphorodithioate, or combinations thereof. In certain embodiments, at least one of the modifications in the combination comprises a 2′-O-methyl, a 2′-deoxy, a Z base, a phosphorothioate internucleotide linkage, a phosphonoacetate internucleotide linkage, a thiophosphonoacetate internucleotide linkage, or combinations thereof. In certain embodiments, at least one of the modifications in the combination comprises a 2′-F, a 2-thioU, a 4-thioU, a 2-aminoA, a 5-methylC, a 5-methylU, a 5-aminoallylU, or combinations thereof. In certain embodiments, at least one of the modifications in the combination is an “end” modification such as terminal phosphate, a PEG, a terminal amine, a terminal linker such as a hydrocarbon linker, a substituted hydrocarbon linker, a squarate linker, a triazolo linker, an internal linker such as 2-(4-butylamidofluorescein) propane-1,3-diol bis(phosphodiester) linker, a linker conjugated to a dye, a linker conjugated to a non-fluorescent label, a linker conjugated to a tag or a linker conjugated to a solid support such as for example a bead or microarray. In certain embodiments, at least two of the modifications in the combination comprise a 2′-O-methyl nucleotide and phosphorothioate internucleotide linkage, a 2′-O-methyl nucleotide and phosphonoacetate internucleotide linkage, or a 2′-O-methyl nucleotide and thiophosphonoacetate internucleotide linkage. In certain embodiments, at least two of the modifications in the combination comprise a 2′-O-methyl nucleotide and phosphonocarboxylate internucleotide linkage, a 2′-O-methyl nucleotide and phosphonocarboxylate ester internucleotide linkage, a 2′-O-methyl nucleotide and thiophosphonocarboxylate internucleotide linkage, a 2′-O-methyl nucleotide and thiophosphonocarboxylate ester internucleotide linkage, or combinations thereof. In other embodiments, the modifications in the combination further comprise a 2-thiouracil, 2-thiocytosine, 4-thiouracil, 6-thioguanine, 2-aminoadenine, 2-aminopurine, pseudouracil, inosine, 7-deazaguanine, 7-deaza-8-azaguanine, 7-deazaadenine, 7-deaza-8-azaadenine, 5-methylcytosine, 5-methyluracil, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil, 5-allylcytisine, 5-aminoallyl-uracil, 5-aminoallyl-cytosine, or an abasic nucleotide.

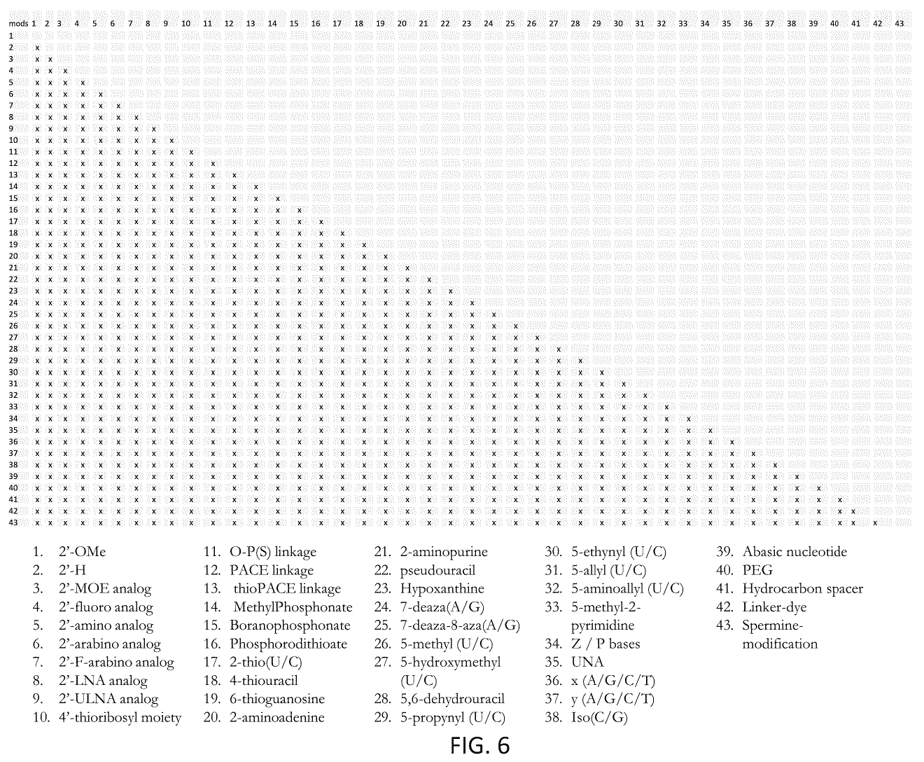

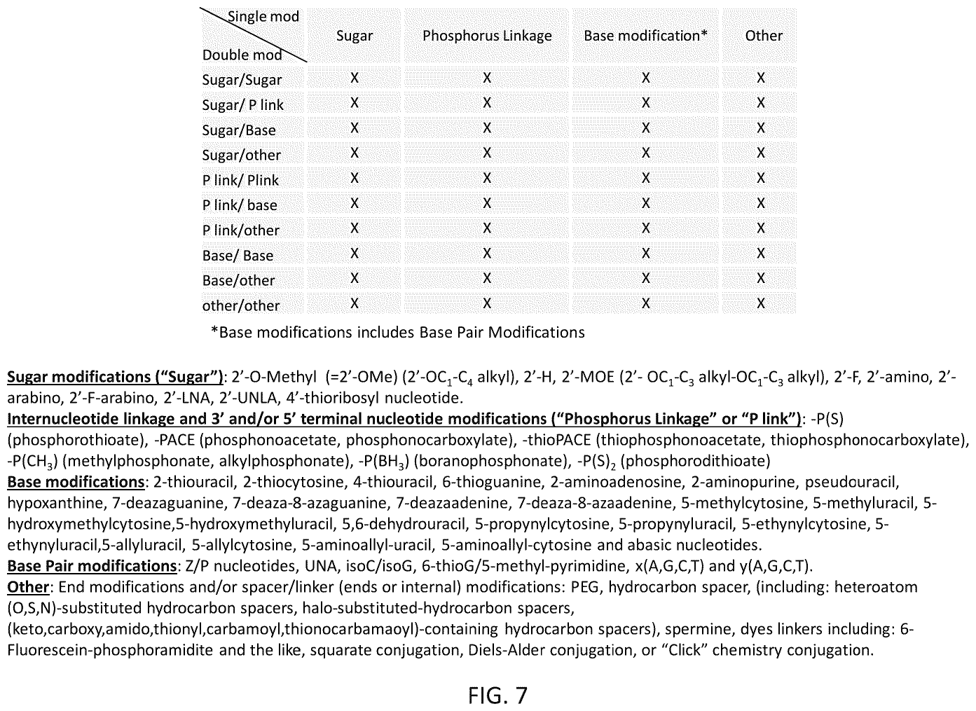

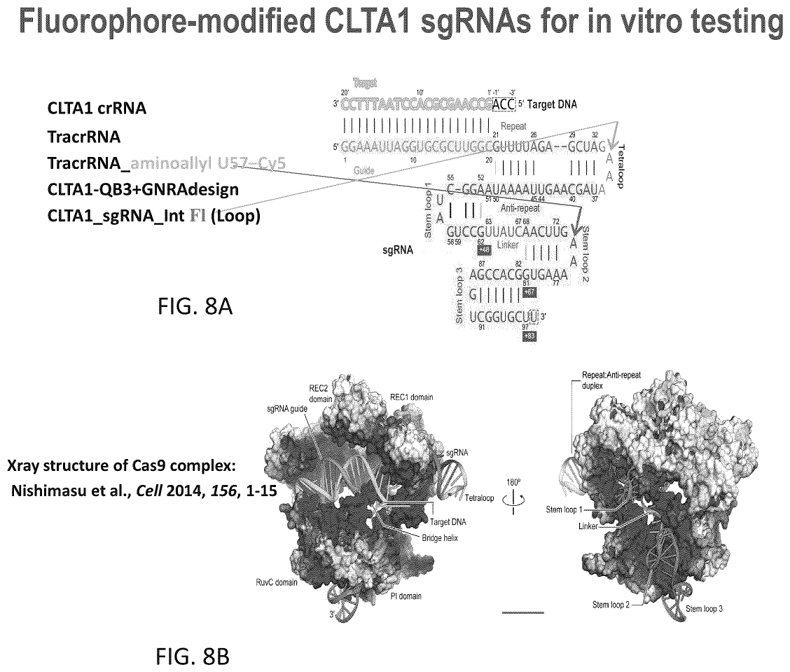

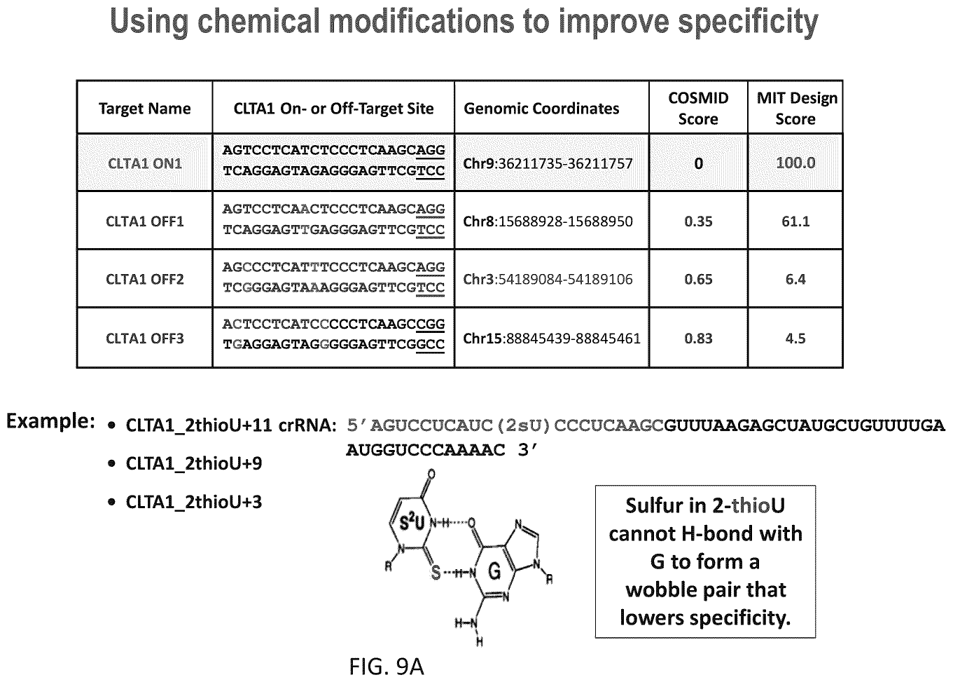

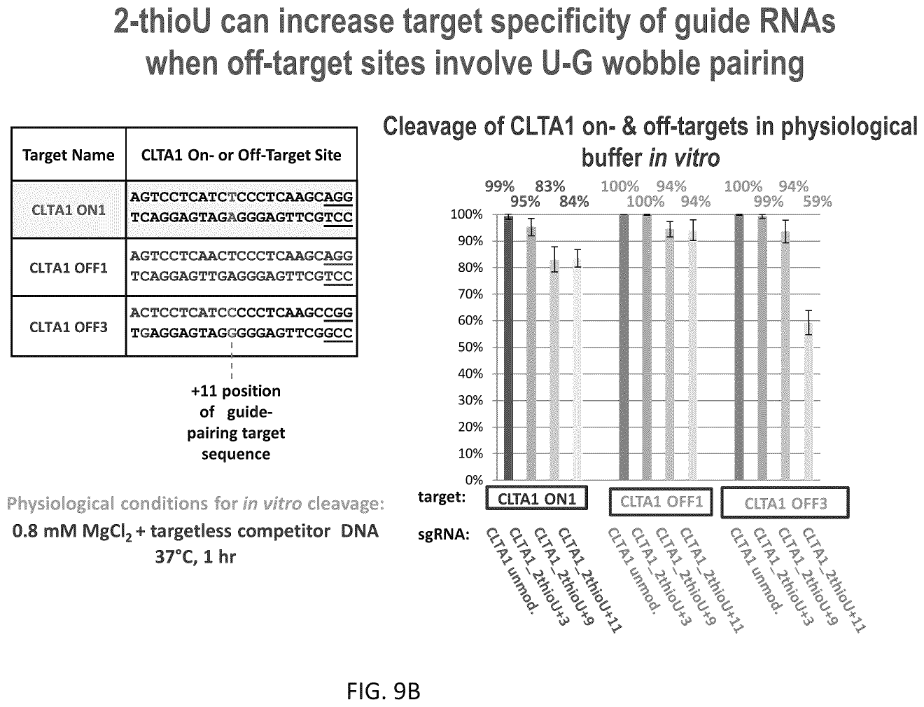

In certain embodiments, at least one of the modifications in the combination comprises a 2′-O-methyl-3′-phosphorothioate. In certain embodiments, at least one of the modifications in the combination comprises a 2′-O-methyl-3′-phosphonoacetate. In certain embodiments, at least one of the modifications in the combination comprises a 2′-O-methyl-3′-thiophosphonoacetate. In certain embodiments, at least one of the modifications in the combination comprises a 2′-halo-3′-phosphorothioate. In certain embodiments, at least one of the modifications in the combination comprises a 2′-halo-3′-phosphonoacetate. In certain embodiments, at least one of the modifications in the combination comprises a 2′-halo-3′-thiophosphonoacetate. In certain embodiments, at least one of the modifications in the combination comprises a 2′-fluoro-3′-phosphorothioate. In certain embodiments, at least one of the modifications in the combination comprises a 2′-fluoro-3′-phosphonoacetate. In certain embodiments, at least one of the modifications in the combination comprises a 2′-fluoro-3′-thiophosphonoacetate. Possible combinations of at least two or three modifications are represented in FIG. 6 and FIG. 7 respectively and are incorporated herein by reference.

In certain embodiments, the guide RNA comprises an oligonucleotide represented by Formula (III) or Formula (IV):

W—Y-Q (III); or

Y—W—X-Q (IV)
wherein Q and W each independently represent a nucleotide or a stretch of nucleotides of the oligonucleotide comprising at least one modification and Y and X each independently represent an unmodified portion of the oligonucleotide.

In certain embodiments, W is within the 5′ portion of the guide RNA. In certain embodiments, W is at least partially within the first five (5) nucleotides of the 5′ portion of the guide RNA. In certain embodiments, W is at least partially within the first three (3) nucleotides of the 5′ portion of the guide RNA. In certain embodiments, W is within the internal region (i.e., between the 5′ end and the 3′ end) of the guide RNA.

In certain embodiments, Q is within the 3′ portion of the guide RNA. In certain embodiments, Q is at least partially within the last five (5) nucleotides of the 3′ portion of the guide RNA. In certain embodiments, Q is at least partially within the last three (3) nucleotides of the 3′ portion of the guide RNA.

In certain embodiments, Q is within the internal region (i.e., between the 5′ end and the 3′ end) of the guide RNA.

In certain embodiments, W comprises an end modification as described above, such as a 5′ end or a 3′ end modification. In certain embodiments, the end modification comprises dimethoxytrityl.

In certain embodiments, at least one of W or Q comprises a modified base as described above. In certain embodiments, at least one of W or Q comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modified bases. In certain embodiments, at least one of W or Q comprises more than one modified base, such as 2, 3, 4, 5, 6, 7, 8, 9, 10, 11, 12, 13, 14, 15, 16, 17, 18, 19 or 20 modified bases.

In certain embodiments, at least one of W or Q comprises a modified sugar as described above. In certain embodiments, at least one of W or Q comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modified sugars. In certain embodiments, at least one of W or Q comprises more than one, such as 2, 3, 4, 5, 6, 7, 8, 9, 10, 11, 12, 13, 14, 15, 16, 17, 18, 19 or 20 modified sugars.

In certain embodiments, at least one of W or Q comprises a modified backbone (i.e., an internucleotide linkage other than a phosphodiester) as described above. In certain embodiments, at least one of W or Q comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 modified internucleotide linkages. In certain embodiments, at least one of W or Q comprises more than one, such as 2, 3, 4, 5, 6, 7, 8, 9, 10, 11, 12, 13, 14, 15, 16, 17, 18, 19 or 20 modified internucleotide linkages.

In certain embodiments, at least one of W or Q comprises a 2′-O-methyl nucleotide, a 2′-F nucleotide, a 2′-amino nucleotide, a 2′-deoxy nucleotide, a 2-thiouridine nucleotide, a 2-aminoadenosine nucleotide, a 6-thioguanosine nucleotide, a 5-methylcytidine nucleotide, a 5-aminoallyluridine nucleotide, a Z nucleotide, a 3′-phosphorothioate internucleotide linkage, a 3′-phosphorothioate internucleotide linkage, a 3′-phosphonoacetate internucleotide linkage, a 3′-phosphonoacetate ester internucleotide linkage, a 3′-thiophosphonoacetate internucleotide linkage, a 3′-thiophosphonoacetate ester internucleotide linkage, a 3′-methylphosphonate internucleotide linkage, a 3′-boranophosphate internucleotide linkage, a 3′-phosphorodithioate internucleotide linkage, or combinations thereof.

In certain embodiments, at least one of W or Q comprises a 2′-O-methyl and a 3′-phosphorothioate group on the same nucleotide. In certain embodiments, at least one of W or Q comprises a 2′-O-methyl and a 3′-phosphonoacetate group linkage on the same nucleotide. In certain embodiments, at least one of W or Q comprises a 2′-O-methyl and 3′-thiophosphonoacetate group on the same nucleotide. In certain embodiments, at least one of W or Q comprises a 2′-F and a 3′-phosphorothioate group on the same nucleotide. In certain embodiments, at least one of W or Q comprises a 2′-F and a 3′-phosphonoacetate group linkage on the same nucleotide. In certain embodiments, at least one of W or Q comprises a 2′-F and 3′-thiophosphonoacetate group on the same nucleotide.

In certain embodiments, at least one of W or Q comprises 1, 2, 3, 4, 5, 6, 7, 8, 9, 10, 11, 12, 13, 14, 15, 16, 17, 18, 19 or 20 modified nucleotides. In certain embodiments, each of the modified nucleotides within at least one of W or Q comprises the same modification or modifications. In certain embodiments, W comprises a modified nucleotide that is different than a modified nucleotide in Q. In certain embodiments, at least one of W or Q comprises two or more modified nucleotides. In certain embodiments, at least one of W or Q comprises three or more modified nucleotides. In certain embodiments, the modified nucleotides are not arranged contiguously in the sequence, or at least not entirely, as one or more unmodified nucleotides may intercede. In certain embodiments, the modified nucleotides are arranged contiguously. In certain embodiments, at least one of W or Q comprises at least one contiguous stretch of modified nucleotides. In certain embodiments, at least one of W or Q comprises a contiguous stretch of at least three (3) modified nucleotides. In certain embodiments, at least one of W or Q comprises a contiguous stretch of at least four (4) modified nucleotides. In certain embodiments, at least one of W or Q comprises a contiguous stretch of at least five (5) modified nucleotides.

In certain embodiments, the guide RNA comprises a nucleotide sequence of Formula (V) or Formula (VI):

MₘNₙM′_{m′}N′_{n′} (Formula V); or

MₘNₙM′_{m′}N′_{n′}M″_{m″} (Formula VI)
wherein each M independently represents a modified ribonucleotide;
wherein each N independently represents an unmodified ribonucleotide;
wherein each M′ independently represents a modified ribonucleotide;
wherein each N′ independently represents an unmodified ribonucleotide;
wherein each M″ independently represents a modified ribonucleotide;
wherein m is an integer between 0 and 40, n is an integer between 0 and 130, m′ is an integer between 0 and 10, n′ is an integer between 0 and 130, m″ is an integer between 0 and 10, provided that m+m′+m″ is greater than or equal to 1 and 50<m+n+m′+n′+m″≤150.

In certain embodiments, each M is independently selected from the group consisting of a 2′-O-methyl ribonucleotide, a 2′-O-methyl-3′-P(S) ribonucleotide, a 2′-O-methyl-3′-PACE ribonucleotide, a 2′-O-methyl-3′-thioPACE ribonucleotide, and a 2′-deoxynucleotide. In certain embodiments, each M is independently selected from the group consisting of a 2′-O-methyl ribonucleotide, a 2′-O-methyl-3′-P(S) ribonucleotide, a 2′-O-methyl-3′-PACE ribonucleotide, and a 2′-O-methyl-3′-thioPACE ribonucleotide. In certain embodiments, each M is independently selected from the group consisting of a 2′-O-methyl-3′-PACE ribonucleotide and a 2′-O-methyl-3′-thioPACE ribonucleotide. In certain embodiments, where m>1, any given M is the same or different from any other M. In certain embodiments, where m>1, each M comprises the same modification or modifications.

In certain embodiments, each M′ is independently selected from the group consisting of a 2′-O-methyl ribonucleotide, a 2′-O-methyl-3′-P(S) ribonucleotide, a 2′-O-methyl-3′-PACE ribonucleotide, a 2′-O-methyl-3′-thioPACE ribonucleotide, and a 2′-deoxynucleotide. In certain embodiments, each M′ is independently selected from the group consisting of a 2′-O-methyl ribonucleotide, a 2′-O-methyl-3′-P(S) ribonucleotide, a 2′-O-methyl-3′-PACE ribonucleotide, and a 2′-O-methyl-3′-thioPACE ribonucleotide. In certain embodiments, each M′ is independently selected from the group consisting of a 2′-O-methyl-3′-PACE ribonucleotide and a 2′-O-methyl-3′-thioPACE ribonucleotide. In certain embodiments, where m′>1, any given M′ is the same or different from any other M′. In certain embodiments, where m′>1, each M′ comprises the same modification or modifications.

In certain embodiments, each M″ is independently selected from the group consisting of a 2′-O-methyl ribonucleotide, a 2′-O-methyl-3′-P(S) ribonucleotide, a 2′-O-methyl-3′-PACE ribonucleotide, a 2′-O-methyl-3′-thioPACE ribonucleotide, and a 2′-deoxynucleotide. In certain embodiments, each M″ is independently selected from the group consisting of a 2′-O-methyl ribonucleotide, a 2′-O-methyl-3′-P(S) ribonucleotide, a 2′-O-methyl-3′-PACE ribonucleotide, and a 2′-O-methyl-3′-thioPACE ribonucleotide. In certain embodiments, each M″ is independently selected from the group consisting of a 2′-O-methyl-3′-PACE ribonucleotide and a 2′-O-methyl-3′-thioPACE ribonucleotide. In certain embodiments, where m″>1, any given M″ is the same or different from any other M″. In certain embodiments, where m′>1, each M″ comprises the same modification or modifications.

In certain embodiments, each M is a 2′-O-methyl-3′-PACE ribonucleotide; m is selected from an integer between 1 and 10; each N is independently selected from the group consisting of A. U. C, and G; n is selected from an integer between 10 and 130; each M′ is independently selected from the group consisting of a 2′-O-methyl ribonucleotide, a 2′-O-methyl-3′-P(S) ribonucleotide, a 2′-O-methyl-3′-PACE ribonucleotide, a 2′-O-methyl-3′-thioPACE ribonucleotide, a 2′-deoxynucleotide, and a Z nucleotide; m′ is selected from an integer between 1 and 10; each N is independently selected from the group consisting of A, U, C, and G; and n′ is selected from an integer between 0 and 130. In certain embodiments, each M′ is a 2′-O-methyl-3′-PACE ribonucleotide. In certain embodiments, each M′ is a 2′-O-methyl-3′-thioPACE ribonucleotide. In certain embodiments, each M′ is a 2′-O-methyl ribonucleotide. In certain embodiments, each M′ is a 2′-O-methyl-3′-P(S) ribonucleotide. In certain embodiments, each M′ is a Z nucleotide.

In certain embodiments, each M is a 2′-O-methyl-3′-thioPACE ribonucleotide; m is selected from an integer between 1 and 10; each N is independently selected from the group consisting of A. U. C. and G; n is selected from an integer between 10 and 130; each M′ is independently selected from the group consisting of a 2′-O-methyl ribonucleotide, a 2′-O-methyl-3′-P(S) ribonucleotide, a 2′-O-methyl-3′-PACE ribonucleotide, a 2′-O-methyl-3′-thioPACE ribonucleotide, a 2′-deoxynucleotide, and a Z nucleotide; m′ is selected from an integer between 1 and 10; each N is independently selected from the group consisting of A. U. C, and G; and n′ is selected from an integer between 0 and 130. In certain embodiments, each M′ is a 2′-O-methyl-3′-PACE ribonucleotide. In certain embodiments, each M′ is a 2′-O-methyl-3′-thioPACE ribonucleotide. In certain embodiments, each M′ is a 2′-O-methyl ribonucleotide. In certain embodiments, each M′ is a 2′-O-methyl-3′-P(S) ribonucleotide. In certain embodiments, each M′ is a Z nucleotide.

In certain embodiments, each M is a 2′-O-methyl-3′-P(S) ribonucleotide; m is selected from an integer between 1 and 10; each N is independently selected from the group consisting of A. U. C, and G; n is selected from an integer between 10 and 130; each M′ is independently selected from the group consisting of a 2′-O-methyl ribonucleotide, a 2′-O-methyl-3′-P(S) ribonucleotide, a 2′-O-methyl-3′-PACE ribonucleotide, a 2′-O-methyl-3′-thioPACE ribonucleotide, a 2′-deoxynucleotide, and a Z nucleotide; m′ is selected from an integer between 1 and 10; each N is independently selected from the group consisting of A, U, C, and G; and n′ is selected from an integer between 0 and 130. In certain embodiments, each M′ is a 2′-O-methyl-3′-PACE ribonucleotide. In certain embodiments, each M′ is a 2′-O-methyl-3′-thioPACE ribonucleotide. In certain embodiments, each M′ is a 2′-O-methyl ribonucleotide. In certain embodiments, each M′ is a 2′-O-methyl-3′-P(S) ribonucleotide. In certain embodiments, each M′ is a Z nucleotide.

In certain embodiments, each M is independently selected from the group consisting of a 2′-O-methyl ribonucleotide, a 2′-O-methyl-3′-P(S) ribonucleotide, a 2′-O-methyl-3′-PACE ribonucleotide, a 2′-O-methyl-3′-thioPACE ribonucleotide, a 2′-deoxynucleotide, and a Z nucleotide; m is selected from an integer between 0 and 10; each N is independently selected from the group consisting of A, U, C, and G; n is selected from an integer between 10 and 15; each M′ is a 2′-O-methyl ribonucleotide; m′ is selected from an integer between 1 and 5; each N is independently selected from the group consisting of A, U, C, and G; and n′ is selected from an integer between 0 and 130. In certain embodiments, each M is a 2′-O-methyl-3′-PACE ribonucleotide. In certain embodiments, each M is a 2′-O-methyl-3′-thioPACE ribonucleotide. In certain embodiments, each M is a 2′-O-methyl ribonucleotide. In certain embodiments, each M is a 2′-O-methyl-3′-P(S) ribonucleotide. In certain embodiments, m is 0; n is selected from an integer between 10 and 15, m′ is selected from an integer between 1 and 5; and n′ is selected from an integer between 0 and 130.

In certain embodiments, at least one of the modifications in the combination is a stability-altering modification. In certain embodiments, at least one of the modifications in the combination increases nuclease resistance of the guide RNA relative to a guide RNA without the modification, thus it enhances the stability of the guide RNA.

In certain embodiments, at least one of the modifications in the combination is a stability-enhancing modification as described above.

In certain embodiments, at least one of the modifications in the combination is a specificity-altering modification as described above.

In certain embodiments, at least one of the modifications in the combination alters RNA base pairing. In certain embodiments, at least one of the modifications in the combination lowers the Tm of a base pairing interaction as described above. In certain embodiments, at least one of the modifications in the combination raises the Tm of a base pairing interaction as described above.

In certain embodiments, at least one of the modifications in the combination alters transfection efficiency of the guide RNA relative to a guide RNA without the modification. In certain embodiments, at least one of the modifications in the combination increases transfection efficiency of the guide RNA relative to a guide RNA without the modification. In certain embodiments, at least one of the modifications in the combination decreases transfection efficiency of the guide RNA relative to a guide RNA without the modification. In certain embodiments, at least one of the transfection-increasing modifications in the combination comprises a phosphonoacetate alkyl ester internucleotide linkage, such as a phosphonoacetate methyl ester internucleotide linkage.

In certain embodiments, at least one of the modifications in the combination enhances stability and specificity of the guide RNA relative to a guide RNA without the modification. In certain embodiments, at least one of the modifications in the combination enhances stability and transfection efficiency of the guide RNA relative to a guide RNA without the modification. In certain embodiments, at least one of the modifications in the combination enhances specificity and transfection efficiency of the guide RNA relative to a guide RNA without the modification.

In certain embodiments, at least one of the modifications in the combination alters the secondary structure of the guide RNA. This modification alters the base-pairing of any of the RNA/RNA internal duplexes in the guide RNA. Some of these modifications increase the base pairing of the RNA/RNA structure or alternatively increase the Tm of the RNA/RNA duplex, whereas other modifications decrease the base pairing (or Tm) of the RNA/RNA duplex or duplexes. Such modifications include base modified nucleotides, particularly UNA nucleotides such as the 2-thiouridine and 2-aminoadenosine pair, the Z/P nucleotide pair, the isoC/isoG pair, the 6-thioG/5-methylpyrimidine pair, and nucleotides with modifications on the sugar or the internucleotide linkages as discussed before.

In certain embodiments, the combination includes at least one modification or a set of modifications that increases nucleases resistance (i.e., stability) with at least one modification or a set of modifications that increases specificity (i.e., reduces off-target effects). In certain embodiments, the combination includes at least one modification or a set of modifications that increases nucleases resistance (i.e., stability) with at least one modification or a set of modifications that raises the Tm of some bases pairing in the guide RNA. In certain embodiments, the combination includes at least one modification or a set of modifications that increases nucleases resistance (i.e., stability) with at least one modification or a set of modifications that lowers the Tm of some bases pairing of the guide RNA. In certain embodiments, the combination includes at least one modification or a set of modifications that increases nuclease resistance (i.e., stability), at least one modification or a set of modifications that increases the Tm of some bases paring in the guide RNA, and at least one modification or a set of modifications that decreases the Tm of some base paring elsewhere in the guide RNA. In certain embodiments, the combination includes at least one modification or a set of modifications that increases nuclease resistance (i.e., stability) and at least one modification or a set of modifications that increases the binding of the guide RNA to Cas protein. In certain embodiments, the combination includes at least one modification or a set of modifications that increases nuclease resistance (i.e., stability) and at least one modification or a set of modifications that decreases the binding of the guide RNA to Cas protein. In certain embodiments, the guide RNA comprises a combination of the different types of modifications.

### D. Guide RNA Structure

In certain embodiments, the guide RNA is able to form a complex with a CRISPR-associated-protein. In certain embodiments, the CRISPR-associated protein is provided by or is derived from a CRISPR-Cas type II system, which has an RNA-guided polynucleotide binding and/or nuclease activity. In certain embodiments, the CRISPR-associated protein is Cas9, a Cas9 mutant, or a Cas9 variant. In certain embodiments, the CRISPR-associated protein is the Cas9 nuclease from Streptococcus pyogenes. In certain embodiments, the CRISPR-associated protein is the Cas9 nuclease from Streptococcus thermophilus. In certain embodiments, the CRISPR-associated protein is the Cas9 nuclease from Staphylococcus aureus. In certain embodiments, the synthetic guide RNA or a synthetic guide RNA: CRISPR-associated protein complex maintains functionality of natural guide RNA or a complex that does not have modified nucleotides. In certain embodiments, the functionality includes binding a target polynucleotide. In certain embodiments, the functionality includes nicking a target polynucleotide. In certain embodiments, the functionality includes cleaving a target polynucleotide. In certain embodiments, the target polynucleotide is within a nucleic acid in vitro. In certain embodiments, the target polynucleotide is within the genome of a cell in vivo or in vitro (such as in cultured cells or cells isolated from an organism). In certain embodiments, the target polynucleotide is a protospacer in DNA.

In certain embodiments, the crRNA segment comprises from 25 to 80 nucleotides. In certain embodiments, the crRNA segment comprises a guide sequence that is capable of hybridizing to a target sequence. In certain embodiments, the guide sequence is complementary to the target sequence or a portion thereof. In certain embodiments, the guide sequence comprises from 15 to 30 nucleotides. In certain embodiments, the crRNA segment comprises a stem sequence. In certain embodiments, the stem sequence comprises from 10 to 50 nucleotides. In certain embodiments, the crRNA segment comprises a 5′-overhang sequence. In certain embodiments, the 5′-overhang sequence comprises from 1 to 10 nucleotides, alternatively 1 to 5 nucleotides, alternatively 1, 2 or 3 nucleotides. In certain embodiments, the crRNA comprises both (i) a guide sequence that is capable of hybridizing to a target sequence and (ii) a stem sequence. In certain embodiments, the crRNA comprises (i) a 5′-overhang sequence, (ii) a guide sequence that is capable of hybridizing to a target sequence, and (iii) a stem sequence. In certain embodiments wherein the crRNA segment comprises a stem sequence, the tracrRNA segment comprises a nucleotide sequence that is partially or completely complementary to the stem sequence of the crRNA segment. In certain embodiments, the tracrRNA segment comprises at least one more duplex structure.

In certain embodiments, the guide RNA is a single guide RNA. In certain embodiments, the guide RNA is a single guide RNA, wherein the crRNA segment and the tracrRNA segment are linked through a loop L. In certain embodiments, the loop L comprises 1, 2, 3, 4, 5, 6, 7, 8, 9, or 10 nucleotides. In certain embodiments, the loop L comprises a nucleotide sequence of GNRA, wherein N represents A. C. G, or U and R represents A or G. In certain embodiments, the loop L comprises a nucleotide sequence of GAAA. In certain embodiments, the guide RNA comprises more than one loop.

The guide RNA comprises a 5′ portion (i.e., the 5′ half) and a 3′ portion (i.e., the 3′ half). In certain embodiments, the crRNA segment is 5′ (i.e., upstream) of the tracrRNA segment. In certain embodiments, the tracrRNA segment is 5′ relative to the crRNA segment.

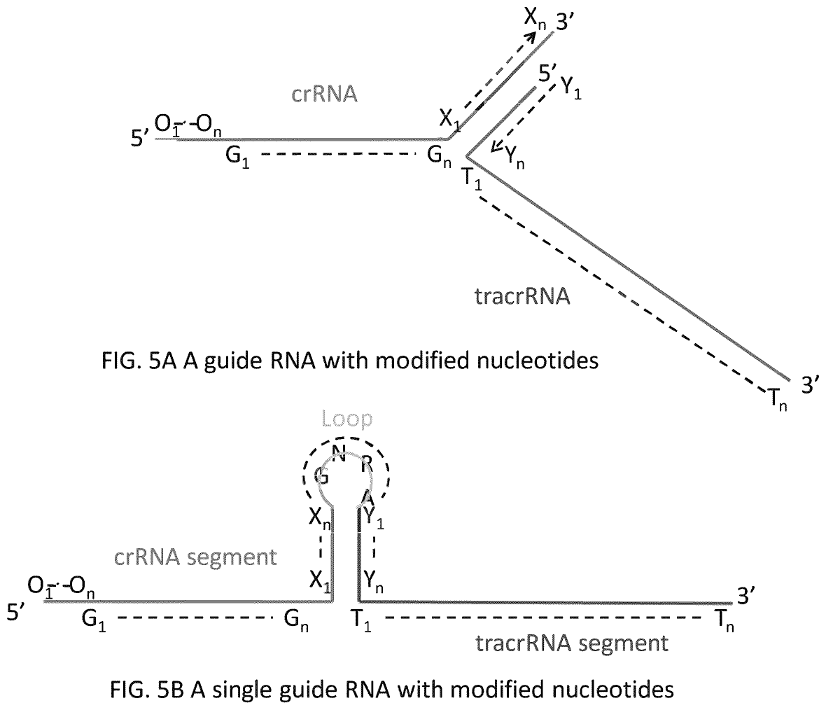

In certain embodiments, the guide RNA comprises at least two separate RNA strands, for example, a crRNA strand and a separate tracrRNA strand. See, for example, FIG. 5A. In certain embodiments, each of the strands is a synthetic strand comprising one or more modifications. In certain embodiments, at least one of the strands is a synthetic strand comprising one or more modifications. In certain embodiments, the strands function together to guide binding, nicking, or cleaving of a target polynucleotide by a Cas protein, such as Cas9. In certain embodiments, the crRNA sequence and the tracrRNA sequence are on separate stands and hybridize to each other via two complementary sequences to form a stem or duplex.

In certain embodiments, the guide RNA is a single guide RNA comprising a crRNA sequence and a tracrRNA sequence. See, for example, FIG. 5B. In certain embodiments, the crRNA sequence and the tracrRNA sequence are connected by a loop sequence or “loop.” In certain embodiments, a single guide RNA comprises a 5′ portion and a 3′ portion, wherein the crRNA sequence is upstream of the tracrRNA sequence.

In certain embodiments, the total length of the two RNA pieces can be about 50-220 (e.g., about 55-200, 60-190, 60-180, 60-170, 60-160, 60-150, 60-140, 60-130, and 60-120) nucleotides in length, such as about 60, 70, 80, 90, 100, 110, 120, 130, 140, 150, 160, 170, 180, 190, 200, or 220 nucleotides in length. Similarly, the single guide RNA (e.g., FIG. 5B) can be about 50-220 (e.g., about 55-200, 60-190, 60-180, 60-170, 60-160, 60-150, 60-140, 60-130, and 60-120) nucleotides in length, such as about 60, 70, 80, 90, 100, 110, 120, 130, 140, 150, 160, 170, 180, 190, 200, or 220 nucleotides in length.

As shown in FIGS. 5A and 5B, the synthetic guide RNA comprises (i) a crRNA sequence that comprises (a) a guide sequence (e.g., segment G1-Gn, where each G represents a nucleotide in the guide sequence) capable of hybridizing to a target sequence in a nucleic acid, (b) a first stem sequence (e.g., segment X1-Xn, where each X represents a nucleotide in the first stem sequence) capable of hybridizing partially or completely to a second stem sequence, and, optionally (c) a 5′-overhang sequence (e.g., segment 01-On, where each O represents a nucleotide in the overhang sequence), and (ii) a tracrRNA sequence that comprises the second stem sequence (e.g., segment Y1-Yn, where each Y represents a nucleotide in the second stem sequence). The tracrRNA sequence further comprises segment T1-Tn, where each T represents a nucleotide in the tracrRNA sequence. The synthetic guide RNA shown in FIG. 5A includes one or more modifications. Likewise, the synthetic guide RNA shown in FIG. 5B includes one or more modifications. In certain embodiments, the modification is located at any point along the length of the crRNA, the tracrRNA, or the single guide RNA comprising a crRNA segment, a tracrRNA segment, and, optionally, a loop. In certain embodiments, any nucleotide represented by O, G, X, Y, or T in the synthetic guide RNA shown in FIGS. 5A and 5B may be a modified nucleotide. The guide RNA shown in FIG. 5B represents a single guide RNA (sgRNA) where the crRNA segment and the tracrRNA segment are connected by a loop having the sequence GNRA, wherein N represents A, C, G, or U, and R represents A or G.

In certain embodiments, the crRNA segment of the guide RNA is 25-70 (e.g., 30-60, 35-50, or 40-45) nucleotides in length. In certain embodiments, the guide sequence is 12-30 (e.g., 16-25, 17-20, or 15-18) nucleotides in length. In some embodiments, a 5′ portion of the crRNA does not hybridize or only partially hybridizes with the target sequence. For example, there can be a 5′-overhang on the crRNA segment.

In certain embodiments, the single guide RNA comprises a central portion including the stem sequence of the crRNA segment, the stem sequence of the tracrRNA segment, and, optionally, a loop that covalently connects the crRNA segment to the tracrRNA segment. In certain embodiments, the central segment of the single guide RNA is 8-60 (e.g., 10-55, 10-50, or 20-40) nucleotides in length.

In certain embodiments, the tracrRNA segment of the guide RNA is 10-130 (e.g., 10-125, 10-100, 10-75, 10-50, or 10-25) nucleotides in length. In certain embodiments, the tracrRNA segment includes one or more hairpin or duplex structures in addition to any hairpin or duplex structure in the central segment.

In certain embodiments, the tracrRNA is truncated compared to a reference tracrRNA, such as a naturally existing mature tracrRNA. A range of lengths has been shown to function in both the separate type (FIG. 5A) and the chimeric sgRNA type (FIG. 5B). For example, in certain embodiments, tracrRNA may be truncated from its 3′ end by at least 1, 2, 3, 4, 5, 6, 7, 8, 9, 10, 15, 20, 25, 30, 35 or 40 nts. In certain embodiments, the tracrRNA molecule may be truncated from its 5′ end by at least 1, 2, 3, 4, 5, 6, 7, 8, 9, 10, 15, 20, 25, 30, 35, 40, 45, 50, 55, 60, 65, 70, 75 or 80 nts. In certain embodiments, the tracrRNA molecule may be truncated from both the 5′ and 3′ end, e.g., by at least 1, 2, 3, 4, 5, 6, 7, 8, 9, 10, 15 or 20 nts from the 5′ end and at least 1, 2, 3, 4, 5, 6, 7, 8, 9, 10, 15, 20, 25, 30, 35 or 40 nts from the 3′ end. See, e.g., Jinek et al. (2012) Science, 337, 816-21; Mali et al. (2013) Science, 339:6121, 823-6; Cong et al. (2013) Science, 339:6121, 819-23; and Hwang et al. (2013) Nat. Biotechnol. 31:3, 227-9; Jinek et al. (2013) eLife. 2, e00471. In certain embodiments, the tracrRNA is untruncated.

In certain embodiments, the disclosed modifications are in the crRNA segment or the tracrRNA segment or both. In certain embodiments, the disclosed modifications are in the guide sequence of the crRNA segment. In certain embodiments, the disclosed modifications are in the stem sequence of the crRNA segment. In certain embodiments, the disclosed modifications are in the 5′-overhang sequence of the crRNA segment. In certain embodiments, the disclosed modifications are in the stem sequence of the tracrRNA segment. In certain embodiments, the disclosed modifications are in the loop sequence of the guide RNA. In certain embodiments, the disclosed modifications are in the 5′ portion of the guide RNA. In certain embodiments, the disclosed modifications are in the 3′ portion of the guide RNA. In certain embodiments, the disclosed modifications are in the 5′ portion of the guide RNA and the 3′ portion of the guide RNA.

### E. Synthesis of Guide RNA

In certain embodiments, guide RNAs, including single guide RNAs (sgRNAs; see FIGS. 1 and 5B) are produced by chemical synthesis using the art of synthetic organic chemistry. A guide RNA that comprises any nucleotide other than the four predominant ribonucleotides, namely A, C, G, and U, whether unnatural or natural, such as a pseudouridine, inosine or a deoxynucleotide, possesses a chemical modification or substitution at the nucleotide which is chemically/structurally distinct from any of the four predominant nucleotides in RNAs.

The synthetic guide RNAs described herein can be chemically synthesized. For example, the synthetic guide RNAs can be synthesized using TC chemistry by the method described in Dellinger et al. (2011) J. Am. Chem. Soc., 133, 11540, U.S. Pat. No. 8,202,983, and US Patent Application 2010/0076183A1, the contents of which are incorporated by reference in their entireties. “TC chemistry” refers to the composition and methods of using RNA monomeric nucleotide precursors protected on the 2′-hydroxyl moiety by a thionocarbamate protecting group, to synthesize unmodified RNA or modified RNA comprising one or more modified nucleotides. The ability to chemically synthesize relatively long RNAs (as long as 200 mers or more) using TC-RNA chemistry allows one to produce guide RNAs with special features capable of outperforming those enabled by the four predominant ribonucleotides (A, C, G and U). Some synthetic guide RNAs described herein can also be made using methods known in the art that include in vitro transcription and cell-based expression. For example, 2′-fluoro NTPs can be incorporated into synthetic guide RNAs produced by cell-based expression.

Synthesis of guide RNAs can also be accomplished by chemical or enzymatic synthesis of RNA sequences that are subsequently ligated together by enzymes, or chemically ligated by chemical ligation, including but not limited to cyanogen bromide chemistry, “click” chemistry as published by R. Kumar et al. (2007) J. Am. Chem. Soc., 129, 6859-64, or squarate conjugation chemistry as described by K. Hill in WO2013176844 titled “Compositions and methods for conjugating oligonucleotides.”

As further described below, a guide RNA disclosed herein, including those comprising modified nucleotides and/or modified internucleotide linkages, can be used to perform various CRISPR-mediated functions (including but not limited to editing genes, regulating gene expression, cleaving target sequences, and binding to target sequences) in vitro or in vivo, such as in cell-free assays, in intact cells, or in whole organisms. For in vitro or in vivo applications, the RNA can be delivered into cells or whole organisms in any manner known in the art.

### Libraries and Arrays

In one aspect, the present invention provides a set or library of multiple guide RNAs. In certain embodiments, the library contains two or more guide RNAs disclosed herein. The library can contain from about 10 to about 107 individual members, e.g., about 10 to about 102, about 102 to about 103, about 103 to about 105, from about 105 to about 107 members. An individual member of the library differs from other members of the library at least in the guide sequence, i.e., the DNA targeting segment of the gRNA. On the other hand, in certain embodiments, each individual member of a library can contain the same or substantially the same nucleotide sequence for the tracrRNA segment as all the other members of the library. In this way, the library can comprise members that target different polynucleotides or different sequences in one or more polynucleotides.

In certain embodiments, the library comprises at least 102 unique guide sequences. In certain embodiments, the library comprises at least 103 unique guide sequences. In certain embodiments, the library comprises at least 104 unique guide sequences. In certain embodiments, the library comprises at least 105 unique guide sequences. In certain embodiments, the library comprises at least 106 unique guide sequences. In certain embodiments, the library comprises at least 107 unique guide sequences. In certain embodiments, the library targets at least 10 different polynucleotides. In certain embodiments, the library targets at least 102 different polynucleotides. In certain embodiments, the library targets at least 103 different polynucleotides. In certain embodiments, the library targets at least 104 different polynucleotides. In certain embodiments, the library targets at least 105 different polynucleotides. In certain embodiments, the library targets at least 106 different polynucleotides. In certain embodiments, the library targets at least 107 different polynucleotides.

In certain embodiments, the library comprises a collection of guide RNAs having the same sequence and the same modifications in a progressively shifted window that moves across the sequence of the members in the library. In certain embodiments, the windows collectively cover the entire length of the RNA.

In certain embodiments, the library allows one to conduct high-throughput, multi-target genomic manipulations and analyses. In certain embodiments, only the DNA-targeting segments of the guide RNAs are varied, while the Cas protein-binding segment is the same. In certain embodiments, a first portion of the library comprises guide RNAs possessing a Cas-binding segment that recognizes, binds and directs a particular Cas protein and a second portion of the library comprises a different Cas-binding segment that recognizes, binds and directs a different Cas protein (e.g., a Cas protein from a different species), thereby allowing the library to function with two or more orthogonal Cas proteins. In certain embodiments, induced expression of a first orthogonal Cas protein utilizes the portion of the library which interacts with the first orthogonal Cas protein. In certain embodiments, induced expression of a first and second orthogonal Cas protein utilizes the portions of the library which interact with the first and second orthogonal Cas proteins, respectively. In certain embodiments, induced expression of the first and second orthogonal Cas proteins occur at different times. Accordingly, one can carry out large-scale gene editing or gene regulation by specifically manipulating or modifying multiple targets as specified in the library.

In certain embodiments, the library is an “arrayed” library, namely a collection of different features or pools of features in an addressable arrangement. For example, features of an array can be selectively cleaved and transferred to a microtiter plate such that each well in the plate contains a known feature or a known pool of features. In some other embodiments, the library is synthesized in a 48-columns or in a 96-columns microtiter plate format or in a 384-columns plate.

In certain embodiments, synthesis of the guide RNA of this invention may be conducted on a solid support having a surface to which chemical entities may bind. In some embodiments, guide RNAs being synthesized are attached, directly or indirectly, to the same solid support and may form part of an array. An “array” is a collection of separate molecules of known monomeric sequence each arranged in a spatially defined and a physically addressable manner, such that the location of each sequence is known. An “array.” or “microarray” used interchangeably herein includes any one-dimensional, two-dimensional or substantially two-dimensional (as well as a three-dimensional) arrangement of addressable regions bearing a particular chemical moiety or moieties (such as ligands, e.g., biopolymers such as polynucleotide or oligonucleotide sequences (nucleic acids), polypeptides (e.g., proteins), carbohydrates, lipids, etc.) associated with that region. An array is “addressable” when it has multiple regions of different moieties (e.g., different polynucleotide sequences) such that a region (i.e., a “feature” of the array) at a particular predetermined location (i.e., an “address”) on the array will detect a particular target or class of targets (although a feature may incidentally detect non-targets of that feature). Array features are typically, but need not be, separated by intervening spaces. The number of features that can be contained on an array will largely be determined by the surface area of the substrate, the size of a feature and the spacing between features. Arrays can have densities of up to several hundred thousand or more features per cm2, such as 2,500 to 200,000 features/cm2. The features may or may not be covalently bonded to the substrate.

Suitable solid supports may have a variety of forms and compositions and derive from naturally occurring materials, naturally occurring materials that have been synthetically modified, or synthetic materials. Examples of suitable support materials include, but are not limited to, silicas, silicon and silicon oxides, teflons, glasses, polysaccharides such as agarose (e.g., Sepharose® from Pharmacia) and dextran (e.g., Sephadex® and Sephacryl®, also from Pharmacia), polyacrylamides, polystyrenes, polyvinyl alcohols, copolymers of hydroxyethyl methacrylate and methyl methacrylate, and the like. In some embodiments, the solid support is a plurality of beads.

The initial monomer of the guide RNAs to be synthesized on the substrate surface can be bound to a linker which in turn is bound to a surface hydrophilic group, e.g., a surface hydroxyl moiety present on a silica substrate. In some embodiments, a universal linker is used. In some other embodiments, the initial monomer is reacted directly with, e.g., a surface hydroxyl moiety. Alternatively, guide RNAs can be synthesized first according to the present invention, and attached to a solid substrate post-synthesis by any method known in the art. Thus, the present invention can be used to prepare arrays of guide RNAs wherein the oligonucleotides are either synthesized on the array, or attached to the array substrate post-synthesis. Subsequently, the guide RNAs or a pool or a plurality of pools of guide RNAs can optionally and selectively be cleaved from the array substrate and be used as a library or libraries.

### IV. Cas Proteins

As mentioned above, a functional CRISPR-Cas system also requires a protein component (e.g., a Cas protein, which may be a Cas nuclease) that provides a desired activity, such as target binding or target nicking/cleaving. In certain embodiments, the desired activity is target binding. In certain embodiments, the desired activity is target nicking or target cleaving. In certain embodiments, the desired activity also includes a function provided by a polypeptide that is covalently fused to a Cas protein, as disclosed herein. In certain embodiments, the desired activity also includes a function provided by a polypeptide that is covalently fused to a nuclease-deficient Cas protein, as disclosed herein. Examples of such a desired activity include a transcription regulation activity (either activation or repression), an epigenetic modification activity, or a target visualization/identification activity, as described below. The Cas protein can be introduced into an in vitro or in vivo system as a purified or non-purified (i) Cas protein or (ii) mRNA encoded for expression of the Cas protein or (iii) linear or circular DNA encoded for expression of the protein. Any of these 3 methods of providing the Cas protein are well known in the art and are implied interchangeably when mention is made herein of a Cas protein or use of a Cas protein. In certain embodiments, the Cas protein is constitutively expressed from mRNA or DNA. In certain embodiments, the expression of Cas protein from mRNA or DNA is inducible or induced.

In certain embodiments, the Cas protein is chemically synthesized (see e.g., Creighton, “Proteins: Structures and Molecular Principles,” W. H. Freeman & Co., NY, 1983), or produced by recombinant DNA technology as described herein. For additional guidance, skilled artisans may consult Frederick M. Ausubel et al., “Current Protocols in Molecular Biology,” John Wiley & Sons, 2003; and Sambrook et al., “Molecular Cloning, A Laboratory Manual,” Cold Spring Harbor Press, Cold Spring Harbor, NY, 2001).

In certain embodiments, the Cas protein is provided in purified or isolated form. In certain embodiments, the Cas protein is provided at about 80%, about 90%, about 95%, or about 99% purity. In certain embodiments, the Cas protein is provided as part of a composition. In certain embodiments, the Cas protein is provided in aqueous compositions suitable for use as, or inclusion in, a composition for an RNA-guided nuclease reaction. Those of skill in the art are well aware of the various substances that can be included in such nuclease reaction compositions.

In certain embodiments, a Cas protein is provided as a recombinant polypeptide. In certain examples, the recombinant polypeptide is prepared as a fusion protein. For example, in certain embodiments, a nucleic acid encoding the Cas protein is linked to another nucleic acid encoding a fusion partner, e.g., glutathione-s-transferase (GST), 6×-His epitope tag, or M13 Gene 3 protein. Suitable host cells can be used to expresses the fusion protein. In certain embodiments, the fusion protein is isolated by methods known in the art. In certain embodiments, the fusion protein can be further treated, e.g., by enzymatic digestion, to remove the fusion partner and obtain the Cas protein. Alternatively, Cas protein: guide RNA complexes can be made with recombinant technology using a host cell system or an in vitro translation-transcription system known in the art. Details of such systems and technology can be found in e.g., WO2014144761, WO2014144592, WO2013176772, US20140273226, and US20140273233, the contents of which are incorporated herein by reference in their entireties.

### Wild Type Cas Proteins

In certain embodiments, a Cas protein comprises a protein derived from a CRISPR-Cas type I, type II, or type III system, which has an RNA-guided polynucleotide binding and/or nuclease activity. Non-limiting examples of suitable Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966. See e.g., WO2014144761, WO2014144592, WO2013176772, US20140273226, and US20140273233, the contents of which are incorporated herein by reference in their entireties.

In certain embodiments, the Cas protein is derived from a type II CRISPR-Cas system. In certain embodiments, the Cas protein is or is derived from a Cas9 protein. In certain embodiments, the Cas protein is or is derived from a bacterial Cas9 protein, including those identified in WO2014144761. In certain embodiments, the Cas protein is or is derived from a Streptococcus sp. or Staphylococcus sp. Cas9 protein. In certain embodiments, the Cas protein is or is derived from the Streptococcus thermophilus Cas9 protein. In certain embodiments, the Cas protein is or is derived from a the Streptococcus pyogenes Cas9 protein. In certain embodiments, the Cas protein is or is derived from the Staphylococcus aureus Cas9 protein. In certain embodiments, the Cas protein is or is derived from the Streptococcus thermophilus Cas9 protein.

In certain embodiments, the wild type Cas protein is a Cas9 protein. In certain embodiments, the wild type Cas9 protein is the Cas9 protein from S. pyogenes (SEQ ID NO: 1). In certain embodiments, the protein or polypeptide can comprise, consist of, or consist essentially of a fragment of SEQ ID NO: 1.

In general, a Cas protein includes at least one RNA binding domain, which interacts with the guide RNA. In certain embodiments, the Cas protein is modified to increase nucleic acid binding affinity and/or specificity, alter an enzymatic activity, and/or change another property of the protein. For example, nuclease (i.e., DNase, RNase) domains of the Cas protein can be modified, mutated, deleted, or inactivated. Alternatively, the Cas protein can be truncated to remove domains that are not essential for the function of the protein. In certain embodiments, the Cas protein is truncated or modified to optimize the activity of the effector domain. In certain embodiments, the Cas protein includes a nuclear localization sequence (NLS) that effects importation of the NLS-tagged Cas protein into the nucleus of a living cell. In certain embodiments, the Cas protein includes two or more modifications.

### Mutant Cas Proteins

In some embodiments, the Cas protein can be a mutant of a wild type Cas protein (such as Cas9) or a fragment thereof. In other embodiments, the Cas protein can be derived from a mutant Cas protein. For example, the amino acid sequence of the Cas9 protein can be modified to alter one or more properties (e.g., nuclease activity, binding affinity, stability to proteases, etc.) of the protein. Alternatively, domains of the Cas9 protein not involved in RNA-guided cleavage can be eliminated from the protein such that the modified Cas9 protein is smaller than the wild type Cas9 protein. For example, reducing the size of the Cas9 coding sequence can allow it to fit within a transfection vector that otherwise cannot accommodate the wild type sequence, such as the AAV vector among others. In some embodiments, the present system utilizes the Cas9 protein from S. pyogenes, either as encoded in bacteria or codon-optimized for expression in eukaryotic cells. Shown below is the amino acid sequence of wild type S. pyogenes Cas9 protein sequence (SEQ ID No. 1, Uniprot No. Q99ZW2).

MDKKYSIGLDIGTNSVGWAVITDEYKVPSKKFKVLGNTDR
HSIKKNLIGALLFDSGETAEATRLKRTARRRYTRRKNRIC
YLQEIFSNEMAKVDDSFFHRLEESFLVEEDKKHERHPIFG
NIVDEVAYHEKYPTIYHLRKKLVDSTDKADLRLIYLALAH
MIKFRGHFLIEGDLNPDNSDVDKLFIQLVQTYNQLFEENP
INASGVDAKAILSARLSKSRRLENLIAQLPGEKKNGLFGN
LIALSLGLTPNFKSNFDLAEDAKLQLSKDTYDDDLDNLLA
QIGDQYADLFLAAKNLSDAILLSDILRVNTEITKAPLSAS
MIKRYDEHHQDLTLLKALVRQQLPEKYKEIFFDQSKNGYA
GYIDGGASQEEFYKFIKPILEKMDGTEELLVKLNREDLLR
KQRTFDNGSIPHQIHLGELHAILRRQEDFYPFLKDNREKI
EKILTFRIPYYVGPLARGNSRFAWMTRKSEETITPWNFEE
VVDKGASAQSFIERMTNFDKNLPNEKVLPKHSLLYEYFTV
YNELTKVKYVTEGMRKPAFLSGEQKKAIVDLLFKTNRKVT
VKQLKEDYFKKIECFDSVEISGVEDRFNASLGTYHDLLKI
IKDKDFLDNEENEDILEDIVLTLTLFEDREMIEERLKTYA
HLFDDKVMKQLKRRRYTGWGRLSRKLINGIRDKQSGKTIL
DFLKSDGFANRNFMQLIHDDSLTFKEDIQKAQVSGQGDSL
HEHIANLAGSPAIKKGILQTVKVVDELVKVMGRHKPENIV
IEMARENQTTQKGQKNSRERMKRIEEGIKELGSQILKEHP
VENTQLQNEKLYLYYLQNGRDMYVDQELDINRLSDYDVDH
IVPQSFLKDDSIDNKVLTRSDKNRGKSDNVPSEEVVKKMK
NYWRQLLNAKLITQRKFDNLTKAERGGLSELDKAGFIKRQ
LVETRQITKHVAQILDSRMNTKYDENDKLIREVKVITLKS
KLVSDFRKDFQFYKVREINNYHHAHDAYLNAVVGTALIKK
YPKLESEFVYGDYKVYDVRKMIAKSEQEIGKATAKYFFYS
NIMNFFKTEITLANGEIRKRPLIETNGETGEIVWDKGRDF
ATVRKVLSMPQVNIVKKTEVQTGGFSKESILPKRNSDKLI
ARKKDWDPKKYGGFDSPTVAYSVLVVAKVEKGKSKKLKSV
KELLGITIMERSSFEKNPIDFLEAKGYKEVKKDLIIKLPK
YSLFELENGRKRMLASAGELQKGNELALPSKYVNFLYLAS
HYEKLKGSPEDNEQKQLFVEQHKHYLDEIIEQISEFSKRV
ILADANLDKVLSAYNKHRDKPIREQAENIIHLFTLTNLGA
PAAFKYFDTTIDRKRYTSTKEVLDATLIHQSITGLYETRI
DLSQLGGD

A Cas9 protein generally has at least two nuclease (e.g., DNase) domains. For example, a Cas9 protein can have a RuvC-like nuclease domain and an HNH-like nuclease domain. The RuvC and HNH domains work together to cut both strands in a target site to make a double-stranded break in the target polynucleotide. (Jinek et al., Science, 337:816-821). In certain embodiments, a mutant Cas9 protein is modified to contain only one functional nuclease domain (either a RuvC-like or an HNH-like nuclease domain). For example, in certain embodiments, the mutant Cas9 protein is modified such that one of the nuclease domains is deleted or mutated such that it is no longer functional (i.e., the nuclease activity is absent). In some embodiments where one of the nuclease domains is inactive, the mutant is able to introduce a nick into a double-stranded polynucleotide (such protein is termed a “nickase”) but not able to cleave the double-stranded polynucleotide. For example, an aspartate to alanine (D10A) conversion in a RuvC-like domain converts the Cas9-derived protein into a nickase. Likewise, a histidine to alanine (H840A) conversion in a HNH domain converts the Cas9-derived protein into a nickase. Likewise, an asparagine to alanine (N863A) conversion in a HNH domain converts the Cas9-derived protein into a nickase.

In certain embodiments, both the RuvC-like nuclease domain and the HNH-like nuclease domain are modified or eliminated such that the mutant Cas9 protein is unable to nick or cleave the target polynucleotide. In certain embodiments, all nuclease domains of the Cas9-derived protein are modified or eliminated such that the Cas9-derived protein lacks all nuclease activity. In certain embodiments, a Cas9 protein that lacks some or all nuclease activity relative to a wild-type counterpart, nevertheless, maintains target recognition activity to a greater or lesser extent.

In any of the above-described embodiments, any or all of the nuclease domains can be inactivated by one or more deletion mutations, insertion mutations, and/or substitution mutations using well-known methods, such as site-directed mutagenesis, PCR-mediated mutagenesis, and total gene synthesis, as well as other methods known in the art.

In certain embodiments, the “Cas mutant” or “Cas variant” is at least 50% (e.g., any number between 50% and 100%, inclusive, e.g., 50%, 60%, 70%, 80%, 90%, 95%, 98%, and 99%) identical to SEQ ID NO: 1. In certain embodiments, the “Cas mutant” or “Cas variant” binds to an RNA molecule (e.g., a sgRNA). In certain embodiments, the “Cas mutant” or “Cas variant” is targeted to a specific polynucleotide sequence via the RNA molecule.

### Fusion Proteins

In certain embodiments, the Cas protein is fused to another protein or polypeptide heterologous to the Cas protein to create a fusion protein. In certain embodiments, the heterologous sequence includes one or more effector domains, such as a cleavage domain, a transcriptional activation domain, a transcriptional repressor domain, or an epigenetic modification domain. Additional examples of the effector domain include a nuclear localization signal, cell-penetrating or translocation domain, or a marker domain. In certain embodiments, the effector domain is located at the N-terminal, the C-terminal, or in an internal location of the fusion protein. In certain embodiments, the Cas protein of the fusion protein is or is derived from a Cas9 protein. In certain embodiments, the Cas protein of the fusion protein is or is derived from a modified or mutated Cas protein in which all the nuclease domains have been inactivated or deleted. In certain embodiments, the Cas protein of the fusion protein is or is derived from a modified or mutated Cas protein that lacks nuclease activity. In certain embodiments, the RuvC and/or HNH domains of the Cas protein are modified or mutated such that they no longer possess nuclease activity.

### Cleavage Domains

In certain embodiments, the effector domain of the fusion protein is a cleavage domain. As used herein, a “cleavage domain” refers to a domain that cleaves DNA. The cleavage domain can be obtained from any endonuclease or exonuclease. Non-limiting examples of endonucleases from which a cleavage domain can be derived include restriction endonucleases and homing endonucleases. See, for example, New England Biolabs Catalog or Belfort et al. (1997) Nucleic Acids Res. 25, 3379-88. Additional enzymes that cleave DNA are known (e.g., 51 Nuclease; mung bean nuclease; pancreatic DNase I; micrococcal nuclease; yeast HO endonuclease). See also Linn et al. (eds.) “Nucleases,” Cold Spring Harbor Laboratory Press, 1993. One or more of these enzymes (or functional fragments thereof) can be used as a source of cleavage domains.

In certain embodiments, the cleavage domain can be derived from a type II-S endonuclease. Type II-S endonucleases cleave DNA specifically at sites that are typically several base pairs away from the DNA recognition site of the endonuclease and, as such, have separable recognition and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MbolI, and SapI. In certain embodiments, the cleavage domain of the fusion protein is a FokI cleavage domain or a fragment or derivative thereof. See Miller et al. (2007) Nat. Biotechnol. 25, 778-85; Szczpek et al. (2007) Nat. Biotechnol. 25, 786-93; Doyon et al. (2011) Nat. Methods, 8, 74-81.

### Transcriptional Activation Domains

In certain embodiments, the effector domain of the fusion protein is a transcriptional activation domain. In general, a transcriptional activation domain interacts with transcriptional control elements and/or transcriptional regulatory proteins (i.e., transcription factors, RNA polymerases, etc.) to increase and/or activate transcription of a gene. In certain embodiments, the transcriptional activation domain is a herpes simplex virus VP16 activation domain, VP64 (which is a tetrameric derivative of VP16), a NFκB p65 activation domain, p53 activation domains 1 and 2, a CREB (cAMP response element binding protein) activation domain, an E2A activation domain, or an NFAT (nuclear factor of activated T-cells) activation domain. In certain embodiments, the transcriptional activation domain is Gal4, Gcn4, MLL, Rtg3, Gln3, Oaf1, Pip2, Pdr1, Pdr3, Pho4, or Leu3. The transcriptional activation domain may be wild type, or it may be a modified or truncated version of the original transcriptional activation domain.

### Transcriptional Repressor Domains

In certain embodiments, the effector domain of the fusion protein is a transcriptional repressor domain. In general, a transcriptional repressor domain interacts with transcriptional control elements and/or transcriptional regulatory proteins (i.e., transcription factors, RNA polymerases, etc.) to decrease and/or prohibit transcription of a gene. In certain embodiments, the transcriptional repressor domains is inducible CAMP early repressor (ICER) domains, Kruppel-associated box A (KRAB-A) repressor domains, YY1 glycine rich repressor domains, Sp1-like repressors, E (spI) repressors, IκB repressor, or MeCP2.

### Epigenetic Modification Domains

In certain embodiments, the effector domain of the fusion protein is an epigenetic modification domain. In general, epigenetic modification domains alter gene expression by modifying the histone structure and/or chromosomal structure. In certain embodiments, the epigenetic modification domains is a histone acetyltransferase domain, a histone deacetylase domain, a histone methyltransferase domain, a histone demethylase domain, a DNA methyltransferase domain, or a DNA demethylase domain.

### Additional Domains

In certain embodiments, the fusion protein further comprises at least one additional domain. Non-limiting examples of suitable additional domains include nuclear localization signals (NLSs), cell-penetrating or translocation domains, and marker domains. An NLS generally comprises a stretch of basic amino acids. See, e.g., Lange et al. (2007) J. Biol. Chem., 282, 5101-5. For example, in certain embodiments, the NLS is a monopartite sequence, such as PKKKRKV (SEQ ID NO: 2) or PKKKRRV (SEQ ID NO: 3). In certain embodiments, the NLS is a bipartite sequence. In certain embodiments, the NLS is KRPAATKKAGQAKKKK (SEQ ID NO: 4).

In certain embodiments, the fusion protein comprises at least one cell-penetrating domain. In certain embodiments, the cell-penetrating domain is a cell-penetrating peptide sequence derived from the HIV-1 TAT protein. As an example, the TAT cell-penetrating sequence can be GRKKRRQRRRPPQPKKKRKV (SEQ ID NO: 5). In certain embodiments, the cell-penetrating domain is TLM (PLSSIFSRIGDPPKKKRKV; SEQ ID NO: 6), a cell-penetrating peptide sequence derived from the human hepatitis B virus. In certain embodiments, the cell-penetrating domain is MPG (GALFLGWLGAAGSTMGAPKKKRKV; SEQ ID NO: 7 or GALFLGFLGAAGSTMGAWSQPKKKRKV; SEQ ID NO: 8). In certain embodiments, the cell-penetrating domain is Pep-1 (KETWWETWWTEWSQPKKKRKV; SEQ ID NO: 9), VP22, a cell penetrating peptide from Herpes simplex virus, or a polyarginine peptide sequence.

In certain embodiments, the fusion protein comprises at least one marker domain. Non-limiting examples of marker domains include fluorescent proteins, purification tags, and epitope tags. In certain embodiments, the marker domain is a fluorescent protein. Non limiting examples of suitable fluorescent proteins include green fluorescent proteins (e.g., GFP. GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1), blue fluorescent proteins (e.g. EBFP, EBFP2, Azurite, mKalama1, GFPuv, Sapphire, T-sapphire), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), orange fluorescent proteins (mOrange, mKO, Kusabira-Orange, Monomeric Kusabira-Orange, mTangerine, tdTomato) and any other suitable fluorescent protein. In certain embodiments, the marker domain is a purification tag and/or an epitope tag. Exemplary tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, biotin carboxyl carrier protein (BCCP), and calmodulin.

### V. Uses and Methods

In one aspect, the present invention provides a method for cleaving a target polynucleotide with a Cas protein. The method comprises contacting the target polynucleotide with (i) a guide RNA or a set of guide RNA molecules described herein, and (ii) a Cas protein. In certain embodiments, the method results in a double-strand break in the target polynucleotide. In certain embodiments, the Cas protein is a Cas protein having a single-strand nicking activity. In certain embodiments, the method results in a single-strand break in the target polynucleotide. In certain embodiments, a complex comprising a guide RNA and Cas protein having a single-strand nicking activity is used for sequence-targeted single-stranded DNA cleavage, i.e., nicking.

In one aspect, the present invention provides a method for cleaving two or more target polynucleotides with a Cas protein. The method comprises contacting the target polynucleotides with (i) a set of guide RNA molecules described herein, and (ii) a Cas protein. In certain embodiments, the method results in double-strand breaks in the target polynucleotides. In certain embodiments, the Cas protein is a Cas protein having a single-strand nicking activity. In certain embodiments, the method results in single-strand breaks in the target polynucleotides. In certain embodiments, a complex comprising a guide RNA and Cas protein having a single-strand nicking activity is used for sequence-targeted single-stranded DNA cleavage, i.e., nicking.

In one aspect, the present invention provides a method for binding a target polynucleotide with a Cas protein. The method comprises contacting the target polynucleotide with (i) a guide RNA or a set of guide RNA molecules described herein and (ii) a Cas protein, to result in binding of the target polynucleotide with the Cas protein. In certain embodiments, the Cas protein is a Cas variant. In certain embodiments, the Cas variant lacks some or all nuclease activity relative to a counterpart wild-type Cas protein.

In one aspect, the present invention provides a method for binding two or more target polynucleotides with a Cas protein. The method comprises contacting the target polynucleotides with (i) a set of RNA molecules described herein and (ii) a Cas protein, to result in binding of the target polynucleotides with the Cas protein. In certain embodiments, the Cas protein is a Cas variant. In certain embodiments, the Cas variant lacks some or all nuclease activity relative to a counterpart wild-type Cas protein.

In one aspect, the present invention provides a method for targeting a Cas protein to a target polynucleotide. The method comprises contacting the Cas protein with a guide RNA or a set of guide RNA molecules described herein. In certain embodiments, the method results in formation of a guide RNA:Cas protein complex. In certain embodiments, the Cas protein is a wild type Cas9 protein. In certain embodiments, the Cas protein is a mutant or variant of a Cas9 protein. In certain embodiments, the Cas protein is a Cas protein having a single-strand nicking activity. In certain embodiments, the Cas protein is a Cas protein lacking nuclease activity (e.g., a nuclease-deficient mutant of Cas protein). In certain embodiments, the Cas protein is part of a fusion protein (e.g., a fusion protein comprising (i) the Cas protein and (ii) a heterologous polypeptide).

In one aspect, the present invention provides a method for targeting a Cas protein to two or more target polynucleotides. The method comprises contacting the Cas protein with a set of guide RNA molecules described herein. In certain embodiments, the method results in formation of a guide RNA:Cas protein complex. In certain embodiments, the Cas protein is a wild type Cas9 protein. In certain embodiments, the Cas protein is a mutant or variant of a Cas9 protein. In certain embodiments, the Cas protein is a Cas protein having a single-strand nicking activity. In certain embodiments, the Cas protein is a Cas protein lacking nuclease activity (e.g., a nuclease-deficient mutant of Cas protein). In certain embodiments, the Cas protein is part of a fusion protein (e.g., a fusion protein comprising (i) the Cas protein or and (ii) a heterologous polypeptide).

In certain embodiments, the guide RNA is introduced into a cell by transfection. Techniques for RNA transfection are known in the art and include electroporation and lipofection. Effective techniques for RNA transfection depend mostly on cell type. See, e.g., Lujambio et al. (Spanish National Cancer Centre) Cancer Res. February 2007, which describes transfection of HTC-116 colon cancer cells and uses Oligofectamine (Invitrogen) for transfection of commercially obtained, modified miRNA or precursor miRNA. See also, Cho et al. (Seoul National Univ.) Nat. Biotechnol. March 2013, which describes transfection of K562 cells and uses 4D Nucleofection™ (Lonza) electroporation for transfection of transcribed sgRNAs (about 60 nts long). Techniques for transfection of RNA are also known in the art. For example, therapeutic RNA has been delivered in non-pathogenic E. coli coated with Invasin protein (to facilitate uptake into cells expressing β-1 integrin protein) and with the E. coli encoded to express listeriolysin O pore-forming protein to permit the shRNA to pass from the E. coli into the cytoplasm. See also Cho et al. (Seoul National Univ.) Nat. Biotechnol. March 2013.

In certain embodiments, the guide RNA is introduced or delivered into cells. Technologies that can be used for delivery of guide RNA include those that utilize encapsulation by biodegradable polymers, liposomes, or nanoparticles. Such polymers, liposomes, and nanoparticles can be delivered intravenously. In certain embodiments, for in vivo delivery, guide RNA can be injected into a tissue site or administered systemically. In vivo delivery can also be effected by a beta-glucan delivery system, such as those described in U.S. Pat. Nos. 5,032,401 and 5,607,677, and U.S. Publication No. 2005/0281781, which are hereby incorporated by reference in their entirety. In certain embodiments, guide RNA or a delivery vehicle containing guide RNA is targeted to a particular tissue or body compartment. For example, in certain embodiments, to target exogenous RNA to other tissues, synthetic carriers are decorated with cell-specific ligands or aptamers for receptor uptake, e.g., RNA encased in cyclodextrin nanoparticles coated with PEG and functionalized with human transferrin protein for uptake via the transferrin receptor which is highly expressed in tumor cells. Further approaches are described herein below or known in the art.

The present invention has been tested in human cells as described in Hendel et al., Nat. Biotechnol. (2015) 33:9, 985-9 (which is incorporated in this application in its entirety). In the cited work, modified guide RNA was introduced into K562 cells, human primary T cells, and CD34+ hematopoietic stem and progenitor cells (HSPCs). The modified guide RNA significantly enhanced genome editing efficiencies in human cells, including human primary T cells and CD34+HSPCs as compared to unmodified guide RNA.

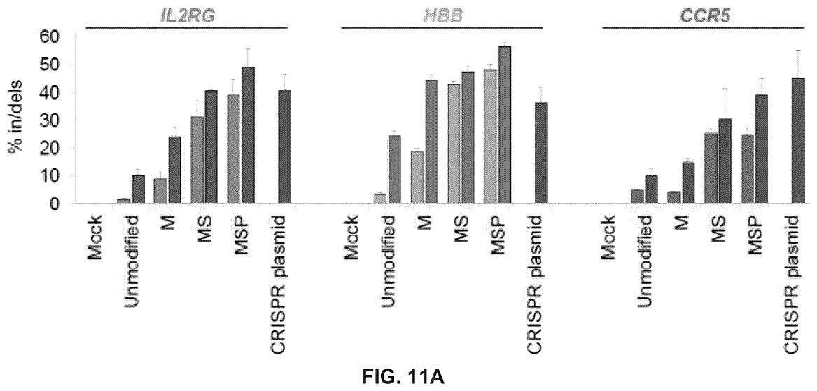

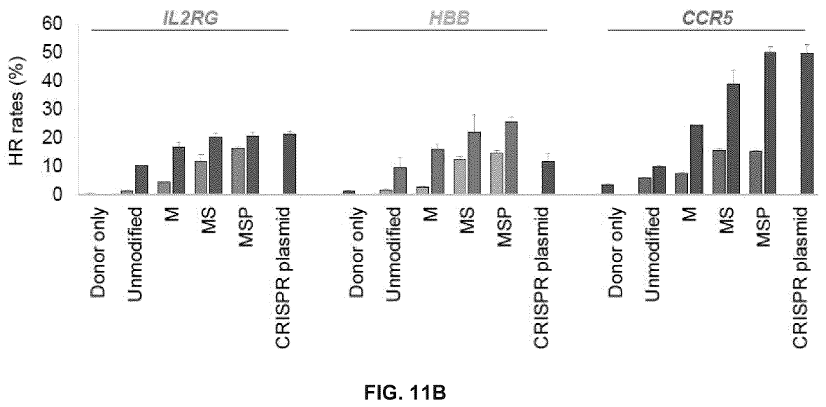

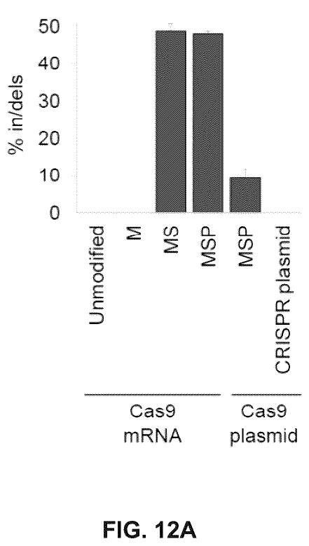

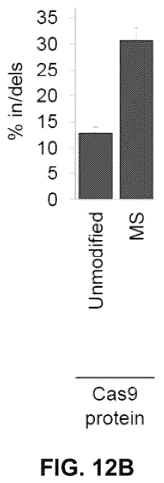

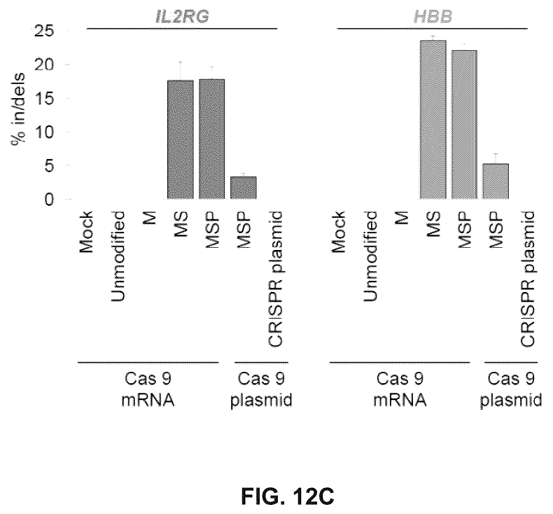

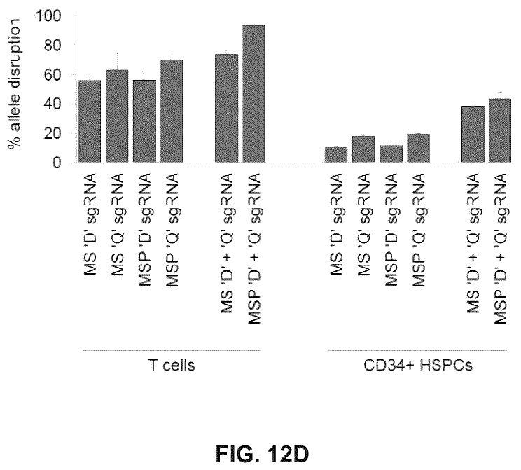

FIGS. 11A and 11B illustrate experimental results showing that gene disruption in human cell lines can be achieved by high frequencies of indels or by cleavage-stimulated homologous recombination using synthesized and chemically modified sgRNAs disclosed herein. Gene disruption by mutagenic NHEJ was measured by deep sequencing of PCR amplicons (FIG. 12A) or gene addition by HR at the three loci IL2RG, HBB and CCR5 in K562 cells induced by Cas9 in combination with synthetic sgRNAs (FIG. 12B). The synthetic sgRNAs were delivered at 1 μg (light shade) or 20 μg (dark shade) per 1 million cells. Cas9 was expressed from a plasmid (2 μg) and for HR experiments 5 μg of GFP-encoding donor plasmid was included. As a positive control, 2 μg of sgRNA plasmid encoding both the sgRNA and the Cas9 protein was used (gray bars). Bars represent average values+s.e.m., n=3.

FIGS. 12A, 12B, 12C and 12D illustrate experimental results showing that chemically modified sgRNAs as described herein can be used to achieve high frequencies of gene disruption or targeted genome editing in stimulated primary human T cells and CD34+ hematopoietic stem and progenitor cells (HSPCs).

FIG. 12A illustrates results from primary human T cells nucleofected with 10 μg of a synthetic CCR5 sgRNAs and either 15 μg Cas9 mRNA or 1 μg Cas9-encoding plasmid. 1 μg sgRNA plasmid encoding both the sgRNA and Cas9 protein was included for comparison. The bars represent average indel frequencies for three different donors+s.e.m., n=6, as measured by TIDE (tracking of indels by decomposition) analysis of PCR amplicons spanning the sgRNA target site, and using a mock-treated sample as control reference. Delivery of Cas9 mRNA with the unmodified or the M-modified sgRNA, and nucleofection of the plasmid encoding both the sgRNA and Cas9, did not give rise to allele modification frequencies above background. Co-transfection of the MSP-modified sgRNA with DNA expression plasmid for Cas9 generated 9.3% indel frequency. Cas9 mRNA with either the MS- or MSP-modified sgRNA generated 48.7% and 47.9% indel frequencies, respectively.

FIG. 12B illustrates results from stimulated T cells. The cells were nucleofected as above, but with 15 μg Cas9 protein complexed with a 2.5 molar excess of the indicated synthetic CCR5 sgRNAs. Indel frequencies were measured by TIDE analysis. The bars represent average indel frequencies for three different donors+s.e.m., n=6. A 2.4-fold improvement in indel frequencies of the MS-modified sgRNA over the unmodified sgRNA (30.7% vs. 12.8%) was observed for chemically modified sgRNAs when delivered complexed with Cas9 protein. These results establish that chemically modified sgRNAs can be used for genome editing of stimulated T cells when delivered complexed with Cas9 protein.

FIG. 12C illustrates results from human peripheral blood CD34+HSPCs. 500,000 mobilized cells were nucleofected with 10 μg of the indicated synthetic sgRNAs targeting IL2RG or HBB and either 15 μg Cas9 mRNA or 1 μg Cas9 plasmid. 1 μg of sgRNA plasmid encoding both the sgRNA and Cas9 protein was included for comparison. Bars represent average indel frequencies+s.e.m., n=3, as measured by T7 endonuclease cleavage assay. Indels were not detected at either locus using the unmodified or M-modified sgRNAs when co-transfected with Cas9 mRNA. However, the IL2RG MS- and MSP-modified sgRNAs showed 17.5% and 17.7% indel frequencies, respectively, and 23.4% and 22.0%, respectively, for the HBB MS- and MSP-modified sgRNAs.

FIG. 12D illustrates results from stimulated T cells or mobilized human peripheral blood CD34+HSPCs. One million cells were nucleofected with 15 μg Cas9 mRNA and 10 μg of the indicated synthetic CCR5 sgRNAs. A recent study showed that the simultaneous use of two sgRNAs could improve gene disruption in human primary T cells and in CD34+HSPCs. See, e.g., Mandal et al. (2014) Cell Stem Cell, 15, 643-52. MS- and MSP-modified CCR5 sgRNAs were chemically synthesized with the sequences reported in Mandal study (termed ‘D’ and ‘Q’), which cut 205 base pairs apart. When used in combination, the amount of each sgRNA was 5 μg. Indel frequencies for samples with single sgRNAs were measured by TIDE analysis as above and allele disruption frequencies for samples with two sgRNAs were measured by sequencing of cloned PCR products. The bars represent average indel frequencies+s.e.m., n=3. In T cells, the ‘D’ sgRNA alone gave rise to 56.0% and 56.3% indels for the MS- and MSP-modified sgRNA, respectively, and the ‘Q’ sgRNA gave rise to 62.6% and 69.6% indels, respectively. When used in combination, the frequencies of allele modification increased, as we observed 73.9% and 93.1% indels for the MS- and MSP-modified sgRNAs, respectively, of which the majority of the modification events were deletions between the two sgRNA target sites. In CD34+HSPCs, observations were similar though the overall frequencies were lower. For the ‘D’ sgRNA, allele modification frequencies of 9.8% and 11.2% were observed for the MS- and MSP-modified sgRNA, respectively, and 17.8% and 19.2% for the ‘Q’ sgRNA. When used in combination the frequencies increased to 37.8% and 43.0% for the MS- and MSP-modified sgRNAs, respectively. This shows that the use of two chemically modified sgRNAs is a highly effective way to facilitate gene disruption in primary human T cells and CD34+HSPCs.

Examples of other uses include genomic editing and gene expression regulation as described below.

### Genomic Editing

In one aspect, the present invention provides a method for genomic editing to modify a DNA sequence in vivo or in vitro (“in vitro” includes, without being limited to, a cell-free system, a cell lysate, an isolated component of a cell, and a cell outside of a living organism). The DNA sequence may comprise a chromosomal sequence, an episomal sequence, a plasmid, a mitochondrial DNA sequence, or a functional intergenic sequence, such as an enhancer sequence or a DNA sequence for a non-coding RNA. The method comprises contacting the DNA sequence with (i) a guide RNA or a set of guide RNA molecules described herein, and (ii) a Cas protein. In certain embodiments, the DNA sequence is contacted outside of a cell. In certain embodiments, the DNA sequence is located in the genome within a cell and is contacted in vitro or in vivo. In certain embodiments, the cell is within an organism or tissue. In certain embodiments, the cell is a human cell, a non-human mammalian cell, a stem cell, a non-mammalian vertebrate cell, an invertebrate cell, a plant cell, a single cell organism, or an embryo. In certain embodiments, the guide RNA aids in targeting the Cas protein to a targeted site in the DNA sequence. In certain embodiments, the Cas protein cleaves at least one strand of the DNA sequence at the targeted site. In certain embodiments, the Cas protein cleaves both strands of the DNA sequence at the targeted site.

In certain embodiments, the method further comprises introducing the Cas protein into a cell or another system. In certain embodiments, the Cas protein is introduced as a purified or non-purified protein. In certain embodiments, the Cas protein is introduced via an mRNA encoding the Cas protein. In certain embodiments, the Cas protein is introduced via a linear or circular DNA encoding the Cas protein. In certain embodiments, the cell or system comprises a Cas protein or a nucleic acid encoding a Cas protein.

In certain embodiments, a double-stranded break can be repaired via an error-prone, non-homologous end-joining (“NHEJ”) repair process. In certain embodiments, a double-stranded break can be repaired by a homology-directed repair (HDR) process such that a donor sequence in a donor polynucleotide can be integrated into or exchanged with the targeted DNA sequence.

In certain embodiments, the method further comprises introducing at least one donor polynucleotide into the cell or system. In certain embodiments, the donor polynucleotide comprises at least one homologous sequence having substantial sequence identity with a sequence on either side of the targeted site in the DNA sequence. In certain embodiments, the donor polynucleotide comprises a donor sequence that can be integrated into or exchanged with the DNA sequence via homology-directed repair, such as homologous recombination.

In certain embodiments, the donor polynucleotide includes an upstream homologous sequence and a downstream homologous sequence, each of which have substantial sequence identity to sequences located upstream and downstream, respectively, of the targeted site in the DNA sequence. These sequence similarities permit, for example, homologous recombination between the donor polynucleotide and the targeted DNA sequence such that the donor sequence can be integrated into (or exchanged with) the DNA sequence targeted.

In certain embodiments, the target site(s) in the DNA sequence spans or is adjacent to a mutation, e.g., point mutation, a translocation or an inversion which may cause or be associated with a disorder. In certain embodiments, the method comprises correcting the mutation by introducing into the cell or system at least one donor polynucleotide comprising (i) a wild type counterpart of the mutation and (ii) at least one homologous sequence having substantial sequence identity with a sequence on one side of the targeted site in the DNA sequence. In certain embodiments, the donor polynucleotide comprises a homologous sequence having substantial sequence identity with a sequence on both sides of the targeted site in the DNA sequence.

In certain embodiments, the donor polynucleotide comprises an exogenous sequence that can be integrated into or exchanged with the targeted DNA sequence via a homology-directed repair process, such as homologous recombination. In certain embodiments, the exogenous sequence comprises a protein coding gene, which, optionally, is operably linked to an exogenous promoter control sequence. Thus, in certain embodiments, upon integration of the exogenous sequence, a cell can express a protein encoded by the integrated gene. In certain embodiments, the exogenous sequence is integrated into the targeted DNA sequence such that its expression in the recipient cell or system is regulated by the exogenous promoter control sequence. Integration of an exogenous gene into the targeted DNA sequence is termed a “knock in.” In other embodiments, the exogenous sequence can be a transcriptional control sequence, another expression control sequence, an RNA coding sequence, and the like.

In certain embodiments, the donor polynucleotide comprises a sequence that is essentially identical to a portion of the DNA sequence at or near the targeted site, but comprises at least one nucleotide change. For example, in certain embodiments, the donor sequence comprises a modified or mutated version of the DNA sequence at or near the targeted site such that, upon integration or exchange with the targeted site, the resulting sequence at the targeted site comprises at least one nucleotide change. In certain embodiments, the at least one nucleotide change is an insertion of one or more nucleotides, a deletion of one or more nucleotides, a substitution of one or more nucleotides, or combinations thereof. As a consequence of the integration of the modified sequence, the cell may produce a modified gene product from the targeted DNA sequence.

In certain embodiments, the methods are for multiplex applications. In certain embodiments, the methods comprise introducing a library of guide RNAs into the cell or system. In certain embodiments, the library comprises at least 100 unique guide sequences. In certain embodiments, the library comprises at least 1,000 unique guide sequences. In certain embodiments, the library comprises at least 10,000 unique guide sequences. In certain embodiments, the library comprises at least 100,000 unique guide sequences. In certain embodiments, the library comprises at least 1,000,000 unique guide sequences. In certain embodiments, the library targets at least 10 different polynucleotides or at least 10 different sequences within one or more polynucleotides. In certain embodiments, the library targets at least 100 different polynucleotides or at least 100 different sequences within one or more polynucleotides. In certain embodiments, the library targets at least 1,000 different polynucleotides or at least 1,000 different sequences within one or more polynucleotides. In certain embodiments, the library targets at least 10,000 different polynucleotides or at least 10,000 different sequences within one or more polynucleotides. In certain embodiments, the library targets at least 100.000 different polynucleotides or at least 100,000 different sequences within one or more polynucleotides. In certain embodiments, the library targets at least 1,000,000 different polynucleotides or at least 1,000,000 different sequences within one or more polynucleotides.

### Genomic Editing in Human and Mammalian Cells

Embodiments of the present invention are useful in methods for genomic editing to modify a target polynucleotide, for example a DNA sequence, in a mammalian cell.

In certain embodiments, the DNA sequence is a chromosomal sequence. In certain embodiments, the DNA sequence is a protein-coding sequence. In certain embodiments, the DNA sequence is a functional intergenic sequence, such as an enhancer sequence or a non-coding sequence. In certain embodiments, the DNA is part of a human gene. In some such embodiments, the human gene is the clathrin light chain (CLTA1) gene, the human interleukin 2 receptor gamma (IL2RG) gene, the human cytotoxic T-lymphocyte-associated protein 4 (CLTA4) gene, the human protocadherin alpha 4 (PCDHA4) gene, the human engrailed homeobox 1 (EN1) gene, the human hemoglobin beta (HBB) gene, which can harbor mutations responsible for sickle cell anemia and thalassemias, or the human chemokine (C—C motif) receptor 5 (CCR5) gene which encodes a co-receptor of HIV.

In certain embodiments, the mammalian cell is a human cell. In some such embodiments, the human cell is a primary human cell. In further embodiments, the primary human cell is a human primary T cell. The human primary T cell may be stimulated or unstimulated. In certain embodiments, the human cell is a stem/progenitor cell, such as a CD34+ hematopoietic stem and progenitor cell (HSPC). In certain embodiments, the human cell is from a cultured cell line, for example such as can be obtained commercially. Exemplary cell lines include K562 cells, a human myelogenous leukemia line.

In certain embodiments, the cell is within a living organism. In certain other embodiments, the cell is outside of a living organism.

The method comprises contacting the DNA sequence with (i) a guide RNA or a set of guide RNA molecules described herein, and (ii) a Cas protein.

In certain embodiments, the method further comprises introducing or delivering the guide RNA into the cell. In some such embodiments, the guide RNA is introduced into a cell by transfection. Techniques for RNA transfection are known in the art and include electroporation and lipofection. In other embodiments, the guide RNA is introduced into a cell (and, more particularly, a cell nucleus) by nucleofection. Techniques for nucleofection are known in the art and may utilize nucleofection devices such as the Lonza Nucleofector 2b or the Lonza 4D-Nucleofector and associated reagents.

In certain embodiments, the method further comprises introducing or delivering the Cas protein into the cell. In some such embodiments, the Cas protein is introduced as a purified or non-purified protein. In other embodiments, the Cas protein is introduced via an mRNA encoding the Cas protein. In some such embodiments, the mRNA encoding the Cas protein is introduced into the cell by transfection. In other embodiments, the mRNA encoding the Cas protein is introduced into a cell (and, more particularly, a cell nucleus) by nucleofection.

In certain embodiments, the method employs ribonucleoprotein (RNP)-based delivery such that the Cas protein is introduced into the cell in a complex with the guide RNA. For example, a Cas9 protein may be complexed with a guide RNA in a Cas9:gRNA complex, which allows for co-delivery of the gRNA and Cas protein. For example, the Cas:gRNA complex may be nucleofected into cells.

In certain embodiments, the method employs an all-RNA delivery platform. For example, in some such embodiments, the guide RNA and the mRNA encoding the Cas protein are introduced into the cell simultaneously or substantially simultaneously (e.g., by co-transfection or co-nucleofection). In certain embodiments, co-delivery of Cas mRNA and modified gRNA results in higher editing frequencies as compared to co-delivery of Cas mRNA and unmodified gRNA. In particular, gRNA having 2′-O-methyl-3′-phosphorothioate (MS), or 2′-O-methyl-3′-thioPACE (MSP) incorporated at three terminal nucleotides at both the 5′ and 3′ ends, provide higher editing frequencies as compared to unmodified gRNA.

In certain embodiments, the guide RNA and the mRNA encoding the Cas protein are introduced into the cell sequentially; that is, the guide RNA and the mRNA encoding the Cas protein are introduced into the cell at different times. The time period between the introduction of each agent may range from a few minutes (or less) to several hours or days. For example, in some such embodiments, gRNA is delivered first, followed by delivery of Cas mRNA 4, 8, 12 or 24 hours later. In other such embodiments, Cas mRNA is delivered first, followed by delivery of gRNA 4, 8, 12 or 24 hours later. In some particular embodiments, delivery of modified gRNA first, followed by delivery of Cas mRNA results in higher editing frequencies as compared to delivery of unmodified gRNA followed by delivery of Cas mRNA.

In certain embodiments, the gRNA is introduced into the cell together with a DNA plasmid encoding the Cas protein. In some such embodiments, the gRNA and the DNA plasmid encoding the Cas protein are introduced into the cell by nucleofection. In some particular embodiments, an RNP-based delivery platform or an all-RNA delivery platform provides lower cytotoxicity in primary cells than a DNA plasmid-based delivery system.

In certain embodiments, the method provides significantly enhanced genome editing efficiencies in human cells, including human primary T cells and CD34+HSPCs.

In certain embodiments, modified gRNA increases the frequency of insertions or deletions (indels), which may be indicative of mutagenic NHEJ and gene disruption, relative to unmodified gRNA. In particular, modified gRNA having 2′-O-methyl-3′-phosphorothioate (MS) or 2′-O-methyl-3′-thioPACE (MSP) incorporated at three terminal nucleotides at both the 5′ and 3′ ends, increases the frequency of indels relative to unmodified gRNA.

In certain embodiments, co-delivery of modified gRNA and Cas mRNA to human primary T cells increases the frequency of indels as compared to co-delivery of unmodified gRNA and Cas mRNA. In particular, modified gRNA having 2′-O-methyl-3′-phosphorothioate (MS) or 2′-O-methyl-3′-thioPACE (MSP) incorporated at three terminal nucleotides at both the 5′ and 3′ ends, increases the frequency of indels in human primary T cells relative to unmodified gRNA.

In certain embodiments, modified gRNA improves gRNA stability relative to unmodified gRNA. As one example, gRNA having 2′-O-methyl (M) incorporated at three terminal nucleotides at both the 5′ and 3′ ends, modestly improves stability against nucleases and also improves base pairing thermostability over unmodified gRNA. As another example, gRNA having 2′-O-methyl-3′-phosphorothioate (MS) or 2′-O-methyl-3′-thioPACE (MSP) incorporated at three terminal nucleotides at both the 5′ and 3′ ends, dramatically improves stability against nucleases relative to unmodified gRNA. It is contemplated that gRNA end modifications enhance intracellular stability against exonucleases, thus enabling increased efficacy of genome editing when Cas mRNA and gRNA are co-delivered or sequentially delivered into human cells.

In certain embodiments, modified gRNA stimulates gene targeting, which, in turn, allows for gene editing by, for example, homologous recombination or NHEJ. In particular, gRNA having 2′-O-methyl-3′-phosphorothioate (MS), or 2′-O-methyl-3′-thioPACE (MSP) incorporated at three terminal nucleotides at both the 5′ and 3′ ends, stimulates higher levels of homologous recombination than unmodified gRNA.

In certain embodiments, modified gRNA retains high specificity. In certain embodiments, the ratio of on-target to off-target indel frequencies is improved with modified gRNA as compared to unmodified gRNA. In certain embodiments, modified gRNA delivered in an RNP complex with a Cas protein provides significantly better on-target:off-target ratios compared to a DNA plasmid-based delivery system.

### Gene Expression Regulation

In certain embodiments, the guide RNA described herein is used for regulating transcription or expression of a gene of interest. For example, in certain embodiments, a fusion protein comprising a Cas protein (e.g., a nuclease-deficient Cas9) and a transcription activator polypeptide is used to increase transcription of a gene. Similarly, in certain embodiments, a fusion protein comprising a Cas protein (e.g., a nuclease-deficient Cas9) and a repressor polypeptide is used to knock-down gene expression by interfering with transcription of the gene.

In at least one aspect, the present invention provides a method for regulating the expression of a gene of interest in vivo or in vitro. The method comprises introducing into a cell or another system (i) a synthetic guide RNA described herein, and (ii) a fusion protein. In certain embodiments, the fusion protein comprises a Cas protein and an effector domain, such as a transcriptional activation domain, a transcriptional repressor domain, or an epigenetic modification domain. In certain embodiments, the fusion protein comprises a mutated Cas protein, such as a Cas9 protein that is a null nuclease. In certain embodiments, the Cas protein contains one or more mutations, such as D10A, H840A and/or N863A.

In certain embodiments, the fusion protein is introduced into the cell or system as a purified or non-purified protein. In certain embodiments, the fusion protein is introduced into the cell or system via an mRNA encoding the fusion protein. In certain embodiments, the fusion protein is introduced into the cell or system via a linear or circular DNA encoding the fusion protein.

In certain embodiments, the guide RNA aids in directing the fusion protein to a specific target polynucleotide comprising a chromosomal sequence, an episomal sequence, a plasmid, a mitochondrial DNA sequence, or a functional intergenic sequence, such as an enhancer or the DNA sequence for a non-coding RNA. In certain embodiments, the effector domain regulates expression of a sequence in the target polynucleotide. A guide RNA for modulating gene expression can be designed to target any desired endogenous gene or sequence encoding a functional RNA. A genomic target sequence can be selected in proximity of the transcription start site of the endogenous gene, or alternatively, in proximity of the translation initiation site of the endogenous gene. In certain embodiments, the target sequence is in a region of the DNA that is traditionally termed the “promoter proximal” region of a gene. In certain embodiments, the target sequence lies in a region from about 1,000 base pairs upstream of the transcription start site to about 1,000 base pairs downstream of the transcription start site. In certain embodiments, the target sequence is remote from the start site for transcription of the gene (e.g., on another chromosome).

In certain embodiments, the methods are for multiplex applications. In certain embodiments, the methods comprise introducing a library of guide RNAs into the cell or system. In certain embodiments, the library comprises at least 100, at least 1,000, at least 10,000, at least 100,000, or at least 1,000,000 unique guide sequences. In certain embodiments, the library targets at least 10 different polynucleotides or at least 10 different sequences within one or more polynucleotides. In certain embodiments, the library targets at least 100 different polynucleotides or at least 100 different sequences within one or more polynucleotides. In certain embodiments, the library targets at least 1,000 different polynucleotides or at least 1,000 different sequences within one or more polynucleotides. In certain embodiments, the library targets at least 10.000 different polynucleotides or at least 10.000 different sequences within one or more polynucleotides. In certain embodiments, the library targets at least 100,000 different polynucleotides or at least 100,000 different sequences within one or more polynucleotides. In certain embodiments, the library targets at least 1,000,000 different polynucleotides or at least 1,000,000 different sequences within one or more polynucleotides.

### Kits

In one aspect, the present invention provides kits containing reagents for performing the above-described methods, including producing gRNA: Cas protein complex and/or supporting its activity for binding, nicking or cleaving target polynucleotide. In certain embodiments, one or more of the reaction components, e.g., one or more guide RNAs and Cas proteins, for the methods disclosed herein, can be supplied in the form of a kit for use. In certain embodiments, the kit comprises a Cas protein or a nucleic acid encoding the Cas protein, and one or more guide RNAs described herein or a set or library of guide RNAs. In certain embodiments, the kit includes one or more other reaction components. In certain embodiments, an appropriate amount of one or more reaction components is provided in one or more containers or held on a substrate.

Examples of additional components of the kits include, but are not limited to, one or more different polymerases, one or more host cells, one or more reagents for introducing foreign nucleic acid into host cells, one or more reagents (e.g., probes or PCR primers) for detecting expression of the guide RNA and/or the Cas mRNA or protein or for verifying the status of the target nucleic acid, and buffers, transfection reagents or culture media for the reactions (in 1× or more concentrated forms). In certain embodiments, the kit includes one or more of the following components: biochemical and physical supports; terminating, modifying and/or digesting reagents; osmolytes; and apparati for reaction, transfection and/or detection.

The reaction components used can be provided in a variety of forms. For example, the components (e.g., enzymes, RNAs, probes and/or primers) can be suspended in an aqueous solution or bound to a bead or as a freeze-dried or lyophilized powder or pellet. In the latter case, the components, when reconstituted, form a complete mixture of components for use in an assay. The kits of the invention can be provided at any suitable temperature. For example, for storage of kits containing protein components or complexes thereof in a liquid, it is preferred that they are provided and maintained below 0° C. preferably at about −20° C. possibly in a freeze-resistant solution containing glycerol or other suitable antifreeze.

A kit or system may contain, in an amount sufficient for at least one assay, any combination of the components described herein. In some applications, one or more reaction components may be provided in pre-measured single use amounts in individual, typically disposable, tubes or equivalent containers. With such an arrangement, a RNA-guided nuclease reaction can be performed by adding a target nucleic acid, or a sample or cell containing the target nucleic acid, to the individual tubes directly. The amount of a component supplied in the kit can be any appropriate amount and may depend on the market to which the product is directed. The container(s) in which the components are supplied can be any conventional container that is capable of holding the supplied form, for instance, microfuge tubes, microtiter plates, ampoules, bottles, or integral testing devices, such as fluidic devices, cartridges, lateral flow, or other similar devices.

The kits can also include packaging materials for holding the container or combination of containers. Typical packaging materials for such kits and systems include solid matrices (e.g., glass, plastic, paper, foil, micro-particles and the like) that hold the reaction components or detection probes in any of a variety of configurations (e.g., in a vial, microtiter plate well, microarray, and the like). The kits may further include instructions recorded in a tangible form for use of the components.

### EXAMPLES

### Example 1

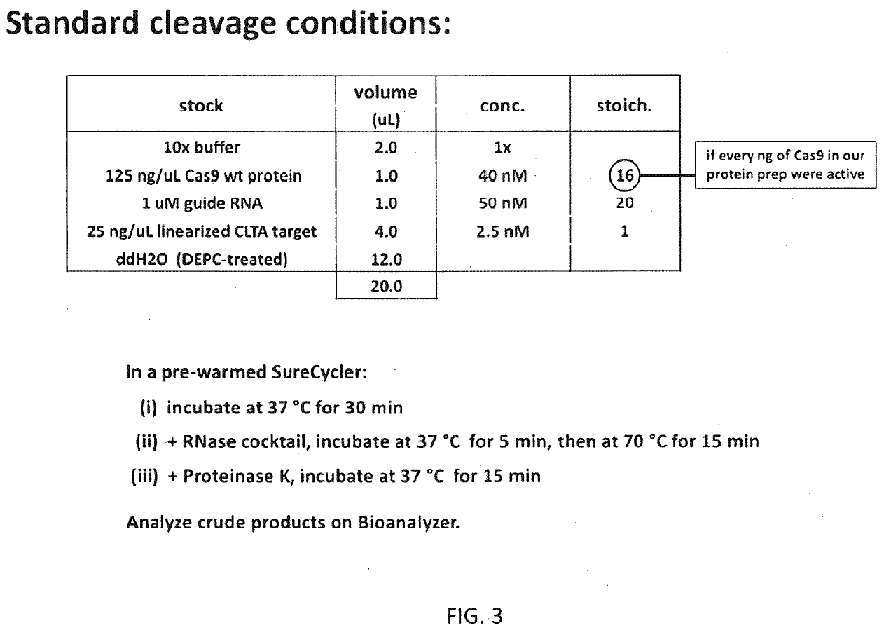

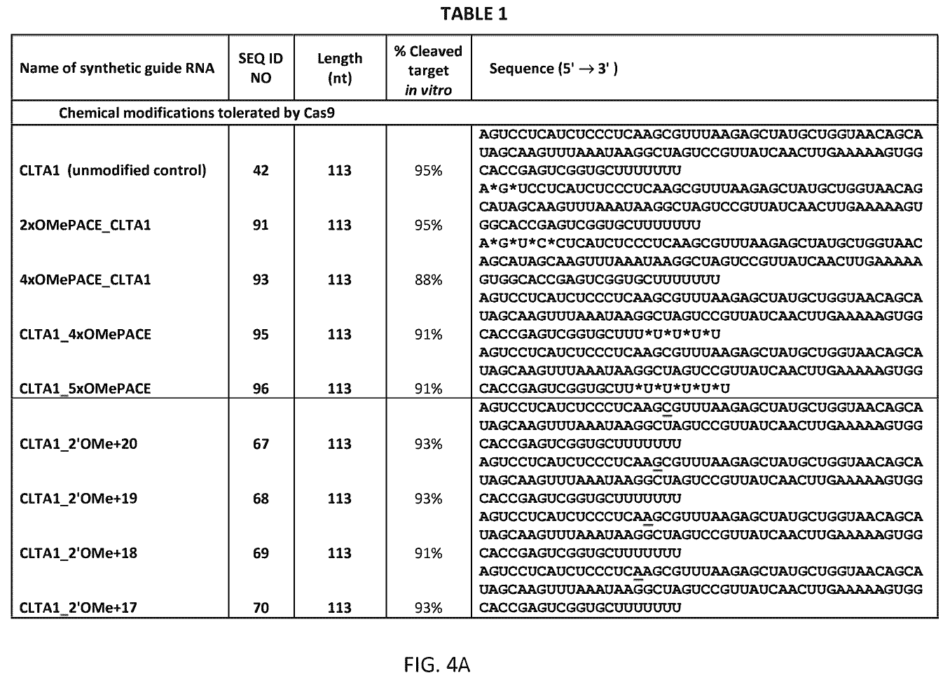

To evaluate the ability of the chemically synthesized guide RNAs to target and cleave a DNA target sequence, an in vitro cleavage assay was developed. Briefly, as shown in FIG. 3, ˜4-kb PAM-addressable DNA targets were prepared by preparative PCR amplification of plasmid-borne human sequences (here, a sequence from the human clathrin light chain CLTA gene). In a 20-uL reaction volume, 50 fmoles of linearized DNA target in the presence of 50 nM sgRNA, 39 nM recombinant purified Cas9 protein (S. pyogenes; Agilent) and 10 mM MgCl2 at pH 7.6 was incubated at 37° C. for 30 min. Upon completion, 0.5 uL of RNace It (Agilent) was added, and incubation was continued at 37° C. for 5 min and then at 70° C. for 15 min. Subsequently 0.5 μL of Proteinase K (Mol. Bio. grade, NEB) was added and incubated at 37° C. for 15 min. Aliquots were loaded into a DNA 7500 LabChip and were analyzed on a Bioanalyzer 2200. The workup steps served to release Cas9 from binding to target DNA, which were assayed for cleavage.

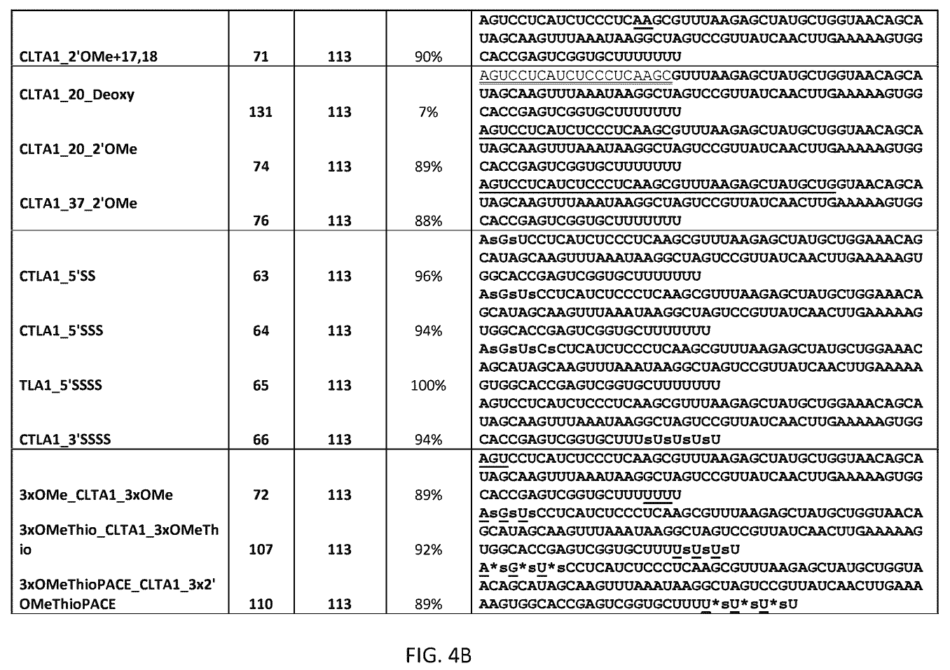

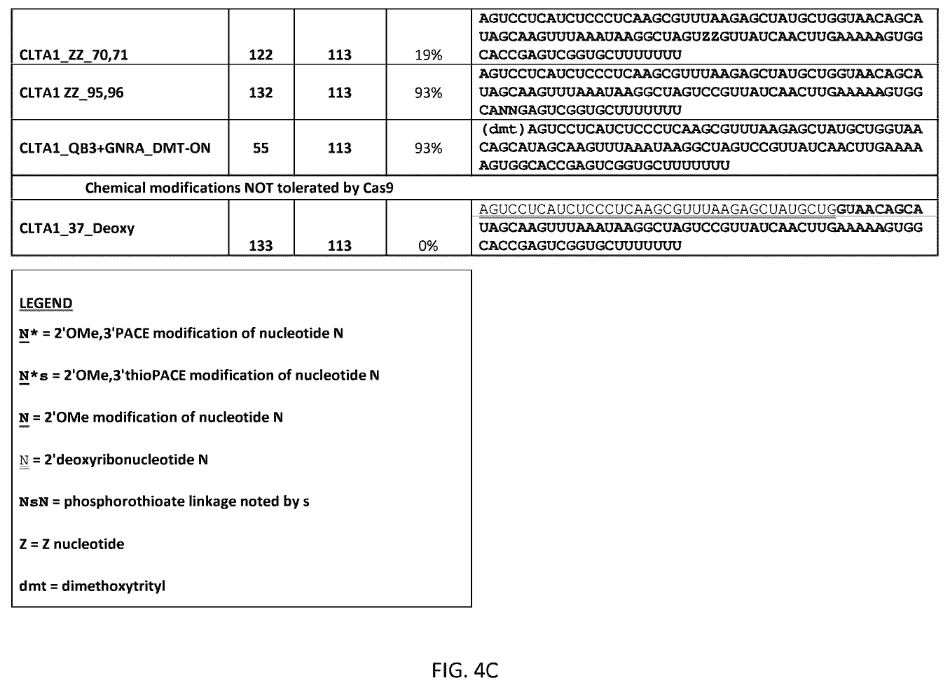

A series of guide RNAs as listed in FIG. 4 were chemically synthesized. Briefly, individual RNA strands were synthesized and HPLC purified. All oligonucleotides were quality control approved on the basis of chemical purity by HPLC analysis and full-length strand purity by mass spectrometry analysis. Each of these guide RNAs was designed to target the human CLTA gene.

The results are shown in FIG. 4. As shown in the Table 1 of FIG. 4, all but one of the chemically synthesized guide RNAs targeted and cleaved the CLTA-encoded DNA target sequence with significant cleavage rates. The one exception was “CLTA_37_Deoxy” guide RNA, which had a contiguous sequence of 37 deoxyribonucleotides at its 5′ end.

As disclosed herein, a variety of chemical modifications were tested at specific positions in the sequence of a guide RNA. Surprisingly, the tested positions in the guide sequence of the guide RNA (a.k.a. the spacer sequence in the guide RNA) tolerated most of the modifications tested, including combinations of multiple modifications within single nucleotides in the guide RNA, even when modifications were instantiated in the target-binding sequences.

The results revealed that guide RNAs containing modifications at specific positions were tolerated by active Cas protein and gRNA: Cas protein complexes, as the modifications did not prevent target-specific cleavage of the target polynucleotide. In all the guide RNA sequences listed in the Table 1 of FIG. 4, the first 20 nucleotides at the 5′ end are complementary to the target sequence in target DNA. The modifications that were tested and found to be tolerated at specific positions include 2′-O-methylribonucleotide (=2′OMe), 2′-deoxyribonucleotide, racemic phosphorothioate internucleotide linkage(s) (═P(S)), 3′-phosphonoacetate (=PACE), 3′-thiophosphonoacetates (=thioPACE), Z nucleotides, and combinations of these.

It is contemplated that the chemical modifications disclosed and tested herein, particularly at the tested positions (as listed in the Table 1 of FIG. 4), will be tolerated at equivalent positions in a variety of guide RNAs. In certain embodiments, the chemical modifications disclosed and tested herein are tolerated in any position in a guide RNA.

As disclosed herein, chemically modified nucleotides were incorporated into guide RNAs in an effort to improve certain properties. Such properties include improved nuclease resistance of the guide RNA, reduced off-target effects of a gRNA: Cas protein complex (also known as improved specificity), improved efficacy of gRNA: Cas protein complex when cleaving, nicking or binding a target polynucleotide, improved transfection efficiency, and/or improved organelle localization such as nuclear localization.

While the use of modified RNA is known (e.g., to block nucleolytic degradation in certain applications), it is widely known that one cannot simply incorporate modifications at any or all positions in an RNA sequence and expect it to function, particularly when the RNA sequence needs to complex with a protein or an enzyme to exert certain functions. Thus, it was not predictable whether these guide RNAs could tolerate chemical modifications at a variety of nucleotide positions while performing sufficient or improved function in a CRISPR-Cas system. In fact, it was unexpected that the guide RNA can tolerate specific modifications to the extent instantiated and tested, especially at several of the positions tested.

### Example 2

To evaluate the ability of the chemically synthesized guide RNAs to target and cleave a DNA target sequence, an in vitro cleavage assay similar to that described in Example 1 was used. Target DNA constructs were for human DNA targets (sequences from the human clathrin light chain (CLTA1) gene, the human Interleukin 2 Receptor Gamma (IL2RG) gene, the human cytotoxic T-lymphocyte-associated protein 4 (CLTA4) gene, the human protocadherin alpha 4 (PCDHA4) gene, and the human engrailed homeobox 1 (EN1) gene), along with off-target DNA constructs differing from the target DNA by one or more nucleotides.

Table 3 sets forth the guide RNA constructs and their sequences, along with DNA constructs used for assessing the ability of those guide RNA constructs to target and cleave. In all the guide RNA sequences listed in the Table 3, the first 20 nucleotides at the 5′ end are complementary to the target sequence in target DNA. ON target constructs comprise the 20 nt target sequence. OFF target constructs comprise most of the same 20 nucleotides as the target DNA, with 1, 2 or 3 nucleotide differences. Accordingly, the guide RNA is mostly, but not completely, complementary to the sequence of the OFF target constructs. The OFF target constructs are based on gene sequences known to occur in the human genome.

TABLE 3

| | | | | |
|---|---|---|---|---|
| Entry | | Target DNA | | RNA |
| # | Guide RNA Construct | Construct | RNA sequence (5′ → 3′) | length |

2-piece dual-guide scaffold
Unmodified dual-guide RNA (dgRNA)

| | | | | |
|---|---|---|---|---|
| 1 | CLTA1 crRNA + tracrRNA | CLTA1 ON1- | AGUCCUCAUCUCCCUCAAGCGUUUAAGAGCUAUGCUGUUUUGAAUGGUCC | 56 + 86 |
| | | target | CAAAAC (SEQ ID NO: 25) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 2 | CLTA1 crRNA + tracrRNA | CLTA1 ON1- | AGUCCUCAUCUCCCUCAAGCGUUUAAGAGCUAUGCUGUUUUGAAUGGUCC | 56 + 86 |
| | | target | CAAAAC (SEQ ID NO: 25) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 3 | CLTA1 crRNA + tracrRNA | CLTA1 OFF1- | AGUCCUCAUCUCCCUCAAGCGUUUAAGAGCUAUGCUGUUUUGAAUGGUCC | 56 + 86 |
| | | target | CAAAAC (SEQ ID NO: 25) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 4 | CLTA1 crRNA + tracrRNA | CLTA1 OFF1- | AGUCCUCAUCUCCCUCAAGCGUUUAAGAGCUAUGCUGUUUUGAAUGGUCC | 56 + 86 |
| | | target | CAAAAC (SEQ ID NO: 25) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 5 | CLTA1 crRNA + tracrRNA | CLTA1 OFF2- | AGUCCUCAUCUCCCUCAAGCGUUUAAGAGCUAUGCUGUUUUGAAUGGUCC | 56 + 86 |
| | | target | CAAAAC (SEQ ID NO: 25) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 6 | CLTA1 crRNA + tracrRNA | CLTA1 OFF2- | AGUCCUCAUCUCCCUCAAGCGUUUAAGAGCUAUGCUGUUUUGAAUGGUCC | 56 + 86 |
| | | target | CAAAAC (SEQ ID NO: 25) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 7 | CLTA1 crRNA + tracrRNA | CLTA1 OFF3- | AGUCCUCAUCUCCCUCAAGCGUUUAAGAGCUAUGCUGUUUUGAAUGGUCC | 56 + 86 |
| | | target | CAAAAC (SEQ ID NO: 25) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 8 | CLTA1 crRNA + tracrRNA | CLTA1 OFF3- | AGUCCUCAUCUCCCUCAAGCGUUUAAGAGCUAUGCUGUUUUGAAUGGUCC | 56 + 86 |
| | | target | CAAAAC (SEQ ID NO: 25) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 9 | CLTA1 crRNA + tracrRNA | CLTA1 | AGUCCUCAUCUCCCUCAAGCGUUUAAGAGCUAUGCUGUUUUGAAUGGUCC | 56 + 86 |
| | | target | CAAAAC (SEQ ID NO: 25) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 10 | IL2RG_crRNA | IL2RGmg ON- | UGGUAAUGAUGGCUUCAACAGUUUUAGAGCUAUGCUGUUUUGAAUGGUC | 56 + 86 |
| | tracrRNA | target | CCAAAAC (SEQ ID NO: 27) | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 28) | |

Fluorophore-coupled dgRNA

| | | | | |
|---|---|---|---|---|
| 11 | CLTA1 crRNA | CLTA1 ON1- | AGUCCUCAUCUCCCUCAAGCGUUUAAGAGCUAUGCUGUUUUGAAUGGUCC | 56 + 86 |
| | tracrRNA_aminoallyl- | target | CAAAAC (SEQ ID NO: 29) + | |
| | U57+Cy5 | | GGAACCAUUCAAAACAGCAUAGCAAGUUUAAAUAAGGCUAGUCCGUUAUCA | |
| | | | ACUUG(aminoallyIU+Cy5)AAAAGUGGCACCGAGUCGGUGCUUUUUUU | |
| | | | (SEQ ID NO: 30) | |

2′OMethyl-modified dgRNA

| | | | | |
|---|---|---|---|---|
| 12 | IL2RG_crRNA_5′, 3′- | IL2RGmg ON- | UGGUAAUGAUGGCUUCAACAGUUUUAGAGCUAUGCUGUUUUGAAUGGUC | 56 + 86 |
| | 3x(2′OMe) | target | CCAAAAC (SEQ ID NO: 31) + | |
| | tracrRNA_5′, 3′- | | GGAACCAUUCAAAACAGCAUAGCAAGUUUAAAUAAGGCUAGUCCGUUAUCA | |
| | 3x(2′OMe) | | ACUUGUAAAAGUGGCACCGAGUCGGUGCUUUUUUU (SEQ ID NO: 32) | |

2′OMethyl, 3′Phosphorothioate-modified dgRNA

| | | | | |
|---|---|---|---|---|
| 13 | IL2RG_crRNA_5′, 3′- | IL2RGmg ON- | UsGsGsUAAUGAUGGCUUCAACAGUUUUAGAGCUAUGCUGUUUUGAAUGG | 56 + 86 |
| | 3x(2′OMe, 3′P(S)) | target | UCCCAAsAsAsC (SEQ ID NO: 33) + | |
| | tracrRNA_5′, 3′- | | GsGsAsACCAUUCAAAACAGCAUAGCAAGUUUAAAUAAGGCUAGUCCGUUAU | |
| | 3x(2′OMe, 3′P(S)) | | CAACUUGUAAAAGUGGCACCGAGUCGGUGCUUUUsUsUsU (SEQ ID NO: | |
| | | | 34) | |

2′OMethyl, 3′PhosphorothioPACE-modified dgRNA

| | | | | |
|---|---|---|---|---|
| 14 | IL2RG_crRNA_5′, 3′- | IL2RGmg ON- | U*sG*sG*sUAAUGAUGGCUUCAACAGUUUUAGAGCUAUGCUGUUUUGAAU | 56 + 86 |
| | 3x(2′OMe, 3′thioPACE) | target | GGUCCCAA*sA*sA*sC (SEQ ID NO: 35) + | |
| | tracrRNA_5′, 3′- | | G*sG*sA*sACCAUUCAAAACAGCAUAGCAAGUUUAAAUAAGGCUAGUCCGU | |
| | 3x(2′OMe, 3′thioPACE) | | UAUCAACUUGUAAAAGUGGCACCGAGUCGGUGCUUUU*sU*sU*sU (SEQ | |
| | | | ID NO: 36) | 56 + 86 |
| 15 | IL2RG_crRNA_5′, 3′- | IL2RGmg ON- | U*sGGUAAUGAUGGCUUCAACAGUUUUAGAGCUAUGCUGUUUUGAAUGGU | |
| | 1x(2′OMe, 3′thioPACE) | target | CCCAAAA*sC (SEQ ID NO: 37) + | |
| | tracrRNA_5′, 3′- | | G*sGAACCAUUCAAAACAGCAUAGCAAGUUUAAAUAAGGCUAGUCCGUUAU | |
| | 1x(2′OMe, 3′thioPACE) | | CAACUUGUAAAAGUGGCACCGAGUCGGUGCUUUUUU*sU (SEQ ID NO: | |
| | | | 38) | |

2-thioU-modified dgRNA

| | | | | |
|---|---|---|---|---|
| 16 | CLTA1_2thioU + 3 | CLTA1 ON1- | AG(2sU)CCUCAUCUCCCUCAAGCGUUUAAGAGCUAUGCUGUUUUGAAUGGU | 56 + 86 |
| | crRNA + tracrRNA | target | CCCAAAAC (SEQ ID NO: 39) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 17 | CLTA1_2thioU + 3 | CLTA1 ON1- | AG(2sU)CCUCAUCUCCCUCAAGCGUUUAAGAGCUAUGCUGUUUUGAAUGGU | 56 + 86 |
| | crRNA + tracrRNA | target | CCCAAAAC (SEQ ID NO: 39) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 18 | CLTA1_2thioU + 3 | CLTA1 OFF1- | AG(2sU)CCUCAUCUCCCUCAAGCGUUUAAGAGCUAUGCUGUUUUGAAUGGU | 56 + 86 |
| | crRNA + tracrRNA | target | CCCAAAAC (SEQ ID NO: 39) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 19 | CLTA1_2thioU + 3 | CLTA1 OFF1- | AG(2sU)CCUCAUCUCCCUCAAGCGUUUAAGAGCUAUGCUGUUUUGAAUGGU | 56 + 86 |
| | crRNA + tracrRNA | target | CCCAAAAC (SEQ ID NO: 39) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 20 | CLTA1_2thioU + 3 | CLTA1 OFF2- | AG(2sU)CCUCAUCUCCCUCAAGCGUUUAAGAGCUAUGCUGUUUUGAAUGGU | 56 + 86 |
| | crRNA + tracrRNA | target | CCCAAAAC (SEQ ID NO: 39) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 21 | CLTA1_2thioU + 3 | CLTA1 OFF2- | AG(2sU)CCUCAUCUCCCUCAAGCGUUUAAGAGCUAUGCUGUUUUGAAUGGU | 56 + 86 |
| | crRNA + tracrRNA | target | CCCAAAAC (SEQ ID NO: 39) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 22 | CLTA1_2thioU + 3 | CLTA1 OFF3- | AG(2sU)CCUCAUCUCCCUCAAGCGUUUAAGAGCUAUGCUGUUUUGAAUGGU | 56 + 86 |
| | crRNA + tracrRNA | target | CCCAAAAC (SEQ ID NO: 39) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 23 | CLTA1_2thioU + 3 | CLTA1 OFF3- | AG(2sU)CCUCAUCUCCCUCAAGCGUUUAAGAGCUAUGCUGUUUUGAAUGGU | 56 + 86 |
| | crRNA + tracrRNA | target | CCCAAAAC (SEQ ID NO: 39) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 24 | CLTA1_2thioU + 9 | CLTA1 ON1- | AGUCCUCA(2sU)CUCCCUCAAGCGUUUAAGAGCUAUGCUGUUUUGAAUGGU | 56 + 86 |
| | crRNA + tracrRNA | target | CCCAAAAC (SEQ ID NO: 40) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 25 | CLTA1_2thioU + 9 | CLTA1 ON1- | AGUCCUCA(2sU)CUCCCUCAAGCGUUUAAGAGCUAUGCUGUUUUGAAUGGU | 56 + 86 |
| | crRNA + tracrRNA | target | CCCAAAAC (SEQ ID NO: 40) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 26 | CLTA1_2thioU + 9 | CLTA1 OFF1- | AGUCCUCA(2sU)CUCCCUCAAGCGUUUAAGAGCUAUGCUGUUUUGAAUGGU | 56 + 86 |
| | crRNA + tracrRNA | target | CCCAAAAC (SEQ ID NO: 40) | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 27 | CLTA1_2thioU + 9 | CLTA1 OFF1- | AGUCCUCA(2sU)CUCCCUCAAGCGUUUAAGAGCUAUGCUGUUUUGAAUGGU | 56 + 86 |
| | crRNA + tracrRNA | target | CCCAAAAC (SEQ ID NO: 40) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 28 | CLTA1_2thioU + 9 | CLTA1 OFF2- | AGUCCUCA(2sU)CUCCCUCAAGCGUUUAAGAGCUAUGCUGUUUUGAAUGGU | 56 + 86 |
| | crRNA + tracrRNA | target | CCCAAAAC (SEQ ID NO: 40) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 29 | CLTA1_2thioU + 9 | CLTA1 OFF2- | AGUCCUCA(2sU)CUCCCUCAAGCGUUUAAGAGCUAUGCUGUUUUGAAUGGU | 56 + 86 |
| | crRNA + tracrRNA | target | CCCAAAAC (SEQ ID NO: 40) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 30 | CLTA1_2thioU + 9 | CLTA1 OFF3- | AGUCCUCA(2sU)CUCCCUCAAGCGUUUAAGAGCUAUGCUGUUUUGAAUGGU | 56 + 86 |
| | crRNA + tracrRNA | target | CCCAAAAC (SEQ ID NO: 40) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 31 | CLTA1_2thioU + 9 | CLTA1 OFF3- | AGUCCUCA(2sU)CUCCCUCAAGCGUUUAAGAGCUAUGCUGUUUUGAAUGGU | 56 + 86 |
| | crRNA + tracrRNA | target | CCCAAAAC (SEQ ID NO: 40) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 32 | CLTA1_2thioU + 11 | CLTA1 ON1- | AGUCCUCAUC(2sU)CCCUCAAGCGUUUAAGAGCUAUGCUGUUUUGAAUGGU | 56 + 86 |
| | crRNA + tracrRNA | target | CCCAAAAC (SEQ ID NO: 41) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 33 | CLTA1_2thioU + 11 | CLTA1 ON1- | AGUCCUCAUC(2sU)CCCUCAAGCGUUUAAGAGCUAUGCUGUUUUGAAUGGU | 56 + 86 |
| | crRNA + tracrRNA | target | CCCAAAAC (SEQ ID NO: 41) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 34 | CLTA1_2thioU + 11 | CLTA1 OFF1- | AGUCCUCAUC(2sU)CCCUCAAGCGUUUAAGAGCUAUGCUGUUUUGAAUGGU | 56 + 86 |
| | crRNA + tracrRNA | target | CCCAAAAC (SEQ ID NO: 41) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 35 | CLTA1_2thioU + 11 | CLTA1 OFF1- | AGUCCUCAUC(2sU)CCCUCAAGCGUUUAAGAGCUAUGCUGUUUUGAAUGGU | 56 + 86 |
| | crRNA + tracrRNA | target | CCCAAAAC (SEQ ID NO: 41) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 36 | CLTA1_2thioU + 11 | CLTA1 OFF2- | AGUCCUCAUC(2sU)CCCUCAAGCGUUUAAGAGCUAUGCUGUUUUGAAUGGU | 56 + 86 |
| | crRNA + tracrRNA | target | CCCAAAAC (SEQ ID NO: 41) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 37 | CLTA1_2thioU + 11 | ″ | AGUCCUCAUC(2sU)CCCUCAAGCGUUUAAGAGCUAUGCUGUUUUGAAUGGU | 56 + 86 |
| | crRNA + tracrRNA | | CCCAAAAC (SEQ ID NO: 41) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 38 | CLTA1_2thioU + 11 | CLTA1 OFF3- | AGUCCUCAUC(2sU)CCCUCAAGCGUUUAAGAGCUAUGCUGUUUUGAAUGGU | 56 + 86 |
| | crRNA + tracrRNA | target | CCCAAAAC (SEQ ID NO: 41) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |
| 39 | CLTA1_2thioU + 11 | ″ | AGUCCUCAUC(2sU)CCCUCAAGCGUUUAAGAGCUAUGCUGUUUUGAAUGGU | 56 + 86 |
| | crRNA + tracrRNA | | CCCAAAAC (SEQ ID NO: 41) + | |
| | | | GGAACCAUUCAAAACAGCAUAGCAAGUUUAAAUAAGGCUAGUCCGUUAUCA | |
| | | | ACUUGUAAAAGUGGCACCGAGUCGGUGCUUUUUUU (SEQ ID NO: 26) | |

Single-guide scaffold
Unmodified single-guide RNA (sgRNA)

| | | | | |
|---|---|---|---|---|
| 40 | CLTA1 sgRNA (Batch #1) | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 42) | |
| 41 | CLTA1 sgRNA (Batch #1) | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 42) | |
| 42 | CLTA1 sgRNA (Batch #2) | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 42) | |
| 43 | CLTA1 sgRNA (Batch #2) | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 42) | |
| 44 | CLTA1 sgRNA (Batch #3) | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 42) | |
| 45 | CLTA1 sgRNA (Batch #3) | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 42) | |
| 46 | CLTA1 sgRNA (Batch #3) | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 42) | |
| 47 | CLTA1 sgRNA (Batch #3) | CLTA1mg | AGUCCUCAUCUCCCUCAAGCGUUUAAGAGCUAUGCUGGUAACAGCAUAGCA | 113 |
| | | ON1-target | AGUUUAAAUAAGGCUAGUCCGUUAUCAACUUGAAAAAGUGGCACCGAGUC | |
| | | | GGUGCUUUUUUU (SEQ ID NO: 42) | |
| 48 | CLTA1 sgRNA (Batch #3) | CLTA1mg | AGUCCUCAUCUCCCUCAAGCGUUUAAGAGCUAUGCUGGUAACAGCAUAGCA | 113 |
| | | ON1-target | AGUUUAAAUAAGGCUAGUCCGUUAUCAACUUGAAAAAGUGGCACCGAGUC | |
| | | | GGUGCUUUUUUU (SEQ ID NO: 42) | |
| 49 | CLTA1 sgRNA (Batch #3) | CLTA1mg | AGUCCUCAUCUCCCUCAAGCGUUUAAGAGCUAUGCUGGUAACAGCAUAGCA | 113 |
| | | ON1-target | AGUUUAAAUAAGGCUAGUCCGUUAUCAACUUGAAAAAGUGGCACCGAGUC | |
| | | | GGUGCUUUUUUU (SEQ ID NO: 42) | |
| 50 | CLTA1 sgRNA (Batch #3) | CLTA1mg | AGUCCUCAUCUCCCUCAAGCGUUUAAGAGCUAUGCUGGUAACAGCAUAGCA | 113 |
| | | OFF1-target | AGUUUAAAUAAGGCUAGUCCGUUAUCAACUUGAAAAAGUGGCACCGAGUC | |
| | | | GGUGCUUUUUUU (SEQ ID NO: 42) | |
| 51 | CLTA1 sgRNA (Batch #3) | CLTA1mg | AGUCCUCAUCUCCCUCAAGCGUUUAAGAGCUAUGCUGGUAACAGCAUAGCA | 113 |
| | | OFF3-target | AGUUUAAAUAAGGCUAGUCCGUUAUCAACUUGAAAAAGUGGCACCGAGUC | |
| | | | GGUGCUUUUUUU (SEQ ID NO: 42) | |
| 52 | CLTA1 sgRNA (crude) | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 42) | |
| 53 | CLTA1_Bos sgRNA | CLTA1mg | AGUCCUCAUCUCCCUCAAGCGUUUUAGAGCUAGUAAUAGCAAGUUAAAAUA | 100 |
| | | ON1-target | AGGCUAGUCCGUUAUCAACUUGAAAAAGUGGCACCGAGUCGGUGCUUUU | |
| | | | (SEQ ID NO: 43) | |
| 54 | CLTA1_Bos sgRNA | CLTA1mg | AGUCCUCAUCUCCCUCAAGCGUUUUAGAGCUAGUAAUAGCAAGUUAAAAUA | 100 |
| | | ON1-target | AGGCUAGUCCGUUAUCAACUUGAAAAAGUGGCACCGAGUCGGUGCUUUU | |
| | | | (SEQ ID NO: 43) | |
| 55 | CLTA1_Bos sgRNA | CLTA1mg | AGUCCUCAUCUCCCUCAAGCGUUUUAGAGCUAGUAAUAGCAAGUUAAAAUA | 100 |
| | | ON1-target | AGGCUAGUCCGUUAUCAACUUGAAAAAGUGGCACCGAGUCGGUGCUUUU | |
| | | | (SEQ ID NO: 43) | |
| 56 | CLTA1_Bos sgRNA | CLTA1mg | AGUCCUCAUCUCCCUCAAGCGUUUUAGAGCUAGUAAUAGCAAGUUAAAAUA | 100 |
| | | OFF1-target | AGGCUAGUCCGUUAUCAACUUGAAAAAGUGGCACCGAGUCGGUGCUUUU | |
| | | | (SEQ ID NO: 43) | |
| 57 | CLTA1_Bos sgRNA | CLTA1mg | AGUCCUCAUCUCCCUCAAGCGUUUUAGAGCUAGUAAUAGCAAGUUAAAAUA | 100 |
| | | OFF3-target | AGGCUAGUCCGUUAUCAACUUGAAAAAGUGGCACCGAGUCGGUGCUUUU | |
| | | | (SEQ ID NO: 43) | |
| 58 | CLTA4 sgRNA | CLTA4 ON- | GCAGAUGUAGUGUUUCCACAGUUUAAGAGCUAUGCUGGAAACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 44) | |
| 59 | CLTA4 sgRNA | CLTA4 ON- | GCAGAUGUAGUGUUUCCACAGUUUAAGAGCUAUGCUGGAAACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 44) | |
| 60 | CLTA4 sgRNA | CLTA4 ON- | GCAGAUGUAGUGUUUCCACAGUUUAAGAGCUAUGCUGGAAACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 44) | |
| 61 | CLTA4 sgRNA | CLTA4mg ON- | GCAGAUGUAGUGUUUCCACAGUUUAAGAGCUAUGCUGGAAACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 44) | |
| 62 | CLTA4 sgRNA | CLTA4mg ON- | GCAGAUGUAGUGUUUCCACAGUUUAAGAGCUAUGCUGGAAACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 44) | |
| 63 | CLTA4 sgRNA | CLTA4mg | GCAGAUGUAGUGUUUCCACAGUUUAAGAGCUAUGCUGGAAACAGCAUAGC | 113 |
| | | OFF5-target | AAGUUUAAAUAAGGCUAGUCCGUUAUCAACUUGAAAAAGUGGCACCGAGU | |
| | | | CGGUGCUUUUUUU (SEQ ID NO: 44) | |
| 64 | CLTA1_Truncated_18mer | CLTA1mg | UCCUCAUCUCCCUCAAGCGUUUAAGAGCUAUGCUGGUAACAGCAUAGCAAG | 111 |
| | | ON1-target | UUUAAAUAAGGCUAGUCCGUUAUCAACUUGAAAAAGUGGCACCGAGUCGG | |
| | | | UGCUUUUUUU (SEQ ID NO: 45) | |
| 65 | CLTA1_Truncated_18mer | CLTA1mg | UCCUCAUCUCCCUCAAGCGUUUAAGAGCUAUGCUGGUAACAGCAUAGCAAG | 111 |
| | | ON1-target | UUUAAAUAAGGCUAGUCCGUUAUCAACUUGAAAAAGUGGCACCGAGUCGG | |
| | | | UGCUUUUUUU (SEQ ID NO: 45) | |
| 66 | CLTA1_Truncated_18mer | CLTA1mg | UCCUCAUCUCCCUCAAGCGUUUAAGAGCUAUGCUGGUAACAGCAUAGCAAG | 111 |
| | | OFF1-target | UUUAAAUAAGGCUAGUCCGUUAUCAACUUGAAAAAGUGGCACCGAGUCGG | |
| | | | UGCUUUUUUU (SEQ ID NO: 45) | |
| 67 | CLTA1_Truncated_18mer | CLTA1mg | UCCUCAUCUCCCUCAAGCGUUUAAGAGCUAUGCUGGUAACAGCAUAGCAAG | 111 |
| | | OFF3-target | UUUAAAUAAGGCUAGUCCGUUAUCAACUUGAAAAAGUGGCACCGAGUCGG | |
| | | | UGCUUUUUUU (SEQ ID NO: 45) | |
| 68 | CLTA1_Truncated_17mer | CLTA1mg | CCUCAUCUCCCUCAAGCGUUUAAGAGCUAUGCUGGUAACAGCAUAGCAAGU | 110 |
| | | ON1-target | UUAAAUAAGGCUAGUCCGUUAUCAACUUGAAAAAGUGGCACCGAGUCGGU | |
| | | | GCUUUUUUU (SEQ ID NO: 46) | |
| 69 | CLTA1_Truncated_17mer | CLTA1mg | CCUCAUCUCCCUCAAGCGUUUAAGAGCUAUGCUGGUAACAGCAUAGCAAGU | 110 |
| | | ON1-target | UUAAAUAAGGCUAGUCCGUUAUCAACUUGAAAAAGUGGCACCGAGUCGGU | |
| | | | GCUUUUUUU (SEQ ID NO: 46) | |
| 70 | CLTA1_Truncated_17mer | CLTA1mg | CCUCAUCUCCCUCAAGCGUUUAAGAGCUAUGCUGGUAACAGCAUAGCAAGU | 110 |
| | | OFF1-target | UUAAAUAAGGCUAGUCCGUUAUCAACUUGAAAAAGUGGCACCGAGUCGGU | |
| | | | GCUUUUUUU (SEQ ID NO: 46) | |
| 71 | CLTA1_Truncated_17mer | CLTA1mg | CCUCAUCUCCCUCAAGCGUUUAAGAGCUAUGCUGGUAACAGCAUAGCAAGU | 110 |
| | | OFF3-target | UUAAAUAAGGCUAGUCCGUUAUCAACUUGAAAAAGUGGCACCGAGUCGGU | |
| | | | GCUUUUUUU (SEQ ID NO: 46) | |
| 72 | CLTA1_1xExtraG | CLTA1mg | GAGUCCUCAUCUCCCUCAAGCGUUUAAGAGCUAUGCUGGUAACAGCAUAGC | 114 |
| | | ON1-target | AAGUUUAAAUAAGGCUAGUCCGUUAUCAACUUGAAAAAGUGGCACCGAGU | |
| | | | CGGUGCUUUUUUU (SEQ ID NO: 47) | |
| 73 | CLTA1_1xExtraG | CLTA1mg | GAGUCCUCAUCUCCCUCAAGCGUUUAAGAGCUAUGCUGGUAACAGCAUAGC | 114 |
| | | ON1-target | AAGUUUAAAUAAGGCUAGUCCGUUAUCAACUUGAAAAAGUGGCACCGAGU | |
| | | | CGGUGCUUUUUUU (SEQ ID NO: 47) | |
| 74 | CLTA1_1xExtraG | CLTA1mg | GAGUCCUCAUCUCCCUCAAGCGUUUAAGAGCUAUGCUGGUAACAGCAUAGC | 114 |
| | | OFF1-target | AAGUUUAAAUAAGGCUAGUCCGUUAUCAACUUGAAAAAGUGGCACCGAGU | |
| | | | CGGUGCUUUUUUU (SEQ ID NO: 47) | |
| 75 | CLTA1_1xExtraG | CLTA1mg | GAGUCCUCAUCUCCCUCAAGCGUUUAAGAGCUAUGCUGGUAACAGCAUAGC | 114 |
| | | OFF3-target | AAGUUUAAAUAAGGCUAGUCCGUUAUCAACUUGAAAAAGUGGCACCGAGU | |
| | | | CGGUGCUUUUUUU (SEQ ID NO: 47) | |
| 76 | CLTA1_2xExtraG | CLTA1mg | GGAGUCCUCAUCUCCCUCAAGCGUUUAAGAGCUAUGCUGGUAACAGCAUAG | 115 |
| | | ON1-target | CAAGUUUAAAUAAGGCUAGUCCGUUAUCAACUUGAAAAAGUGGCACCGAG | |
| | | | UCGGUGCUUUUUUU (SEQ ID NO: 48) | |
| 77 | CLTA1_2xExtraG | CLTA1mg ON1- | GGAGUCCUCAUCUCCCUCAAGCGUUUAAGAGCUAUGCUGGUAACAGCAUAG | 115 |
| | | target | CAAGUUUAAAUAAGGCUAGUCCGUUAUCAACUUGAAAAAGUGGCACCGAG | |
| | | | UCGGUGCUUUUUUU (SEQ ID NO: 48) | |
| 78 | CLTA1_2xExtraG | CLTA1mg | GGAGUCCUCAUCUCCCUCAAGCGUUUAAGAGCUAUGCUGGUAACAGCAUAG | 115 |
| | | OFF1-target | CAAGUUUAAAUAAGGCUAGUCCGUUAUCAACUUGAAAAAGUGGCACCGAG | |
| | | | UCGGUGCUUUUUUU (SEQ ID NO: 48) | |
| 79 | CLTA1_2xExtraG | CLTA1mg | GGAGUCCUCAUCUCCCUCAAGCGUUUAAGAGCUAUGCUGGUAACAGCAUAG | 115 |
| | | OFF3-target | CAAGUUUAAAUAAGGCUAGUCCGUUAUCAACUUGAAAAAGUGGCACCGAG | |
| | | | UCGGUGCUUUUUUU (SEQ ID NO: 48) | |
| 80 | CLTA1_63U, 64U | CLTA1mg ON1- | AGUCCUCAUCUCCCUCAAGCGUUUAAGAGCUAUGCUGGUAACAGCAUAGCA | 113 |
| | | target | AGUUUAAAUAAUUCUAGUCCGUUAUCAACUUGAAAAAGUGGCACCGAGUC | |
| | | | GGUGCUUUUUUU (SEQ ID NO: 49) | |
| 81 | CLTA1_63A, 64A | CLTA1mg ON1- | AGUCCUCAUCUCCCUCAAGCGUUUAAGAGCUAUGCUGGUAACAGCAUAGCA | 113 |
| | | target | AGUUUAAAUAAAACUAGUCCGUUAUCAACUUGAAAAAGUGGCACCGAGUC | |
| | | | GGUGCUUUUUUU (SEQ ID NO: 50) | |
| 82 | CLTA1_63A, 64A, | CLTA1mg ON1- | AGUCCUCAUCUCCCUCAAGCGUUUAAGAGCUAUGCUGGUAACAGCAUAGCA | 113 |
| | 70U, 71U | target | AGUUUAAAUAAAACUAGUUUGUUAUCAACUUGAAAAAGUGGCACCGAGUC | |
| | | | GGUGCUUUUUUU (SEQ ID NO: 51) | |
| 83 | CLTA1_cis-block(1- | CLTA1mg ON1- | GGACUUUUUUUAGUCCUCAUCUCCCUCAAGCGUUUUAGAGCUAGAAAUAG | 111 |
| | 5)_polyU_sgRNA | target | CAAGUUAAAAUAAGGCUAGUCCGUUAUCAACUUGAAAAAGUGGCACCGAG | |
| | | | UCGGUGCUUUU (SEQ ID NO: 52) | |
| 84 | CLTA1_cis-block(1- | CLTA1mg ON1- | GGACUUUUUUUAGUCCUCAUCUCCCUCAAGCGUUUUAGAGCUAGAAAUAG | 111 |
| | 5)_polyU_sgRNA | target | CAAGUUAAAAUAAGGCUAGUCCGUUAUCAACUUGAAAAAGUGGCACCGAG | |
| | | | UCGGUGCUUUU (SEQ ID NO: 52) | |
| 85 | CLTA1_cis-block(1- | CLTA1mg | GGACUUUUUUUAGUCCUCAUCUCCCUCAAGCGUUUUAGAGCUAGAAAUAG | 111 |
| | 5)_polyU_sgRNA | OFF1-target | CAAGUUAAAAUAAGGCUAGUCCGUUAUCAACUUGAAAAAGUGGCACCGAG | |
| | | | UCGGUGCUUUU (SEQ ID NO: 52) | |
| 86 | CLTA1_cis-block(1- | CLTA1mg | GGACUUUUUUUAGUCCUCAUCUCCCUCAAGCGUUUUAGAGCUAGAAAUAG | 111 |
| | 5)_polyU_sgRNA | OFF3-target | CAAGUUAAAAUAAGGCUAGUCCGUUAUCAACUUGAAAAAGUGGCACCGAG | |
| | | | UCGGUGCUUUU (SEQ ID NO: 52) | |
| 87 | CLTA1_cis-block | CLTA1mg ON1- | GAUGAGGACUUUUUUUAGUCCUCAUCUCCCUCAAGCGUUUUAGAGCUAGA | 116 |
| | (1-10)_polyU_sgRNA | target | AAUAGCAAGUUAAAAUAAGGCUAGUCCGUUAUCAACUUGAAAAAGUGGCA | |
| | | | CCGAGUCGGUGCUUUU (SEQ ID NO: 53) | |
| 88 | CLTA1_cis-block | CLTA1mg ON1- | GAUGAGGACUUUUUUUAGUCCUCAUCUCCCUCAAGCGUUUUAGAGCUAGA | 116 |
| | (1-10)_polyU_sgRNA | target | AAUAGCAAGUUAAAAUAAGGCUAGUCCGUUAUCAACUUGAAAAAGUGGCA | |
| | | | CCGAGUCGGUGCUUUU (SEQ ID NO: 53) | |
| 89 | CLTA1_cis-block | CLTA1mg | GAUGAGGACUUUUUUUAGUCCUCAUCUCCCUCAAGCGUUUUAGAGCUAGA | 116 |
| | (1-10)_polyU_sgRNA | OFF1-target | AAUAGCAAGUUAAAAUAAGGCUAGUCCGUUAUCAACUUGAAAAAGUGGCA | |
| | | | CCGAGUCGGUGCUUUU (SEQ ID NO: 53) | |
| 90 | CLTA1_cis-block | CLTA1mg | GAUGAGGACUUUUUUUAGUCCUCAUCUCCCUCAAGCGUUUUAGAGCUAGA | 116 |
| | (1-10)_polyU_sgRNA | OFF3-target | AAUAGCAAGUUAAAAUAAGGCUAGUCCGUUAUCAACUUGAAAAAGUGGCA | |
| | | | CCGAGUCGGUGCUUUU (SEQ ID NO: 53) | |
| 91 | CLTA1_cis-block | CLTA1mg ON1- | GCUUGUUUUUUAGUCCUCAUCUCCCUCAAGCGUUUUAGAGCUAGAAAUAG | 111 |
| | (16-20)_polyU_sgRNA | target | CAAGUUAAAAUAAGGCUAGUCCGUUAUCAACUUGAAAAAGUGGCACCGAG | |
| | | | UCGGUGCUUUU (SEQ ID NO: 54) | |
| 92 | CLTA1_cis-block | CLTA1mg ON1- | GCUUGUUUUUUAGUCCUCAUCUCCCUCAAGCGUUUUAGAGCUAGAAAUAG | 111 |
| | (16-20)_polyU_sgRNA | target | CAAGUUAAAAUAAGGCUAGUCCGUUAUCAACUUGAAAAAGUGGCACCGAG | |
| | | | UCGGUGCUUUU (SEQ ID NO: 54) | |
| 93 | CLTA1_cis-block | CLTA1mg | GCUUGUUUUUUAGUCCUCAUCUCCCUCAAGCGUUUUAGAGCUAGAAAUAG | 111 |
| | (16-20)_polyU_sgRNA | OFF1-target | CAAGUUAAAAUAAGGCUAGUCCGUUAUCAACUUGAAAAAGUGGCACCGAG | |
| | | | UCGGUGCUUUU (SEQ ID NO: 54) | |
| 94 | CLTA1_cis-block | CLTA1mg | GCUUGUUUUUUAGUCCUCAUCUCCCUCAAGCGUUUUAGAGCUAGAAAUAG | 111 |
| | (16-20)_polyU_sgRNA | OFF3-target | CAAGUUAAAAUAAGGCUAGUCCGUUAUCAACUUGAAAAAGUGGCACCGAG | |
| | | | UCGGUGCUUUU (SEQ ID NO: 54) | |

DMT-modified sgRNA

| | | | | |
|---|---|---|---|---|
| 95 | CLTA1_DMT-ON sgRNA | CLTA1 ON1- | (dmt)AGUCCUCAUCUCCCUCAAGCGUUUAAGAGCUAUGCUGGUAACAGCAU | 113 |
| | | target | AGCAAGUUUAAAUAAGGCUAGUCCGUUAUCAACUUGAAAAAGUGGCACCG | |
| | | | AGUCGGUGCUUUUUUU (SEQ ID NO: 55) | |
| 96 | CLTA1_DMT-ON/OFF | CLTA1 ON1- | AGUCCUCAUCUCCCUCAAGCGUUUAAGAGCUAUGCUGGUAACAGCAUAGCA | 113 |
| | sgRNA | target | AGUUUAAAUAAGGCUAGUCCGUUAUCAACUUGAAAAAGUGGCACCGAGUC | |
| | | | GGUGCUUUUUUU (SEQ ID NO: 56) | |

Fluorophore-modified sgRNA

| | | | | |
|---|---|---|---|---|
| 97 | CLTA1_IntFl_sgLoop | CLTA1 ON1- | AGUCCUCAUCUCCCUCAAGCGUUUAAGAGCUAUGCUGG(Fl)AACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 57) | |
| 98 | CLTA1_IntFl_sgLoop | CLTA1mg ON1- | AGUCCUCAUCUCCCUCAAGCGUUUAAGAGCUAUGCUGG(Fl)AACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 57) | |
| 99 | CLTA1_IntFl_sgLoop | CLTA1mg ON1- | AGUCCUCAUCUCCCUCAAGCGUUUAAGAGCUAUGCUGG(Fl)AACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 57) | |
| 100 | CLTA1_IntFl_sgLoop | CLTA1mg | AGUCCUCAUCUCCCUCAAGCGUUUAAGAGCUAUGCUGG(Fl)AACAGCAUAGC | 113 |
| | | OFF1-target | AAGUUUAAAUAAGGCUAGUCCGUUAUCAACUUGAAAAAGUGGCACCGAGU | |
| | | | CGGUGCUUUUUUU (SEQ ID NO: 57) | |
| 101 | CLTA1_IntFl_sgLoop | CLTA1mg | AGUCCUCAUCUCCCUCAAGCGUUUAAGAGCUAUGCUGG(Fl)AACAGCAUAGC | 113 |
| | | OFF3-target | AAGUUUAAAUAAGGCUAGUCCGUUAUCAACUUGAAAAAGUGGCACCGAGU | |
| | | | CGGUGCUUUUUUU (SEQ ID NO: 57) | |
| 102 | CLTA1_IntFl_sgLoop_5′, | CLTA1mg ON1- | AGUCCUCAUCUCCCUCAAGCGUUUAAGAGCUAUGCUGG(Fl)AACAGCAUAGC | 113 |
| | 3′-3x(2′OMe) | target | AAGUUUAAAUAAGGCUAGUCCGUUAUCAACUUGAAAAAGUGGCACCGAGU | |
| | | | CGGUGCUUUUUUU (SEQ ID NO: 58) | |
| 103 | CLTA1_IntFl_sgLoop_5′, | CLTA1mg ON1- | AGUCCUCAUCUCCCUCAAGCGUUUAAGAGCUAUGCUGG(Fl)AACAGCAUAGC | 113 |
| | 3′-3x(2′OMe) | target | AAGUUUAAAUAAGGCUAGUCCGUUAUCAACUUGAAAAAGUGGCACCGAGU | |
| | | | CGGUGCUUUUUUU (SEQ ID NO: 58) | |
| 104 | CLTA1_IntFl_sgLoop_5′, | CLTA1mg | AGUCCUCAUCUCCCUCAAGCGUUUAAGAGCUAUGCUGG(Fl)AACAGCAUAGC | 113 |
| | 3′-3x(2′OMe) | OFF1-target | AAGUUUAAAUAAGGCUAGUCCGUUAUCAACUUGAAAAAGUGGCACCGAGU | |
| | | | CGGUGCUUUUUUU (SEQ ID NO: 58) | |
| 105 | CLTA1_IntFl_sgLoop_5′, | CLTA1mg | AGUCCUCAUCUCCCUCAAGCGUUUAAGAGCUAUGCUGG(Fl)AACAGCAUAGC | 113 |
| | 3′-3x(2′OMe) | OFF3-target | AAGUUUAAAUAAGGCUAGUCCGUUAUCAACUUGAAAAAGUGGCACCGAGU | |
| | | | CGGUGCUUUUUUU (SEQ ID NO: 58) | |
| 106 | CLTA1_IntFl_sgLoop_5′, | CLTA1mg ON1- | AsGsUsCCUCAUCUCCCUCAAGCGUUUAAGAGCUAUGCUGG(Fl)AACAGCAUA | 113 |
| | 3′-3x(2′OMe, 3′P(S)) | target | GCAAGUUUAAAUAAGGCUAGUCCGUUAUCAACUUGAAAAAGUGGCACCGA | |
| | | | GUCGGUGCUUUUsUsUsU (SEQ ID NO: 59) | |
| 107 | CLTA1_IntFl_sgLoop_5′, | CLTA1mg ON1- | AsGsUsCCUCAUCUCCCUCAAGCGUUUAAGAGCUAUGCUGG(Fl)AACAGCAUA | 113 |
| | 3′-3x(2′OMe, 3′P(S)) | target | GCAAGUUUAAAUAAGGCUAGUCCGUUAUCAACUUGAAAAAGUGGCACCGA | |
| | | | GUCGGUGCUUUUsUsUsU (SEQ ID NO: 59) | |
| 108 | CLTA1_IntFl_sgLoop_5′, | CLTA1mg | AsGsUsCCUCAUCUCCCUCAAGCGUUUAAGAGCUAUGCUGG(Fl)AACAGCAUA | 113 |
| | 3′-3x(2′OMe, 3′P(S)) | OFF1-target | GCAAGUUUAAAUAAGGCUAGUCCGUUAUCAACUUGAAAAAGUGGCACCGA | |
| | | | GUCGGUGCUUUUsUsUsU (SEQ ID NO: 59) | |
| 109 | CLTA1_IntFl_sgLoop_5′, | CLTA1mg | AsGsUsCCUCAUCUCCCUCAAGCGUUUAAGAGCUAUGCUGG(Fl)AACAGCAUA | 113 |
| | 3′-3x(2′OMe, 3′P(S)) | OFF3-target | GCAAGUUUAAAUAAGGCUAGUCCGUUAUCAACUUGAAAAAGUGGCACCGA | |
| | | | GUCGGUGCUUUUsUsUsU (SEQ ID NO: 59) | |
| 110 | CLTA1_IntFl_sgLoop_5′, | CLTA1mg ON1- | A*sG*sU*sCCUCAUCUCCCUCAAGCGUUUAAGAGCUAUGCUGG(Fl)AACAGC | 113 |
| | 3′-3x(2′OMe, 3′thioPACE) | target | AUAGCAAGUUUAAAUAAGGCUAGUCCGUUAUCAACUUGAAAAAGUGGCAC | |
| | | | CGAGUCGGUGCUUUU*sU*sU*sU (SEQ ID NO: 60) | |
| 111 | CLTA1_IntFl_sgLoop_5′, | CLTA1mg ON1- | A*sG*sU*sCCUCAUCUCCCUCAAGCGUUUAAGAGCUAUGCUGG(Fl)AACAGC | 113 |
| | 3′-3x(2′OMe, 3′thioPACE) | target | AUAGCAAGUUUAAAUAAGGCUAGUCCGUUAUCAACUUGAAAAAGUGGCAC | |
| | | | CGAGUCGGUGCUUUU*sU*sU*sU (SEQ ID NO: 60) | |
| 112 | CLTA1_IntFl_sgLoop_5′, | CLTA1mg | A*sG*sU*sCCUCAUCUCCCUCAAGCGUUUAAGAGCUAUGCUGG(Fl)AACAGC | 113 |
| | 3′-3x(2′OMe, 3′thioPACE) | OFF1-target | AUAGCAAGUUUAAAUAAGGCUAGUCCGUUAUCAACUUGAAAAAGUGGCAC | |
| | | | CGAGUCGGUGCUUUU*sU*sU*sU (SEQ ID NO: 60) | |
| 113 | CLTA1_IntFl_sgLoop_5′, | CLTA1mg | A*sG*sU*sCCUCAUCUCCCUCAAGCGUUUAAGAGCUAUGCUGG(Fl)AACAGC | 113 |
| | 3′-3x(2′OMe, 3′thioPACE) | OFF3-target | AUAGCAAGUUUAAAUAAGGCUAGUCCGUUAUCAACUUGAAAAAGUGGCAC | |
| | | | CGAGUCGGUGCUUUU*sU*sU*sU (SEQ ID NO: 60) | |
| 114 | CLTA4_3xFl- | CLTA4mg ON- | Uₒ*s(Flₒ)GCAGAUGUAGUGUUUCCACAGUUUaAGAGCUAGUAAUAGCAAGU | 102 |
| | Int_1x(2′OMe, | target | UuAAAUAAGGCUAGUCCGUUA(Fl)CAACUUGAAAAAGUGGCACCGAGUCGG | |
| | 3′thioPACE) | | UGCU(Fl)U*sU (SEQ ID NO: 61) | |
| 115 | CLTA4_3xFl- | CLTA4mg | Uₒ*s(Flₒ)GCAGAUGUAGUGUUUCCACAGUUUaAGAGCUAGUAAUAGCAAGU | 102 |
| | Int_1x(2′OMe, | OFF5-target | UuAAAUAAGGCUAGUCCGUUA(Fl)CAACUUGAAAAAGUGGCACCGAGUCGG | |
| | 3′thioPACE) | | UGCU(Fl)U*sU (SEQ ID NO: 61) | |
| 116 | CLTA4_3xFl- | CLTA4mg ON- | G*sCAGAUGUAGUGUUUCCACAGUUUaAGAGCUAG(Fl)AAUAGCAAGUUuAA | 100 |
| | Loops_1x(2′OMe, | target | AUAAGGCUAGUCCGUUAUCAACUUG(Fl)AAAAGUGGCACCGAG(Fl)CGGUGC | |
| | 3′thioPACE) | | UUU*sU (SEQ ID NO: 62) | |
| 117 | CLTA4_3xFl- | CLTA4mg | G*sCAGAUGUAGUGUUUCCACAGUUUaAGAGCUAG(Fl)AAUAGCAAGUUuAA | 100 |
| | Loops_1x(2′OMe, | OFF5-target | AUAAGGCUAGUCCGUUAUCAACUUG(Fl)AAAAGUGGCACCGAG(Fl)CGGUGC | |
| | 3′thioPACE) | | UUU*sU (SEQ ID NO: 62) | |

3′Phosphorothioate-modified sgRNA

| | | | | |
|---|---|---|---|---|
| 118 | CLTA1_5′-2xP(S) sgRNA | CLTA1 ON1- | AsGsUCCUCAUCUCCCUCAAGCGUUUAAGAGCUAUGCUGGAAACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 63) | |
| 119 | CLTA1_5′-3xP(S) sgRNA | CLTA1 ON1- | AsGsUsCCUCAUCUCCCUCAAGCGUUUAAGAGCUAUGCUGGAAACAGCAUAG | 113 |
| | | target | CAAGUUUAAAUAAGGCUAGUCCGUUAUCAACUUGAAAAAGUGGCACCGAG | |
| | | | UCGGUGCUUUUUUU (SEQ ID NO: 64) | |
| 120 | CLTA1_5′-4xP(S) sgRNA | CLTA1 ON1- | AsGsUsCsCUCAUCUCCCUCAAGCGUUUAAGAGCUAUGCUGGAAACAGCAUA | 113 |
| | | target | GCAAGUUUAAAUAAGGCUAGUCCGUUAUCAACUUGAAAAAGUGGCACCGA | |
| | | | GUCGGUGCUUUUUUU (SEQ ID NO: 65) | |
| 121 | CLTA1_3′-4xP(S) sgRNA | CLTA1 ON1- | AGUCCUCAUCUCCCUCAAGCGUUUAAGAGCUAUGCUGGAAACAGCAUAGCA | 113 |
| | | target | AGUUUAAAUAAGGCUAGUCCGUUAUCAACUUGAAAAAGUGGCACCGAGUC | |
| | | | GGUGCUUUsUsUsUsU (SEQ ID NO: 66) | |

2′OMethyl-modified sgRNA

| | | | | |
|---|---|---|---|---|
| 122 | CLTA1_2′OMe + 20 sgRNA | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 67) | |
| 123 | CLTA1_2′OMe + 19 sgRNA | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 68) | |
| 124 | CLTA1_2′OMe + 19 sgRNA | CLTA1mg ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 68) | |
| 125 | CLTA1_2′OMe + 19 sgRNA | CLTA1mg ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 68) | |
| 126 | CLTA1_2′OMe + 19 sgRNA | CLTA1mg | AGUCCUCAUCUCCCUCAAGCGUUUAAGAGCUAUGCUGGUAACAGCAUAGCA | 113 |
| | | OFF1-target | AGUUUAAAUAAGGCUAGUCCGUUAUCAACUUGAAAAAGUGGCACCGAGUC | |
| | | | GGUGCUUUUUUU (SEQ ID NO: 68) | |
| 127 | CLTA1_2′OMe + 19 sgRNA | CLTA1mg | AGUCCUCAUCUCCCUCAAGCGUUUAAGAGCUAUGCUGGUAACAGCAUAGCA | 113 |
| | | OFF3-target | AGUUUAAAUAAGGCUAGUCCGUUAUCAACUUGAAAAAGUGGCACCGAGUC | |
| | | | GGUGCUUUUUUU (SEQ ID NO: 68) | |
| 128 | CLTA1_2′OMe + 18 sgRNA | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 69) | |
| 129 | CLTA1_2′OMe + 18 sgRNA | CLTA1mg ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 69) | |
| 130 | CLTA1_2′OMe + 18 sgRNA | CLTA1mg ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 69) | |
| 131 | CLTA1_2′OMe + 18 sgRNA | CLTA1mg | AGUCCUCAUCUCCCUCAAGCGUUUAAGAGCUAUGCUGGUAACAGCAUAGCA | 113 |
| | | OFF1-target | AGUUUAAAUAAGGCUAGUCCGUUAUCAACUUGAAAAAGUGGCACCGAGUC | |
| | | | GGUGCUUUUUUU (SEQ ID NO: 69) | |
| 132 | CLTA1_2′OMe + 18 sgRNA | CLTA1mg | AGUCCUCAUCUCCCUCAAGCGUUUAAGAGCUAUGCUGGUAACAGCAUAGCA | 113 |
| | | OFF3-target | AGUUUAAAUAAGGCUAGUCCGUUAUCAACUUGAAAAAGUGGCACCGAGUC | |
| | | | GGUGCUUUUUUU (SEQ ID NO: 69) | |
| 133 | CLTA1_2′OMe + 17 sgRNA | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 70) | |
| 134 | CLTA1_2′OMe + 17 sgRNA | CLTA1mg ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 70) | |
| 135 | CLTA1_2′OMe + 17 sgRNA | CLTA1mg ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 70) | |
| 136 | CLTA1_2′OMe + 17 sgRNA | CLTA1mg | AGUCCUCAUCUCCCUCAAGCGUUUAAGAGCUAUGCUGGUAACAGCAUAGCA | 113 |
| | | OFF1-target | AGUUUAAAUAAGGCUAGUCCGUUAUCAACUUGAAAAAGUGGCACCGAGUC | |
| | | | GGUGCUUUUUUU (SEQ ID NO: 70) | |
| 137 | CLTA1_2′OMe + 17 sgRNA | CLTA1mg | AGUCCUCAUCUCCCUCAAGCGUUUAAGAGCUAUGCUGGUAACAGCAUAGCA | 113 |
| | | OFF3-target | AGUUUAAAUAAGGCUAGUCCGUUAUCAACUUGAAAAAGUGGCACCGAGUC | |
| | | | GGUGCUUUUUUU (SEQ ID NO: 70) | |
| 138 | CLTA1_2′OMe 17, 18 | CLTA1 ON1- | AGUCCUCAUCUCCCUCAAGCGUUUAAGAGCUAUGCUGGUAACAGCAUAGCA | 113 |
| | sgRNA | target | AGUUUAAAUAAGGCUAGUCCGUUAUCAACUUGAAAAAGUGGCACCGAGUC | |
| | | | GGUGCUUUUUUU (SEQ ID NO: 71) | |
| 139 | CLTA1_2′OMe 17, 18 | CLTA1mg ON1- | AGUCCUCAUCUCCCUCAAGCGUUUAAGAGCUAUGCUGGUAACAGCAUAGCA | 113 |
| | sgRNA | target | AGUUUAAAUAAGGCUAGUCCGUUAUCAACUUGAAAAAGUGGCACCGAGUC | |
| | | | GGUGCUUUUUUU (SEQ ID NO: 71) | |
| 140 | CLTA1_2′OMe+ 17, 18 | CLTA1mg ON1- | AGUCCUCAUCUCCCUCAAGCGUUUAAGAGCUAUGCUGGUAACAGCAUAGCA | 113 |
| | sgRNA | target | AGUUUAAAUAAGGCUAGUCCGUUAUCAACUUGAAAAAGUGGCACCGAGUC | |
| | | | GGUGCUUUUUUU (SEQ ID NO: 71) | |
| 141 | CLTA1_2′OMe 17, 18 | CLTA1mg | AGUCCUCAUCUCCCUCAAGCGUUUAAGAGCUAUGCUGGUAACAGCAUAGCA | 113 |
| | sgRNA | OFF1-target | AGUUUAAAUAAGGCUAGUCCGUUAUCAACUUGAAAAAGUGGCACCGAGUC | |
| | | | GGUGCUUUUUUU (SEQ ID NO: 71) | |
| 142 | CLTA1_2′OMe 17, 18 | CLTA1mg | AGUCCUCAUCUCCCUCAAGCGUUUAAGAGCUAUGCUGGUAACAGCAUAGCA | 113 |
| | sgRNA | OFF3-target | AGUUUAAAUAAGGCUAGUCCGUUAUCAACUUGAAAAAGUGGCACCGAGUC | |
| | | | GGUGCUUUUUUU (SEQ ID NO: 71) | |
| 143 | CLTA1_5′, 3′-3x(2′OMe) | CLTA1 ON1- | AGUCCUCAUCUCCCUCAAGCGUUUAAGAGCUAUGCUGGUAACAGCAUAGCA | 113 |
| | sgRNA | target | AGUUUAAAUAAGGCUAGUCCGUUAUCAACUUGAAAAAGUGGCACCGAGUC | |
| | | | GGUGCUUUUUUU (SEQ ID NO: 72) | |
| 144 | CLTA4_5′, 3′-3x(2′OMe) | CLTA4mg ON- | GCAGAUGUAGUGUUUCCACAGUUUAAGAGCUAUGCUGGAAACAGCAUAGC | 113 |
| | sgRNA | target | AAGUUUAAAUAAGGCUAGUCCGUUAUCAACUUGAAAAAGUGGCACCGAGU | |
| | | | CGGUGCUUUUUUU (SEQ ID NO: 73) | |
| 145 | CLTA4_5′, 3′-3x(2′OMe) | CLTA4mg | GCAGAUGUAGUGUUUCCACAGUUUAAGAGCUAUGCUGGAAACAGCAUAGC | 113 |
| | sgRNA | OFF5-target | AAGUUUAAAUAAGGCUAGUCCGUUAUCAACUUGAAAAAGUGGCACCGAGU | |
| | | | CGGUGCUUUUUUU (SEQ ID NO: 73) | |
| 146 | CLTA1_5′-20x(2′OMe) | CLTA1 ON1- | AGUCCUCAUCUCCCUCAAGCGUUUAAGAGCUAUGCUGGUAACAGCAUAGCA | 113 |
| | sgRNA | target | AGUUUAAAUAAGGCUAGUCCGUUAUCAACUUGAAAAAGUGGCACCGAGUC | |
| | | | GGUGCUUUUUUU (SEQ ID NO: 74) | |
| 147 | CLTA1_5′-20x(2′OMe) | CLTA1mg ON1- | AGUCCUCAUCUCCCUCAAGCGUUUAAGAGCUAUGCUGGUAACAGCAUAGCA | 113 |
| | sgRNA | target | AGUUUAAAUAAGGCUAGUCCGUUAUCAACUUGAAAAAGUGGCACCGAGUC | |
| | | | GGUGCUUUUUUU (SEQ ID NO: 74) | |
| 148 | CLTA1_5′-20x(2′OMe) | CLTA1mg ON1- | AGUCCUCAUCUCCCUCAAGCGUUUAAGAGCUAUGCUGGUAACAGCAUAGCA | 113 |
| | sgRNA | target | AGUUUAAAUAAGGCUAGUCCGUUAUCAACUUGAAAAAGUGGCACCGAGUC | |
| | | | GGUGCUUUUUUU (SEQ ID NO: 74) | |
| 149 | CLTA1_5′-20x(2′OMe) | CLTA1mg | AGUCCUCAUCUCCCUCAAGCGUUUAAGAGCUAUGCUGGUAACAGCAUAGCA | 113 |
| | sgRNA | OFF1-target | AGUUUAAAUAAGGCUAGUCCGUUAUCAACUUGAAAAAGUGGCACCGAGUC | |
| | | | GGUGCUUUUUUU (SEQ ID NO: 74) | |
| 150 | CLTA1_5′-20x(2′OMe) | CLTA1mg | AGUCCUCAUCUCCCUCAAGCGUUUAAGAGCUAUGCUGGUAACAGCAUAGCA | 113 |
| | sgRNA | OFF3-target | AGUUUAAAUAAGGCUAGUCCGUUAUCAACUUGAAAAAGUGGCACCGAGUC | |
| | | | GGUGCUUUUUUU (SEQ ID NO: 74) | |
| 151 | CLTA1_5′-26x(2′OMe) | CLTA1 ON1- | AGUCCUCAUCUCCCUCAAGCGUUUAAGAGCUAUGCUGGUAACAGCAUAGC | 113 |
| | sgRNA | target | AAGUUUAAAUAAGGCUAGUCCGUUAUCAACUUGAAAAAGUGGCACCGAGU | |
| | | | CGGUGCUUUUUUU (SEQ ID NO: 75) | |
| 152 | CLTA1_5′-37x(2′OMe) | CLTA1 ON1- | AGUCCUCAUCUCCCUCAAGCGUUUAAGAGCUAUGCUGGUAACAGCAUAGC | 113 |
| | sgRNA | target | AAGUUUAAAUAAGGCUAGUCCGUUAUCAACUUGAAAAAGUGGCACCGAGU | |
| | | | CGGUGCUUUUUUU (SEQ ID NO: 76) | |
| 153 | CLTA1_41x(2′OMeC/U)_QB3 | CLTA1mg ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 77) | |
| 154 | CLTA1_47x(2′OMeC/U)_QB3 | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 79) | |
| 155 | CLTA1_47x(2′OMeC/U)_QB3 | CLTA1mg ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 79) | |
| 156 | CLTA1_47x(2′OMeC/U)_QB3 | CLTA1mg ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 79) | |
| 157 | CLTA1_47x(2′OMeC/U)_QB3 | CLTA1mg | AGUCCUCAUCUCCCUCAAGCGUUUAAGAGCUAUGCUGGUAACAGCAUAGCA | 113 |
| | | OFF1-target | AGUUUAAAUAAGGCUAGUCCGUUAUCAACUUGAAAAAGUGGCACCGAGUC | |
| | | | GGUGCUUUUUUU (SEQ ID NO: 79) | |
| 158 | CLTA1_47x(2′OMeC/U)_QB3 | CLTA1mg | AGUCCUCAUCUCCCUCAAGCGUUUAAGAGCUAUGCUGGUAACAGCAUAGCA | 113 |
| | | OFF3-target | AGUUUAAAUAAGGCUAGUCCGUUAUCAACUUGAAAAAGUGGCACCGAGUC | |
| | | | GGUGCUUUUUUU (SEQ ID NO: 79) | |
| 159 | CLTA1_47x(2′OMeG/A)_QB3 | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 80) | |
| 160 | CLTA1_47x(2′OMeG/A)_QB3 | CLTA1mg ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 80) | |
| 161 | CLTA1_47x(2′OMeG/A)_QB3 | CLTA1mg ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCACCGAGUC | |
| | | | GGUGCUUUUUUU (SEQ ID NO: 80) | |
| 162 | CLTA1_47x(2′OMeG/A)_QB3 | CLTA1mg | AGUCCUCAUCUCCCUCAAGCGUUUAAGAGCUAUGCUGGUAACAGCAUAGCA | 113 |
| | | OFF1-target | AGUUUAAAUAAGGCUAGUCCGUUAUCAACUUGAAAAAGUGGCACCGAGUC | |
| | | | GGUGCUUUUUUU (SEQ ID NO: 80) | |
| 163 | CLTA1_47x(2′OMeG/A)_QB3 | CLTA1mg | AGUCCUCAUCUCCCUCAAGCGUUUAAGAGCUAUGCUGGUAACAGCAUAGCA | 113 |
| | | OFF3-target | AGUUUAAAUAAGGCUAGUCCGUUAUCAACUUGAAAAAGUGGCACCGAGUC | |
| | | | GGUGCUUUUUUU (SEQ ID NO: 80) | |
| 164 | CLTA1_43x(2′OMeG/A)_Bos | CLTA1 ON1- | AGUCCUCAUCUCCCUCAAGCGUUUUAGAGCUAGUAAUAGCAAGUUAAAAUA | 100 |
| | | target | AGGCUAGUCCGUUAUCAACUUGAAAAAGUGGCACCGAGUCGGUGCUUUU | |
| | | | (SEQ ID NO: 81) | |
| 165 | CLTA1_43x(2′OMeG/A)_Bos | CLTA1mg ON1- | AGUCCUCAUCUCCCUCAAGCGUUUUAGAGCUAGUAAUAGCAAGUUAAAAUA | 100 |
| | | target | AGGCUAGUCCGUUAUCAACUUGAAAAAGUGGCACCGAGUCGGUGCUUUU | |
| | | | (SEQ ID NO: 81) | |
| 166 | CLTA1_43x(2′OMeG/A)_Bos | CLTA1mg ON1- | AGUCCUCAUCUCCCUCAAGCGUUUUAGAGCUAGUAAUAGCAAGUUAAAAUA | 100 |
| | | target | AGGCUAGUCCGUUAUCAACUUGAAAAAGUGGCACCGAGUCGGUGCUUUU | |
| | | | (SEQ ID NO: 81) | |
| 167 | CLTA1_43x(2′OMeG/A)_Bos | CLTA1mg | AGUCCUCAUCUCCCUCAAGCGUUUUAGAGCUAGUAAUAGCAAGUUAAAAUA | 100 |
| | | OFF1-target | AGGCUAGUCCGUUAUCAACUUGAAAAAGUGGCACCGAGUCGGUGCUUUU | |
| | | | (SEQ ID NO: 81) | |
| 168 | CLTA1_43x(2′OMeG/A)_Bos | CLTA1mg | AGUCCUCAUCUCCCUCAAGCGUUUUAGAGCUAGUAAUAGCAAGUUAAAAUA | 100 |
| | | OFF3-target | AGGCUAGUCCGUUAUCAACUUGAAAAAGUGGCACCGAGUCGGUGCUUUU | |
| | | | (SEQ ID NO: 81) | |
| 169 | CLTA4 sgRNA_5′, 3′- | CLTA4 ON- | GCAGAUGUAGUGUUUCCACAGUUUAAGAGCUAUGCUGGAAACAGCAUAGC | 113 |
| | 3x(2′OMe) | target | AAGUUUAAAUAAGGCUAGUCCGUUAUCAACUUGAAAAAGUGGCACCGAGU | |
| | | | CGGUGCUUUUUUU (SEQ ID NO: 82) | |
| 170 | CLTA4 sgRNA_5′, 3′- | CLTA4 ON- | GCAGAUGUAGUGUUUCCACAGUUUAAGAGCUAUGCUGGAAACAGCAUAGC | 113 |
| | 3x(2′OMe) | target | AAGUUUAAAUAAGGCUAGUCCGUUAUCAACUUGAAAAAGUGGCACCGAGU | |
| | | | CGGUGCUUUUUUU (SEQ ID NO: 82) | |
| 171 | CLTA4_47x(2′OMeC/U)_QB3 | CLTA4 ON- | GCAGAUGUAGUGUUUCCACAGUUUAAGAGCUAUGCUGGUAACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 83) | |
| 172 | CLTA4_47x(2′OMeC/U)_QB3 | CLTA4 ON- | GCAGAUGUAGUGUUUCCACAGUUUAAGAGCUAUGCUGGUAACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 83) | |
| 173 | CLTA4_47x(2′OMeC/U)_QB3 | CLTA4 ON- | GCAGAUGUAGUGUUUCCACAGUUUAAGAGCUAUGCUGGUAACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 83) | |
| 174 | CLTA4_49x(2′OMeG/A)_Bos | CLTA4 ON- | GCAGAUGUAGUGUUUCCACAGUUUUAGAGCUAGUAAUAGCAAGUUAAAAU | 100 |
| | | target | AAGGCUAGUCCGUUAUCAACUUGAAAAAGUGGCACCGAGUCGGUGCUUUU | |
| | | | (SEQ ID NO: 84) | |
| 175 | CLTA4_49x(2′OMeG/A)_Bos | CLTA4 ON- | GCAGAUGUAGUGUUUCCACAGUUUUAGAGCUAGUAAUAGCAAGUUAAAAU | 100 |
| | | target | AAGGCUAGUCCGUUAUCAACUUGAAAAAGUGGCACCGAGUCGGUGCUUUU | |
| | | | (SEQ ID NO: 84) | |
| 176 | CLTA4_49x(2′OMeG/A)_Bos | CLTA4 ON- | GCAGAUGUAGUGUUUCCACAGUUUUAGAGCUAGUAAUAGCAAGUUAAAAU | 100 |
| | | target | AAGGCUAGUCCGUUAUCAACUUGAAAAAGUGGCACCGAGUCGGUGCUUUU | |
| | | | (SEQ ID NO: 84) | |
| 177 | CLTA4_39x(2′OMeC/U)_Bos | CLTA4 ON- | GCAGAUGUAGUGUUUCCACAGUUUUAGAGCUAGUAAUAGCAAGUUAAAAU | 100 |
| | | target | AAGGCUAGUCCGUUAUCAACUUGAAAAAGUGGCACCGAGUCGGUGCUUUU | |
| | | | (SEQ ID NO: 85) | |
| 178 | CLTA4_39x(2′OMeC/U)_Bos | CLTA4 ON- | GCAGAUGUAGUGUUUCCACAGUUUUAGAGCUAGUAAUAGCAAGUUAAAAU | 100 |
| | | target | AAGGCUAGUCCGUUAUCAACUUGAAAAAGUGGCACCGAGUCGGUGCUUUU | |
| | | | (SEQ ID NO: 85) | |
| 179 | CLTA4_39x(2′OMeC/U)_Bos | CLTA4 ON- | GCAGAUGUAGUGUUUCCACAGUUUUAGAGCUAGUAAUAGCAAGUUAAAAU | 100 |
| | | target | AAGGCUAGUCCGUUAUCAACUUGAAAAAGUGGCACCGAGUCGGUGCUUUU | |
| | | | (SEQ ID NO: 85) | |

2′Deoxy-modified sgRNA

| | | | | |
|---|---|---|---|---|
| 180 | CLTA1_5′-20x(2′deoxy) | CLTA1 ON1- | AGTCCTCATCTCCCTCAAGCGUUUAAGAGCUAUGCUGGUAACAGCAUAGCAA | 113 |
| | | target | GUUUAAAUAAGGCUAGUCCGUUAUCAACUUGAAAAAGUGGCACCGAGUCG | |
| | | | GUGCUUUUUUU (SEQ ID NO: 86) | |
| 181 | CLTA1_5′-26x(2′deoxy) | CLTA1 ON1- | AGTCCTCATCTCCCTCAAGCGTTTAAGAGCUAUGCUGGUAACAGCAUAGCAAG | 113 |
| | | target | UUUAAAUAAGGCUAGUCCGUUAUCAACUUGAAAAAGUGGCACCGAGUCGG | |
| | | | UGCUUUUUUU (SEQ ID NO: 87) | |
| 182 | CLTA1_5′-37x(2′deoxy) | CLTA1 ON1- | AGTCCTCATCTCCCTCAAGCGTTTAAGAGCTATGCTGGUAACAGCAUAGCAAG | 113 |
| | | target | UUUAAAUAAGGCUAGUCCGUUAUCAACUUGAAAAAGUGGCACCGAGUCGG | |
| | | | UGCUUUUUUU (SEQ ID NO: 88) | |

2′Deoxy, 3′PACE-modified sgRNA

| | | | | |
|---|---|---|---|---|
| 183 | CLTA4_2′deoxy3′PACE + | CLTA4mg ON- | GCAGAUGUAGUGUUU*CCACAGUUUAAGAGCUAUGCUGGUAACAGCAUAG | 113 |
| | 15 | target | CAAGUUUAAAUAAGGCUAGUCCGUUAUCAACUUGAAAAAGUGGCACCGAG | |
| | | | UCGGUGCUUUUUUU (SEQ ID NO: 89) | |
| 184 | CLTA4_2′deoxy3′PACE + | CLTA4mg | GCAGAUGUAGUGUUU*CCACAGUUUAAGAGCUAUGCUGGUAACAGCAUAG | 113 |
| | 15 | OFF5-target | CAAGUUUAAAUAAGGCUAGUCCGUUAUCAACUUGAAAAAGUGGCACCGAG | |
| | | | UCGGUGCUUUUUUU (SEQ ID NO: 89) | |

2′OMethyl, 3′PACE-modified sgRNA

| | | | | |
|---|---|---|---|---|
| 185 | 5′- | CLTA1mg ON1- | A*GUCCUCAUCUCCCUCAAGCGUUUAAGAGCUAUGCUGGUAACAGCAUAGC | 113 |
| | 1x(2′OMe, 3′PACE)_CLTA1 | target | AAGUUUAAAUAAGGCUAGUCCGUUAUCAACUUGAAAAAGUGGCACCGAGU | |
| | sgRNA | | CGGUGCUUUUUUU (SEQ ID NO: 90) | |
| 186 | 5′- | CLTA1mg ON1- | A*GUCCUCAUCUCCCUCAAGCGUUUAAGAGCUAUGCUGGUAACAGCAUAGC | 113 |
| | 1x(2′OMe, 3′PACE)_CLTA1 | target | AAGUUUAAAUAAGGCUAGUCCGUUAUCAACUUGAAAAAGUGGCACCGAGU | |
| | sgRNA | | CGGUGCUUUUUUU (SEQ ID NO: 90) | |
| 187 | 5′- | CLTA1 ON1- | A*G*UCCUCAUCUCCCUCAAGCGUUUAAGAGCUAUGCUGGUAACAGCAUAG | 113 |
| | 2x(2′OMe, 3′PACE)_CLTA1 | target | CAAGUUUAAAUAAGGCUAGUCCGUUAUCAACUUGAAAAAGUGGCACCGAG | |
| | sgRNA | | UCGGUGCUUUUUUU (SEQ ID NO: 91) | |
| 188 | 5′- | CLTA1 ON1- | A*G*UCCUCAUCUCCCUCAAGCGUUUAAGAGCUAUGCUGGUAACAGCAUAG | 113 |
| | 2x(2′OMe, 3′PACE)_CLTA1 | target | CAAGUUUAAAUAAGGCUAGUCCGUUAUCAACUUGAAAAAGUGGCACCGAG | |
| | sgRNA | | UCGGUGCUUUUUUU (SEQ ID NO: 91) | |
| 189 | 5′- | CLTA1mg ON1- | A*G*UCCUCAUCUCCCUCAAGCGUUUAAGAGCUAUGCUGGUAACAGCAUAG | 113 |
| | 2x(2′OMe, 3′PACE)_CLTA1 | target | CAAGUUUAAAUAAGGCUAGUCCGUUAUCAACUUGAAAAAGUGGCACCGAG | |
| | sgRNA | | UCGGUGCUUUUUUU (SEQ ID NO: 91) | |
| 190 | 5′- | CLTA1mg ON1- | A*G*UCCUCAUCUCCCUCAAGCGUUUAAGAGCUAUGCUGGUAACAGCAUAG | 113 |
| | 2x(2′OMe, 3′PACE)_CLTA1 | target | CAAGUUUAAAUAAGGCUAGUCCGUUAUCAACUUGAAAAAGUGGCACCGAG | |
| | sgRNA | | UCGGUGCUUUUUUU (SEQ ID NO: 91) | |
| 191 | 5′- | CLTA1mg ON1- | G*G*A*GUCCUCAUCUCCCUCAAGCGUUUAAGAGCUAUGCUGGUAACAGCA | 113 |
| | 3x(2′OMe, 3′PACE)_CLTA1 | target | UAGCAAGUUUAAAUAAGGCUAGUCCGUUAUCAACUUGAAAAAGUGGCACC | |
| | sgRNA | | GAGUCGGUGCUUUUUUU (SEQ ID NO: 92) | |
| 192 | 5′- | CLTA1mg ON1- | G*G*A*GUCCUCAUCUCCCUCAAGCGUUUAAGAGCUAUGCUGGUAACAGCA | 113 |
| | 3x(2′OMe, 3′PACE)_CLTA1 | target | UAGCAAGUUUAAAUAAGGCUAGUCCGUUAUCAACUUGAAAAAGUGGCACC | |
| | sgRNA | | GAGUCGGUGCUUUUUUU (SEQ ID NO: 92) | |
| 193 | 5′- | CLTA1 ON1- | A*G*U*C*CUCAUCUCCCUCAAGCGUUUAAGAGCUAUGCUGGUAACAGCAU | 113 |
| | 4x(2′OMe, 3′PACE)_CLTA1 | target | AGCAAGUUUAAAUAAGGCUAGUCCGUUAUCAACUUGAAAAAGUGGCACCG | |
| | sgRNA | | AGUCGGUGCUUUUUUU (SEQ ID NO: 93) | |
| 194 | 5′- | CLTA1mg ON1- | A*G*U*C*CUCAUCUCCCUCAAGCGUUUAAGAGCUAUGCUGGUAACAGCAU | 113 |
| | 4x(2′OMe, 3′PACE)_CLTA1 | target | AGCAAGUUUAAAUAAGGCUAGUCCGUUAUCAACUUGAAAAAGUGGCACCG | |
| | sgRNA | | AGUCGGUGCUUUUUUU (SEQ ID NO: 93) | |
| 195 | 5′- | CLTA1mg ON1- | A*G*U*C*CUCAUCUCCCUCAAGCGUUUAAGAGCUAUGCUGGUAACAGCAU | 113 |
| | 4x(2′OMe, 3′PACE)_CLTA1 | target | AGCAAGUUUAAAUAAGGCUAGUCCGUUAUCAACUUGAAAAAGUGGCACCG | |
| | sgRNA | | AGUCGGUGCUUUUUUU (SEQ ID NO: 93) | |
| 196 | 5′- | CLTA1mg ON1- | G*G*A*G*U*CCUCAUCUCCCUCAAGCGUUUAAGAGCUAUGCUGGUAACAG | 113 |
| | 5x(2′OMe, 3′PACE)_CLTA1 | target | CAUAGCAAGUUUAAAUAAGGCUAGUCCGUUAUCAACUUGAAAAAGUGGCA | |
| | sgRNA | | CCGAGUCGGUGCUUUUUUU (SEQ ID NO: 94) | |
| 197 | 5′- | CLTA1mg ON1- | G*G*A*G*U*CCUCAUCUCCCUCAAGCGUUUAAGAGCUAUGCUGGUAACAG | 113 |
| | 5x(2′OMe, 3′PACE)_CLTA1 | target | CAUAGCAAGUUUAAAUAAGGCUAGUCCGUUAUCAACUUGAAAAAGUGGCA | |
| | sgRNA | | CCGAGUCGGUGCUUUUUUU (SEQ ID NO: 94) | |
| 198 | CLTA1_3′- | CLTA1 ON1- | AGUCCUCAUCUCCCUCAAGCGUUUAAGAGCUAUGCUGGUAACAGCAUAGCA | 113 |
| | 4x(2′OMe, 3′PACE) sgRNA | target | AGUUUAAAUAAGGCUAGUCCGUUAUCAACUUGAAAAAGUGGCACCGAGUC | |
| | | | GGUGCUUU*U*U*U*U (SEQ ID NO: 95) | |
| 199 | CLTA1_3′- | CLTA1 ON1- | AGUCCUCAUCUCCCUCAAGCGUUUAAGAGCUAUGCUGGUAACAGCAUAGCA | 113 |
| | 4x(2′OMe, 3′PACE) sgRNA | target | AGUUUAAAUAAGGCUAGUCCGUUAUCAACUUGAAAAAGUGGCACCGAGUC | |
| | | | GGUGCUUU*U*U*U*U (SEQ ID NO: 95) | |
| 200 | CLTA1_3′- | CLTA1mg ON1- | AGUCCUCAUCUCCCUCAAGCGUUUAAGAGCUAUGCUGGUAACAGCAUAGCA | 113 |
| | 4x(2′OMe, 3′PACE) sgRNA | target | AGUUUAAAUAAGGCUAGUCCGUUAUCAACUUGAAAAAGUGGCACCGAGUC | |
| | | | GGUGCUUU*U*U*U*U (SEQ ID NO: 95) | |
| 201 | CLTA1_3′- | CLTA1mg ON1- | AGUCCUCAUCUCCCUCAAGCGUUUAAGAGCUAUGCUGGUAACAGCAUAGCA | 113 |
| | 4x(2′OMe, 3′PACE) sgRNA | target | AGUUUAAAUAAGGCUAGUCCGUUAUCAACUUGAAAAAGUGGCACCGAGUC | |
| | | | GGUGCUUU*U*U*U*U (SEQ ID NO: 95) | |
| 202 | CLTA1_3′- | CLTA1 ON1- | AGUCCUCAUCUCCCUCAAGCGUUUAAGAGCUAUGCUGGUAACAGCAUAGCA | 113 |
| | 5x(2′OMe, 3′PACE) sgRNA | target | AGUUUAAAUAAGGCUAGUCCGUUAUCAACUUGAAAAAGUGGCACCGAGUC | |
| | | | GGUGCUU*U*U*U*U*U (SEQ ID NO: 96) | |
| 203 | CLTA1_3′- | CLTA1 ON1- | AGUCCUCAUCUCCCUCAAGCGUUUAAGAGCUAUGCUGGUAACAGCAUAGCA | 113 |
| | 5x(2′OMe, 3′PACE) sgRNA | target | AGUUUAAAUAAGGCUAGUCCGUUAUCAACUUGAAAAAGUGGCACCGAGUC | |
| | | | GGUGCUU*U*U*U*U*U (SEQ ID NO: 96) | |
| 204 | CLTA1_3′- | CLTA1mg ON1- | AGUCCUCAUCUCCCUCAAGCGUUUAAGAGCUAUGCUGGUAACAGCAUAGCA | 113 |
| | 5x(2′OMe, 3′PACE) sgRNA | target | AGUUUAAAUAAGGCUAGUCCGUUAUCAACUUGAAAAAGUGGCACCGAGUC | |
| | | | GGUGCUU*U*U*U*U*U (SEQ ID NO: 96) | |
| 205 | CLTA1_3′- | CLTA1mg ON1- | AGUCCUCAUCUCCCUCAAGCGUUUAAGAGCUAUGCUGGUAACAGCAUAGCA | 113 |
| | 5x(2′OMe, 3′PACE) sgRNA | target | AGUUUAAAUAAGGCUAGUCCGUUAUCAACUUGAAAAAGUGGCACCGAGUC | |
| | | | GGUGCUU*U*U*U*U*U (SEQ ID NO: 96) | |
| 206 | 5′- | CLTA1 ON1- | Cₒ*A*G*UCCUCAUCUCCCUCAAGCGUUUAAGAGCUAUGCUGGUAACAGCAU | 114 |
| | 3x(2′OMe, 3′PACE)_plus1 | target | AGCAAGUUUAAAUAAGGCUAGUCCGUUAUCAACUUGAAAAAGUGGCACCG | |
| | overhg_CLTA1 | | AGUCGGUGCUUUUUUU (SEQ ID NO: 97) | |
| 207 | 5′- | CLTA1mg ON1- | Cₒ*A*G*UCCUCAUCUCCCUCAAGCGUUUAAGAGCUAUGCUGGUAACAGCAU | 114 |
| | 3x(2′OMe, 3′PACE)_plus1 | target | AGCAAGUUUAAAUAAGGCUAGUCCGUUAUCAACUUGAAAAAGUGGCACCG | |
| | overhg_CLTA1 | | AGUCGGUGCUUUUUUU (SEQ ID NO: 97) | |
| 208 | 5′- | CLTA1mg ON1- | Cₒ*A*G*UCCUCAUCUCCCUCAAGCGUUUAAGAGCUAUGCUGGUAACAGCAU | 114 |
| | 3x(2′OMe, 3′PACE)_plus1 | target | AGCAAGUUUAAAUAAGGCUAGUCCGUUAUCAACUUGAAAAAGUGGCACCG | |
| | overhg_CLTA1 | | AGUCGGUGCUUUUUUU (SEQ ID NO: 97) | |
| 209 | 5′- | CLTA1 ON1- | Gₒ*A*G*UCCUCAUCUCCCUCAAGCGUUUAAGAGCUAUGCUGGUAACAGCAU | 114 |
| | 3x(2′OMe, 3′PACE)_plus1 | target | AGCAAGUUUAAAUAAGGCUAGUCCGUUAUCAACUUGAAAAAGUGGCACCG | |
| | NC_overhg_CLTA1 | | AGUCGGUGCUUUUUUU (SEQ ID NO: 98 | |
| 210 | 5′- | CLTA1mg ON1- | Gₒ*A*G*UCCUCAUCUCCCUCAAGCGUUUAAGAGCUAUGCUGGUAACAGCAU | 114 |
| | 3x(2′OMe, 3′PACE)_plus1 | target | AGCAAGUUUAAAUAAGGCUAGUCCGUUAUCAACUUGAAAAAGUGGCACCG | |
| | NC_overhg_CLTA1 | | AGUCGGUGCUUUUUUU (SEQ ID NO: (SEQ ID NO: 98) | |
| 211 | 5′- | CLTA1mg ON1- | Gₒ*A*G*UCCUCAUCUCCCUCAAGCGUUUAAGAGCUAUGCUGGUAACAGCAU | 114 |
| | 3x(2′OMe, 3′PACE)_plus1 | target | AGCAAGUUUAAAUAAGGCUAGUCCGUUAUCAACUUGAAAAAGUGGCACCG | |
| | NC_overhg_CLTA1 | | AGUCGGUGCUUUUUUU (SEQ ID NO: 98) | |
| 212 | 5′- | CLTA1 ON1- | Uₒ*Cₒ*A*G*U*CCUCAUCUCCCUCAAGCGUUUAAGAGCUAUGCUGGUAACA | 115 |
| | 5x(2′OMe, 3′PACE)_plus2 | target | GCAUAGCAAGUUUAAAUAAGGCUAGUCCGUUAUCAACUUGAAAAAGUGGC | |
| | overhg_CLTA1 | | ACCGAGUCGGUGCUUUUUUU (SEQ ID NO: 99) | |
| 213 | 5′- | CLTA1mg ON1- | Uₒ*Cₒ*A*G*U*CCUCAUCUCCCUCAAGCGUUUAAGAGCUAUGCUGGUAACA | 115 |
| | 5x(2′OMe, 3′PACE)_plus2 | target | GCAUAGCAAGUUUAAAUAAGGCUAGUCCGUUAUCAACUUGAAAAAGUGGC | |
| | overhg_CLTA1 | | ACCGAGUCGGUGCUUUUUUU (SEQ ID NO: 99) | |
| 214 | 5′- | CLTA1mg ON1- | Uₒ*Cₒ*A*G*U*CCUCAUCUCCCUCAAGCGUUUAAGAGCUAUGCUGGUAACA | 115 |
| | 5x(2′OMe, 3′PACE)_plus2 | target | GCAUAGCAAGUUUAAAUAAGGCUAGUCCGUUAUCAACUUGAAAAAGUGGC | |
| | overhg_CLTA1 | | ACCGAGUCGGUGCUUUUUUU (SEQ ID NO: 99) | |
| 215 | 5′- | CLTA1 ON1- | Aₒ*Gₒ*A*G*U*CCUCAUCUCCCUCAAGCGUUUAAGAGCUAUGCUGGUAACA | 115 |
| | 5x(2′OMe, 3′PACE)_plus2 | target | GCAUAGCAAGUUUAAAUAAGGCUAGUCCGUUAUCAACUUGAAAAAGUGGC | |
| | NC_overhg_CLTA1 | | ACCGAGUCGGUGCUUUUUUU (SEQ ID NO: 100) | |
| 216 | 5′- | CLTA1mg ON1- | Aₒ*Gₒ*A*G*U*CCUCAUCUCCCUCAAGCGUUUAAGAGCUAUGCUGGUAACA | 115 |
| | 5x(2′OMe, 3′PACE)_plus2 | target | GCAUAGCAAGUUUAAAUAAGGCUAGUCCGUUAUCAACUUGAAAAAGUGGC | |
| | NC_overhg_CLTA1 | | ACCGAGUCGGUGCUUUUUUU (SEQ ID NO: 100) | |
| 217 | 5′- | CLTA1mg ON1- | Aₒ*Gₒ*A*G*U*CCUCAUCUCCCUCAAGCGUUUAAGAGCUAUGCUGGUAACA | 115 |
| | 5x(2′OMe, 3′PACE)_plus2 | target | GCAUAGCAAGUUUAAAUAAGGCUAGUCCGUUAUCAACUUGAAAAAGUGGC | |
| | NC_overhg_CLTA1 | | ACCGAGUCGGUGCUUUUUUU (SEQ ID NO: 100) | |
| 218 | 5′- | CLTA1 ON1- | Cₒ*Uₒ*Cₒ*A*G*U*C*CUCAUCUCCCUCAAGCGUUUAAGAGCUAUGCUGGUA | 116 |
| | 7x(2′OMe, 3′PACE)_plus3 | target | ACAGCAUAGCAAGUUUAAAUAAGGCUAGUCCGUUAUCAACUUGAAAAAGU | |
| | overhg_CLTA1_3′- | | GGCACCGAGUCGGUGCUUU*U*U*U*U (SEQ ID NO: 101) | |
| | 4x(2′OMe, 3′PACE) | | | |
| 219 | 5′- | CLTA1mg ON1- | Cₒ*Uₒ*Cₒ*A*G*U*C*CUCAUCUCCCUCAAGCGUUUAAGAGCUAUGCUGGUA | 116 |
| | 7x(2′OMe, 3′PACE)_plus3 | target | ACAGCAUAGCAAGUUUAAAUAAGGCUAGUCCGUUAUCAACUUGAAAAAGU | |
| | overhg_CLTA1_3′- | | GGCACCGAGUCGGUGCUUU*U*U*U*U (SEQ ID NO: 101) | |
| | 4x(2′OMe, 3′PACE) | | | |
| 220 | 5′- | CLTA1mg ON1- | Cₒ*Uₒ*Cₒ*A*G*U*C*CUCAUCUCCCUCAAGCGUUUAAGAGCUAUGCUGGUA | 116 |
| | 7x(2′OMe, 3′PACE)_plus3 | target | ACAGCAUAGCAAGUUUAAAUAAGGCUAGUCCGUUAUCAACUUGAAAAAGU | |
| | overhg_CLTA1_3′- | | GGCACCGAGUCGGUGCUUU*U*U*U*U (SEQ ID NO: 101) | |
| | 4x(2′OMe, 3′PACE) | | | |
| 221 | 5′- | CLTA1 ON1- | Cₒ*Uₒ*Cₒ*A*G*U*C*CUCAUCUCCCUCAAGCGUUUAAGAGCUAUGCUGGUA | 116 |
| | 7x(2′OMe, 3′PACE)_plus3 | target | ACAGCAUAGCAAGUUUAAAUAAGGCUAGUCCGUUAUCAACUUGAAAAAGU | |
| | NC_overhg_CLTA1_3′- | | GGCACCGAGUCGGUGCUUU*U*U*U*U (SEQ ID NO: 101) | |
| | 4x(2′OMe, 3′PACE) | | | |
| 222 | 5′- | CLTA1mg ON1- | Cₒ*Uₒ*Cₒ*A*G*U*C*CUCAUCUCCCUCAAGCGUUUAAGAGCUAUGCUGGUA | 116 |
| | 7x(2′OMe, 3′PACE)_plus3 | target | ACAGCAUAGCAAGUUUAAAUAAGGCUAGUCCGUUAUCAACUUGAAAAAGU | |
| | NC_overhg_CLTA1_3′- | | GGCACCGAGUCGGUGCUUU*U*U*U*U (SEQ ID NO: 101) | |
| | 4x(2′OMe, 3′PACE) | | | |
| 223 | 5′- | CLTA1mg ON1- | Cₒ*Uₒ*Cₒ*A*G*U*C*CUCAUCUCCCUCAAGCGUUUAAGAGCUAUGCUGGUA | 116 |
| | 7x(2′OMe, 3′PACE)_plus3 | target | ACAGCAUAGCAAGUUUAAAUAAGGCUAGUCCGUUAUCAACUUGAAAAAGU | |
| | NC_overhg_CLTA1_3′- | | GGCACCGAGUCGGUGCUUU*U*U*U*U (SEQ ID NO: 101) | |
| | 4x(2′OMe, 3′PACE) | | | |
| 224 | CLTA1_2′OMe, 3′PACE + | CLTA1 ON1- | AGUCCUCAUCUCCCUCAAGC*GUUUAAGAGCUAUGCUGGUAACAGCAUAGC | 113 |
| | 20 sgRNA | target | AAGUUUAAAUAAGGCUAGUCCGUUAUCAACUUGAAAAAGUGGCACCGAGU | |
| | | | CGGUGCUUUUUUU (SEQ ID NO: 102 | |
| 225 | CLTA1_2′OMe, 3′PACE + | CLTA1mg ON1- | AGUCCUCAUCUCCCUCAAGC*GUUUAAGAGCUAUGCUGGUAACAGCAUAGC | 113 |
| | 20 sgRNA | target | AAGUUUAAAUAAGGCUAGUCCGUUAUCAACUUGAAAAAGUGGCACCGAGU | |
| | | | CGGUGCUUUUUUU (SEQ ID NO: 102) | |
| 226 | CLTA1_2′OMe, 3′PACE + | CLTA1mg ON1- | AGUCCUCAUCUCCCUCAAGC*GUUUAAGAGCUAUGCUGGUAACAGCAUAGC | 113 |
| | 20 sgRNA | target | AAGUUUAAAUAAGGCUAGUCCGUUAUCAACUUGAAAAAGUGGCACCGAGU | |
| | | | CGGUGCUUUUUUU (SEQ ID NO: 102) | |
| 227 | CLTA1_2′OMePACE + 19 | CLTA1 ON1- | AGUCCUCAUCUCCCUCAAG*CGUUUAAGAGCUAUGCUGGUAACAGCAUAGC | 113 |
| | sgRNA | target | AAGUUUAAAUAAGGCUAGUCCGUUAUCAACUUGAAAAAGUGGCACCGAGU | |
| | | | CGGUGCUUUUUUU (SEQ ID NO: 103) | |
| 228 | CLTA1_2′OMePACE + 19 | CLTA1mg ON1- | AGUCCUCAUCUCCCUCAAG*CGUUUAAGAGCUAUGCUGGUAACAGCAUAGC | 113 |
| | sgRNA | target | AAGUUUAAAUAAGGCUAGUCCGUUAUCAACUUGAAAAAGUGGCACCGAGU | |
| | | | CGGUGCUUUUUUU (SEQ ID NO: 103) | |
| 229 | CLTA1_2′OMePACE + 19 | CLTA1mg ON1- | AGUCCUCAUCUCCCUCAAG*CGUUUAAGAGCUAUGCUGGUAACAGCAUAGC | 113 |
| | sgRNA | target | AAGUUUAAAUAAGGCUAGUCCGUUAUCAACUUGAAAAAGUGGCACCGAGU | |
| | | | CGGUGCUUUUUUU (SEQ ID NO: 103) | |
| 230 | CLTA1_2′OMePACE + 19 | CLTA1mg | AGUCCUCAUCUCCCUCAAG*CGUUUAAGAGCUAUGCUGGUAACAGCAUAGC | 113 |
| | sgRNA | OFF1-target | AAGUUUAAAUAAGGCUAGUCCGUUAUCAACUUGAAAAAGUGGCACCGAGU | |
| | | | CGGUGCUUUUUUU (SEQ ID NO: 103) | |
| 231 | CLTA1_2′OMePACE + 19 | CLTA1mg | AGUCCUCAUCUCCCUCAAG*CGUUUAAGAGCUAUGCUGGUAACAGCAUAGC | 113 |
| | sgRNA | OFF3-target | AAGUUUAAAUAAGGCUAGUCCGUUAUCAACUUGAAAAAGUGGCACCGAGU | |
| | | | CGGUGCUUUUUUU (SEQ ID NO: 103) | |
| 232 | CLTA1_2′OMePACE + 18 | CLTA1 ON1- | AGUCCUCAUCUCCCUCAA*GCGUUUAAGAGCUAUGCUGGUAACAGCAUAGC | 113 |
| | sgRNA | target | AAGUUUAAAUAAGGCUAGUCCGUUAUCAACUUGAAAAAGUGGCACCGAGU | |
| | | | CGGUGCUUUUUUU (SEQ ID NO: 104) | |
| 233 | CLTA1_2′OMePACE + 18 | CLTA1mg ON1- | AGUCCUCAUCUCCCUCAA*GCGUUUAAGAGCUAUGCUGGUAACAGCAUAGC | 113 |
| | sgRNA | target | AAGUUUAAAUAAGGCUAGUCCGUUAUCAACUUGAAAAAGUGGCACCGAGU | |
| | | | CGGUGCUUUUUUU (SEQ ID NO: 104) | |
| 234 | CLTA1_2′OMePACE + 18 | CLTA1mg ON1- | AGUCCUCAUCUCCCUCAA*GCGUUUAAGAGCUAUGCUGGUAACAGCAUAGC | 113 |
| | sgRNA | target | AAGUUUAAAUAAGGCUAGUCCGUUAUCAACUUGAAAAAGUGGCACCGAGU | |
| | | | CGGUGCUUUUUUU (SEQ ID NO: 104) | |
| 235 | CLTA1_2′OMePACE + 17 | CLTA1 ON1- | AGUCCUCAUCUCCCUCA*AGCGUUUAAGAGCUAUGCUGGUAACAGCAUAGC | 113 |
| | sgRNA | target | AAGUUUAAAUAAGGCUAGUCCGUUAUCAACUUGAAAAAGUGGCACCGAGU | |
| | | | CGGUGCUUUUUUU (SEQ ID NO: 105) | |
| 236 | CLTA1_2′OMePACE + 17 | CLTA1mg ON1- | AGUCCUCAUCUCCCUCA*AGCGUUUAAGAGCUAUGCUGGUAACAGCAUAGC | 113 |
| | sgRNA | target | AAGUUUAAAUAAGGCUAGUCCGUUAUCAACUUGAAAAAGUGGCACCGAGU | |
| | | | CGGUGCUUUUUUU (SEQ ID NO: 105) | |
| 237 | CLTA1_2′OMePACE + 17 | CLTA1mg ON1- | AGUCCUCAUCUCCCUCA*AGCGUUUAAGAGCUAUGCUGGUAACAGCAUAGC | 113 |
| | sgRNA | target | AAGUUUAAAUAAGGCUAGUCCGUUAUCAACUUGAAAAAGUGGCACCGAGU | |
| | | | CGGUGCUUUUUUU (SEQ ID NO: 105) | |
| 238 | CLTA1_2′OMePACE | CLTA1 ON1- | AGUCCUCAUCUCCCUCA*A*GCGUUUAAGAGCUAUGCUGGUAACAGCAUAG | 113 |
| | 8 sgRNA | target | CAAGUUUAAAUAAGGCUAGUCCGUUAUCAACUUGAAAAAGUGGCACCGAG | |
| | | | UCGGUGCUUUUUUU (SEQ ID NO: 106) | |
| 239 | CLTA1_2′OMePACE | CLTA1mg ON1- | AGUCCUCAUCUCCCUCA*A*GCGUUUAAGAGCUAUGCUGGUAACAGCAUAG | 113 |
| | 8 sgRNA | target | CAAGUUUAAAUAAGGCUAGUCCGUUAUCAACUUGAAAAAGUGGCACCGAG | |
| | | | UCGGUGCUUUUUUU (SEQ ID NO: 106) | |
| 240 | CLTA1_2′OMePACE | CLTA1mg ON1- | AGUCCUCAUCUCCCUCA*A*GCGUUUAAGAGCUAUGCUGGUAACAGCAUAG | 113 |
| | 8 sgRNA | target | CAAGUUUAAAUAAGGCUAGUCCGUUAUCAACUUGAAAAAGUGGCACCGAG | |
| | | | UCGGUGCUUUUUUU (SEQ ID NO: 106) | |

2′OMethyl, 3′Phosphorothioate-modified sgRNA

| | | | | |
|---|---|---|---|---|
| 241 | CLTA1_5′, 3′-3x(2′OMe, | CLTA1 ON1- | AsGsUsCCUCAUCUCCCUCAAGCGUUUAAGAGCUAUGCUGGUAACAGCAUAG | 113 |
| | 3′P(S)) | target | CAAGUUUAAAUAAGGCUAGUCCGUUAUCAACUUGAAAAAGUGGCACCGAG | |
| | | | UCGGUGCUUUUsUsUsU (SEQ ID NO: 107) | |
| 242 | CLTA1_5′, 3′-3x(2′OMe, | CLTA1mg ON1- | AsGsUsCCUCAUCUCCCUCAAGCGUUUAAGAGCUAUGCUGGUAACAGCAUAG | 113 |
| | 3′P(S)) | target | CAAGUUUAAAUAAGGCUAGUCCGUUAUCAACUUGAAAAAGUGGCACCGAG | |
| | | | UCGGUGCUUUUsUsUsU (SEQ ID NO: 107) | |
| 243 | CLTA1_5′, 3′-3x(2′OMe, | CLTA1mg ON1- | AsGsUsCCUCAUCUCCCUCAAGCGUUUAAGAGCUAUGCUGGUAACAGCAUAG | 113 |
| | 3′P(S)) | target | CAAGUUUAAAUAAGGCUAGUCCGUUAUCAACUUGAAAAAGUGGCACCGAG | |
| | | | UCGGUGCUUUUsUsUsU (SEQ ID NO: 107) | |
| 244 | CLTA1_5′, 3′-3x(2′OMe, | CLTA1mg | AsGsUsCCUCAUCUCCCUCAAGCGUUUAAGAGCUAUGCUGGUAACAGCAUAG | 113 |
| | 3′P(S)) | OFF1-target | CAAGUUUAAAUAAGGCUAGUCCGUUAUCAACUUGAAAAAGUGGCACCGAG | |
| | | | UCGGUGCUUUUsUsUsU (SEQ ID NO: 107) | |
| 245 | CLTA1_5′, 3′-3x(2′OMe, | CLTA1mg | AsGsUsCCUCAUCUCCCUCAAGCGUUUAAGAGCUAUGCUGGUAACAGCAUAG | 113 |
| | 3′P(S)) | OFF3-target | CAAGUUUAAAUAAGGCUAGUCCGUUAUCAACUUGAAAAAGUGGCACCGAG | |
| | | | UCGGUGCUUUUsUsUsU (SEQ ID NO: 107) | |
| 246 | CLTA4_5′, 3′-3x(2′OMe, | CLTA4 target | GsCsAsGAUGUAGUGUUUCCACAGUUUAAGAGCUAUGCUGGAAACAGCAUA | 113 |
| | 3′P(S)) | | GCAAGUUUAAAUAAGGCUAGUCCGUUAUCAACUUGAAAAAGUGGCACCGA | |
| | | | GUCGGUGCUUUUsUsUsU (SEQ ID NO: 131) | |
| 247 | CLTA4_5′, 3′-3x(2′OMe, | CLTA4 target | GsCsAsGAUGUAGUGUUUCCACAGUUUAAGAGCUAUGCUGGAAACAGCAUA | 113 |
| | 3′P(S)) | | GCAAGUUUAAAUAAGGCUAGUCCGUUAUCAACUUGAAAAAGUGGCACCGA | |
| | | | GUCGGUGCUUUUsUsUsU (SEQ ID NO: 131) | |
| 248 | CLTA4_5′, 3′-3x(2′OMe, | CLTA4mg ON- | GsCsAsGAUGUAGUGUUUCCACAGUUUAAGAGCUAUGCUGGAAACAGCAUA | 113 |
| | 3′P(S)) | target | GCAAGUUUAAAUAAGGCUAGUCCGUUAUCAACUUGAAAAAGUGGCACCGA | |
| | | | GUCGGUGCUUUUsUsUsU (SEQ ID NO: 131) | |
| 249 | CLTA4_5′, 3′-3x(2′OMe, | CLTA4mg ON- | GsCsAsGAUGUAGUGUUUCCACAGUUUAAGAGCUAUGCUGGAAACAGCAUA | 113 |
| | 3′P(S)) | target | GCAAGUUUAAAUAAGGCUAGUCCGUUAUCAACUUGAAAAAGUGGCACCGA | |
| | | | GUCGGUGCUUUUsUsUsU (SEQ ID NO: 131) | |
| 250 | CLTA4_5′, 3′-3x(2′OMe, | CLTA4mg ON- | GsCsAsGAUGUAGUGUUUCCACAGUUUAAGAGCUAUGCUGGAAACAGCAUA | 113 |
| | 3′P(S)) | target | GCAAGUUUAAAUAAGGCUAGUCCGUUAUCAACUUGAAAAAGUGGCACCGA | |
| | | | GUCGGUGCUUUUsUsUsU (SEQ ID NO: 131) | |
| 251 | CLTA4_5′, 3′-3x(2′OMe, | CLTA4mg | GsCsAsGAUGUAGUGUUUCCACAGUUUAAGAGCUAUGCUGGAAACAGCAUA | 113 |
| | 3′P(S)) | OFF5-target | GCAAGUUUAAAUAAGGCUAGUCCGUUAUCAACUUGAAAAAGUGGCACCGA | |
| | | | GUCGGUGCUUUUsUsUsU (SEQ ID NO: 131) | |
| 252 | CLTA4_5′-3x(2′OMe, | CLTA4mg ON- | GsCsAsGAUGUAGUGUUUCCACAGUUUAAGAGCUAUGCUGGAAACAGCAUA | 113 |
| | 3′P(S)), 3′-5x(2′OMe, | target | GCAAGUUUAAAUAAGGCUAGUCCGUUAUCAACUUGAAAAAGUGGCACCGA | |
| | 3′P(S)) | | GUCGGUGCUUsUsUsUsUsU (SEQ ID NO: 108) | |
| 253 | CLTA4_5′-3x(2′OMe, | CLTA4mg ON- | GsCsAsGAUGUAGUGUUUCCACAGUUUAAGAGCUAUGCUGGAAACAGCAUA | 113 |
| | 3′P(S)), 3′-5x(2′OMe, | target | GCAAGUUUAAAUAAGGCUAGUCCGUUAUCAACUUGAAAAAGUGGCACCGA | |
| | 3′P(S)) | | GUCGGUGCUUsUsUsUsUsU (SEQ ID NO: 108) | |
| 254 | CLTA4_5′-3x(2′OMe, | CLTA4mg ON- | GsCsAsGAUGUAGUGUUUCCACAGUUUAAGAGCUAUGCUGGAAACAGCAUA | 113 |
| | 3′P(S)), 3′-5x(2′OMe, | target | GCAAGUUUAAAUAAGGCUAGUCCGUUAUCAACUUGAAAAAGUGGCACCGA | |
| | 3′P(S)) | | GUCGGUGCUUsUsUsUsUsU (SEQ ID NO: 108) | |
| 255 | CLTA4_5′, 3′-5x(2′OMe, | CLTA4mg ON- | GsCsAsGsAsUGUAGUGUUUCCACAGUUUAAGAGCUAUGCUGGAAACAGCAU | 113 |
| | 3′P(S)) | target | AGCAAGUUUAAAUAAGGCUAGUCCGUUAUCAACUUGAAAAAGUGGCACCG | |
| | | | AGUCGGUGCUUsUsUsUsUsU (SEQ ID NO: 109) | |
| 256 | CLTA4_5′, 3′-5x(2′OMe, | CLTA4mg ON- | GsCsAsGsAsUGUAGUGUUUCCACAGUUUAAGAGCUAUGCUGGAAACAGCAU | 113 |
| | 3′P(S)) | target | AGCAAGUUUAAAUAAGGCUAGUCCGUUAUCAACUUGAAAAAGUGGCACCG | |
| | | | AGUCGGUGCUUsUsUsUsUsU (SEQ ID NO: 109) | |
| 257 | CLTA4_5′, 3′-5x(2′OMe, | CLTA4mg | GsCsAsGsAsUGUAGUGUUUCCACAGUUUAAGAGCUAUGCUGGAAACAGCAU | 113 |
| | 3′P(S)) | OFF5-target | AGCAAGUUUAAAUAAGGCUAGUCCGUUAUCAACUUGAAAAAGUGGCACCG | |
| | | | AGUCGGUGCUUsUsUsUsUsU (SEQ ID NO: 109) | |

2′OMethyl, 3′PhosphorothioPACE-modified sgRNA

| | | | | |
|---|---|---|---|---|
| 258 | CLTA1_5′, 3′-3x(2′OMe, | CLTA1 ON1- | A*sG*sU*sCCUCAUCUCCCUCAAGCGUUUAAGAGCUAUGCUGGUAACAGCA | 113 |
| | 3′thioPACE) | target | UAGCAAGUUUAAAUAAGGCUAGUCCGUUAUCAACUUGAAAAAGUGGCACC | |
| | | | GAGUCGGUGCUUUU*sU*sU*sU (SEQ ID NO: 110) | |
| 259 | CLTA1_5′, 3′-3x(2′OMe, | CLTA1mg ON1- | A*sG*sU*sCCUCAUCUCCCUCAAGCGUUUAAGAGCUAUGCUGGUAACAGCA | 113 |
| | 3′thioPACE) | target | UAGCAAGUUUAAAUAAGGCUAGUCCGUUAUCAACUUGAAAAAGUGGCACC | |
| | | | GAGUCGGUGCUUUU*sU*sU*sU (SEQ ID NO: 110) | |
| 260 | CLTA1_5′, 3′-3x(2′OMe, | CLTA1mg ON1- | A*sG*sU*sCCUCAUCUCCCUCAAGCGUUUAAGAGCUAUGCUGGUAACAGCA | 113 |
| | 3′thioPACE) | target | UAGCAAGUUUAAAUAAGGCUAGUCCGUUAUCAACUUGAAAAAGUGGCACC | |
| | | | GAGUCGGUGCUUUU*sU*sU*sU (SEQ ID NO: 110) | |
| 261 | CLTA1_5′, 3′-3x(2′OMe, | CLTA1mg | A*sG*sU*sCCUCAUCUCCCUCAAGCGUUUAAGAGCUAUGCUGGUAACAGCA | 113 |
| | 3′thioPACE) | OFF1-target | UAGCAAGUUUAAAUAAGGCUAGUCCGUUAUCAACUUGAAAAAGUGGCACC | |
| | | | GAGUCGGUGCUUUU*sU*sU*sU (SEQ ID NO: 110) | |
| 262 | CLTA1_5′, 3′-3x(2′OMe, | CLTA1mg | A*sG*sU*sCCUCAUCUCCCUCAAGCGUUUAAGAGCUAUGCUGGUAACAGCA | 113 |
| | 3′thioPACE) | OFF3-target | UAGCAAGUUUAAAUAAGGCUAGUCCGUUAUCAACUUGAAAAAGUGGCACC | |
| | | | GAGUCGGUGCUUUU*sU*sU*sU (SEQ ID NO: 110) | |
| 263 | CLTA1_5′, 3′-1x(2′OMe, | CLTA1mg ON1- | A*sGUCCUCAUCUCCCUCAAGCGUUUAAGAGCUAUGCUGGUAACAGCAUAG | 113 |
| | 3′thioPACE) | target | CAAGUUUAAAUAAGGCUAGUCCGUUAUCAACUUGAAAAAGUGGCACCGAG | |
| | | | UCGGUGCUUUUUU*sU (SEQ ID NO: 111) | |
| 264 | CLTA1_5′, 3′-1x(2′OMe, | CLTA1mg ON1- | A*sGUCCUCAUCUCCCUCAAGCGUUUAAGAGCUAUGCUGGUAACAGCAUAG | 113 |
| | 3′thioPACE) | target | CAAGUUUAAAUAAGGCUAGUCCGUUAUCAACUUGAAAAAGUGGCACCGAG | |
| | | | UCGGUGCUUUUUU*sU (SEQ ID NO: 111) | |
| 265 | CLTA1_5′, 3′-1x(2′OMe, | CLTA1mg | A*sGUCCUCAUCUCCCUCAAGCGUUUAAGAGCUAUGCUGGUAACAGCAUAG | 113 |
| | 3′thioPACE) | OFF1-target | CAAGUUUAAAUAAGGCUAGUCCGUUAUCAACUUGAAAAAGUGGCACCGAG | |
| | | | UCGGUGCUUUUUU*sU (SEQ ID NO: 111) | |
| 266 | CLTA1_5′, 3′-1x(2′OMe, | CLTA1mg | A*sGUCCUCAUCUCCCUCAAGCGUUUAAGAGCUAUGCUGGUAACAGCAUAG | 113 |
| | 3′thioPACE) | OFF3-target | CAAGUUUAAAUAAGGCUAGUCCGUUAUCAACUUGAAAAAGUGGCACCGAG | |
| | | | UCGGUGCUUUUUU*sU (SEQ ID NO: 111) | |
| 267 | CLTA1_5′, 3′-3x(2′OMe, | CLTA1 ON1- | A*sG*sU*sCCUCAUCUCCCUCAAGCGUUUAAGAGCUAUGCUGGUAACAGCA | 75 |
| | 3′thioPACE) _75mer | target | UAGCAAGUUUAAAUAAGGCUAGUCCG*sU*sU*sU (SEQ ID NO: 112) | |
| 268 | CLTA1_5′, 3′-1x(2′OMe, | CLTA1mg ON1- | A*sGUCCUCAUCUCCCUCAAGCGUUUAAGAGCUAUGCUGGUAACAGCAUAG | 74 |
| | 3′thioPACE) _74mer | target | CAAGUUUAAAUAAGGCUAGUCCGU*sU (SEQ ID NO: 132) | |
| 269 | CLTA1_5′, 3′-1x(2′OMe, | CLTA1mg ON1- | A*sGUCCUCAUCUCCCUCAAGCGUUUAAGAGCUAUGCUGGUAACAGCAUAG | 75 |
| | 3′thioPACE) _75mer | target | CAAGUUUAAAUAAGGCUAGUCCGUU*sA (SEQ ID NO: 133) | |
| 270 | CLTA1_5′, 3′-1x(2′OMe, | CLTA1mg ON1- | A*sGUCCUCAUCUCCCUCAAGCGUUUAAGAGCUAUGCUGGUAACAGCAUAG | 77 |
| | 3′thioPACE) _77mer | target | CAAGUUUAAAUAAGGCUAGUCCGUUAU*sC (SEQ ID NO: 113) | |
| 271 | CLTA1_5′, 3′-1x(2′OMe, | CLTA1mg ON1- | G*sAGUCCUCAUCUCCCUCAAGCGUUUAAGAGCUAUGCUGGUAACAGCAUA | 78 |
| | 3′thioPACE) _77mer+G | target | GCAAGUUUAAAUAAGGCUAGUCCGUUAU*sC (SEQ ID NO: 114) | |
| 272 | CLTA4_5′, 3′-3x(2′OMe, | CLTA4 ON- | G*sC*sA*sGAUGUAGUGUUUCCACAGUUUAAGAGCUAUGCUGGAAACAGCA | 113 |
| | 3′thioPACE) | target | UAGCAAGUUUAAAUAAGGCUAGUCCGUUAUCAACUUGAAAAAGUGGCACC | |
| | | | GAGUCGGUGCUUUU*sU*sU*sU (SEQ ID NO: 115) | |
| 273 | CLTA4_5′, 3′-3x(2′OMe, | CLTA4 ON- | G*sC*sA*sGAUGUAGUGUUUCCACAGUUUAAGAGCUAUGCUGGAAACAGCA | 113 |
| | 3′thioPACE) | target | UAGCAAGUUUAAAUAAGGCUAGUCCGUUAUCAACUUGAAAAAGUGGCACC | |
| | | | GAGUCGGUGCUUUU*sU*sU*sU (SEQ ID NO: 115) | |
| 274 | CLTA4_5′, 3′-3x(2′OMe, | CLTA4 ON- | G*sC*sA*sGAUGUAGUGUUUCCACAGUUUAAGAGCUAUGCUGGAAACAGCA | 113 |
| | 3′thioPACE) | target | UAGCAAGUUUAAAUAAGGCUAGUCCGUUAUCAACUUGAAAAAGUGGCACC | |
| | | | GAGUCGGUGCUUUU*sU*sU*sU (SEQ ID NO: 115) | |
| 275 | CLTA4_5′, 3′-3x(2′OMe, | CLTA4mg | G*sC*sA*sGAUGUAGUGUUUCCACAGUUUAAGAGCUAUGCUGGAAACAGCA | 113 |
| | 3′thioPACE) | OFF5-target | UAGCAAGUUUAAAUAAGGCUAGUCCGUUAUCAACUUGAAAAAGUGGCACC | |
| | | | GAGUCGGUGCUUUU*sU*sU*sU (SEQ ID NO: 115) | |
| 276 | CLTA4_5′, 3′-1x(2′OMe, | CLTA4mg ON- | G*sCAGAUGUAGUGUUUCCACAGUUUAAGAGCUAUGCUGGAAACAGCAUAG | 113 |
| | 3′thioPACE) | target | CAAGUUUAAAUAAGGCUAGUCCGUUAUCAACUUGAAAAAGUGGCACCGAG | |
| | | | UCGGUGCUUUUUU*sU (SEQ ID NO: 116) | |
| 277 | CLTA4_5′, 3′-1x(2′OMe, | CLTA4mg ON- | G*sCAGAUGUAGUGUUUCCACAGUUUAAGAGCUAUGCUGGAAACAGCAUAG | 113 |
| | 3′thioPACE) | target | CAAGUUUAAAUAAGGCUAGUCCGUUAUCAACUUGAAAAAGUGGCACCGAG | |
| | | | UCGGUGCUUUUUU*sU (SEQ ID NO: 116) | |
| 278 | CLTA4_5′, 3′-1x(2′OMe, | CLTA4mg ON- | G*sCAGAUGUAGUGUUUCCACAGUUUAAGAGCUAUGCUGGAAACAGCAUAG | 113 |
| | 3′thioPACE) | target | CAAGUUUAAAUAAGGCUAGUCCGUUAUCAACUUGAAAAAGUGGCACCGAG | |
| | | | UCGGUGCUUUUUU*sU (SEQ ID NO: 116) | |
| 279 | CLTA4_5′, 3′-1x(2′OMe, | target | G*sCAGAUGUAGUGUUUCCACAGUUUAAGAGCUAUGCUGGAAACAGCAUAG | |
| | 3′thioPACE) | | CAAGUUUAAAUAAGGCUAGUCCGUUAUCAACUUGAAAAAGUGGCACCGAG | |
| | | | UCGGUGCUUUUUU*sU (SEQ ID NO: 116) | |

2-aminoA-modified sgRNA (including unmodified controls)

| | | | | |
|---|---|---|---|---|
| 280 | EN1 | EN1mg ON- | GAUGUUGUCGAUGAAAAAGUGUUUAAGAGCUAUGCUGGUAACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 117) | |
| 281 | EN1 | EN1mg OFF- | GAUGUUGUCGAUGAAAAAGUGUUUAAGAGCUAUGCUGGUAACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 117) | |
| 282 | EN1_2aminoA+16 | EN1mg ON- | GAUGUUGUCGAUGAA(2aA)AAGUGUUUAAGAGCUAUGCUGGUAACAGCAU | 113 |
| | | target | AGCAAGUUUAAAUAAGGCUAGUCCGUUAUCAACUUGAAAAAGUGGCACCG | |
| | | | AGUCGGUGCUUUUUUU (SEQ ID NO: 118) | |
| 283 | EN1_2aminoA+16 | EN1mg OFF- | GAUGUUGUCGAUGAA(2aA)AAGUGUUUAAGAGCUAUGCUGGUAACAGCAU | 113 |
| | | target | AGCAAGUUUAAAUAAGGCUAGUCCGUUAUCAACUUGAAAAAGUGGCACCG | |
| | | | AGUCGGUGCUUUUUUU (SEQ ID NO: 118) | |
| 284 | PCDHA4 | PCDHA4mg ON- | GAUUUAGACGAAGGAUUGAAGUUUAAGAGCUAUGCUGGUAACAGCAUAGC | 113 |
| | | target | AAGUUUAAAUAAGGCUAGUCCGUUAUCAACUUGAAAAAGUGGCACCGAGU | |
| | | | CGGUGCUUUUUUU (SEQ ID NO: 119) | |
| 285 | PCDHA4 | PCDHA4mg | GAUUUAGACGAAGGAUUGAAGUUUAAGAGCUAUGCUGGUAACAGCAUAGC | 113 |
| | | OFF-target | AAGUUUAAAUAAGGCUAGUCCGUUAUCAACUUGAAAAAGUGGCACCGAGU | |
| | | | CGGUGCUUUUUUU (SEQ ID NO: 119) | |
| 286 | PCDHA4_2aminoA+15 | PCDHA4mg ON- | GAUUUAGACGAAGG(2aA)UUGAAGUUUAAGAGCUAUGCUGGUAACAGCAU | 113 |
| | | target | AGCAAGUUUAAAUAAGGCUAGUCCGUUAUCAACUUGAAAAAGUGGCACCG | |
| | | | AGUCGGUGCUUUUUUU (SEQ ID NO: 120) | |
| 287 | PCDHA4_2aminoA+15 | PCDHA4mg | GAUUUAGACGAAGG(2aA)UUGAAGUUUAAGAGCUAUGCUGGUAACAGCAU | 113 |
| | | OFF-target | AGCAAGUUUAAAUAAGGCUAGUCCGUUAUCAACUUGAAAAAGUGGCACCG | |
| | | | AGUCGGUGCUUUUUUU (SEQ ID NO: 120) | |

5-methylU-modified sgRNA

| | | | | |
|---|---|---|---|---|
| 288 | CLTA4_21x(5-MeU) | CLTA4mg ON- | GCAGA(5mU)G(5mU)AG(5mU)G(5mU)(5mU)(5mU)CCACAGUUUAAGAGC | 113 |
| | | target | (5mU)A(5mU)GC(5mU)GG(5mU)AACAGCA(5mU)AGCAAGUUUAAAUAAGG | |
| | | | CUAGUCCGUUAUCAAC(5mU)(5mU)GAAAAAG(5mU)GGCACCGAGUCGG(5 | |
| | | | mU)GC(5mU)(5mU)(5mU)(5mU)(5mU)(5mU)U (SEQ ID NO: 121) | |
| 289 | CLTA4_21x(5-MeU) | CLTA4mg | GCAGA(5mU)G(5mU)AG(5mU)G(5mU)(5mU)(5mU)CCACAGUUUAAGAGC | 113 |
| | | OFF5-target | (5mU)A(5mU)GC(5mU)GG(5mU)AACAGCA(5mU)AGCAAGUUUAAAUAAGG | |
| | | | CUAGUCCGUUAUCAAC(5mU)(5mU)GAAAAAG(5mU)GGCACCGAGUCGG | |
| | | | (5mU)GC(5mU)(5mU)(5mU)(5mU)(5mU)(5mU)U (SEQ ID NO: 121) | |

Z base-modified sgRNA

| | | | | |
|---|---|---|---|---|
| 290 | CLTA1_ZZ_70, 71 | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUZZGUUAUCAACUUGAAAAAGUGGCACCGAGUC | |
| | | | GGUGCUUUUUUU (SEQ ID NO: 122) | |
| 291 | CLTA1_ZZ_95, 96 | CLTA1 ON1- | AGUCCUCAUCUCCCUCAAGCGUUUAAGAGCUAUGCUGGUAACAGCAUAGCA | 113 |
| | | target | AGUUUAAAUAAGGCUAGUCCGUUAUCAACUUGAAAAAGUGGCAZZGAGUC | |
| | | | GGUGCUUUUUUU (SEQ ID NO: 122) | |

sgRNA modified to disfavor
misfolding

| | | | | |
|---|---|---|---|---|
| 292 | CLTA1_opti_short_5′, | CLTA1mg ON1- | A*sGUCCUCAUCUCCCUCAAGCGUUUAAGAGCUAGUAAUAGCAAGUUUAAA | 100 |
| | 3′-1x(2′OMe, | target | UAAGGUUAAUCCGUUAUCAACAAGAAAUUGUGGCACCGAGUCGGUGCUUU | |
| | 3′thioPACE)_2′OMe_54, 57 | | *sU (SEQ ID NO: 123) | |
| 293 | CLTA1_opti_short_5′, | CLTA1mg ON1- | A*sGUCCUCAUCUCCCUCAAGCGUUUAAGAGCUAUGCUGGUAACAGCAUAG | 113 |
| | 3′-1x(2′OMe, | target | CAAGUUUAAAUAAGGUUAAUCCGUUAUCAACAAGAAAUUGUGGCACCGAG | |
| | 3′thioPACE)_2′OMe_64, 67 | | UCGGUGCUUUUUU*sU (SEQ ID NO: 124) | |

N = 2′OMe
N = 2′deoxy
Ns = 3′P(S)
N*= 3′-PACE
N*s = 3′-thioPACE
N* = 2′OMe, 3′-PACE
N*s = 2′OMe, 3′-thioPACE
Ns = 2′OMe, 3′P(S)
Nₒ = 5′-overhang (5′ to the 20-nt guide sequence); “NC” means the overhang is not complementary to protospacer-adjacent sequence
(2sU) = 2-thioU
(2aA) = 2-aminoA
(5mU) = 5-methylU
Z = Z base
IntFl = Fluorophore incorporated at an internal position in the RNA sequence

The DNA target constructs in Table 3 had the following sequences:

| | |
|---|---|
| CLTA1 ON1- | AGAATTTAACTGTGGTCACATTTGCTTTATCGACTGGCTTCATCTCACAGCTCATC |
| target: | TTACGCAAGTTCGATGAGTATGCCAGTCACTTTCAATTTGGTTGAATGTTCCCGTG |
| | ACATGCGAGTTCTGTCGACCATGTGCCGCGGATTGAATTCCTCAAGGGTGGTGATA |
| | GATGCTACGGTGGTGATGCGCATGCGCTCAGTCCTCATCTCCCTCAAGCAGGCCCC |
| | GCTGGTGGGTCGGAGTCCCTAGTGAAGCCACCAATATAGTGGTCGTGTCAAGCAAC |
| | TGTCCACGCTCCACCCTCGAGGTCGTAACATAAACGTACTAAGGCACGAGTAAACA |
| | AGATCGATAGCAAGAACATGGTATAGACTGACGGAGAGCTCGCCATTAGTCTGA |
| | (SEQ ID NO: 10) |
| CLTA1 OFF1- | AGAATTTAACTGTGGTCACATTTGCTTTATCGACTGGCTTCATCTCACAGCTCATC |
| target: | TTACGCAAGTTCGATGAGTATGCCAGTCACTTTCAATTTGGTTGAATGTTCCCGTG |
| | ACATGCGAGTTCTGTCGACCATGTGCCGCGGATTGAATTCCTCAAGGGTGGTGATA |
| | GATGCTACGGTGGTGATGCGTATGCACTCAGTCCTCAACTCCCTCAAGCAGGCGAC |
| | CCCTGGGGGTCGGAGTCCCTAGTGAAGCCACCAATATAGTGGTCGTGTCAAGCAAC |
| | TGTCCACGCTCCACCCTCGAGGTCGTAACATAAACGTACTAAGGCACGAGTAAACA |
| | AGATCGATAGCAAGAACATGGTATAGACTGACGGAGAGCTCGCCATTAGTCTGA |
| | (SEQ ID NO: 11) |
| CLTA1 OFF2- | AGAATTTAACTGTGGTCACATTTGCTTTATCGACTGGCTTCATCTCACAGCTCATC |
| target: | TTACGCAAGTTCGATGAGTATGCCAGTCACTTTCAATTTGGTTGAATGTTCCCGTG |
| | ACATGCGAGTTCTGTCGACCATGTGCCGCGGATTGAATTCCTCAAGGGTGGTGATA |
| | GATGCTACGGTGGTGATGCAATAAATTTCAGCCCTCATTTCCCTCAAGCAGGGGTT |
| | ACTTTAGGGTCGGAGTCCCTAGTGAAGCCACCAATATAGTGGTCGTGTCAAGCAAC |
| | TGTCCACGCTCCACCCTCGAGGTCGTAACATAAACGTACTAAGGCACGAGTAAACA |
| | AGATCGATAGCAAGAACATGGTATAGACTGACGGAGAGCTCGCCATTAGTCTGA |
| | (SEQ ID NO: 12) |
| CLTA1 OFF3- | AGAATTTAACTGTGGTCACATTTGCTTTATCGACTGGCTTCATCTCACAGCTCATC |
| target: | TTACGCAAGTTCGATGAGTATGCCAGTCACTTTCAATTTGGTTGAATGTTCCCGTG |
| | ACATGCGAGTTCTGTCGACCATGTGCCGCGGATTGAATTCCTCAAGGGTGGTGATA |
| | GATGCTACGGTGGTGATGCTCTCCAGCCCACTCCTCATCCCCCTCAAGCCGGTCCC |
| | AGGCTGGGGTCGGAGTCCCTAGTGAAGCCACCAATATAGTGGTCGTGTCAAGCAAC |
| | TGTCCACGCTCCACCCTCGAGGTCGTAACATAAACGTACTAAGGCACGAGTAAACA |
| | AGATCGATAGCAAGAACATGGTATAGACTGACGGAGAGCTCGCCATTAGTCTGA |
| | (SEQ ID NO: 13) |
| CLTA1mg ON1- | GCGTTTCTGGGTGAGCAAAAACAGGAAGGCAAAATGCCGCAAAAAAGGGAATAAGG |
| target: | GCGACACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAATA |
| | AACAAATAGGGGTTCCGCGCACATTTCCCCGAAAAGTGCCACCTAAATTGTAAGCG |
| | TTAATATTTTGTTAAAATTCGCGTTAAATTTTTGTTAAATCAGCTCATTTTTTAAC |
| | CAATAGGCCGAAATCGGCAAAATCCCTTATAAATCAAAAGAATAGACCGAGATAGG |
| | GTTGAGTGTTGTTCCAGTTTGGAACAAGAGTCCACTATTAAAGAACGTGGACTCCA |
| | ACGTCAAAGGGCGAAAAACCGTCTATCAGGGCGATGGCCCACTACGTGAACCATCA |
| | CCCTAATCAAGTTTTTTGGGGTCGAGGTGCCGTAAAGCACTAAATCGGAACCCTAA |
| | AGGGAGCCCCCGATTTAGAGCTTGACGGGGAAAGCCGGCGAACGTGGCGAGAAAGG |
| | AAGGGAAGAAAGCGAAAGGAGCGGGCGCTAGGGCGCTGGCAAGTGTAGCGGTCACG |
| | CTGCGCGTAACCACCACACCCGCCGCGCTTAATGCGCCGCTACAGGGCGCGTCCCA |
| | TTCGCCATTCAGGCTGCGCAACTGTTGGGAAGGGCGATCGGTGCGGGCCTCTTCGC |
| | TATTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAGCGCGCGTAATA |
| | CGACTCACTATAGGGCGAATTGGGTACGATCGATGCGGCCTCGCAGGCCAAAGATG |
| | TCTCCCGCATGCGCTCAGTCCTCATCTCCCTCAAGCAGGCCCTGCTGGTGCACTGA |
| | AGAGCCACCCTGTGCGCGTGATATGCAGCTCCAGCTTTTGTTCCCTTTAGTGAGGG |
| | TTAATTGCGCGCTTGGCGTAATCATGGTCATAGCTGTTTCCTGTGTGAAATTGTTA |
| | TCCGCTCACAATTCCACACAACATACGAGCCGGAAGCATAAAGTGTAAAGCCTGGG |
| | GTGCCTAATGAGTGAGCTAACTCACATTAATTGCGTTGCGCTCACTGCCCGCTTTC |
| | CAGTCGGGAAACCTGTCGTGCCAGCTGCATTAATGAATCGGCCAACGCGCGGGGAG |
| | AGGCGGTTTGCGTATTGGGCGCTCTTCCGCTTCCTCGCTCACTGACTCGCTGCGCT |
| | CGGTCGTTCGGCTGCGGCGAGCGGTATCAGCTCACTCAAAGGCGGTAATACGGTTA |
| | TCCACAGAATCAGGGGATAACGCAGGAAAGAACATGTGAGCAAAAGGCCAGCAAAA |
| | GGCCAGGAACCGTAAAAAGGCCGCGTTGCTGGCGTTTTTCCATAGGCTCCGCCCCC |
| | CTGACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTTCC |
| | GACCCTGCCGCTTACCGGATACCTGTCCGCCTTTCTCCCTTCGGGAAGCGTGGCGC |
| | TTTCTCATAGCTCACGCTGTAGGTATCTCAGTTCGGTGTAGGTCGTTCGCTCCAAG |
| | CTGGGCTGTGTGCACGAACCCCCCGTTCAGCCCGACCGCTGCGCCTTATCCGGTAA |
| | CTATCGTCTTGAGTCCAACCCGGTAAGACACGACTTATCGCCACTGGCAGCAGCCA |
| | CTGGTAACAGGATTAGCAGAGCGAGGTATGTAGGCGGTGCTACAGAGTTCTTGAAG |
| | TGGTGGCCTAACTACGGCTACACTAGAAGGACAGTATTTGGTATCTGCGCTCTGCT |
| | GAAGCCAGTTACCTTCGGAAAAAGAGTTGGTAGCTCTTGATCCGGCAAACAAACCA |
| | CCGCTGGTAGCGGTGGTTTTTTTGTTTGCAAGCAGCAGATTACGCGCAGAAAAAAA |
| | GGATCTCAAGAAGATCCTTTGATCTTTTCTACGGGGTCTGACGCTCAGTGGAACGA |
| | AAACTCACGTTAAGGGATTTTGGTCATGAGATTATCAAAAAGGATCTTCACCTAGA |
| | TCCTTTTAAATTAAAAATGAAGTTTTAAATCAATCTAAAGTATATATGAGTAAACT |
| | TGGTCTGACAGTTACCAATGCTTAATCAGTGAGGCACCTATCTCAGCGATCTGTCT |
| | ATTTCGTTCATCCATAGTTGCCTGACTCCCCGTCGTGTAGATAACTACGATACGGG |
| | AGGGCTTACCATCTGGCCCCAGTGCTGCAATGATACCGCGAGACCCACGCTCACCG |
| | GCTCCAGATTTATCAGCAATAAACCAGCCAGCCGGAAGGGCCGAGCGCAGAAGTGG |
| | TCCTGCAACTTTATCCGCCTCCATCCAGTCTATTAATTGTTGCCGGGAAGCTAGAG |
| | TAAGTAGTTCGCCAGTTAATAGTTTGCGCAACGTTGTTGCCATTGCTACAGGCATC |
| | GTGGTGTCACGCTCGTCGTTTGGTATGGCTTCATTCAGCTCCGGTTCCCAACGATC |
| | AAGGCGAGTTACATGATCCCCCATGTTGTGCAAAAAAGCGGTTAGCTCCTTCGGTC |
| | CTCCGATCGTTGTCAGAAGTAAGTTGGCCGCAGTGTTATCACTCATGGTTATGGCA |
| | GCACTGCATAATTCTCTTACTGTCATGCCATCCGTAAGATGCTTTTCTGTGACTGG |
| | TGAGTACTCAACCAAGTCATTCTGAGAATAGTGTATGCGGCGACCGAGTTGCTCTT |
| | GCCCGGCGTCAATACGGGATAATACCGCGCCACATAGC (SEQ ID NO: 14) |
| CLTA1mg OFF1- | GCGTTTCTGGGTGAGCAAAAACAGGAAGGCAAAATGCCGCAAAAAAGGGAATAAGG |
| target: | GCGACACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAATA |
| | AACAAATAGGGGTTCCGCGCACATTTCCCCGAAAAGTGCCACCTAAATTGTAAGCG |
| | TTAATATTTTGTTAAAATTCGCGTTAAATTTTTGTTAAATCAGCTCATTTTTTAAC |
| | CAATAGGCCGAAATCGGCAAAATCCCTTATAAATCAAAAGAATAGACCGAGATAGG |
| | GTTGAGTGTTGTTCCAGTTTGGAACAAGAGTCCACTATTAAAGAACGTGGACTCCA |
| | ACGTCAAAGGGCGAAAAACCGTCTATCAGGGCGATGGCCCACTACGTGAACCATCA |
| | CCCTAATCAAGTTTTTTGGGGTCGAGGTGCCGTAAAGCACTAAATCGGAACCCTAA |
| | AGGGAGCCCCCGATTTAGAGCTTGACGGGGAAAGCCGGCGAACGTGGCGAGAAAGG |
| | AAGGGAAGAAAGCGAAAGGAGCGGGCGCTAGGGCGCTGGCAAGTGTAGCGGTCACG |
| | CTGCGCGTAACCACCACACCCGCCGCGCTTAATGCGCCGCTACAGGGCGCGTCCCA |
| | TTCGCCATTCAGGCTGCGCAACTGTTGGGAAGGGCGATCGGTGCGGGCCTCTTCGC |
| | TATTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAGCGCGCGTAATA |
| | CGACTCACTATAGGGCGAATTGGGTACGATCGATGCGGCCTCGCAGGGCAAAGAGG |
| | TCTCCTGTATGCACTCAGTCCTCAACTCCCTCAAGCAGGCGACCCTTGGTGCACTG |
| | ACAAACCGCTCCTGCGCGTGATATGCAGCTCCAGCTTTTGTTCCCTTTAGTGAGGG |
| | TTAATTGCGCGCTTGGCGTAATCATGGTCATAGCTGTTTCCTGTGTGAAATTGTTA |
| | TCCGCTCACAATTCCACACAACATACGAGCCGGAAGCATAAAGTGTAAAGCCTGGG |
| | GTGCCTAATGAGTGAGCTAACTCACATTAATTGCGTTGCGCTCACTGCCCGCTTTC |
| | CAGTCGGGAAACCTGTCGTGCCAGCTGCATTAATGAATCGGCCAACGCGCGGGGAG |
| | AGGCGGTTTGCGTATTGGGCGCTCTTCCGCTTCCTCGCTCACTGACTCGCTGCGCT |
| | CGGTCGTTCGGCTGCGGCGAGCGGTATCAGCTCACTCAAAGGCGGTAATACGGTTA |
| | TCCACAGAATCAGGGGATAACGCAGGAAAGAACATGTGAGCAAAAGGCCAGCAAAA |
| | GGCCAGGAACCGTAAAAAGGCCGCGTTGCTGGCGTTTTTCCATAGGCTCCGCCCCC |
| | CTGACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTTCC |
| | GACCCTGCCGCTTACCGGATACCTGTCCGCCTTTCTCCCTTCGGGAAGCGTGGCGC |
| | TTTCTCATAGCTCACGCTGTAGGTATCTCAGTTCGGTGTAGGTCGTTCGCTCCAAG |
| | CTGGGCTGTGTGCACGAACCCCCCGTTCAGCCCGACCGCTGCGCCTTATCCGGTAA |
| | CTATCGTCTTGAGTCCAACCCGGTAAGACACGACTTATCGCCACTGGCAGCAGCCA |
| | CTGGTAACAGGATTAGCAGAGCGAGGTATGTAGGCGGTGCTACAGAGTTCTTGAAG |
| | TGGTGGCCTAACTACGGCTACACTAGAAGGACAGTATTTGGTATCTGCGCTCTGCT |
| | GAAGCCAGTTACCTTCGGAAAAAGAGTTGGTAGCTCTTGATCCGGCAAACAAACCA |
| | CCGCTGGTAGCGGTGGTTTTTTTGTTTGCAAGCAGCAGATTACGCGCAGAAAAAAA |
| | GGATCTCAAGAAGATCCTTTGATCTTTTCTACGGGGTCTGACGCTCAGTGGAACGA |
| | AAACTCACGTTAAGGGATTTTGGTCATGAGATTATCAAAAAGGATCTTCACCTAGA |
| | TCCTTTTAAATTAAAAATGAAGTTTTAAATCAATCTAAAGTATATATGAGTAAACT |
| | TGGTCTGACAGTTACCAATGCTTAATCAGTGAGGCACCTATCTCAGCGATCTGTCT |
| | ATTTCGTTCATCCATAGTTGCCTGACTCCCCGTCGTGTAGATAACTACGATACGGG |
| | AGGGCTTACCATCTGGCCCCAGTGCTGCAATGATACCGCGAGACCCACGCTCACCG |
| | GCTCCAGATTTATCAGCAATAAACCAGCCAGCCGGAAGGGCCGAGCGCAGAAGTGG |
| | TCCTGCAACTTTATCCGCCTCCATCCAGTCTATTAATTGTTGCCGGGAAGCTAGAG |
| | TAAGTAGTTCGCCAGTTAATAGTTTGCGCAACGTTGTTGCCATTGCTACAGGCATC |
| | GTGGTGTCACGCTCGTCGTTTGGTATGGCTTCATTCAGCTCCGGTTCCCAACGATC |
| | AAGGCGAGTTACATGATCCCCCATGTTGTGCAAAAAAGCGGTTAGCTCCTTCGGTC |
| | CTCCGATCGTTGTCAGAAGTAAGTTGGCCGCAGTGTTATCACTCATGGTTATGGCA |
| | GCACTGCATAATTCTCTTACTGTCATGCCATCCGTAAGATGCTTTTCTGTGACTGG |
| | TGAGTACTCAACCAAGTCATTCTGAGAATAGTGTATGCGGCGACCGAGTTGCTCTT |
| | GCCCGGCGTCAATACGGGATAATACCGCGCCACATAGC (SEQ ID NO: 15) |
| CLTA1mg_OFF3 | GCGTTTCTGGGTGAGCAAAAACAGGAAGGCAAAATGCCGCAAAAAAGGGAATAAGG |
| target: | GCGACACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAATA |
| | AACAAATAGGGGTTCCGCGCACATTTCCCCGAAAAGTGCCACCTAAATTGTAAGCG |
| | TTAATATTTTGTTAAAATTCGCGTTAAATTTTTGTTAAATCAGCTCATTTTTTAAC |
| | CAATAGGCCGAAATCGGCAAAATCCCTTATAAATCAAAAGAATAGACCGAGATAGG |
| | GTTGAGTGTTGTTCCAGTTTGGAACAAGAGTCCACTATTAAAGAACGTGGACTCCA |
| | ACGTCAAAGGGCGAAAAACCGTCTATCAGGGCGATGGCCCACTACGTGAACCATCA |
| | CCCTAATCAAGTTTTTTGGGGTCGAGGTGCCGTAAAGCACTAAATCGGAACCCTAA |
| | AGGGAGCCCCCGATTTAGAGCTTGACGGGGAAAGCCGGCGAACGTGGCGAGAAAGG |
| | AAGGGAAGAAAGCGAAAGGAGCGGGCGCTAGGGCGCTGGCAAGTGTAGCGGTCACG |
| | CTGCGCGTAACCACCACACCCGCCGCGCTTAATGCGCCGCTACAGGGCGCGTCCCA |
| | TTCGCCATTCAGGCTGCGCAACTGTTGGGAAGGGCGATCGGTGCGGGCCTCTTCGC |
| | TATTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAGCGCGCGTAATA |
| | CGACTCACTATAGGGCGAATTGGGTACGATCGATGCGGCCTCAGGAGAGGGAGCCA |
| | TGCTCATCTCCAGCCCACTCCTCATCCCCCTCAAGCCGGTCCCAGGCTGAGAGGCT |
| | AAAGCTTGTCTTTGCGCGTGATATGCAGCTCCAGCTTTTGTTCCCTTTAGTGAGGG |
| | TTAATTGCGCGCTTGGCGTAATCATGGTCATAGCTGTTTCCTGTGTGAAATTGTTA |
| | TCCGCTCACAATTCCACACAACATACGAGCCGGAAGCATAAAGTGTAAAGCCTGGG |
| | GTGCCTAATGAGTGAGCTAACTCACATTAATTGCGTTGCGCTCACTGCCCGCTTTC |
| | CAGTCGGGAAACCTGTCGTGCCAGCTGCATTAATGAATCGGCCAACGCGCGGGGAG |
| | AGGCGGTTTGCGTATTGGGCGCTCTTCCGCTTCCTCGCTCACTGACTCGCTGCGCT |
| | CGGTCGTTCGGCTGCGGCGAGCGGTATCAGCTCACTCAAAGGCGGTAATACGGTTA |
| | TCCACAGAATCAGGGGATAACGCAGGAAAGAACATGTGAGCAAAAGGCCAGCAAAA |
| | GGCCAGGAACCGTAAAAAGGCCGCGTTGCTGGCGTTTTTCCATAGGCTCCGCCCCC |
| | CTGACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTTCC |
| | GACCCTGCCGCTTACCGGATACCTGTCCGCCTTTCTCCCTTCGGGAAGCGTGGCGC |
| | TTTCTCATAGCTCACGCTGTAGGTATCTCAGTTCGGTGTAGGTCGTTCGCTCCAAG |
| | CTGGGCTGTGTGCACGAACCCCCCGTTCAGCCCGACCGCTGCGCCTTATCCGGTAA |
| | CTATCGTCTTGAGTCCAACCCGGTAAGACACGACTTATCGCCACTGGCAGCAGCCA |
| | CTGGTAACAGGATTAGCAGAGCGAGGTATGTAGGCGGTGCTACAGAGTTCTTGAAG |
| | TGGTGGCCTAACTACGGCTACACTAGAAGGACAGTATTTGGTATCTGCGCTCTGCT |
| | GAAGCCAGTTACCTTCGGAAAAAGAGTTGGTAGCTCTTGATCCGGCAAACAAACCA |
| | CCGCTGGTAGCGGTGGTTTTTTTGTTTGCAAGCAGCAGATTACGCGCAGAAAAAAA |
| | GGATCTCAAGAAGATCCTTTGATCTTTTCTACGGGGTCTGACGCTCAGTGGAACGA |
| | AAACTCACGTTAAGGGATTTTGGTCATGAGATTATCAAAAAGGATCTTCACCTAGA |
| | TCCTTTTAAATTAAAAATGAAGTTTTAAATCAATCTAAAGTATATATGAGTAAACT |
| | TGGTCTGACAGTTACCAATGCTTAATCAGTGAGGCACCTATCTCAGCGATCTGTCT |
| | ATTTCGTTCATCCATAGTTGCCTGACTCCCCGTCGTGTAGATAACTACGATACGGG |
| | AGGGCTTACCATCTGGCCCCAGTGCTGCAATGATACCGCGAGACCCACGCTCACCG |
| | GCTCCAGATTTATCAGCAATAAACCAGCCAGCCGGAAGGGCCGAGCGCAGAAGTGG |
| | TCCTGCAACTTTATCCGCCTCCATCCAGTCTATTAATTGTTGCCGGGAAGCTAGAG |
| | TAAGTAGTTCGCCAGTTAATAGTTTGCGCAACGTTGTTGCCATTGCTACAGGCATC |
| | GTGGTGTCACGCTCGTCGTTTGGTATGGCTTCATTCAGCTCCGGTTCCCAACGATC |
| | AAGGCGAGTTACATGATCCCCCATGTTGTGCAAAAAAGCGGTTAGCTCCTTCGGTC |
| | CTCCGATCGTTGTCAGAAGTAAGTTGGCCGCAGTGTTATCACTCATGGTTATGGCA |
| | GCACTGCATAATTCTCTTACTGTCATGCCATCCGTAAGATGCTTTTCTGTGACTGG |
| | TGAGTACTCAACCAAGTCATTCTGAGAATAGTGTATGCGGCGACCGAGTTGCTCTT |
| | GCCCGGCGTCAATACGGGATAATACCGCGCCACATAGC (SEQ ID NO: 16) |
| CLTA4 ON- | GCCGGGCAAGAGCAACTCGGTCGCCGCATACACTATTCTCAGAATGACTTGGTTGA |
| target: | GTACTCACCAGTCACAGAAAAGCATCTTACGGATGGCATGACAGTAAGAGAATTAT |
| | GCAGTGCTGCCATAACCATGAGTGATAACACTGCGGCCAACTTACTTCTGACAACG |
| | ATCGGAGGACCGAAGGAGCTAACCGCTTTTTTGCACAACATGGGGGATCATGTAAC |
| | TCGCCTTGATCGTTGGGAACCGGAGCTGAATGAAGCCATACCAAACGACGAGCGTG |
| | ACACCACGATGCCTGTAGCAATGGCAACAACGTTGCGCAAACTATTAACTGGCGAA |
| | CTACTTACTCTAGCTTCCCGGCAACAATTAATAGACTGGATGGAGGCGGATAAAGT |
| | TGCAGGACCACTTCTGCGCTCGGCCCTTCCGGCTGGCTGGTTTATTGCTGATAAAT |
| | CTGGAGCCGGTGAGCGTGGGTCTCGCGGTATCATTGCAGCACTGGGGCCAGATGGT |
| | AAGCCCTCCCGTATCGTAGTTATCTACACGACGGGGAGTCAGGCAACTATGGATGA |
| | ACGAAATAGACAGATCGCTGAGATAGGTGCCTCACTGATTAAGCATTGGTAACTGT |
| | CAGACCAAGTTTACTCATATATACTTTAGATTGATTTAAAACTTCATTTTTAATTT |
| | AAAAGGATCTAGGTGAAGATCCTTTTTGATAATCTCATGACCAAAATCCCTTAACG |
| | TGAGTTTTCGTTCCACtGaGCGTCAGACCCCGTAGAAAAGATCAAAGGATCTTCTT |
| | GAGATCCTTTTTTTCTGCGCGTAATCTGCTGCTTGCAAACAAAAAAACCACCGCTA |
| | CCAGCGGTGGTTTGTTTGCCGGATCAAGAGCTACCAACTCTTTTTCCGAAGGTAAC |
| | TGGCTTCAGCAGAGCGCAGATACCAAATACTGTTCTTCTAGTGTAGCCGTAGTTAG |
| | GCCACCACTTCAAGAACTCTGTAGCACCGCCACATACCTCGCTCTGCTAATCCTGT |
| | TACCAGTGGCTGCTTGCCAGTGGCGATAAGTCGTGTCTTACCGGGTTGGACTCAAG |
| | ACGATAGTTACCGGATAAGGCGCAGCGGTCGGGCTGAACGGGGGGTTCGTGCACAC |
| | AGCCCAGCTTGGAGCGAACGACCTACACCGAACTGAGATACCTACAGCGTGAGCTA |
| | TGAGAAAGCGCCACGCTTCCCGAAGGGAGAAAGGCGGACAGGTATCCGGTAAGCGG |
| | CAGGGTCGGAACAGGAGAGCGCACGAGGGAGCTTCCAGGGGGAAACGCCTGGTATC |
| | TTTATAGTCCTGTCGGGTTTCGCCACCTCTGACTTGAGCGTCGATTTTTGTGATGC |
| | TCGTCAGGGGGGCGGAGCCTATGGAAAAACGCCAGCAACGCGGCCTTTTTACGGTT |
| | CCTGGCCTTTTGCTGGCCTTTTGCTCACATGTTCTTTCCTGCGTTATCCCCTGATT |
| | CTGTGGATAACCGTATTACCGCCTTTGAGTGAGCTGATACCGCTCGCCGCAGCCGA |
| | ACGACCGAGCGCAGCGAGTCAGTGAGCGAGGAAGCGGAAGAGCGCCCAATACGCAA |
| | ACCGCCTCTCCCCGCGCGTTGGCCGATTCATTAATGCAGCTGGCACGACAGGTTTC |
| | CCGACTGGAAAGCGGGCAGTGAGCGCAACGCAATTAATGTGAGTTAGCTCACTCAT |
| | TAGGCACCCCAGGCTTTACACTTTATGCTTCCGGCTCGTATGTTGTGTGGAATTGT |
| | GAGCGGATAACAATTTCACACAGGAAACAGCTATGACCATGATTACGCCAAGCGCG |
| | CAATTAACCCTCACTAAAGGGAACAAAAGCTGGGTACCGGGCCCCCCCTCGACACC |
| | AGTTGCATTCGATTCCTGTTTGTAATTGTCCAATTCCTGCAGCCCGGGGGATCGGC |
| | AGATGTAGTGTTTCCACAGGGGATCCACTAGTTCTAGAGCGGCCGCCACCGCGGTG |
| | GAGCTCCAATTCGCCCTATAGTGAGTCGTATTACGCGCGCTCACTGGCCGTCGTTT |
| | TACAACGTCGTGACTGGGAAAACCCTGGCGTTACCCAACTTAATCGCCTTGCAGCA |
| | CATCCCCCTTTCGCCAGCTGGCGTAATAGCGAAGAGGCCCGCACCGATCGCCCTTC |
| | CCAACAGTTGCGCAGCCTGAATGGCGAATGGAAATTGTAAGCGTTAATATTTTGTT |
| | AAAATTCGCGTTAAATTTTTGTTAAATCAGCTCATTTTTTAACCAATAGGCCGAAA |
| | TCGGCAAAATCCCTTATAAATCAAAAGAATAGACCGAGATAGGGTTGAGTGTTGTT |
| | CCAGTTTGGAACAAGAGTCCACTATTAAAGAACGTGGACTCCAACGTCAAAGGGCG |
| | AAAAACCGTCTATCAGGGCGATGGCCCACTACGTGAACCATCACCCTAATCAAGTT |
| | TTTTGGGGTCGAGGTGCCGTAAAGCACTAAATCGGAACCCTAAAGGGAGCCCCCGA |
| | TTTAGAGCTTGACGGGGAAAGCCGGCGAACGTGGCGAGAAAGGAAGGGAAGAAAGC |
| | GAAAGGAGCGGGCGCTAGGGCGCTGGCAAGTGTAGCGGTCACGCTGCGCGTAACCA |
| | CCACACCCGCCGCGCTTAATGCGCCGCTACAGGGCGCGTCAGGTGGCACTTTTCGG |
| | GGAAATGTGCGCGGAACCCCTATTTGTTTATTTTTCTAAATACATTCAAATATGTA |
| | TCCGCTCATGAGACAATAACCCTGATAAATGCTTCAATAATATTGAAAAAGGAAGA |
| | GTATGAGTATTCAACATTTCCGTGTCGCCCTTATTCCCTTTTTTGCGGCATTTTGC |
| | CTTCCTGTTTTTGCTCACCCAGAAACGCTGGTGAAAGTAAAAGATGCTGAAGATCA |
| | GTTGGGTGCACGAGTGGGTTACATCGAACTGGATCTCAACAGCGGTAAGATCCTTG |
| | AGAGTTTTCGCCCCGAAGAACGTTTTCCAATGATGAGCACTTTTAAAGTTCTGCTA |
| | TGTGGCGCGGTATTATCCCGTATTGAC (SEQ ID NO: 17) |
| CLTA4mg ON- | GCGTTTCTGGGTGAGCAAAAACAGGAAGGCAAAATGCCGCAAAAAAGGGAATAAGG |
| target: | GCGACACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAATA |
| | AACAAATAGGGGTTCCGCGCACATTTCCCCGAAAAGTGCCACCTAAATTGTAAGCG |
| | TTAATATTTTGTTAAAATTCGCGTTAAATTTTTGTTAAATCAGCTCATTTTTTAAC |
| | CAATAGGCCGAAATCGGCAAAATCCCTTATAAATCAAAAGAATAGACCGAGATAGG |
| | GTTGAGTGTTGTTCCAGTTTGGAACAAGAGTCCACTATTAAAGAACGTGGACTCCA |
| | ACGTCAAAGGGCGAAAAACCGTCTATCAGGGCGATGGCCCACTACGTGAACCATCA |
| | CCCTAATCAAGTTTTTTGGGGTCGAGGTGCCGTAAAGCACTAAATCGGAACCCTAA |
| | AGGGAGCCCCCGATTTAGAGCTTGACGGGGAAAGCCGGCGAACGTGGCGAGAAAGG |
| | AAGGGAAGAAAGCGAAAGGAGCGGGCGCTAGGGCGCTGGCAAGTGTAGCGGTCACG |
| | CTGCGCGTAACCACCACACCCGCCGCGCTTAATGCGCCGCTACAGGGCGCGTCCCA |
| | TTCGCCATTCAGGCTGCGCAACTGTTGGGAAGGGCGATCGGTGCGGGCCTCTTCGC |
| | TATTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAGCGCGCGTAATA |
| | CGACTCACTATAGGGCGAATTGGGTACGATCGATGCGGCCTCAAGAGCTTCACTGA |
| | GTAGGATTAAGATATTGCAGATGTAGTGTTTCCACAGGGTGGCTCTTCAGTGCACC |
| | AGCGGAACCTGCTGCGCGTGATATGCAGCTCCAGCTTTTGTTCCCTTTAGTGAGGG |
| | TTAATTGCGCGCTTGGCGTAATCATGGTCATAGCTGTTTCCTGTGTGAAATTGTTA |
| | TCCGCTCACAATTCCACACAACATACGAGCCGGAAGCATAAAGTGTAAAGCCTGGG |
| | GTGCCTAATGAGTGAGCTAACTCACATTAATTGCGTTGCGCTCACTGCCCGCTTTC |
| | CAGTCGGGAAACCTGTCGTGCCAGCTGCATTAATGAATCGGCCAACGCGCGGGGAG |
| | AGGCGGTTTGCGTATTGGGCGCTCTTCCGCTTCCTCGCTCACTGACTCGCTGCGCT |
| | CGGTCGTTCGGCTGCGGCGAGCGGTATCAGCTCACTCAAAGGCGGTAATACGGTTA |
| | TCCACAGAATCAGGGGATAACGCAGGAAAGAACATGTGAGCAAAAGGCCAGCAAAA |
| | GGCCAGGAACCGTAAAAAGGCCGCGTTGCTGGCGTTTTTCCATAGGCTCCGCCCCC |
| | CTGACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTTCC |
| | GACCCTGCCGCTTACCGGATACCTGTCCGCCTTTCTCCCTTCGGGAAGCGTGGCGC |
| | TTTCTCATAGCTCACGCTGTAGGTATCTCAGTTCGGTGTAGGTCGTTCGCTCCAAG |
| | CTGGGCTGTGTGCACGAACCCCCCGTTCAGCCCGACCGCTGCGCCTTATCCGGTAA |
| | CTATCGTCTTGAGTCCAACCCGGTAAGACACGACTTATCGCCACTGGCAGCAGCCA |
| | CTGGTAACAGGATTAGCAGAGCGAGGTATGTAGGCGGTGCTACAGAGTTCTTGAAG |
| | TGGTGGCCTAACTACGGCTACACTAGAAGGACAGTATTTGGTATCTGCGCTCTGCT |
| | GAAGCCAGTTACCTTCGGAAAAAGAGTTGGTAGCTCTTGATCCGGCAAACAAACCA |
| | CCGCTGGTAGCGGTGGTTTTTTTGTTTGCAAGCAGCAGATTACGCGCAGAAAAAAA |
| | GGATCTCAAGAAGATCCTTTGATCTTTTCTACGGGGTCTGACGCTCAGTGGAACGA |
| | AAACTCACGTTAAGGGATTTTGGTCATGAGATTATCAAAAAGGATCTTCACCTAGA |
| | TCCTTTTAAATTAAAAATGAAGTTTTAAATCAATCTAAAGTATATATGAGTAAACT |
| | TGGTCTGACAGTTACCAATGCTTAATCAGTGAGGCACCTATCTCAGCGATCTGTCT |
| | ATTTCGTTCATCCATAGTTGCCTGACTCCCCGTCGTGTAGATAACTACGATACGGG |
| | AGGGCTTACCATCTGGCCCCAGTGCTGCAATGATACCGCGAGACCCACGCTCACCG |
| | GCTCCAGATTTATCAGCAATAAACCAGCCAGCCGGAAGGGCCGAGCGCAGAAGTGG |
| | TCCTGCAACTTTATCCGCCTCCATCCAGTCTATTAATTGTTGCCGGGAAGCTAGAG |
| | TAAGTAGTTCGCCAGTTAATAGTTTGCGCAACGTTGTTGCCATTGCTACAGGCATC |
| | GTGGTGTCACGCTCGTCGTTTGGTATGGCTTCATTCAGCTCCGGTTCCCAACGATC |
| | AAGGCGAGTTACATGATCCCCCATGTTGTGCAAAAAAGCGGTTAGCTCCTTCGGTC |
| | CTCCGATCGTTGTCAGAAGTAAGTTGGCCGCAGTGTTATCACTCATGGTTATGGCA |
| | GCACTGCATAATTCTCTTACTGTCATGCCATCCGTAAGATGCTTTTCTGTGACTGG |
| | TGAGTACTCAACCAAGTCATTCTGAGAATAGTGTATGCGGCGACCGAGTTGCTCTT |
| | GCCCGGCGTCAATACGGGATAATACCGCGCCACATAGC (SEQ ID NO: 18) |
| CLTA4mg OFF5- | GCGTTTCTGGGTGAGCAAAAACAGGAAGGCAAAATGCCGCAAAAAAGGGAATAAGG |
| target: | GCGACACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAATA |
| | AACAAATAGGGGTTCCGCGCACATTTCCCCGAAAAGTGCCACCTAAATTGTAAGCG |
| | TTAATATTTTGTTAAAATTCGCGTTAAATTTTTGTTAAATCAGCTCATTTTTTAAC |
| | CAATAGGCCGAAATCGGCAAAATCCCTTATAAATCAAAAGAATAGACCGAGATAGG |
| | GTTGAGTGTTGTTCCAGTTTGGAACAAGAGTCCACTATTAAAGAACGTGGACTCCA |
| | ACGTCAAAGGGCGAAAAACCGTCTATCAGGGCGATGGCCCACTACGTGAACCATCA |
| | CCCTAATCAAGTTTTTTGGGGTCGAGGTGCCGTAAAGCACTAAATCGGAACCCTAA |
| | AGGGAGCCCCCGATTTAGAGCTTGACGGGGAAAGCCGGCGAACGTGGCGAGAAAGG |
| | AAGGGAAGAAAGCGAAAGGAGCGGGCGCTAGGGCGCTGGCAAGTGTAGCGGTCACG |
| | CTGCGCGTAACCACCACACCCGCCGCGCTTAATGCGCCGCTACAGGGCGCGTCCCA |
| | TTCGCCATTCAGGCTGCGCAACTGTTGGGAAGGGCGATCGGTGCGGGCCTCTTCGC |
| | TATTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAGCGCGCGTAATA |
| | CGACTCACTATAGGGCGAATTGGGTACGATCGATGCGGCCTCTCTGAATAGAGTTG |
| | GGAAGAGATGCATACAACATATGTAGTATTTCCACAGGGAATACAATGGACAAATG |
| | ACCTCAAGAGCAGGCGCGTGATATGCAGCTCCAGCTTTTGTTCCCTTTAGTGAGGG |
| | TTAATTGCGCGCTTGGCGTAATCATGGTCATAGCTGTTTCCTGTGTGAAATTGTTA |
| | TCCGCTCACAATTCCACACAACATACGAGCCGGAAGCATAAAGTGTAAAGCCTGGG |
| | GTGCCTAATGAGTGAGCTAACTCACATTAATTGCGTTGCGCTCACTGCCCGCTTTC |
| | CAGTCGGGAAACCTGTCGTGCCAGCTGCATTAATGAATCGGCCAACGCGCGGGGAG |
| | AGGCGGTTTGCGTATTGGGCGCTCTTCCGCTTCCTCGCTCACTGACTCGCTGCGCT |
| | CGGTCGTTCGGCTGCGGCGAGCGGTATCAGCTCACTCAAAGGCGGTAATACGGTTA |
| | TCCACAGAATCAGGGGATAACGCAGGAAAGAACATGTGAGCAAAAGGCCAGCAAAA |
| | GGCCAGGAACCGTAAAAAGGCCGCGTTGCTGGCGTTTTTCCATAGGCTCCGCCCCC |
| | CTGACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTTCC |
| | GACCCTGCCGCTTACCGGATACCTGTCCGCCTTTCTCCCTTCGGGAAGCGTGGCGC |
| | TTTCTCATAGCTCACGCTGTAGGTATCTCAGTTCGGTGTAGGTCGTTCGCTCCAAG |
| | CTGGGCTGTGTGCACGAACCCCCCGTTCAGCCCGACCGCTGCGCCTTATCCGGTAA |
| | CTATCGTCTTGAGTCCAACCCGGTAAGACACGACTTATCGCCACTGGCAGCAGCCA |
| | CTGGTAACAGGATTAGCAGAGCGAGGTATGTAGGCGGTGCTACAGAGTTCTTGAAG |
| | TGGTGGCCTAACTACGGCTACACTAGAAGGACAGTATTTGGTATCTGCGCTCTGCT |
| | GAAGCCAGTTACCTTCGGAAAAAGAGTTGGTAGCTCTTGATCCGGCAAACAAACCA |
| | CCGCTGGTAGCGGTGGTTTTTTTGTTTGCAAGCAGCAGATTACGCGCAGAAAAAAA |
| | GGATCTCAAGAAGATCCTTTGATCTTTTCTACGGGGTCTGACGCTCAGTGGAACGA |
| | AAACTCACGTTAAGGGATTTTGGTCATGAGATTATCAAAAAGGATCTTCACCTAGA |
| | TCCTTTTAAATTAAAAATGAAGTTTTAAATCAATCTAAAGTATATATGAGTAAACT |
| | TGGTCTGACAGTTACCAATGCTTAATCAGTGAGGCACCTATCTCAGCGATCTGTCT |
| | ATTTCGTTCATCCATAGTTGCCTGACTCCCCGTCGTGTAGATAACTACGATACGGG |
| | AGGGCTTACCATCTGGCCCCAGTGCTGCAATGATACCGCGAGACCCACGCTCACCG |
| | GCTCCAGATTTATCAGCAATAAACCAGCCAGCCGGAAGGGCCGAGCGCAGAAGTGG |
| | TCCTGCAACTTTATCCGCCTCCATCCAGTCTATTAATTGTTGCCGGGAAGCTAGAG |
| | TAAGTAGTTCGCCAGTTAATAGTTTGCGCAACGTTGTTGCCATTGCTACAGGCATC |
| | GTGGTGTCACGCTCGTCGTTTGGTATGGCTTCATTCAGCTCCGGTTCCCAACGATC |
| | AAGGCGAGTTACATGATCCCCCATGTTGTGCAAAAAAGCGGTTAGCTCCTTCGGTC |
| | CTCCGATCGTTGTCAGAAGTAAGTTGGCCGCAGTGTTATCACTCATGGTTATGGCA |
| | GCACTGCATAATTCTCTTACTGTCATGCCATCCGTAAGATGCTTTTCTGTGACTGG |
| | TGAGTACTCAACCAAGTCATTCTGAGAATAGTGTATGCGGCGACCGAGTTGCTCTT |
| | GCCCGGCGTCAATACGGGATAATACCGCGCCACATAGC (SEQ ID NO: 19) |
| IL2RGmg_ON | GCGTTTCTGGGTGAGCAAAAACAGGAAGGCAAAATGCCGCAAAAAAGGGAATAAGG |
| target: | GCGACACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAATA |
| | AACAAATAGGGGTTCCGCGCACATTTCCCCGAAAAGTGCCACCTAAATTGTAAGCG |
| | TTAATATTTTGTTAAAATTCGCGTTAAATTTTTGTTAAATCAGCTCATTTTTTAAC |
| | CAATAGGCCGAAATCGGCAAAATCCCTTATAAATCAAAAGAATAGACCGAGATAGG |
| | GTTGAGTGTTGTTCCAGTTTGGAACAAGAGTCCACTATTAAAGAACGTGGACTCCA |
| | ACGTCAAAGGGCGAAAAACCGTCTATCAGGGCGATGGCCCACTACGTGAACCATCA |
| | CCCTAATCAAGTTTTTTGGGGTCGAGGTGCCGTAAAGCACTAAATCGGAACCCTAA |
| | AGGGAGCCCCCGATTTAGAGCTTGACGGGGAAAGCCGGCGAACGTGGCGAGAAAGG |
| | AAGGGAAGAAAGCGAAAGGAGCGGGCGCTAGGGCGCTGGCAAGTGTAGCGGTCACG |
| | CTGCGCGTAACCACCACACCCGCCGCGCTTAATGCGCCGCTACAGGGCGCGTCCCA |
| | TTCGCCATTCAGGCTGCGCAACTGTTGGGAAGGGCGATCGGTGCGGGCCTCTTCGC |
| | TATTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAGCGCGCGTAATA |
| | CGACTCACTATAGGGCGAATTGGGTACGATCGATGCGGCCTCGGGCAGCTGCAGGA |
| | ATAAGAGGGATGTGAATGGTAATGATGGCTTCAACATGGCGCTTGCTCTTCATTCC |
| | CTGGGTGTAGTCTGCGCGTGATATGCAGCTCCAGCTTTTGTTCCCTTTAGTGAGGG |
| | TTAATTGCGCGCTTGGCGTAATCATGGTCATAGCTGTTTCCTGTGTGAAATTGTTA |
| | TCCGCTCACAATTCCACACAACATACGAGCCGGAAGCATAAAGTGTAAAGCCTGGG |
| | GTGCCTAATGAGTGAGCTAACTCACATTAATTGCGTTGCGCTCACTGCCCGCTTTC |
| | CAGTCGGGAAACCTGTCGTGCCAGCTGCATTAATGAATCGGCCAACGCGCGGGGAG |
| | AGGCGGTTTGCGTATTGGGCGCTCTTCCGCTTCCTCGCTCACTGACTCGCTGCGCT |
| | CGGTCGTTCGGCTGCGGCGAGCGGTATCAGCTCACTCAAAGGCGGTAATACGGTTA |
| | TCCACAGAATCAGGGGATAACGCAGGAAAGAACATGTGAGCAAAAGGCCAGCAAAA |
| | GGCCAGGAACCGTAAAAAGGCCGCGTTGCTGGCGTTTTTCCATAGGCTCCGCCCCC |
| | CTGACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTTCC |
| | GACCCTGCCGCTTACCGGATACCTGTCCGCCTTTCTCCCTTCGGGAAGCGTGGCGC |
| | TTTCTCATAGCTCACGCTGTAGGTATCTCAGTTCGGTGTAGGTCGTTCGCTCCAAG |
| | CTGGGCTGTGTGCACGAACCCCCCGTTCAGCCCGACCGCTGCGCCTTATCCGGTAA |
| | CTATCGTCTTGAGTCCAACCCGGTAAGACACGACTTATCGCCACTGGCAGCAGCCA |
| | CTGGTAACAGGATTAGCAGAGCGAGGTATGTAGGCGGTGCTACAGAGTTCTTGAAG |
| | TGGTGGCCTAACTACGGCTACACTAGAAGGACAGTATTTGGTATCTGCGCTCTGCT |
| | GAAGCCAGTTACCTTCGGAAAAAGAGTTGGTAGCTCTTGATCCGGCAAACAAACCA |
| | CCGCTGGTAGCGGTGGTTTTTTTGTTTGCAAGCAGCAGATTACGCGCAGAAAAAAA |
| | GGATCTCAAGAAGATCCTTTGATCTTTTCTACGGGGTCTGACGCTCAGTGGAACGA |
| | AAACTCACGTTAAGGGATTTTGGTCATGAGATTATCAAAAAGGATCTTCACCTAGA |
| | TCCTTTTAAATTAAAAATGAAGTTTTAAATCAATCTAAAGTATATATGAGTAAACT |
| | TGGTCTGACAGTTACCAATGCTTAATCAGTGAGGCACCTATCTCAGCGATCTGTCT |
| | ATTTCGTTCATCCATAGTTGCCTGACTCCCCGTCGTGTAGATAACTACGATACGGG |
| | AGGGCTTACCATCTGGCCCCAGTGCTGCAATGATACCGCGAGACCCACGCTCACCG |
| | GCTCCAGATTTATCAGCAATAAACCAGCCAGCCGGAAGGGCCGAGCGCAGAAGTGG |
| | TCCTGCAACTTTATCCGCCTCCATCCAGTCTATTAATTGTTGCCGGGAAGCTAGAG |
| | TAAGTAGTTCGCCAGTTAATAGTTTGCGCAACGTTGTTGCCATTGCTACAGGCATC |
| | GTGGTGTCACGCTCGTCGTTTGGTATGGCTTCATTCAGCTCCGGTTCCCAACGATC |
| | AAGGCGAGTTACATGATCCCCCATGTTGTGCAAAAAAGCGGTTAGCTCCTTCGGTC |
| | CTCCGATCGTTGTCAGAAGTAAGTTGGCCGCAGTGTTATCACTCATGGTTATGGCA |
| | GCACTGCATAATTCTCTTACTGTCATGCCATCCGTAAGATGCTTTTCTGTGACTGG |
| | TGAGTACTCAACCAAGTCATTCTGAGAATAGTGTATGCGGCGACCGAGTTGCTCTT |
| | GCCCGGCGTCAATACGGGATAATACCGCGCCACATAGC (SEQ ID NO: 20) |
| EN1mg_ON | GCGTTTCTGGGTGAGCAAAAACAGGAAGGCAAAATGCCGCAAAAAAGGGAATAAGG |
| target: | GCGACACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAATA |
| | AACAAATAGGGGTTCCGCGCACATTTCCCCGAAAAGTGCCACCTAAATTGTAAGCG |
| | TTAATATTTTGTTAAAATTCGCGTTAAATTTTTGTTAAATCAGCTCATTTTTTAAC |
| | CAATAGGCCGAAATCGGCAAAATCCCTTATAAATCAAAAGAATAGACCGAGATAGG |
| | GTTGAGTGTTGTTCCAGTTTGGAACAAGAGTCCACTATTAAAGAACGTGGACTCCA |
| | ACGTCAAAGGGCGAAAAACCGTCTATCAGGGCGATGGCCCACTACGTGAACCATCA |
| | CCCTAATCAAGTTTTTTGGGGTCGAGGTGCCGTAAAGCACTAAATCGGAACCCTAA |
| | AGGGAGCCCCCGATTTAGAGCTTGACGGGGAAAGCCGGCGAACGTGGCGAGAAAGG |
| | AAGGGAAGAAAGCGAAAGGAGCGGGCGCTAGGGCGCTGGCAAGTGTAGCGGTCACG |
| | CTGCGCGTAACCACCACACCCGCCGCGCTTAATGCGCCGCTACAGGGCGCGTCCCA |
| | TTCGCCATTCAGGCTGCGCAACTGTTGGGAAGGGCGATCGGTGCGGGCCTCTTCGC |
| | TATTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAGCGCGCGTAATA |
| | CGACTCACTATAGGGCGAATTGGGTACGATCGATGCGGCCTCCTCCTTACTGCAGC |
| | CGAAGTCCGGCCTCAGGATGTTGTCGATGAAAAAGTTGGTGGTGCGGTGCAGCTGG |
| | GCCGCTGGCTGCGGCGCGTGATATGCAGCTCCAGCTTTTGTTCCCTTTAGTGAGGG |
| | TTAATTGCGCGCTTGGCGTAATCATGGTCATAGCTGTTTCCTGTGTGAAATTGTTA |
| | TCCGCTCACAATTCCACACAACATACGAGCCGGAAGCATAAAGTGTAAAGCCTGGG |
| | GTGCCTAATGAGTGAGCTAACTCACATTAATTGCGTTGCGCTCACTGCCCGCTTTC |
| | CAGTCGGGAAACCTGTCGTGCCAGCTGCATTAATGAATCGGCCAACGCGCGGGGAG |
| | AGGCGGTTTGCGTATTGGGCGCTCTTCCGCTTCCTCGCTCACTGACTCGCTGCGCT |
| | CGGTCGTTCGGCTGCGGCGAGCGGTATCAGCTCACTCAAAGGCGGTAATACGGTTA |
| | TCCACAGAATCAGGGGATAACGCAGGAAAGAACATGTGAGCAAAAGGCCAGCAAAA |
| | GGCCAGGAACCGTAAAAAGGCCGCGTTGCTGGCGTTTTTCCATAGGCTCCGCCCCC |
| | CTGACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTTCC |
| | GACCCTGCCGCTTACCGGATACCTGTCCGCCTTTCTCCCTTCGGGAAGCGTGGCGC |
| | TTTCTCATAGCTCACGCTGTAGGTATCTCAGTTCGGTGTAGGTCGTTCGCTCCAAG |
| | CTGGGCTGTGTGCACGAACCCCCCGTTCAGCCCGACCGCTGCGCCTTATCCGGTAA |
| | CTATCGTCTTGAGTCCAACCCGGTAAGACACGACTTATCGCCACTGGCAGCAGCCA |
| | CTGGTAACAGGATTAGCAGAGCGAGGTATGTAGGCGGTGCTACAGAGTTCTTGAAG |
| | TGGTGGCCTAACTACGGCTACACTAGAAGGACAGTATTTGGTATCTGCGCTCTGCT |
| | GAAGCCAGTTACCTTCGGAAAAAGAGTTGGTAGCTCTTGATCCGGCAAACAAACCA |
| | CCGCTGGTAGCGGTGGTTTTTTTGTTTGCAAGCAGCAGATTACGCGCAGAAAAAAA |
| | GGATCTCAAGAAGATCCTTTGATCTTTTCTACGGGGTCTGACGCTCAGTGGAACGA |
| | AAACTCACGTTAAGGGATTTTGGTCATGAGATTATCAAAAAGGATCTTCACCTAGA |
| | TCCTTTTAAATTAAAAATGAAGTTTTAAATCAATCTAAAGTATATATGAGTAAACT |
| | TGGTCTGACAGTTACCAATGCTTAATCAGTGAGGCACCTATCTCAGCGATCTGTCT |
| | ATTTCGTTCATCCATAGTTGCCTGACTCCCCGTCGTGTAGATAACTACGATACGGG |
| | AGGGCTTACCATCTGGCCCCAGTGCTGCAATGATACCGCGAGACCCACGCTCACCG |
| | GCTCCAGATTTATCAGCAATAAACCAGCCAGCCGGAAGGGCCGAGCGCAGAAGTGG |
| | TCCTGCAACTTTATCCGCCTCCATCCAGTCTATTAATTGTTGCCGGGAAGCTAGAG |
| | TAAGTAGTTCGCCAGTTAATAGTTTGCGCAACGTTGTTGCCATTGCTACAGGCATC |
| | GTGGTGTCACGCTCGTCGTTTGGTATGGCTTCATTCAGCTCCGGTTCCCAACGATC |
| | AAGGCGAGTTACATGATCCCCCATGTTGTGCAAAAAAGCGGTTAGCTCCTTCGGTC |
| | CTCCGATCGTTGTCAGAAGTAAGTTGGCCGCAGTGTTATCACTCATGGTTATGGCA |
| | GCACTGCATAATTCTCTTACTGTCATGCCATCCGTAAGATGCTTTTCTGTGACTGG |
| | TGAGTACTCAACCAAGTCATTCTGAGAATAGTGTATGCGGCGACCGAGTTGCTCTT |
| | GCCCGGCGTCAATACGGGATAATACCGCGCCACATAGC (SEQ ID NO: 21) |
| EN1mg_OFF | GCGTTTCTGGGTGAGCAAAAACAGGAAGGCAAAATGCCGCAAAAAAGGGAATAAGG |
| target: | GCGACACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAATA |
| | AACAAATAGGGGTTCCGCGCACATTTCCCCGAAAAGTGCCACCTAAATTGTAAGCG |
| | TTAATATTTTGTTAAAATTCGCGTTAAATTTTTGTTAAATCAGCTCATTTTTTAAC |
| | CAATAGGCCGAAATCGGCAAAATCCCTTATAAATCAAAAGAATAGACCGAGATAGG |
| | GTTGAGTGTTGTTCCAGTTTGGAACAAGAGTCCACTATTAAAGAACGTGGACTCCA |
| | ACGTCAAAGGGCGAAAAACCGTCTATCAGGGCGATGGCCCACTACGTGAACCATCA |
| | CCCTAATCAAGTTTTTTGGGGTCGAGGTGCCGTAAAGCACTAAATCGGAACCCTAA |
| | AGGGAGCCCCCGATTTAGAGCTTGACGGGGAAAGCCGGCGAACGTGGCGAGAAAGG |
| | AAGGGAAGAAAGCGAAAGGAGCGGGCGCTAGGGCGCTGGCAAGTGTAGCGGTCACG |
| | CTGCGCGTAACCACCACACCCGCCGCGCTTAATGCGCCGCTACAGGGCGCGTCCCA |
| | TTCGCCATTCAGGCTGCGCAACTGTTGGGAAGGGCGATCGGTGCGGGCCTCTTCGC |
| | TATTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAGCGCGCGTAATA |
| | CGACTCACTATAGGGCGAATTGGGTACGATCGATGCGGCCTCGTCCTTTCGCCGGC |
| | CGAACTCGGGCCGCAGGATGTTGTCGATGAAGAAGTTGGTGATGCGGTGCGGGTGC |
| | TGGTGGTTGCCGGGCGCGTGATATGCAGCTCCAGCTTTTGTTCCCTTTAGTGAGGG |
| | TTAATTGCGCGCTTGGCGTAATCATGGTCATAGCTGTTTCCTGTGTGAAATTGTTA |
| | TCCGCTCACAATTCCACACAACATACGAGCCGGAAGCATAAAGTGTAAAGCCTGGG |
| | GTGCCTAATGAGTGAGCTAACTCACATTAATTGCGTTGCGCTCACTGCCCGCTTTC |
| | CAGTCGGGAAACCTGTCGTGCCAGCTGCATTAATGAATCGGCCAACGCGCGGGGAG |
| | AGGCGGTTTGCGTATTGGGCGCTCTTCCGCTTCCTCGCTCACTGACTCGCTGCGCT |
| | CGGTCGTTCGGCTGCGGCGAGCGGTATCAGCTCACTCAAAGGCGGTAATACGGTTA |
| | TCCACAGAATCAGGGGATAACGCAGGAAAGAACATGTGAGCAAAAGGCCAGCAAAA |
| | GGCCAGGAACCGTAAAAAGGCCGCGTTGCTGGCGTTTTTCCATAGGCTCCGCCCCC |
| | CTGACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTTCC |
| | GACCCTGCCGCTTACCGGATACCTGTCCGCCTTTCTCCCTTCGGGAAGCGTGGCGC |
| | TTTCTCATAGCTCACGCTGTAGGTATCTCAGTTCGGTGTAGGTCGTTCGCTCCAAG |
| | CTGGGCTGTGTGCACGAACCCCCCGTTCAGCCCGACCGCTGCGCCTTATCCGGTAA |
| | CTATCGTCTTGAGTCCAACCCGGTAAGACACGACTTATCGCCACTGGCAGCAGCCA |
| | CTGGTAACAGGATTAGCAGAGCGAGGTATGTAGGCGGTGCTACAGAGTTCTTGAAG |
| | TGGTGGCCTAACTACGGCTACACTAGAAGGACAGTATTTGGTATCTGCGCTCTGCT |
| | GAAGCCAGTTACCTTCGGAAAAAGAGTTGGTAGCTCTTGATCCGGCAAACAAACCA |
| | CCGCTGGTAGCGGTGGTTTTTTTGTTTGCAAGCAGCAGATTACGCGCAGAAAAAAA |
| | GGATCTCAAGAAGATCCTTTGATCTTTTCTACGGGGTCTGACGCTCAGTGGAACGA |
| | AAACTCACGTTAAGGGATTTTGGTCATGAGATTATCAAAAAGGATCTTCACCTAGA |
| | TCCTTTTAAATTAAAAATGAAGTTTTAAATCAATCTAAAGTATATATGAGTAAACT |
| | TGGTCTGACAGTTACCAATGCTTAATCAGTGAGGCACCTATCTCAGCGATCTGTCT |
| | ATTTCGTTCATCCATAGTTGCCTGACTCCCCGTCGTGTAGATAACTACGATACGGG |
| | AGGGCTTACCATCTGGCCCCAGTGCTGCAATGATACCGCGAGACCCACGCTCACCG |
| | GCTCCAGATTTATCAGCAATAAACCAGCCAGCCGGAAGGGCCGAGCGCAGAAGTGG |
| | TCCTGCAACTTTATCCGCCTCCATCCAGTCTATTAATTGTTGCCGGGAAGCTAGAG |
| | TAAGTAGTTCGCCAGTTAATAGTTTGCGCAACGTTGTTGCCATTGCTACAGGCATC |
| | GTGGTGTCACGCTCGTCGTTTGGTATGGCTTCATTCAGCTCCGGTTCCCAACGATC |
| | AAGGCGAGTTACATGATCCCCCATGTTGTGCAAAAAAGCGGTTAGCTCCTTCGGTC |
| | CTCCGATCGTTGTCAGAAGTAAGTTGGCCGCAGTGTTATCACTCATGGTTATGGCA |
| | GCACTGCATAATTCTCTTACTGTCATGCCATCCGTAAGATGCTTTTCTGTGACTGG |
| | TGAGTACTCAACCAAGTCATTCTGAGAATAGTGTATGCGGCGACCGAGTTGCTCTT |
| | GCCCGGCGTCAATACGGGATAATACCGCGCCACATAGC (SEQ ID NO: 22) |
| PCDHA4mg_ ON | GCGTTTCTGGGTGAGCAAAAACAGGAAGGCAAAATGCCGCAAAAAAGGGAATAAGG |
| target: | GCGACACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAATA |
| | AACAAATAGGGGTTCCGCGCACATTTCCCCGAAAAGTGCCACCTAAATTGTAAGCG |
| | TTAATATTTTGTTAAAATTCGCGTTAAATTTTTGTTAAATCAGCTCATTTTTTAAC |
| | CAATAGGCCGAAATCGGCAAAATCCCTTATAAATCAAAAGAATAGACCGAGATAGG |
| | GTTGAGTGTTGTTCCAGTTTGGAACAAGAGTCCACTATTAAAGAACGTGGACTCCA |
| | ACGTCAAAGGGCGAAAAACCGTCTATCAGGGCGATGGCCCACTACGTGAACCATCA |
| | CCCTAATCAAGTTTTTTGGGGTCGAGGTGCCGTAAAGCACTAAATCGGAACCCTAA |
| | AGGGAGCCCCCGATTTAGAGCTTGACGGGGAAAGCCGGCGAACGTGGCGAGAAAGG |
| | AAGGGAAGAAAGCGAAAGGAGCGGGCGCTAGGGCGCTGGCAAGTGTAGCGGTCACG |
| | CTGCGCGTAACCACCACACCCGCCGCGCTTAATGCGCCGCTACAGGGCGCGTCCCA |
| | TTCGCCATTCAGGCTGCGCAACTGTTGGGAAGGGCGATCGGTGCGGGCCTCTTCGC |
| | TATTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAGCGCGCGTAATA |
| | CGACTCACTATAGGGCGAATTGGGTACGATCGATGCGGCCTCGGAACATTGGTAAT |
| | TAAACTTAACGCCTCAGATTTAGACGAAGGATTGAATGGGGACATTGTTTATTCAT |
| | TCTCGAATGATACGCGCGTGATATGCAGCTCCAGCTTTTGTTCCCTTTAGTGAGGG |
| | TTAATTGCGCGCTTGGCGTAATCATGGTCATAGCTGTTTCCTGTGTGAAATTGTTA |
| | TCCGCTCACAATTCCACACAACATACGAGCCGGAAGCATAAAGTGTAAAGCCTGGG |
| | GTGCCTAATGAGTGAGCTAACTCACATTAATTGCGTTGCGCTCACTGCCCGCTTTC |
| | CAGTCGGGAAACCTGTCGTGCCAGCTGCATTAATGAATCGGCCAACGCGCGGGGAG |
| | AGGCGGTTTGCGTATTGGGCGCTCTTCCGCTTCCTCGCTCACTGACTCGCTGCGCT |
| | CGGTCGTTCGGCTGCGGCGAGCGGTATCAGCTCACTCAAAGGCGGTAATACGGTTA |
| | TCCACAGAATCAGGGGATAACGCAGGAAAGAACATGTGAGCAAAAGGCCAGCAAAA |
| | GGCCAGGAACCGTAAAAAGGCCGCGTTGCTGGCGTTTTTCCATAGGCTCCGCCCCC |
| | CTGACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTTCC |
| | GACCCTGCCGCTTACCGGATACCTGTCCGCCTTTCTCCCTTCGGGAAGCGTGGCGC |
| | TTTCTCATAGCTCACGCTGTAGGTATCTCAGTTCGGTGTAGGTCGTTCGCTCCAAG |
| | CTGGGCTGTGTGCACGAACCCCCCGTTCAGCCCGACCGCTGCGCCTTATCCGGTAA |
| | CTATCGTCTTGAGTCCAACCCGGTAAGACACGACTTATCGCCACTGGCAGCAGCCA |
| | CTGGTAACAGGATTAGCAGAGCGAGGTATGTAGGCGGTGCTACAGAGTTCTTGAAG |
| | TGGTGGCCTAACTACGGCTACACTAGAAGGACAGTATTTGGTATCTGCGCTCTGCT |
| | GAAGCCAGTTACCTTCGGAAAAAGAGTTGGTAGCTCTTGATCCGGCAAACAAACCA |
| | CCGCTGGTAGCGGTGGTTTTTTTGTTTGCAAGCAGCAGATTACGCGCAGAAAAAAA |
| | GGATCTCAAGAAGATCCTTTGATCTTTTCTACGGGGTCTGACGCTCAGTGGAACGA |
| | AAACTCACGTTAAGGGATTTTGGTCATGAGATTATCAAAAAGGATCTTCACCTAGA |
| | TCCTTTTAAATTAAAAATGAAGTTTTAAATCAATCTAAAGTATATATGAGTAAACT |
| | TGGTCTGACAGTTACCAATGCTTAATCAGTGAGGCACCTATCTCAGCGATCTGTCT |
| | ATTTCGTTCATCCATAGTTGCCTGACTCCCCGTCGTGTAGATAACTACGATACGGG |
| | AGGGCTTACCATCTGGCCCCAGTGCTGCAATGATACCGCGAGACCCACGCTCACCG |
| | GCTCCAGATTTATCAGCAATAAACCAGCCAGCCGGAAGGGCCGAGCGCAGAAGTGG |
| | TCCTGCAACTTTATCCGCCTCCATCCAGTCTATTAATTGTTGCCGGGAAGCTAGAG |
| | TAAGTAGTTCGCCAGTTAATAGTTTGCGCAACGTTGTTGCCATTGCTACAGGCATC |
| | GTGGTGTCACGCTCGTCGTTTGGTATGGCTTCATTCAGCTCCGGTTCCCAACGATC |
| | AAGGCGAGTTACATGATCCCCCATGTTGTGCAAAAAAGCGGTTAGCTCCTTCGGTC |
| | CTCCGATCGTTGTCAGAAGTAAGTTGGCCGCAGTGTTATCACTCATGGTTATGGCA |
| | GCACTGCATAATTCTCTTACTGTCATGCCATCCGTAAGATGCTTTTCTGTGACTGG |
| | TGAGTACTCAACCAAGTCATTCTGAGAATAGTGTATGCGGCGACCGAGTTGCTCTT |
| | GCCCGGCGTCAATACGGGATAATACCGCGCCACATAGC (SEQ ID NO: 23) |
| PCDHA4mg_OFF | GCGTTTCTGGGTGAGCAAAAACAGGAAGGCAAAATGCCGCAAAAAAGGGAATAAGG |
| target: | GCGACACGGAAATGTTGAATACTCATACTCTTCCTTTTTCAATATTATTGAAGCAT |
| | TTATCAGGGTTATTGTCTCATGAGCGGATACATATTTGAATGTATTTAGAAAAATA |
| | AACAAATAGGGGTTCCGCGCACATTTCCCCGAAAAGTGCCACCTAAATTGTAAGCG |
| | TTAATATTTTGTTAAAATTCGCGTTAAATTTTTGTTAAATCAGCTCATTTTTTAAC |
| | CAATAGGCCGAAATCGGCAAAATCCCTTATAAATCAAAAGAATAGACCGAGATAGG |
| | GTTGAGTGTTGTTCCAGTTTGGAACAAGAGTCCACTATTAAAGAACGTGGACTCCA |
| | ACGTCAAAGGGCGAAAAACCGTCTATCAGGGCGATGGCCCACTACGTGAACCATCA |
| | CCCTAATCAAGTTTTTTGGGGTCGAGGTGCCGTAAAGCACTAAATCGGAACCCTAA |
| | AGGGAGCCCCCGATTTAGAGCTTGACGGGGAAAGCCGGCGAACGTGGCGAGAAAGG |
| | AAGGGAAGAAAGCGAAAGGAGCGGGCGCTAGGGCGCTGGCAAGTGTAGCGGTCACG |
| | CTGCGCGTAACCACCACACCCGCCGCGCTTAATGCGCCGCTACAGGGCGCGTCCCA |
| | TTCGCCATTCAGGCTGCGCAACTGTTGGGAAGGGCGATCGGTGCGGGCCTCTTCGC |
| | TATTACGCCAGCTGGCGAAAGGGGGATGTGCTGCAAGGCGATTAAGTTGGGTAACG |
| | CCAGGGTTTTCCCAGTCACGACGTTGTAAAACGACGGCCAGTGAGCGCGCGTAATA |
| | CGACTCACTATAGGGCGAATTGGGTACGATCGATGCGGCCTCGGAACGCTGGTGAT |
| | TCATCCCAATGCCTCAGATTTAGACGAAGGCTTGAATGGGGATATTATTTACTCCT |
| | TCTCCAGTGATGTGCGCGTGATATGCAGCTCCAGCTTTTGTTCCCTTTAGTGAGGG |
| | TTAATTGCGCGCTTGGCGTAATCATGGTCATAGCTGTTTCCTGTGTGAAATTGTTA |
| | TCCGCTCACAATTCCACACAACATACGAGCCGGAAGCATAAAGTGTAAAGCCTGGG |
| | GTGCCTAATGAGTGAGCTAACTCACATTAATTGCGTTGCGCTCACTGCCCGCTTTC |
| | CAGTCGGGAAACCTGTCGTGCCAGCTGCATTAATGAATCGGCCAACGCGCGGGGAG |
| | AGGCGGTTTGCGTATTGGGCGCTCTTCCGCTTCCTCGCTCACTGACTCGCTGCGCT |
| | CGGTCGTTCGGCTGCGGCGAGCGGTATCAGCTCACTCAAAGGCGGTAATACGGTTA |
| | TCCACAGAATCAGGGGATAACGCAGGAAAGAACATGTGAGCAAAAGGCCAGCAAAA |
| | GGCCAGGAACCGTAAAAAGGCCGCGTTGCTGGCGTTTTTCCATAGGCTCCGCCCCC |
| | CTGACGAGCATCACAAAAATCGACGCTCAAGTCAGAGGTGGCGAAACCCGACAGGA |
| | CTATAAAGATACCAGGCGTTTCCCCCTGGAAGCTCCCTCGTGCGCTCTCCTGTTCC |
| | GACCCTGCCGCTTACCGGATACCTGTCCGCCTTTCTCCCTTCGGGAAGCGTGGCGC |
| | TTTCTCATAGCTCACGCTGTAGGTATCTCAGTTCGGTGTAGGTCGTTCGCTCCAAG |
| | CTGGGCTGTGTGCACGAACCCCCCGTTCAGCCCGACCGCTGCGCCTTATCCGGTAA |
| | CTATCGTCTTGAGTCCAACCCGGTAAGACACGACTTATCGCCACTGGCAGCAGCCA |
| | CTGGTAACAGGATTAGCAGAGCGAGGTATGTAGGCGGTGCTACAGAGTTCTTGAAG |
| | TGGTGGCCTAACTACGGCTACACTAGAAGGACAGTATTTGGTATCTGCGCTCTGCT |
| | GAAGCCAGTTACCTTCGGAAAAAGAGTTGGTAGCTCTTGATCCGGCAAACAAACCA |
| | CCGCTGGTAGCGGTGGTTTTTTTGTTTGCAAGCAGCAGATTACGCGCAGAAAAAAA |
| | GGATCTCAAGAAGATCCTTTGATCTTTTCTACGGGGTCTGACGCTCAGTGGAACGA |
| | AAACTCACGTTAAGGGATTTTGGTCATGAGATTATCAAAAAGGATCTTCACCTAGA |
| | TCCTTTTAAATTAAAAATGAAGTTTTAAATCAATCTAAAGTATATATGAGTAAACT |
| | TGGTCTGACAGTTACCAATGCTTAATCAGTGAGGCACCTATCTCAGCGATCTGTCT |
| | ATTTCGTTCATCCATAGTTGCCTGACTCCCCGTCGTGTAGATAACTACGATACGGG |
| | AGGGCTTACCATCTGGCCCCAGTGCTGCAATGATACCGCGAGACCCACGCTCACCG |
| | GCTCCAGATTTATCAGCAATAAACCAGCCAGCCGGAAGGGCCGAGCGCAGAAGTGG |
| | TCCTGCAACTTTATCCGCCTCCATCCAGTCTATTAATTGTTGCCGGGAAGCTAGAG |
| | TAAGTAGTTCGCCAGTTAATAGTTTGCGCAACGTTGTTGCCATTGCTACAGGCATC |
| | GTGGTGTCACGCTCGTCGTTTGGTATGGCTTCATTCAGCTCCGGTTCCCAACGATC |
| | AAGGCGAGTTACATGATCCCCCATGTTGTGCAAAAAAGCGGTTAGCTCCTTCGGTC |
| | CTCCGATCGTTGTCAGAAGTAAGTTGGCCGCAGTGTTATCACTCATGGTTATGGCA |
| | GCACTGCATAATTCTCTTACTGTCATGCCATCCGTAAGATGCTTTTCTGTGACTGG |
| | TGAGTACTCAACCAAGTCATTCTGAGAATAGTGTATGCGGCGACCGAGTTGCTCTT |
| | GCCCGGCGTCAATACGGGATAATACCGCGCCACATAGC (SEQ ID NO: 24) |

In a 20-uL reaction volume, 50 fmoles of linearized DNA target in the presence of 50 nM sgRNA, 39 nM recombinant purified Cas9 protein (S. pyogenes; Agilent) and 10 mM or 0.8 mM MgCl2 at pH 7.6 was incubated at 37° C. for 30 min. Upon completion, 0.5 μL of RNace It (Agilent) was added, and incubation was continued at 37° C. for 5 min and then at 70° C. for 15 min. Subsequently 0.5 μL of Proteinase K (Mol. Bio. grade, NEB) was added and incubated at 37° C. for 15 min. Aliquots were loaded into a DNA 1000 or DNA 7500 LabChip and were analyzed on a Bioanalyzer 2200, or alternatively were loaded into a Genomic DNA ScreenTape and were analyzed on a TapeStation. The workup steps served to release Cas9 from binding of target DNA, which was assayed for cleavage. Cleavage yields were calculated by the formula: a/(a+b)×100 where a is the sum of the band intensities of the two cleavage products and b is the remaining uncleaved DNA if present. A cleavage percentage of 100% means that all of the target DNA construct was cleaved.

A series of guide RNAs were chemically synthesized. The guide RNA oligomers were synthesized on an ABI 394 Synthesizer (Life Technologies, Carlsbad, CA, USA) using 2′-O-thionocarbamate-protected nucleoside phosphoramidites according to procedures described in Dellinger et al. (2011) J. Am. Chem. Soc., 133, 11540-56. 2′—O-methyl phosphoramidites were incorporated into RNA oligomers under the same conditions as the 2′-O-thionocarbamate protected phosphoramidites. The 2′-O-methyl-3′-O-(diisopropylamino)phosphonoacetic acid-1,1-dimethylcyanoethyl ester-5′-O-dimethoxytrityl nucleosides used for synthesis of thiophosphonoacetate (thioPACE)-modified RNAs were synthesized essentially according to published methods. See Dellinger et al. (2003) J. Am. Chem. Soc., 125, 940-50; and Threlfall et al. (2012) Org. Biomol. Chem., 10, 746-54. For phosphorothioate-containing oligomers, the iodine oxidation step after the coupling reaction was replaced by a sulfurization step using a 0.05 M solution of 3-((N,N-dimethylaminomethylidene)amino)-3H-1,2,4-dithiazole-5-thione in a pyridine-acetonitrile (3:2) mixture for 6 min.

All the oligonucleotides were purified using reversed-phase high-performance liquid chromatography (HPLC) and analyzed by liquid chromatography-mass spectrometry (LC-MS) using an Agilent 1290 Infinity series LC system coupled to an Agilent 6520 Q-TOF (time-of-flight) mass spectrometer (Agilent Technologies, Santa Clara, CA, USA). The yields for the synthesis and purification of the sgRNAs were estimated using deconvolution of mass spectra obtained from LC-MS-derived total ion chromatograms. The chemical synthesis of the 100-mer sgRNAs typically yielded 25-35% full-length product from a nominal 1 micromole scale synthesis. Reversed-phase HPLC purification using ion pairing buffer conditions typically gave 20% yield from the crude product with an estimated purity of the final sgRNA in the range of 90% to 95%.

The results are shown in Table 4. “% Target cleaved” indicates the percentage of the target DNA construct which was cleaved. Experiments were run with and without addition of a molar excess of targetless competitor DNA (tcDNA) which potentially competes with the target DNA, so the potential impact of the added nonspecific DNA upon the assay could be seen.

TABLE 4

| | | | | | | |
|---|---|---|---|---|---|---|
| | | | | | | % CV |
| Entry | [Mg²⁺] | | % Target | % | % Cleaved | CON- |
| # | (mM) | tcDNA | cleaved | CV | CONTROL | TROL |

2-piece dual-guide scaffold
Unmodified dual-guide RNA (dgRNA)

| | | | | |
|---|---|---|---|---|
| 1 | 0.8 | N | 99% | — |
| 2 | 0.8 | Y | 99% | 5% |
| 3 | 0.8 | N | 96% | — |
| 4 | 0.8 | Y | 100% | 5% |
| 5 | 0.8 | N | 96% | — |
| 6 | 0.8 | Y | 0% | 5% |
| 7 | 0.8 | N | 99% | — |
| 8 | 0.8 | Y | 100% | 5% |
| 9 | 10 | N | 94%, 93% | — |
| 10 | 0.8 | Y | 88% | — |

Fluorophore-coupled dgRNA

| | | | | | | |
|---|---|---|---|---|---|---|
| 11 | 10 | N | 92%, 93% | — | 94%, 93% | — |

2′OMethyl-modified dgRNA

| | | | | | | |
|---|---|---|---|---|---|---|
| 12 | 0.8 | Y | 87% | — | 88% | — |

2′OMethyl,3′Phosphorothioate-modified dgRNA

| | | | | | | |
|---|---|---|---|---|---|---|
| 13 | 0.8 | Y | 87% | — | 88% | — |

2′OMethyl,3′PhosphorothioPACE-modified dgRNA

| | | | | | | |
|---|---|---|---|---|---|---|
| 14 | 0.8 | Y | 89% | — | 88% | — |
| 15 | 0.8 | Y | 86% | — | 88% | — |

2-thioU-modified dgRNA

| | | | | | | |
|---|---|---|---|---|---|---|
| 16 | 0.8 | N | 96% | — | 99% | — |
| 17 | 0.8 | Y | 95% | 5% | 99% | 5% |
| 18 | 0.8 | N | 95% | — | 96% | — |
| 19 | 0.8 | Y | 100% | 5% | 100% | 5% |
| 20 | 0.8 | N | 97% | — | 96% | — |
| 21 | 0.8 | Y | 0% | 5% | 0% | 5% |
| 22 | 0.8 | N | 98% | — | 99% | — |
| 23 | 0.8 | Y | 99% | 5% | 100% | 5% |
| 24 | 0.8 | N | 94% | — | 99% | — |
| 25 | 0.8 | Y | 83% | 5% | 99% | 5% |
| 26 | 0.8 | N | 93% | — | 96% | — |
| 27 | 0.8 | Y | 94% | 5% | 100% | 5% |
| 28 | 0.8 | N | 90% | — | 96% | — |
| 29 | 0.8 | Y | 0% | 5% | 0% | 5% |
| 30 | 0.8 | N | 95% | — | 99% | — |
| 31 | 0.8 | Y | 94% | 5% | 100% | 5% |
| 32 | 0.8 | N | 92% | — | 99% | — |
| 33 | 0.8 | Y | 84% | 5% | 99% | 5% |
| 34 | 0.8 | N | 90% | — | 96% | — |
| 35 | 0.8 | Y | 94% | 5% | 100% | 5% |
| 36 | 0.8 | N | 70% | — | 96% | — |
| 37 | 0.8 | Y | 0% | 5% | 0% | 5% |
| 38 | 0.8 | N | 96% | — | 99% | — |
| 39 | 0.8 | Y | 59% | 5% | 100% | 5% |

Single-guide scaffold
Unmodified single-guide RNA (sgRNA)

| | | | | | | |
|---|---|---|---|---|---|---|
| 40 | 10 | N | 93% | — | | |
| 41 | 10 | N | 94% | — | | |
| 42 | 10 | N | 94% | — | | |
| 43 | 10 | N | 92% | — | | |
| 44 | 10 | N | 90%, 92% | — | | |
| 45 | 10 | N | 92% | — | | |
| 46 | 10 | N | 93% | — | | |
| 47 | 0.8 | N | 86% | — | | |
| 48 | 0.8 | N | 87% | — | | |
| 49 | 0.8 | Y | 87% | — | | |
| 50 | 0.8 | N | 82% | — | | |
| 51 | 0.8 | N | 92% | — | | |
| 52 | 10 | N | 60% | — | | |
| 53 | 0.8 | N | 90% | — | | |
| 54 | 0.8 | N | 90% | — | | |
| 55 | 0.8 | Y | 79% | — | | |
| 56 | 0.8 | N | 79% | — | | |
| 57 | 0.8 | N | 94% | — | | |
| 58 | 10 | N | 73% | — | | |
| 59 | 0.8 | N | 84% | — | | |
| 60 | 0.8 | Y | ≥85% | — | | |
| 61 | 0.8 | Y | 89% | — | | |
| 62 | 0.8 | N | 87%, 82% | — | | |
| 63 | 0.8 | N | 23%, 22% | — | | |
| 64 | 0.8 | N | 78% | — | 87% | — |
| 65 | 0.8 | Y | 76% | — | 87% | — |
| 66 | 0.8 | N | 65% | — | 87% | — |
| 67 | 0.8 | N | 81% | — | 87% | — |
| 68 | 0.8 | N | 85% | — | 87% | — |
| 69 | 0.8 | Y | 71% | — | 87% | — |
| 70 | 0.8 | N | 32% | — | 87% | — |
| 71 | 0.8 | N | 84% | — | 87% | — |
| 72 | 0.8 | N | 91% | — | 87% | — |
| 73 | 0.8 | Y | 79% | — | 87% | — |
| 74 | 0.8 | N | 88% | — | 87% | — |
| 75 | 0.8 | N | 93% | — | 87% | — |
| 76 | 0.8 | N | 87% | — | 87% | — |
| 77 | 0.8 | Y | 79% | — | 87% | — |
| 78 | 0.8 | N | 89% | — | 87% | — |
| 79 | 0.8 | N | 88% | — | 87% | — |
| 80 | 0.8 | N | 3% | — | 86% | — |
| 81 | 0.8 | N | 5% | — | 86% | — |
| 82 | 0.8 | N | 89% | — | 86% | — |
| 83 | 0.8 | N | 68% | — | 87% | — |
| 84 | 0.8 | Y | 50% | — | 87% | — |
| 85 | 0.8 | N | 69% | — | 87% | — |
| 86 | 0.8 | N | 69% | — | 87% | — |
| 87 | 0.8 | N | 76% | — | 87% | — |
| 88 | 0.8 | Y | 42% | — | 87% | — |
| 89 | 0.8 | N | 72% | — | 87% | — |
| 90 | 0.8 | N | 78% | — | 87% | — |
| 91 | 0.8 | N | 85% | — | 87% | — |
| 92 | 0.8 | Y | 51% | — | 87% | — |
| 93 | 0.8 | N | 82% | — | 87% | — |
| 94 | 0.8 | ″ | 83% | — | 87% | — |

DMT-modified sgRNA

| | | | | | | |
|---|---|---|---|---|---|---|
| 95 | 10 | N | 93% | — | 92% | — |
| 96 | 10 | N | 93% | — | 92% | — |

Fluorophore-modified sgRNA

| | | | | | | |
|---|---|---|---|---|---|---|
| 97 | 10 | N | 91%, 91% | — | 90%, 92% | — |
| 98 | 0.8 | N | 86% | — | 87% | — |
| 99 | 0.8 | Y | 77% | — | 87% | — |
| 100 | 0.8 | N | 87% | — | 87% | — |
| 101 | 0.8 | N | 86% | — | 87% | — |
| 102 | 0.8 | N | 91% | — | 87% | — |
| 103 | 0.8 | Y | 82% | — | 87% | — |
| 104 | 0.8 | N | 90% | — | 87% | — |
| 105 | 0.8 | N | 92% | — | 87% | — |
| 106 | 0.8 | N | 91% | — | 87% | — |
| 107 | 0.8 | Y | 82% | — | 87% | — |
| 108 | 0.8 | N | 90% | — | 87% | — |
| 109 | 0.8 | N | 91% | — | 87% | — |
| 110 | 0.8 | N | 92% | — | 87% | — |
| 111 | 0.8 | Y | 84% | — | 87% | — |
| 112 | 0.8 | N | 92% | — | 87% | — |
| 113 | 0.8 | N | 89% | — | 87% | — |
| 114 | 0.8 | N | 84%, 84% | — | 87%, 82% | — |
| 115 | 0.8 | N | 12%, 6% | — | 23%, 22% | — |
| 116 | 0.8 | N | 93%, 90% | — | 87%, 82% | — |
| 117 | 0.8 | N | 8%, 9% | — | 23%, 22% | — |

3′Phosphorothioate-modified sgRNA

| | | | | | | |
|---|---|---|---|---|---|---|
| 118 | 10 | N | 95% | — | 90%, 92% | — |
| 119 | 10 | N | 94% | — | 90%, 92% | — |
| 120 | 10 | N | 97% | — | 90%, 92% | — |
| 121 | 10 | N | 94% | — | 90%, 92% | — |

2′OMethyl-modified sgRNA

| | | | | | | |
|---|---|---|---|---|---|---|
| 122 | 10 | N | 91% | — | 94% | — |
| 123 | 10 | N | 92% | — | 93% | — |
| 124 | 0.8 | N | 86% | — | 87% | — |
| 125 | ″ | Y | 77% | — | 87% | — |
| 126 | ″ | N | 85% | — | 87% | — |
| 127 | 0.8 | N | 88% | — | 87% | — |
| 128 | 10 | N | 92% | — | 94% | — |
| 129 | 0.8 | N | 83% | — | 87% | — |
| 130 | 0.8 | Y | 78% | — | 87% | — |
| 131 | 0.8 | N | 83% | — | 87% | — |
| 132 | 0.8 | N | 85% | — | 87% | — |
| 133 | 10 | N | 92% | — | 94% | — |
| 134 | 0.8 | N | 86% | — | 87% | — |
| 135 | 0.8 | Y | 78% | — | 87% | — |
| 136 | 0.8 | N | 83% | — | 87% | — |
| 137 | 0.8 | N | 88% | — | 87% | — |
| 138 | 10 | N | 91% | — | 94% | — |
| 139 | 0.8 | N | 84% | — | 87% | — |
| 140 | 0.8 | Y | 81% | — | 87% | — |
| 141 | 0.8 | N | 83% | — | 87% | — |
| 142 | 0.8 | N | 87% | — | 87% | — |
| 143 | 10 | N | 89% | — | 92% | — |
| 144 | 0.8 | N | 91%, 88% | — | 87%, 82% | — |
| 145 | 0.8 | N | 24%, 25% | — | 23%, 22% | — |
| 146 | 10 | N | 93%, 92% | — | 90%, 92% | — |
| 147 | 0.8 | N | 22% | — | 87% | — |
| 148 | 0.8 | Y | 3% | — | 87% | — |
| 149 | 0.8 | N | 12% | — | 87% | — |
| 150 | 0.8 | N | 5% | — | 87% | — |
| 151 | 10 | N | 0%, 0% | — | 90%, 92% | — |
| 152 | 10 | N | 0%, 0% | — | 90%, 92% | — |
| 153 | 0.8 | N | 85% | — | 86% | — |
| 154 | 0.8 | N | 87% | — | 86% | — |
| 155 | 0.8 | N | 89% | — | 87% | — |
| 156 | 0.8 | Y | 78% | — | 87% | — |
| 157 | 0.8 | N | 84% | — | 87% | — |
| 158 | 0.8 | N | 93% | — | 87% | — |
| 159 | 0.8 | N | 90% | — | 86% | — |
| 160 | 0.8 | N | 90% | — | 87% | — |
| 161 | 0.8 | Y | 86% | — | 87% | — |
| 162 | 0.8 | N | 90% | — | 87% | — |
| 163 | 0.8 | N | 91% | — | 87% | — |
| 164 | 0.8 | N | 92% | — | 90% | — |
| 165 | 0.8 | N | 89% | — | 87% | — |
| 166 | 0.8 | Y | 80% | — | 87% | — |
| 167 | 0.8 | N | 90% | — | 87% | — |
| 168 | 0.8 | N | 94% | — | 87% | — |
| 169 | 0.8 | N | 90% | — | 84% | — |
| 170 | 0.8 | Y | ≥85% | — | ≥85% | — |
| 171 | 0.8 | N | 7% | — | 84% | — |
| 172 | 0.8 | Y | 0% | — | ≥85% | — |
| 173 | 10 | N | 15% | — | 73% | — |
| 174 | 0.8 | N | 85% | — | 84% | — |
| 175 | 0.8 | Y | 75% | — | ≥85% | — |
| 176 | 10 | N | 86% | — | 73% | — |
| 177 | 0.8 | ″ | 0% | — | 84% | — |
| 178 | 0.8 | Y | 0% | — | ≥85% | — |
| 179 | 10 | N | 15% | — | 73% | — |

2′Deoxy-modified sgRNA

| | | | | | | |
|---|---|---|---|---|---|---|
| 180 | 10 | N | 27%, 19% | — | 90%, 92% | — |
| 181 | 10 | N | 0%, 0% | — | 90%, 92% | — |
| 182 | 10 | N | 0%, 0% | — | 90%, 92% | — |

2′Deoxy,3′PACE-modified sgRNA

| | | | | | | |
|---|---|---|---|---|---|---|
| 183 | 0.8 | N | 72%, 77% | — | 87%, 82% | — |
| 184 | 0.8 | N | 8%, 9% | — | 23%, 22% | — |

2′OMethyl,3′PACE-modified sgRNA

| | | | | | | |
|---|---|---|---|---|---|---|
| 185 | 0.8 | N | 82% | — | 87% | — |
| 186 | 0.8 | Y | 72% | — | 87% | — |
| 187 | 10 | N | 95% | — | 93% | — |
| 188 | 10 | N | 95% | — | 94% | — |
| 189 | 0.8 | N | 91% | — | 87% | — |
| 190 | 0.8 | Y | 84% | — | 87% | — |
| 191 | 0.8 | N | 85% | — | 87% | — |
| 192 | 0.8 | Y | 77% | — | 87% | — |
| 193 | 10 | N | 88% | — | 94% | — |
| 194 | 0.8 | N | 70% | — | 87% | — |
| 195 | 0.8 | Y | 56% | — | 87% | — |
| 196 | 0.8 | N | 40% | — | 87% | — |
| 197 | 0.8 | Y | 23% | — | 87% | — |
| 198 | 10 | N | 88% | — | 93% | — |
| 199 | 10 | N | 89% | — | 94% | — |
| 200 | 0.8 | N | 84% | — | 87% | — |
| 201 | 0.8 | Y | 75% | — | 87% | — |
| 202 | 10 | N | 90% | — | 93% | — |
| 203 | 10 | N | 90% | — | 94% | — |
| 204 | 0.8 | N | 86% | — | 87% | — |
| 205 | 0.8 | Y | 82% | — | 87% | — |
| 206 | 10 | N | 88% | — | 93% | — |
| 207 | 0.8 | N | 82% | — | 87% | — |
| 208 | 0.8 | Y | 78% | — | 87% | — |
| 209 | 10 | N | 77% | — | 93% | — |
| 210 | 0.8 | N | 71% | — | 87% | — |
| 211 | ″ | Y | 69% | — | ″ | — |
| 212 | 10 | N | 80% | — | 93% | — |
| 213 | 0.8 | N | 56% | — | 87% | — |
| 214 | 0.8 | Y | 41% | — | ″ | — |
| 215 | 10 | N | 78% | — | 93% | — |
| 216 | 0.8 | N | 58% | — | 87% | — |
| 217 | 0.8 | Y | 44% | — | ″ | — |
| 218 | 10 | N | 80% | — | 93% | — |
| 219 | 0.8 | N | 39% | — | 87% | — |
| 220 | 0.8 | Y | 13% | — | ″ | — |
| 221 | 10 | N | 74% | — | 93% | — |
| 222 | 0.8 | N | 36% | — | 87% | — |
| 223 | 0.8 | Y | 19% | — | 87% | — |
| 224 | 10 | N | 86% | — | 93% | — |
| 225 | 0.8 | N | 84% | — | 87% | — |
| 226 | 0.8 | Y | 80% | — | ″ | — |
| 227 | 10 | N | 88% | — | 93% | — |
| 228 | 0.8 | N | 83% | — | 87% | — |
| 229 | 0.8 | Y | 82% | — | 87% | — |
| 230 | 0.8 | N | 80% | — | 87% | — |
| 231 | 0.8 | N | 84% | — | 87% | — |
| 232 | 10 | N | 88% | — | 93% | — |
| 233 | 0.8 | N | 85% | — | 87% | — |
| 234 | 0.8 | Y | 73% | — | 87% | — |
| 235 | 10 | N | 82% | — | 93% | — |
| 236 | 0.8 | N | 89% | — | 87% | — |
| 237 | 0.8 | Y | 76% | — | 87% | — |
| 238 | 10 | N | 65% | — | 93% | — |
| 239 | 0.8 | N | 84% | — | 87% | — |
| 240 | 0.8 | Y | 56% | — | 87% | — |

2′OMethyl,3′Phosphorothioate-modified sgRNA

| | | | | | | |
|---|---|---|---|---|---|---|
| 241 | 10 | N | 92% | — | 92% | — |
| 242 | 0.8 | N | 84% | — | 87% | — |
| 243 | 0.8 | Y | 88% | — | 87% | — |
| 244 | 0.8 | N | 85% | — | 87% | — |
| 245 | 0.8 | N | 91% | — | 87% | — |
| 246 | 0.8 | N | 91% | — | 84% | — |
| 247 | 0.8 | Y | ≥85% | — | ≥85% | — |
| 248 | 0.8 | N | 84% | — | 84% | — |
| 249 | 0.8 | Y | 90% | — | 89% | — |
| 250 | 0.8 | N | 90%, 87% | — | 87%, 82% | — |
| 251 | 0.8 | N | 16%, 19% | — | 23%, 22% | — |
| 252 | 0.8 | N | 93% | — | 89% | — |
| 253 | 0.8 | N | 90%, 90% | — | 87%, 82% | — |
| 254 | 0.8 | N | 17%, 22% | — | 23%, 22% | — |
| 255 | 0.8 | N | 93% | — | 89% | — |
| 256 | 0.8 | N | 91%, 91% | — | 87%, 82% | — |
| 257 | 0.8 | N | 13%, 16% | — | 23%, 22% | — |

2′OMethyl,3′PhosphorothioPACE-modified sgRNA

| | | | | | | |
|---|---|---|---|---|---|---|
| 258 | 10 | N | 89% | — | 92% | — |
| 259 | 0.8 | N | 84% | — | 87% | — |
| 260 | 0.8 | Y | 80% | — | 87% | — |
| 261 | 0.8 | N | 77% | — | 87% | — |
| 262 | 0.8 | N | 83% | — | 87% | — |
| 263 | 0.8 | N | 92% | — | 87% | — |
| 264 | 0.8 | Y | 79% | — | 87% | — |
| 265 | 0.8 | N | 88% | — | 87% | — |
| 266 | 0.8 | N | 94% | — | 87% | — |
| 267 | 10 | N | 74% | — | 93% | — |
| 268 | 0.8 | N | 11% | — | 86% | — |
| 269 | 0.8 | N | 15% | — | ″ | — |
| 270 | 0.8 | N | 49% | — | ″ | — |
| 271 | 0.8 | N | 31% | — | ″ | — |
| 272 | 0.8 | N | 91% | — | 84% | — |
| 273 | 0.8 | Y | 77% | — | ≥85% | — |
| 274 | 0.8 | N | 90%, 91% | — | 87%, 82% | — |
| 275 | 0.8 | N | 9%, 8% | — | 23%, 22% | — |
| 276 | 0.8 | N | 90% | — | 84% | — |
| 277 | 0.8 | Y | ≥85% | — | ≥85% | — |
| 278 | 0.8 | N | 86%, 88% | — | 87%, 82% | — |
| 279 | 0.8 | N | 11%, 7% | — | 23%, 22% | — |

2-aminoA-modified sgRNA (including unmodified controls)

| | | | | | | |
|---|---|---|---|---|---|---|
| 280 | 0.8 | Y | 88%, 88% | — | | |
| 281 | 0.8 | N | 76%, 75% | — | | |
| 282 | 0.8 | N | 87%, 91% | — | 88%, 88% | — |
| 283 | 0.8 | N | 90%, 90% | — | 76%, 75% | — |
| 284 | 0.8 | N | 85%, 87% | — | — | — |
| 285 | 0.8 | N | 88%, 88% | — | — | — |
| 286 | 0.8 | N | 93%, 96% | — | 85%, 87% | — |
| 287 | 0.8 | N | 82%, 79% | — | 88%, 88% | — |

5-methylU-modified sgRNA

| | | | | | | |
|---|---|---|---|---|---|---|
| 288 | 0.8 | N | 86%, 83% | — | 87%, 82% | — |
| 289 | 0.8 | N | 11%, 11% | — | 23%, 22% | — |

Z base-modified sgRNA

| | | | | | | |
|---|---|---|---|---|---|---|
| 290 | 10 | N | 19% | — | 92% | — |
| 291 | 10 | N | 93% | — | ″ | — |

sgRNA modified to disfavor misfolding

| | | | | | | |
|---|---|---|---|---|---|---|
| 292 | 0.8 | N | 93% | — | 90% | — |
| 293 | 0.8 | N | 93% | — | 86% | — |

The results revealed that guide RNAs containing modifications at specific positions were tolerated by active Cas protein and gRNA: Cas protein complexes, as the modifications did not prevent target-specific cleavage of the on-target polynucleotide. The modifications that were tested and found to be tolerated at specific positions include 2′-O-methylribonucleotide (=2′OMe), 2′-deoxyribonucleotide, racemic phosphorothioate internucleotide linkage, 3′-phosphonoacetate (=PACE), 3′-thiophosphonoacetate (=thioPACE), Z nucleotide, 2-thiouracil, 2-aminoadenine, 5-methyluracil, 5-aminoallyluracil coupled to Cy5 fluorophore, 2-(4-butylamidofluorescein) propane-1,3-diol bis(phosphodiester) linker, and combinations of these.

It is contemplated that the chemical modifications disclosed and tested herein, particularly at the tested positions (as listed in Tables 3 and 4), will be tolerated at equivalent positions in a variety of guide RNAs.

As disclosed herein, chemically modified nucleotides were incorporated into guide RNAs in an effort to improve certain properties. Such properties include improved nuclease resistance of the guide RNA (also known as improved stability), reduced off-target effects of a gRNA: Cas protein complex (also known as improved specificity), improved efficacy of gRNA: Cas protein complex when cleaving, nicking or binding a target polynucleotide, improved transfection efficiency, and/or improved organelle localization.

The assay results in Tables 3 and 4 indicate that: (1) In guide RNAs, many positions can tolerate a variety of chemical modifications; (2) 5′ and 3′ ends of guide RNAs will tolerate a wide variety of end-protecting modifications, and such modifications are useful to inhibit exonucleolytic RNA degradation; (3) 2-ThioU can be used to deter off-target interactions involving G-U wobble pairings, thereby increasing the specificity of guide pairing by inhibiting off-target hybridization interactions; (4) 5′ Extensions are generally well-tolerated; (5) Surface exposed regions of the guide RNA (as inferred from published crystal structures) are tolerant of extensively modifying U's to 5-methylU's, which potentially make the modified RNA more likely to elude immune responses such as stimulated by unmodified RNA; and (6) For RNA folding, G-C pairs are stronger and more stable than A-U pairs. At least one guide RNA is tolerant of replacing some G-C pairs with 2′-O-methylA-2′-O-methylU pairs that are more stable thermodynamically than unmodified A-U pairs.

More particularly, the present example shows that 2′-O-methyl modifications are tolerated at the 5′ and 3′ ends of dual-guide RNAs (as shown by entry 12 in Tables 3 and 4) and single-guide RNAs (entries 143-146, 169-170), thus allowing end-protection to stabilize gRNAs against exonucleases. 2′-O-methyl modifications are tolerated at most but not all internal positions, thus allowing stabilization against various nucleases including endonucleases (entries 146, 153-168, 174-179). However, the present example also demonstrates that not every position in guide RNAs will tolerate 2′-O-methyls (as shown by entries 151-152 and 171-173), suggesting that too many consecutive 2′-O-methyl modifications at the 5′ end (e.g., 26 or more consecutive 2′-O-methyl-modified nucleotides), or too many 2′-O-methyl modifications of C and U nucleotides downstream (3′) of the 5′-terminal 20mer guide sequence is not well tolerated (e.g., the inhibitory effect of one or both 2′-O-methyluracils at sequence positions +56 and +69 in entries 171-173 as revealed by the positions tested in entries 154-156).

The present example shows that 2′-O-methyl modifications throughout the 20mer guide sequence are tolerated during in vitro uses in buffer containing 10 mM Mg2+ (entry 146), but such extensive modification is not well tolerated under physiological conditions (entries 147-150) as present in cells. Thus, in some embodiments, a gRNA comprising 15 or more, alternatively 17 or more, alternatively 18 or more, alternatively 20 2′—O-methyl modifications throughout the 20mer guide sequence is used for in vitro methods as described herein, such as genomic editing to modify a DNA sequence in vitro, regulating the expression of a gene of interest in vitro, cleaving a DNA target sequence in vitro, and other uses.

The present example shows that extensive incorporation of 2′-deoxy modifications is not well tolerated and can be substantially completely inhibitory (entries 180-182). However, 2′-deoxy modifications can be well-tolerated at some locations (entry 183), therefore such modification can be useful for inhibiting nucleases.

The present example also shows that fluorophore or dye labels are tolerated in every loop of the three known stem-loops in CRISPR-Cas9 guide RNAs (entry 116). Such labels are also tolerated in a 5′ overhang on the guide sequence (entry 114), tolerated at additional locations in sgRNAs (entry 114), and tolerated in a loop in tracrRNA used in dual-guide applications (entry 11). In this example, two different types of fluorophores were tested: a phosphodiester-linked fluorophore (no ribose ring) that essentially takes the place of a nucleotide (entries 114 & 116), and a dye label (Cy5) covalently coupled to 5-aminoallylU incorporated in a guide RNA (entry 11).

The present example also shows that Z bases are tolerated in synthetic guide RNAs, particularly as modifications of synthetic guide RNAs in which some C's are replaced with Z bases (entries 290-291). The present example also shows that several other bases are tolerated at various positions, as shown in Tables 3 and 4.

The present example further shows that the 5′ and 3′ ends of guide RNAs can tolerate a wide variety of end-protecting modifications. Such modifications can be used to inhibit exonucleolytic RNA degradation. Support for the tolerance of such modifications can be found in Hendel et al., Nat. Biotechnol. (2015) 33:9, 985-9. Additional support for modifications at the 5′ and 3′ ends of guide RNAs is provided by entries 143-144, 185-223, 241-257, 258-266, and 272-279 in Tables 3 and 4. In some embodiments, the guide RNA comprises 7 or fewer modified nucleotides at a 5′ end or a 3′ end or at each of 5′ and 3′ ends, alternatively 6 or fewer, alternatively 5 or fewer, alternatively 4 or fewer, alternatively 3 or fewer, alternatively 2 or fewer, alternatively 1. Dual-guide RNAs can be protected similarly (entries 12-15).

The present example further shows that 2-thioU can be used to deter off-target interactions involving G-U wobble pairings, thereby increasing the specificity of guide sequence pairing by inhibiting off-target hybridization interactions (entries 16-39). One of the base pairs involved in hybridization between the guide RNA and CLTA1 OFF-target 3 (also referred to as “CLTA1 OFF3-target” or “CLTA1 OFF3”) is a G-U wobble pair. Replacing the corresponding U in the guide RNA with a 2-thioU reduces cleavage from 100% (entry 8) to 59% (entry 39). Replacing other U's with 2-thioU's (e.g., at sequence position +3 or +9, entries 23 and 31) does not have the same effect, presumably because those U's do not involve G-U wobble pairing when fully hybridized to each of the OFF-target sites tested. Accordingly, 2-thioU can increase target specificity of guide RNAs when off-target sites involve G-U wobble pairing.

The present example also shows that 5′-overhang sequences attached to the guide sequence are generally well-tolerated (see entries 83-95, 114, and 206-223). For example, a bulky dimethoxytrityl (dmt) group at the 5′ end was well tolerated (entry 95). The chromatographic properties of dmt can be used to facilitate purification of full-length synthetic RNAs from incompletely elongated byproducts which are generally produced during synthesis. Accordingly, in some embodiments, the synthetic guide RNA comprises a 5′-overhang sequence, for example, comprising a short polynucleotide sequence of 15 or fewer nucleotides which is complementary to the guide sequence and is covalently linked at its 3′ end to the 5′ end of the guide sequence by a polymeric linker such as a polynucleotide or similar phosphodiester-based linker, in which the linker can be 5 or more consecutive uridine nucleotides, alternatively 6 or 7.

The present example also shows that surface exposed regions of the guide RNA (as inferred from crystal structures published by others) are tolerant of extensively modifying uracils nucleotides to 5-methyluracils (5-methylU's) (entry 288), which can make the modified RNA more likely to elude immune responses such as stimulated by unmodified RNA. In particular, the 5′ and 3′ ends of a synthetic guide RNA are potentially immunostimulatory, and the present example shows that 5′ and 3′ ends are tolerant of 5-methylU modifications (entry 288).

The present example also shows that a synthetic guide RNA is tolerant of replacing some G-C pairs with 2′-O-methylA-2′-O-methylU pairs which are more stable thermodynamically than unmodified A-U pairs (see the non-terminal-2′-O-methylU and complementary-2′-O-methylA modifications in entries 292-293). This is advantageous because it is known that, for folded RNAs, G-C pairs are stronger and more stable than A-U pairs. Replacement of G-C pairs with such thermostabilized A-U pairs in synthetic guide RNAs allows for improved folding of active structures by preventing misfolded structures that involve unintended G-C pair(s), as can be predicted by RNA folding algorithms in common use.

### Exemplary Embodiments

Exemplary embodiments provided in accordance with the presently disclosed subject matter include, but are not limited to, the claims and the following embodiments:

A1. A synthetic guide RNA comprising:
a crRNA segment comprising (i) a guide sequence capable of hybridizing to a target sequence, (ii) a stem sequence; and
a tracrRNA segment comprising a nucleotide sequence that is partially or completely complementary to the stem sequence, wherein the synthetic guide RNA comprises at least one modified nucleotide, and wherein the synthetic guide RNA has gRNA functionality.

A2. The synthetic guide RNA of embodiment A1, comprising a 2′-deoxy moiety.

A3. The synthetic guide RNA of embodiment A1 or A2, comprising a 2′-halo moiety selected from 2′-fluoro, 2′-chloro, 2′-bromo and 2′-iodo.

A4. The synthetic guide RNA of any one of the preceding embodiments, comprising a phosphorothioate group.

A5. The synthetic guide RNA of any one of the preceding embodiments, comprising a PACE group.

A6. The synthetic guide RNA of any one of the preceding embodiments, comprising a thioPACE group.

A7. The synthetic guide RNA of any one of embodiments A2-A6, comprising a 2′-O-methyl moiety.

A8. The synthetic guide RNA of any one of the preceding embodiments, comprising a 2-thiouracil.

A9. The synthetic guide RNA of any one of the preceding embodiments, comprising a 4-thiouracil.

A10. The synthetic guide RNA of any one of the preceding embodiments, comprising a 2-aminoadenine.

A11. The synthetic guide RNA of any one of the preceding embodiments, comprising a hypoxanthine.

A12. The synthetic guide RNA of any one of the preceding embodiments, comprising a 5-methylcytosine.

A13. The synthetic guide RNA of any one of the preceding embodiments, comprising a 5-methyluracil.

A14. The synthetic guide RNA of any one of the preceding embodiments, comprising a 5-aminoallyl-uracil.

A15. The synthetic guide RNA of any one of the preceding embodiments, comprising a Z ribonucleotide.

A16. The synthetic guide RNA of any one of the preceding embodiments, comprising a Z deoxyribonucleotide.

A17. The synthetic guide RNA of any one of the preceding embodiments, comprising a squarate conjugation.

A18. The synthetic guide RNA of any one of the preceding embodiments, comprising a dye linker.

A19. The synthetic guide RNA of embodiment A18, wherein the dye linker is 2-(4-butylamidofluorescein) propane-1,3-diol bis(phosphodiester) linker.

A20. The synthetic guide RNA of any one of the preceding embodiments, comprising a nucleotide with 2′-O-methyl and 3′-phosphorothioate.

A21. The synthetic guide RNA of any one of the preceding embodiments, comprising a nucleotide with 2′-O-methyl and 3″-PACE.

A22. The synthetic guide RNA of any one of the preceding embodiments, comprising a nucleotide with 2′-O-methyl and 3′-thioPACE.

A23. The synthetic guide RNA of any one of the preceding embodiments, comprising a nucleotide with 2′-deoxy and 3′-PACE.

A24. The synthetic guide RNA of any one of the preceding embodiments, comprising a 5-methylcytidine.

A25. The synthetic guide RNA of any one of the preceding embodiments, comprising a methylphosphonate.

A26. The synthetic guide RNA of any one of the preceding embodiments, comprising an ester of PACE, wherein the ester is optionally a methyl ester.

A27. The synthetic guide RNA of any one of the preceding embodiments, comprising a single RNA strand comprising both the cr RNA segment and the tracr RNA segment.

A28. The synthetic guide RNA of any one of embodiments A1-A26, comprising two RNA strands, and the cr RNA segment and the tracr RNA segment are in different RNA strands.

A29. The synthetic guide RNA of any one of the preceding embodiments, comprising a modified nucleotide at a 5′ end, 3′ end, or both 5′ end and 3′ end of each RNA strand.

A30. The synthetic guide RNA of any one of the preceding embodiments, comprising a modified nucleotide in the guide sequence.

A31. The synthetic guide RNA of any one of the preceding embodiments, comprising a modified nucleotide 5′ to the guide sequence.

A32. The synthetic guide RNA of any one of the preceding embodiments, comprising a modified nucleotide in the stem sequence.

A33. The synthetic guide RNA of any one of the preceding embodiments, comprising a modified nucleotide in the scaffold region.

A34. The synthetic guide RNA of any one of the preceding embodiments, comprising at least one unnatural, orthogonal base pair in the scaffold region, wherein the base pair is independently selected from isoG-isoC and Z base-P base.

A35. The synthetic guide RNA of any one of the preceding embodiments, comprising a 2′-amino group.

A36. The synthetic guide RNA of any one of the preceding embodiments, comprising a phosphorodithioate linkage group.

A37. The synthetic guide RNA of any one of the preceding embodiments, comprising a boranophosphate linkage group.

A38. The synthetic guide RNA of any one of the preceding embodiments, comprising an unlocked nucleic acid modification (ULNA).

A39. The synthetic guide RNA of any one of the preceding embodiments, comprising a locked nucleic acid modification (LNA).

A40. The synthetic guide RNA of any one of the preceding embodiments, comprising an unstructured nucleic acid modification (UNA).

A41. The synthetic guide RNA of any one of the preceding embodiments, comprising a pseudoU.

A42. The synthetic guide RNA of any one of the preceding embodiments, comprising a 2′-MOE.

A43. The synthetic guide RNA of any one of the preceding embodiments, comprising a 2′-arabino.

A44. The synthetic guide RNA of any one of the preceding embodiments, comprising a 4′-thioribose.

A45. The synthetic guide RNA of any one of the preceding embodiments, comprising a squarate linkage.

A46. The synthetic guide RNA of any one of the preceding embodiments, comprising a triazolo linkage.

A47. A method for cleaving or nicking a target polynucleotide comprising contacting the target polynucleotide with a CRISPR-associated protein and the synthetic guide RNA of any one of the preceding embodiments, and cleaving or nicking the target polynucleotide.

A48. The method of embodiment A47, wherein the cleaving or nicking takes place in vitro.

A49. The method of embodiment A47, wherein the cleaving or nicking takes place in a cell. A50. The method of embodiment A47, wherein the cleaving or nicking takes place in vivo.

A51. The method of any one of embodiments A47-A50, wherein the CRISPR-associated protein is Cas9.

A52. The method of any one of embodiments A47-A51, wherein the cleaving or nicking results in gene editing.

A53. The method of any one of embodiments A47-A52, wherein the cleaving or nicking results in alteration of gene expression.

A54. A method for binding a target polynucleotide comprising contacting the target polynucleotide with a CRISPR-associated protein and the synthetic guide RNA of any one of the preceding embodiments,

A55. The method of embodiment A54, wherein the CRISPR-associated protein comprises a mutant which does not have a cleavage or nicking activity.

A56. The method of embodiment A54 or A55, wherein the CRISPR-associated protein is a fusion protein comprising a protein component not naturally existing in a CRISPR system.

A57. The method of any one of embodiments A54 to A56, resulting in a change of expression of the target polynucleotide.

A58. The method of any one of embodiments A54 to A57 useful to tag the target polynucleotide.

### Further Exemplary Embodiments

B1. A synthetic guide RNA comprising:
(a) a crRNA segment comprising (i) a guide sequence capable of hybridizing to a target sequence in a polynucleotide, (ii) a stem sequence; and
(b) a tracrRNA segment comprising a nucleotide sequence that is partially or completely complementary to the stem sequence, wherein the synthetic guide RNA comprises one or more modifications, and wherein the synthetic guide RNA has gRNA functionality.

B2. The synthetic guide RNA of embodiment 1, comprising a 2′-O-methyl moiety, a 2′-deoxy moiety, a Z base, a phosphorothioate internucleotide linkage, a phosphonoacetate internucleotide linkage, a thiophosphonoacetate internucleotide linkage, or combinations thereof.

B3. The synthetic guide RNA of embodiment 1 or 2, comprising one or more modifications selected from the group consisting of a 2′-O-methyl nucleotide with a 3′-phosphorothioate group, a 2′-O-methyl nucleotide with a 3′-phosphonocarboxylate group, a 2′-O-methyl nucleotide with a 3′-phosphonoacetate group, a 2′-O-methyl nucleotide with a 3′-thiophosphonocarboxylate group, a 2′-O-methyl nucleotide with a 3′-thiophosphonoacetate group, a 2′-deoxy nucleotide with a 3′-phosphonoacetate group, a 2′-deoxy nucleotide with a 3′-thiophosphonoacetate group, and a Z base.

B4. The synthetic guide RNA of embodiment 1, 2 or 3, comprising one or more modifications selected from the group consisting of a 2′-fluororibosyl, a 2-thiouracil base, a 4-thiouracil base, a 2-aminoadenine base, an hypoxanthine base, a 5-methylcytosine base, a 5-methyluracil base, a methylphosphonate internucleotide linkage, a 5-aminoallyluracil base, a squarate linkage, a triazolo linkage, a dye conjugated to a nucleotide, and combinations thereof.

B5. The synthetic guide RNA of any of the preceding embodiments, comprising a modification selected from the group consisting of a 2′-MOE, 2′-amino, 2′-F-arabino, 2′-LNA, 2′-ULNA, 3′-methylphosphonate, 3′-boranophosphate, 3′-phosphorodithioate, 2′-OMe-3′-P(S)2, 2′—OMe-3′-P(CH3), 2′-OMe-3′-P(BH3), 4′-thioribosyl, L-sugar, 2-thiocytosine, 6-thioguanine, 2-aminopurine, pseudouracil, 7-deazaguanine, 7-deaza-8-azaguanine, 7-deazaadenine, 7-deaza-8-azaadenine, 5-hydroxymethylcytosine, 5-hydroxymethyluracil, 5,6-dehydrouracil, 5-propynylcytosine, 5-propynyluracil, 5-ethynylcytosine, 5-ethynyluracil, 5-allyluracil, 5-allylcytisine, 5-allylaminocytosine, P nucleobase, isocytosine (isoC), isoguanine (isoG), UNA, x(A,G,C,T), y(A,G,C,T), abasic nucleotide, PEG, hydrocarbon linker, halo-substituted hydrocarbon linker, heteroatom (O,N,S)-substituted hydrocarbon linker, (keto, carboxy, amido, thionyl, carbamoyl, or thionocarbamoyl)-containing hydrocarbon linker, spermine linker, and combinations thereof.

B6. The synthetic guide RNA of any one of the preceding embodiments, comprising a stability-enhancing modification.

B7. The synthetic guide RNA of any one of the preceding embodiments, comprising at least two modifications; wherein a first modification is a stability-enhancing modification and a second modification is a specificity-altering modification.

B8. The synthetic guide RNA of embodiment 6 or 7, wherein the stability-enhancing modification is located in the guide sequence.

B9. The synthetic guide RNA of embodiment 6 or 7, wherein the stability-enhancing modification is located upstream of the guide sequence.

B10. The synthetic guide RNA of embodiment 6 or 7, wherein the stability-enhancing modification is located within the first five and/or the last five nucleotides of the crRNA segment.

B11. The synthetic guide RNA of embodiment 6 or 7, wherein the stability-enhancing modification is located in the tracrRNA segment.

B12. The synthetic guide RNA of embodiment 6 or 7, wherein the stability-enhancing modification is located within the first five and/or the last five nucleotides of the tracrRNA segment.

B13. The synthetic guide RNA of any one of embodiments 6-12, wherein the stability-enhancing modification comprises a 2′-O-methyl moiety, a 2′-fluoro moiety, or a 2′-deoxy moiety.

B14. The synthetic guide RNA of any one of embodiments 6-13, wherein the stability-enhancing modification comprises a phosphorothioate internucleotide linkage, a phosphonoacetate internucleotide linkage, a thiophosphonoacetate internucleotide linkage, a methylphosphonate internucleotide linkage, a boranophosphate internucleotide linkage, or a phosphorodithioate internucleotide linkage.

B15. The synthetic guide RNA of any one of embodiments 6-14, wherein the stability-enhancing modification comprises a 3′-phosphonoacetate or a 3′-thiophosphonoacetate.

B16. The synthetic guide RNA any one of embodiments 6-15, wherein the stability-enhancing modification comprises a 2′-O-methyl-3′-phosphorothioate nucleotide, a 2′-O-methyl-3′-phosphonoacetate nucleotide, or a 2′-O-methyl-3′-thiophosphonoacetate nucleotide.

B17. The synthetic guide RNA of any one of embodiments 6-16, wherein the stability-enhancing modification comprises a 2′-fluoro-3′-phosphorothioate nucleotide, a 2′-fluoro-3′-phosphonoacetate nucleotide, or a 2′-fluoro-3′-thiophosphonoacetate nucleotide.

B18. The synthetic guide RNA of any one of the preceding embodiments, comprising a specificity-altering modification.

B19. The synthetic guide RNA of embodiment 18, wherein the specificity-altering modification is located in the guide sequence.

B20. The synthetic guide RNA of any one of embodiment 18 or 19, wherein the specificity-altering modification comprises a 2-thiouracil, a 4-thiouracil or a 2-aminoadenine.

B21. The synthetic guide RNA of any one of embodiments 18-20, wherein the specificity-altering modification comprises a phosphorothioate internucleotide linkage, a phosphonoacetate internucleotide linkage, a thiophosphonoacetate internucleotide linkage, a methylphosphonate internucleotide linkage, a boranophosphate internucleotide linkage, or a phosphorodithioate internucleotide linkage.

B22. The synthetic guide RNA of any one of embodiments 18-21, wherein the specificity-altering modification comprises a 3′-phosphonoacetate or a 3′-thiophosphonoacetate.

B23. The synthetic guide RNA of any one of the preceding embodiments, comprising a fluorescent dye or a label.

B24. The synthetic guide RNA of any one of the preceding embodiments, comprising one or more isotopic labels.

B25. The synthetic guide RNA of any one of the preceding embodiments, wherein the guide RNA is conjugated to an oligonucleotide, an aptamer, an amino acid, a peptide, a protein, a steroid, a lipid, a folic acid, a vitamin, a sugar, or an oligosaccharide.

B26. The synthetic guide RNA of any one of the preceding embodiments, wherein the synthetic guide RNA is a single guide RNA, wherein the crRNA segment and the tracrRNA segment are linked through a loop L.

B27. The synthetic guide RNA of embodiment 26, wherein the loop L comprises 1, 2, 3, 4, 5, 6, 7, 8, 9, or 10 nucleotides.

B28. The synthetic guide RNA of embodiment 26 or 27, wherein the loop L comprises a nucleotide sequence of GNRA, wherein N represents A, C, G, or U and R represents A or G.

B29. The synthetic guide RNA of embodiment 26, 27 or 28, wherein the loop L comprises a nucleotide sequence of GAAA.

B30. The synthetic guide RNA of any one of embodiments 26-29, wherein the loop L comprises one or more modified nucleotides.

B31. The synthetic guide RNA of embodiment 30, wherein the loop L comprises a fluorescent dye.

B32. The synthetic guide RNA of embodiment 31, wherein the dye is conjugated to a 2-(4-butylamido-dye) propane-1,3-diol bis(phosphodiester) linker.

B33. The synthetic guide RNA of any one of the preceding embodiments, wherein the crRNA segment is at the 5′ end of the guide RNA.

B34. The synthetic guide RNA of any one of the preceding embodiments, wherein the tracrRNA segment is at the 3′ end of the guide RNA.

B35. The synthetic guide RNA of any of the preceding embodiments, wherein the crRNA segment comprises from 25 to 70 nucleotides.

B36. The synthetic guide RNA of any of the preceding embodiments, wherein the guide sequence comprises from 15 to 30 nucleotides.

B37. The synthetic guide RNA of any of the preceding embodiments, wherein the stem sequence comprises from 10 to 50 nucleotides.

B38. The synthetic guide RNA of any of the preceding embodiments, comprising one or more triazolo linkage(s).

B39. The synthetic guide RNA of any of the preceding embodiments, comprising one or more squarate linkage(s).

B40. The synthetic guide RNA of any of the preceding embodiments, wherein the guide RNA comprises a nucleotide composition of:

MₘNₙ
wherein each N independently represents an unmodified nucleotide and each M is selected from a 2′-O-methyl ribonucleotide, a 2′-O-methyl-3′-P(S) ribonucleotide, a 2′-O-methyl-3′-PACE ribonucleotide, a 2′-O-methyl-3′-thioPACE ribonucleotide, and a 2′-deoxynucleotide;
wherein each M is at any position in the sequence of the guide RNA; and
wherein m is an integer between 1 and 220, and n is an integer between 0 and 219, and 50<m+n≤220.

B41. The synthetic guide RNA of embodiment 38, wherein m+n<150, and each of m and n are independently selected from an integer between 0 and 150, provided that m is not 0.

B42. The synthetic guide RNA of any of the preceding embodiments, wherein the guide RNA comprises a nucleotide sequence of:

MₘNₙM′_{m′}N′_{n′}M″_{m″}
wherein each M, M′ and M″ independently represent a modified nucleotide and each N and N′ independently represent an unmodified ribonucleotide;
wherein any given M is the same or different from any other M, any given N is the same or different from any other N, any given M′ is the same or different from any other M′, any given N′ is the same or different from any other N′, any given M″ is the same or different from any other M″; and
wherein m is an integer between 0 and 40, n is an integer between 20 and 130, m′ is an integer between 0 and 10, n′ is an integer between 0 and 50, m″ is an integer between 0 and 10, provided that m+m′+m″ is greater than or equal to 1.

B43. The synthetic guide RNA of any of the preceding embodiments, wherein the crRNA segment comprises a nucleotide sequence of:

MₘNₙM′_{m′}N′_{n′}
wherein M and M′ each represent a modified nucleotide and N and N′ each represent an unmodified ribonucleotide;
wherein any given M is the same or different from any other M, any given N is the same or different from any other N, any given M′ is the same or different from any other M′, any given N′ is the same or different from any other N′; and
wherein n and n′ are each independently selected from an integer between 0 and 50, and wherein m and m′ are each independently selected from an integer between 0 and 25, provided that m+m′ is greater than or equal to 1.

B44. The synthetic guide RNA of any of the preceding embodiments, wherein the guide sequence comprises a nucleotide sequence of:

MₘNₙM′_{m′}N′_{n′}
wherein M and M′ each represent a modified nucleotide and N and N′ each represent an unmodified ribonucleotide;
wherein any given M is the same or different from any other M, any given N is the same or different from any other N, any given M′ is the same or different from any other M′, any given N′ is the same or different from any other N′; and
wherein m, n, m′, and n′ are each independently selected from an integer between 0 and 40, provided that m+m′ is greater than or equal to 1.

B45. The synthetic guide RNA of any of the preceding embodiments, wherein the tracrRNA segment comprises a nucleotide sequence of:

NₙMₘN′_{n′}M′_{m′}
wherein M and M′ each represent a modified nucleotide and N and N′ each represent an unmodified ribonucleotide;
wherein any given M is the same or different from any other M, any given N is the same or different from any other N, any given M′ is the same or different from any other M′, any given N′ is the same or different from any other N′; and
wherein n is an integer between 0 and 130 m is an integer between 0 and 40, and n′ is an integer between 0 and 130, and m′ is an integer between 0 and 40, provided that m+m′ is greater than or equal to 1.

B46. The synthetic guide RNA of any one of embodiments 40-43, wherein m, m′, m+m′, or m+m′+m″ is 1, 2, 3, 4, 5, 6, 7, 8, 9, 10, 11, 12, 13, 14, 15, 16, 17, 18, 19, or 20.

B47. The synthetic guide RNA of any one of embodiments 40-43, wherein m, m′, m+m′, or m+m′+m″ is 1, 2, 3, 4, 5, or 6.

B48. The synthetic guide RNA of any one of embodiments 40-45, wherein n is 16, 17, 18, or 19.

B49. The synthetic guide RNA of any one of embodiments 40-45, wherein n, n′, or n+n′ is an integer between 75 and 115.

B50. The synthetic guide RNA of any one of embodiments 40-47, wherein each M is independently selected from the group consisting of a 2′-modified nucleotide, a 3′-modified nucleotide, and combinations thereof.

B51. The synthetic guide RNA of embodiment 48, wherein the 2′-modified nucleotide is selected from the group consisting of a 2′-deoxy nucleotide, a 2′-O-methyl nucleotide, a 2′-fluoro nucleotide, and a 2′-O—C1-3alkyl-O—C1-3alkyl nucleotide.

B52. The synthetic guide RNA of embodiment 48, wherein the 3′-modified nucleotide is selected from the group consisting of a 3′-phosphonoacetate nucleotide and a 3′-thiophosphonoacetate nucleotide.

B53. The synthetic guide RNA of embodiment 48, wherein the combination of the 2′-modified nucleotide and the 3′-modified nucleotide comprises a 2′-O-methyl-3′-phosphorothioate nucleotide, a 2′-O-methyl-3′-phosphonoacetate nucleotide, or a 2′-O-methyl-3′-thiophosphonoacetate nucleotide.

B54. A method for cleaving a target polynucleotide comprising contacting the target polynucleotide with a CRISPR-associated protein and the synthetic guide RNA of any one of the preceding embodiments and cleaving the target polynucleotide.

B55. The method of embodiment 52, further comprising contacting the target polynucleotide with an exogenous CRISPR-associated protein.

B56. The method of embodiment 53, wherein the CRISPR-associated protein is Cas9.

B57. The method of any one of embodiments 52-54, wherein the cleavage results in a functional knockout of a target gene.

B58. The method of any one of embodiments 52-55, further comprising repairing the cleaved target polynucleotide by homology-directed repair with an exogenous or endogenous template polynucleotide.

B59. The method of embodiment 56, wherein the exogenous or endogenous template polynucleotide comprises at least one sequence having substantial sequence identity with a sequence on either side of the cleavage site.

B60. The method of any one of embodiments 52-57, further comprising repairing the cleaved target polynucleotide by non-homologous end joining.

B61. The method of any one of embodiments 56-58, wherein the repairing step produces an insertion, deletion, or substitution of one or more nucleotides of the target polynucleotide.

B62. The method of embodiment 59, wherein the insertion, deletion, or substitution results in one or more amino acid changes in a protein expressed from a gene comprising the target polynucleotide.

B63. The method of any one of embodiments 52-60, wherein the target polynucleotide is contacted with the CRISPR-associated protein and the synthetic guide RNA in vitro.

B64. The method of any one of embodiments 52-61, wherein the target polynucleotide contacted with the CRISPR-associated protein and the synthetic guide RNA is within the genome of a cell in vitro or in vivo.

B65. The method of embodiment 62, wherein the cell is isolated from a multicellular source prior to contacting the target polynucleotide with the CRISPR-associated protein and the synthetic guide RNA.

B66. The method of embodiment 63, wherein the source is a plant, an animal, a multicellular protist, or a fungus.

B67. The method of any one of embodiments 62-64, wherein the cell, or a cell derived therefrom, is returned to the source after contacting the target polynucleotide with the CRISPR-associated protein and the synthetic guide RNA.

B68. A method of modifying a target polynucleotide in a cell comprising introducing into the cell the synthetic guide RNA of any one of embodiments 1-51 and introducing into the cell a CRISPR-associated protein or a nucleic acid that expresses a CRISPR-associated protein in the cell.

B69. The method of embodiment 66, wherein the CRISPR-associated-protein is Cas9.

B70. A method of altering expression of at least one gene product in a cell comprising introducing into the cell the synthetic guide RNA of any one of embodiments 1-51 and further introducing into the cell a CRISPR-associated-protein or a nucleic acid that expresses a CRISPR-associated protein in the cell, wherein the cell contains and expresses a DNA molecule having a target sequence and encoding the gene product.

B71. The method of embodiment 68, wherein the CRISPR-associated-protein is Cas9.

B72. The method of embodiment 69, wherein the CRISPR-associated-protein cleaves the DNA molecule.

B73. A set or library of RNA molecules comprising two or more synthetic guide RNAs of any one of embodiments 1-51.

B74. A kit comprising the synthetic guide RNA of any one of embodiments 1-51 or the set or library of RNA molecules of embodiment 71.

B75. The kit of embodiment 72, further comprising a CRISPR-associated protein or a nucleic acid encoding the CRISPR-associated protein.

B76. The kit of embodiment 73, wherein the CRISPR-associated-protein is Cas9.

B77. The synthetic guide RNA, method or kit of any of the preceding embodiments, wherein the synthetic guide RNA comprises an end modification.

B78. The synthetic guide RNA of any of the preceding embodiments, having a single RNA strand or two separate complementary RNA strands, wherein the guide RNA comprises at least one stability-enhancing modification at both ends of each RNA strand.

C1. A synthetic guide RNA comprising:
(a) a crRNA segment comprising (i) a guide sequence capable of hybridizing to a target sequence in a polynucleotide, (ii) a stem sequence; and
(b) a tracrRNA segment comprising a nucleotide sequence that is partially or completely complementary to the stem sequence, wherein the synthetic guide RNA comprises one or more modifications, and wherein the synthetic guide RNA has gRNA functionality.

C2. The synthetic guide RNA of embodiment C1, wherein one or more of the modifications comprises a stability-enhancing modification.

C3. The synthetic guide RNA of embodiment C2, wherein one or more of the stability-enhancing modifications is located in the guide sequence.

C4. The synthetic guide RNA of embodiment C2, wherein the stability-enhancing modification comprises a 2′-O-methyl moiety, a Z base, a 2′-deoxy nucleotide, a phosphorothioate internucleotide linkage, a phosphonoacetate (PACE) internucleotide linkage, or a thiophosphonoacetate (thioPACE) internucleotide linkage, or combinations thereof.

C5. The synthetic guide RNA of any of the foregoing embodiments, comprising less than 26 consecutive 2′-O-methyl modified nucleotides at a 5′ end of the guide RNA.

C6. The synthetic guide RNA of any of the foregoing embodiments, comprising a Z base replacing a cytosine in the synthetic guide RNA.

C7 The synthetic guide RNA of any of the foregoing embodiments, comprising at least one 2-thiouracil at a position corresponding to a uridine that can engage in U-G wobble pairing with a potential off-target sequence.

C8. The synthetic guide RNA of any of the foregoing embodiments, comprising one or more modifications selected from the group consisting of a 2′-O-methyl nucleotide with a 3′-phosphorothioate group, a 2′-O-methyl nucleotide with a 3′-phosphonoacetate group, a 2′-O-methyl nucleotide with a 3′-thiophosphonoacetate group, and a 2′-deoxy nucleotide with a 3′-phosphonoacetate group.

C9. The synthetic guide RNA of any of the foregoing embodiments, comprising at least two modifications.

C10. The synthetic guide RNA any of the foregoing embodiments, comprising up to 50 modifications.

C11. The synthetic guide RNA of any of the foregoing embodiments, comprising a single RNA strand or two separate RNA strands, and one or more modifications at a 5′ end of each RNA strand, at a 3′ end of each RNA strand, or at both a 5′ end and a 3′ end of each RNA strand.

C12. The synthetic guide RNA of any of the foregoing embodiments, comprising 7 or fewer consecutive modified nucleotides at a 5′ end or at a 3′ end or at each of 5′ and 3′ ends.

C13. The synthetic guide RNA of any of the foregoing embodiments, comprising one or more 5-methyluridine nucleotides at one or more of a 5′ end, a 3′ end, or a stem-loop.

C14. The synthetic guide RNA of any of the foregoing embodiments, wherein one or more of the modifications alters base-pairing thermostability.

C15. The synthetic guide RNA of embodiment C14, wherein said one or more modifications enhances the base-pairing thermostability.

C16. The synthetic guide RNA of embodiment C15, wherein said one or more modifications is independently selected from a 2-thiouracil (2-thioU), a 4-thiouracil (4-thioU), a 2-aminoadenine, a 2′-O-methyl, a 2′-fluoro, a 5-methyluridine, a 5-methylcytidine, and a locked nucleic acid modification (LNA).

C17. The synthetic guide RNA of embodiment C15, wherein said one or more modifications decreases the base-pairing thermostability.

C18. The synthetic guide RNA of embodiment C17, wherein said one or more modifications is independently selected from a 2-thiouracil, a 2′-deoxy, a phosphorothioate linkage, a phosphorodithioate linkage, a boranophosphate linkage, a phosphonoacetate linkage, a thiophosphonoacetate linkage, and an unlocked nucleic acid modification (ULNA).

C19. The synthetic guide RNA of any of the foregoing embodiments, comprising one or more 2′-O-methylA-2′-O-methylU base pairs.

C20. The synthetic guide RNA of any of the foregoing embodiments, wherein one or more of the modifications is a specificity-altering modification.

C21. The synthetic guide RNA of embodiment C20, wherein the specificity-altering modification is located in the guide sequence.

C22. The synthetic guide RNA of any of the foregoing embodiments, wherein the specificity-altering modification comprises a 2-thiouracil, a 4-thiouracil, a 2-aminoadenine, a 2′-O-methyl, a 2′-fluoro, a LNA, a phosphorothioate linkage, a phosphorodithioate linkage, a boranophosphate linkage, a phosphonoacetate linkage, a thiophosphonoacetate linkage, an ULNA, a 2′-deoxy, a 5-methyluridine, a 5-methylcytidine, or combinations thereof.

C23. The synthetic guide RNA of any of the foregoing embodiments, comprising a fluorescent dye or a label.

C24. The synthetic guide RNA of embodiment C23, wherein the fluorescent dye or a label is bound to a stem-loop of the synthetic guide RNA.

C25. A method for genomic editing to modify a DNA sequence, or regulating the expression of a gene of interest, or cleaving a target polynucleotide comprising: contacting the DNA sequence, the gene of interest, or the target polynucleotide with a CRISPR-associated protein and the synthetic guide RNA of any of the foregoing embodiments, and editing, regulating or cleaving the DNA sequence, the gene of interest or the target polynucleotide.

C26. The method of embodiment C25, wherein the method is performed in vitro, and the synthetic guide RNA comprises 15 or more 2′-O-methyl modifications throughout the guide sequence.

C27. A set or library of RNA molecules comprising two or more synthetic guide RNAs of any of the foregoing embodiments.

C28. A kit comprising the synthetic guide RNA of any of the foregoing embodiments.

C29. An array of RNA molecules comprising two or more synthetic guide RNAs of any of the foregoing embodiments.

The foregoing description of exemplary or preferred embodiments should be taken as illustrating, rather than as limiting, the present invention as defined by the claims. As will be readily appreciated, numerous variations and combinations of the features set forth above can be utilized without departing from the present invention as set forth in the claims. Such variations are not regarded as a departure from the scope of the invention, and all such variations are intended to be included within the scope of the following claims. All references cited herein are incorporated by reference in their entireties.

1-30. (canceled)

31. A chemically-synthesized single-molecule CRISPR guide RNA (sgRNA) comprising:
a 5′-end and a 3′-end;
one or more chemically modified nucleotides;
a guide sequence that is 17-22 nucleotides long and is capable of hybridizing to a target sequence in a target polynucleotide; and
a scaffold region that can interact with a Cas9 protein to form a gRNA: Cas9 complex,
wherein said complex is capable of cleaving at least about 20% of the target polynucleotide in an in vitro assay wherein the in vitro assay comprises:
about 50 fmoles of linearized target polynucleotide;
about 50 nM sgRNA;
about 39 nM recombinant purified active Cas9 protein; and
0.8 mM MgCl2 at pH 7.6, and is performed at 37° C. for 30 min.

32. The sgRNA of claim 31, wherein said one or more chemically modified nucleotides are selected from the group consisting of 2′-O-methylribonucleotide (=2′-OMe), 2′-deoxyribonucleotide, phosphorothioate nucleotide, phosphonoacetate (=PACE) nucleotide, thiophosphonoacetate (=thioPACE) nucleotide, Z nucleotide, 2-thiouracil nucleotide, 2-aminoadenine nucleotide, 5-methyluracil nucleotide, 5-aminoallyluracil nucleotide coupled to Cy5 fluorophore, 2-(4-butylamidofluorescein) propane-1,3-diol bis(phosphodiester) linked nucleotide, and combinations of thereof.

33. The sgRNA of claim 32, wherein the sgRNA does not comprise more than 20 consecutive 2′-deoxy or 2′-O-methyl modified nucleotides from the 5′-end.

34. The sgRNA of claim 33, wherein the Cas9 protein is from Streptococcus pyogenes.

35. The sgRNA of claim 33, wherein the guide sequence is 20 nucleotides long.

36. The sgRNA of claim 33 wherein the one or more modified nucleotides is located in the guide sequence.

37. The sgRNA of claim 33 wherein the modified nucleotide is located in an overhang 5′ of the guide sequence.

38. The sgRNA of claim 33 wherein the one or more modified nucleotides is located in the scaffold region or in both the guide sequence and the scaffold region.

39. The sgRNA of claim 33 wherein said one or more modified nucleotides is a 2′-O-methyl-3′-phosphorothioate (MS) nucleotide, a 2′-O-methyl-3′-phosphonoacetate (MP) nucleotide, a 2′-O-methyl-3′-thiophosphonoacetate (MSP) nucleotide or a combination thereof.

40. The sgRNA of claim 39, wherein said one or more chemically modified nucleotides comprises at least two modified nucleotides independently selected from MS, MP and MSP within five nucleotides from the 5′-end or the 3′-end, or within five nucleotides from each of said 5′-end and said 3′-end.

41. The sgRNA of claim 31 wherein said one or more chemically modified nucleotides is located within 3 nucleotides from each of the 5′ and the 3′ ends.

42. The sgRNA of claim 33 further comprising a squarate or a triazolo linkage.

43. The sgRNA of claim 31, wherein said complex is capable of cleaving at least about 70% of the target polynucleotide in said in vitro assay.

44. The sgRNA of claim 31, where the sgRNA is at least 100 nucleotides in length.

45. The sgRNA of claim 31 comprising one or more MS, MP or MSP nucleotides within five nucleotides from the 5′-end or the 3′-end, or within five nucleotides from each of said 5′-end and said 3′-end.

46. A ribonucleoprotein (RNP) comprising the sgRNA of claim 31 and a Cas protein.

47. A method of editing, regulating gene expression of, detecting, or cleaving a target polynucleotide, the method comprising:
providing the sgRNA of claim 31 and a Cas protein; and
contacting the target polynucleotide with the guide RNA, thereby editing, regulating gene expression of, detecting, or cleaving the target polynucleotide.

48. The method of claim 47, wherein the Cas protein has double-strand nuclease activity.

49. The method of claim 47, wherein the Cas protein has a single-strand nicking activity.

50. The method of claim 47, wherein the Cas protein lacks nuclease activity.

51. The method of claim 47, wherein the Cas protein is a fusion protein.

52. The method of claim 47, wherein the contacting occurs in a cell.

53. The method of claim 47, wherein the Cas protein is provided to a cell as an mRNA encoding the Cas protein.

54. The method of claim 53, wherein the mRNA is provided to the cell along with the sgRNA.

55. The method of claim 47, wherein the Cas protein is provided to a cell as a protein.

56. The method of claim 55, wherein the Cas protein is provided to the cell along with the sgRNA in a ribonucleoprotein.

57. The method of claim 47, wherein the sgRNA comprises at least two consecutive modified nucleotides independently selected from MS, MP and MSP within five nucleotides from each of the 5′- and the 3′-end.

58. The method of claim 57, wherein the guide sequence is 20 nucleotides long.

59. A set or a library of sgRNAs comprising at least one sgRNA of claim 31.

60. A kit comprising at least one sgRNA of claim 31 and at least one Cas protein and/or at least one mRNA encoding a Cas protein.
